(19) United States
(12) Patent Application Publication
(10) Pub. No.: US 2022/0041597 A1
(43) Pub. Date: Feb. 10, 2022

(54) INHIBITING UBIQUITIN SPECIFIC PEPTIDASE 9X

(71) Applicant: FORMA Therapeutics, Inc., Waterdown, MA (US)

(72) Inventors: Bruce Follows, Littleton, MA (US); Adam Charles Talbot, Watertown, MA (US); Scot Mente, Watertown, MA (US); Sabine Ruppel, Watertown, MA (US); Tatiana Shelekhin, Ridgefield, CT (US); Katherine J. Kayser-Bricker, Branford, CT (US)

(21) Appl. No.: 17/414,687

(22) PCT Filed: Dec. 26, 2019
(86) PCT No.: PCT/US2019/068530

§ 371 Date: Jun. 16, 2021

Related U.S. Application Data
(60) Provisional application No. 62/784,981, filed on Dec. 26, 2018.

(51) Int. Cl. C07D 471/04

(52) U.S. Cl. CPC C07D 471/04

(57) ABSTRACT

FLT3-ITD and FLT3-TKD are the most frequent mutations in acute myeloid leukemia (AML) with the former associated with a poor prognosis. Here we show that inhibition of the deubiquitinase USP9X by its inhibitor WP1130 or EOAI3402143 (G9) induces apoptosis preferentially in cells transformed by these mutant kinases, including FLT3-ITD-positive AML cell line MV4-11 and primary AML cells. Mechanistically, WP1130 induced aggresomal translocation of the mutant kinases, particularly FLT3-ITD in its activated and autophosphorylated conformation, to block the downstream signaling events, which was aggravated by knock down of USP9X. Moreover, USP9X physically associated with FLT3-ITD to inhibit its K63-linked polyubiquitination, while FLT3-ITD induced tyrosine phosphorylation and degradation of USP9X through the ubiquitin/proteasome pathway. WP1130 or G9 also induced oxidative stress to stimulate stress-related MAP kinase pathways and DNA damage responses to activate in cooperation with inhibition of FLT3-ITD signaling the intrinsic mitochondria-mediated apoptotic pathway, which was synergistically enhanced by BH3 mimetics and prevented by overexpression of Bcl-xL or Mcl-1. Thus, USP9X represents a promising target for novel therapies against therapy-resistant FLT3-ITD-positive AML.

### CROSS-REFERENCE TO RELATED APPLICATIONS

The present application claims the benefit of U.S. Provisional Patent Application No. 62/784,981, filed Dec. 26, 2018, which is hereby incorporated herein by reference in its entirety.

### TECHNICAL FIELD

This disclosure relates to novel chemical compositions for inhibiting ubiquitin specific peptidase 9X.

### BACKGROUND

Deubiquitylating enzymes (DUBs) control a number of cellular processes, including the stability and function of a variety of oncoproteins, by reversing ubiquitination. Ubiquitin specific peptidase 9X (USP9X) is a member of the USP family of DUBs and is a key regulator of protein homeostasis for protein substrates including several that are known to be important in cancer. These include oncogenic or protumorigenic proteins and proteins involved in the anti-tumor immune response. These proteins can be important in tumor cells, immune cells, or other cells, such as stromal cells that play a role in cancer. Examples include MCL-1, survivin, ITCH, and CEP55. Overexpression and/or mutation of DUBs and their substrates have been correlated with cancer initiation and progression. USP9X has been suggested to be a negative prognostic factor for several oncology indications and may be associated with decreased overall survival in some cancer types (e.g., esophageal squamous cell carcinoma, non-small cell lung cancer, and multiple myeloma). Targeting USP9X can enhance an anti-tumor immune response through regulation of key maintenance proteins. Therefore, USP9X is a target for cancer drug development, particularly as a means to deplete oncoprotein substrates that have been labeled undruggable and/or through activation of the immune response. There is a need for compounds that inhibit USP9X and are useful for treating diseases and disorders associated with modulation of USP9X.

### SUMMARY

One embodiment of this disclosure relates to compounds of Formula I:

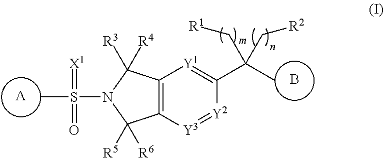

or a pharmaceutically acceptable salt thereof, wherein:
X¹ is NR or O;
Y¹ is CR⁷ or N;
Y² is CR⁸ or N;
Y³ is CR⁹ or N;
   wherein the heteroaryl formed when at least one of Y¹, Y², or Y³ is N may comprise an N-oxide; Ring A is a monocyclic or bicyclic 3- to 12-membered ring,
   wherein the ring is saturated, fully or partially unsaturated, or aromatic, and
   wherein the ring contains 0-4 heteroatoms independently selected from the group consisting of N, O, and S, and
   wherein Ring A is optionally substituted with one or more R^{a};
each R^{a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C1-C6 aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R^{a} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
Ring B is a monocyclic or bicyclic 3- to 12-membered ring,
   wherein the ring is saturated, fully or partially unsaturated, or aromatic, and
   wherein the ring contains 0-4 heteroatoms independently selected from the group consisting of N, O, and S, and
   wherein Ring B is optionally substituted with one or more R^{b};
each R^{b} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R^{b} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
R¹ and R² are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
or R¹ and R² combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
   wherein an optionally substituted R¹ and R² group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
R³, R⁴, R⁵, and R⁶ are each independently selected from the group consisting of —H, optionally substituted C₁-C₆aliphatic, optionally substituted C₃-C₈cycloalkyl, and optionally substituted 3- to 8-membered heterocyclyl containing 1-4 heteroatoms independently selected from the group consisting of N, O, and S,
or R³ and R⁴, or R⁵ and R⁶, or a combination thereof, combine with the carbon to which they are attached to form an optionally substituted C₃-C₅cycloalkyl or an optionally substituted 3- to 8-membered heterocyclyl containing 1-4 heteroatoms independently selected from the group consisting of N, O, and S,
   wherein an optionally substituted R³, R⁴, R⁵, and R⁶ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆ aliphatic;
R⁷, R⁸, and R⁹ are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, and optionally substituted C₁-C₆aliphatic,
   wherein an optionally substituted R⁷, R⁸, and R⁹ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
each R is independently selected from the group consisting of —H, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R group may be optionally substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;
each R′ is independently selected from the group consisting of optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R′ group may be substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;
m is 0, 1, or 2; and
n is 0, 1, or 2.

Another aspect of this invention relates to compounds of Formula I that are USP9X Inhibitors. Unless otherwise indicated, a “USP9X Inhibitor” as used herein refers to a compound of Formula I having one or more of the following characteristics when tested in the Biochemical Assay of Example 3: (i) an IC₅₀ value of ≤0.1 μM and >0.001 μM; (ii) an IC₅₀ value of ≤1 μM and >0.1 μM; (iii) an IC₅₀ value of ≤10 μM and >1 μM; and (iv) an IC₅₀ value of ≤25 μM and >10 μM. In some embodiments, a USP9X Inhibitor is a compound of Formula I having an IC₅₀ value of (i) an IC₅₀ value of ≤0.1 μM and >0.001 μM; (ii) an IC₅₀ value of ≤1 μM and >0.1 μM; or (iii) an IC₅₀ value of <10 μM and >1 μM when tested in the Biochemical Assay of Example 3. In some embodiments, a USP9X Inhibitor is a compound of Formula I having an IC₅₀ value of ≤0.1 μM and >0.001 μM when tested in the Biochemical Assay of Example 3. In some embodiments, a USP9X Inhibitor is a compound of Formula I having an IC₅₀ value of ≤1 μM and >0.1 μM when tested in the Biochemical Assay of Example 3. In some embodiments, a USP9X Inhibitor is a compound of Formula I having an IC₅₀ value ≤10 μM and >1 μM when tested in the Biochemical Assay of Example 3.

### DETAILED DESCRIPTION

One aspect of this disclosure relates to compounds of Formula I:

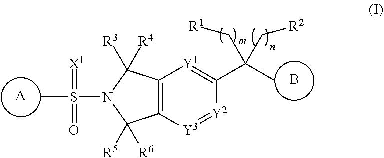

or a pharmaceutically acceptable salt thereof,
wherein Ring A, Ring B, X¹, Y¹, Y², Y³, R¹, R², R³, R⁴, R⁵, R⁶, m, and n are as defined above for Formula I and described in classes and subclasses herein, both singly and in combination.

In some embodiments, compounds of Formula I are provided, wherein:
X¹ is O;
Y¹ is CR⁷ or N;
Y² is CR⁸ or N;
Y³ is CR⁹ or N;
   wherein the heteroaryl formed when at least one of Y¹, Y², or Y³ is N may comprise an N-oxide; Ring A is:
(i) a monocyclic ring selected from C₃-C₈carbocyclyl, phenyl, or 5- to 8-membered heteroaryl,
   wherein the monocyclic ring contains 0-4 heteroatoms independently selected from the group consisting of O, N, and S, and
   wherein the monocyclic ring is optionally substituted with one or more R^{a}; or
(ii) a bicyclic 9- to 12-membered ring comprising a phenyl ring,
   wherein the phenyl ring is fused to an aromatic or partially unsaturated 3- to 8-membered carbocyclic or heterocyclic ring,
   wherein the bicyclic ring contains 0-4 heteroatoms independently selected from the group consisting of O, N, and S, and
   wherein the bicyclic ring is optionally substituted with one or more R^{a};
each R^{a} is independently selected from the group consisting of halogen, —OR, —NRC(O)R′, optionally substituted 3- to 10-membered heterocyclyl containing 1-4 heteroatoms independently selected from N, O, and S, and optionally substituted 5- to 10-membered heteroaryl containing 1-4 heteroatoms independently selected from N, O, and S,
   wherein an optionally substituted R^{a} group may be substituted with one or more halogen;
Ring B is:
(i) a monocyclic ring selected from C₃-C₈carbocyclyl or phenyl,
   wherein the monocyclic ring is optionally substituted with one or more R^{b}; or
(ii) a bicyclic 9- to 12-membered ring comprising a phenyl ring,
   wherein the phenyl ring is fused to an aromatic or partially unsaturated 3- to 8-membered carbocyclic or heterocyclic ring,
   wherein the bicyclic ring contains 0-4 heteroatoms independently selected from the group consisting of O, N, and S, and
   wherein the bicyclic ring is optionally substituted with one or more R^{b};
each R^{b} is independently selected from the group consisting of halogen, —OR, optionally substituted C₁-C₆ aliphatic, and optionally substituted 3- to 10-membered heterocyclyl containing 1-4 heteroatoms independently selected from N, O, and S,
   wherein an optionally substituted R^{b} group may be substituted with one or more substituents independently selected from the group consisting of —NR₂ and C₁-C₆ aliphatic;
R¹ and R² are each independently selected from the group consisting of —H, —OR, —NR₂, —CN, —C(O)NR₂, and C₁-C₆aliphatic;
R³, R⁴, R⁵, and R⁶ are each independently selected from the group consisting of —H and C₁-C₆aliphatic;
R⁷, R⁸, and R⁹ are each independently selected from the group consisting of —H, —OR, and C₁-C₆aliphatic;
each R is independently selected from the group consisting of —H, optionally substituted C₁-C₆aliphatic, and optionally substituted 3- to 10-membered heterocyclyl containing 1-4 heteroatoms independently selected from N, O, and S,
   wherein an optionally substituted R group may be optionally substituted with one or more C₁-C₆aliphatic;
each R′ is independently C₃-C₁₀cycloalkyl;
m is 0, 1, or 2; and
n is 0.

In some embodiments, compounds of Formula I are provided, wherein:
X¹ is O;
Y¹ is CR⁷ or N;
Y² is CR⁸ or N;
Y³ is CR⁹ or N;
   wherein the heteroaryl formed when at least one of Y¹, Y², or Y³ is N may comprise an N-oxide;
Ring A is selected from:

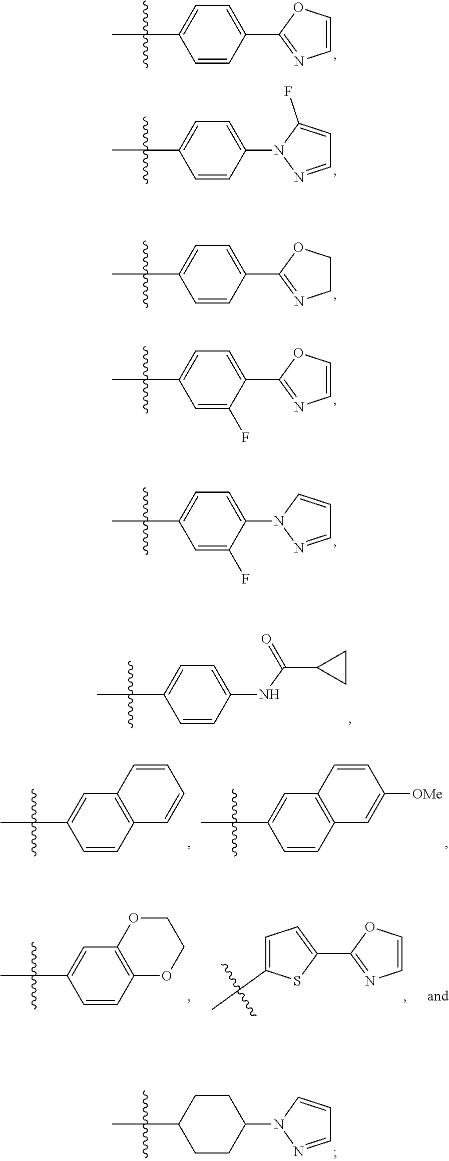

Ring B is selected from:

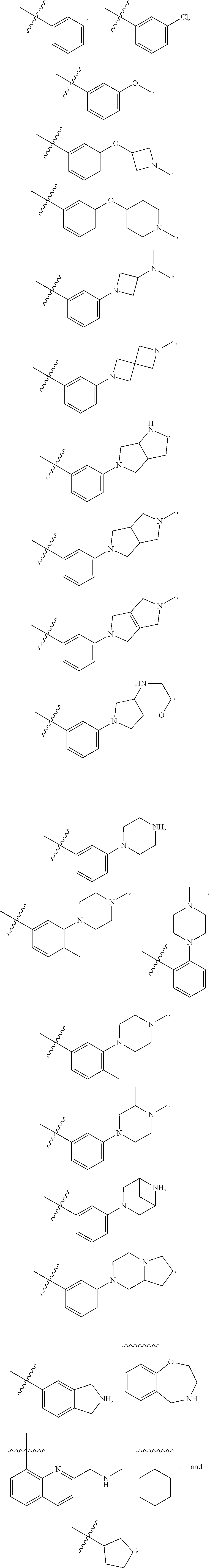

R¹ and R² are each independently selected from the group consisting of —H, —OH, —OMe, —NH₂, —NHMe, —CN, —C(O)NHMe, and methyl;
R³, R⁴, R⁵, and R⁶ are each independently selected from the group consisting of —H and methyl;
R⁷, R⁸, and R⁹ are each independently selected from the group consisting of —H, —OMe, and methyl;
m is 0, 1, or 2; and
n is 0.

In some embodiments, this disclosure provides compounds of Formula II:

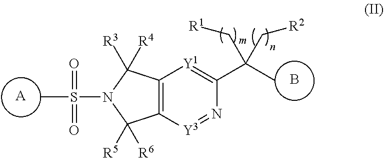

or a pharmaceutically acceptable salt thereof,
wherein Ring A, Ring B, Y¹, Y³, R¹, R², R³, R⁴, R⁵, R⁶, m, and n are as defined above for Formula I and described in classes and subclasses herein, both singly and in combination.

In some embodiments, this disclosure provides compounds of Formula III:

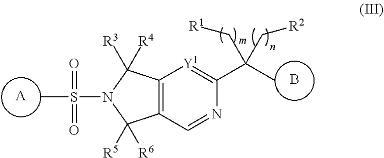

or a pharmaceutically acceptable salt thereof,
wherein Ring A, Ring B, Y¹, R¹, R², R³, R⁴, R⁵, R⁶, m, and n are as defined above for Formula I and described in classes and subclasses herein, both singly and in combination.

In some embodiments, this disclosure provides compounds of Formula IV:

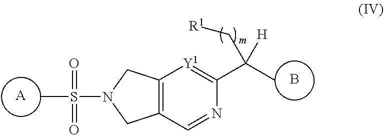

or a pharmaceutically acceptable salt thereof,
wherein Ring A, Ring B, Y¹, R¹, and m are as defined above for Formula I and described in classes and subclasses herein, both singly and in combination.

In some embodiments, this disclosure provides compounds of Formula IV-a:

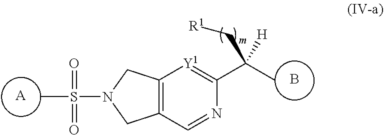

or a pharmaceutically acceptable salt thereof,
wherein Ring A, Ring B, Y¹, R¹, and m are as defined above for Formula I and described in classes and subclasses herein, both singly and in combination.

In some embodiments, this disclosure provides compounds of Formula IV-b:

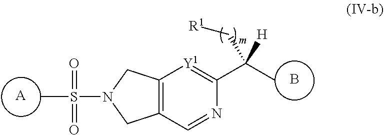

or a pharmaceutically acceptable salt thereof,
wherein Ring A, Ring B, Y¹, R¹, and m are as defined above for Formula I and described in classes and subclasses herein, both singly and in combination.

In some embodiments, this disclosure provides compounds of Formula V:

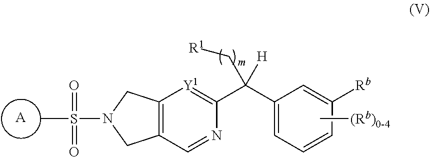

or a pharmaceutically acceptable salt thereof,
wherein Ring A, Y¹, R¹, R^{b}, and m are as defined above for Formula I and described in classes and subclasses herein, both singly and in combination.

In some embodiments, this disclosure provides compounds of Formula VI:

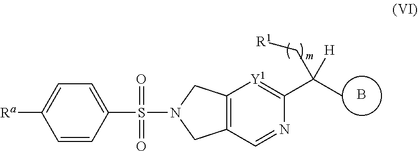

or a pharmaceutically acceptable salt thereof,
wherein Ring B, Y¹, R¹, R^{a}, and m are as defined above for Formula I and described in classes and subclasses herein, both singly and in combination.

In some embodiments, this disclosure provides compounds of Formula VII:

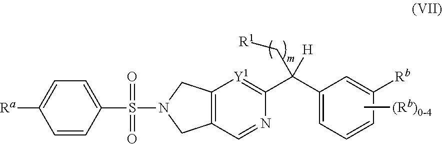

or a pharmaceutically acceptable salt thereof,
wherein Y¹, R¹, R^{a}, R^{b}, and m are as defined above for Formula I and described in classes and subclasses herein, both singly and in combination.

In some embodiments, this disclosure provides compounds of Formula VIII:

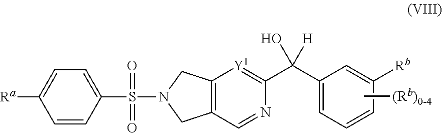

or a pharmaceutically acceptable salt thereof,
wherein Y¹, R^{a}, and R^{b} are as defined above for Formula I and described in classes and subclasses herein, both singly and in combination.

In some embodiments, this disclosure provides compounds of Formula VIII-a:

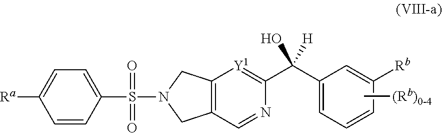

or a pharmaceutically acceptable salt thereof,
wherein Y¹, R^{a}, and R^{b} are as defined above for Formula I and described in classes and subclasses herein, both singly and in combination.

In some embodiments, this disclosure provides compounds of Formula VIII-b:

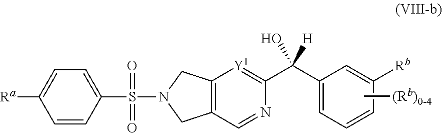

or a pharmaceutically acceptable salt thereof,
wherein Y¹, R^{a}, and R^{b} are as defined above for Formula I and described in classes and subclasses herein, both singly and in combination.

In some embodiments of Formula I, X¹ is NR or O. In some embodiments, X¹ is NR. In some embodiments, X¹ is NH. In some embodiments, X¹ is O.

In some embodiments of Formulas I, II, III, IV, IV-a, IV-b, V, VI, VII, VIII, VIII-a, and VIII-b, Y¹ is CR⁷ or N. In some embodiments, Y¹ is CH or N. In some embodiments, Y¹ is CR⁷. In some embodiments, Y¹ is N. In some embodiments, Y¹ is CH.

In some embodiments of Formula I, Y² is CR⁷ or N. In some embodiments, Y² is CH or N. In some embodiments, Y² is CR⁷. In some embodiments, Y² is N. In some embodiments, Y² is CH. In some embodiments, Y² is N and the resulting heteroaryl comprises an N-oxide.

In some embodiments of Formula I, Y³ is CR⁷ or N. In some embodiments, Y³ is CH or N. In some embodiments, Y³ is CR⁷. In some embodiments, Y³ is N. In some embodiments, Y³ is CH.

In some embodiments of Formula I, Y¹ is CR⁷, Y² is N, and Y³ is CR⁹. In some embodiments, Y¹ is N, Y² is N, and Y³ is CR⁹. In some embodiments, Y¹ is CR⁷, Y² is N, and Y³ is N. In some embodiments, Y¹ is CR⁷, Y² is CR⁸, and Y³ is CR⁹. In some embodiments, Y¹ is CH, Y² is N, and Y³ is CH. In some embodiments, Y¹ is N, Y² is N, and Y³ is CH. In some embodiments, Y¹ is CH, Y² is N, and Y³ is N. In some embodiments, Y¹ is CH, Y² is CH, and Y³ is CH.

In some embodiments of Formulas I, II, III, IV, IV-a, IV-b, and V, Ring A is a monocyclic or bicyclic 3- to 12-membered ring,
wherein the ring is saturated, fully or partially unsaturated, or aromatic, and
wherein the ring contains 0-4 heteroatoms independently selected from the group consisting of N, O, and S, and
wherein Ring A is optionally substituted with one or more R^{a}

In some embodiments of Formulas I, II, III, IV, IV-a, IV-b, and V, Ring A is:
(i) a monocyclic ring selected from C₃-C₈carbocyclyl, 3- to 8-membered heterocyclyl, phenyl, or 5- to 8-membered heteroaryl,
   wherein the monocyclic ring contains 0-4 heteroatoms independently selected from the group consisting of N, O, and S, and
   wherein the monocyclic ring is optionally substituted with one or more R^{a}; or
(ii) a bicyclic 6- to 12-membered ring comprising a C₃-C₁₀carbocyclyl, 3- to 10-membered heterocyclyl, phenyl, or 5- to 8-membered heteroaryl ring,
   wherein the C₃-C₁₀carbocyclyl, 3- to 10-membered heterocyclyl, phenyl, or 5- to 8-membered heteroaryl ring is fused to an aromatic, saturated, or partially unsaturated 3- to 8-membered carbocyclic or heterocyclic ring,
   wherein the bicyclic ring contains 0-4 heteroatoms independently selected from the group consisting of N, O, and S, and
   wherein the bicyclic ring is optionally substituted with one or more R^{a}.

In some embodiments of Formulas I, II, III, IV, IV-a, IV-b, and V, Ring A is:
(i) a monocyclic ring selected from C₃-C₈carbocyclyl, phenyl, or 5- to 8-membered heteroaryl,
   wherein the monocyclic ring contains 0-4 heteroatoms independently selected from the group consisting of O, N, and S, and
   wherein the monocyclic ring is optionally substituted with one or more R^{a}; or
(ii) a bicyclic 9- to 12-membered ring, comprising a phenyl ring,
   wherein the phenyl ring is fused to an aromatic or partially unsaturated 3- to 8-membered carbocyclic or heterocyclic ring,
   wherein the bicyclic ring contains 0-4 heteroatoms independently selected from the group consisting of O, N, and S, and
   wherein the bicyclic ring is optionally substituted with one or more R^{a}.

In some embodiments of Formulas I, II, III, IV, IV-a, IV-b, and V, Ring A is (i) monocyclic C₅-C₆carbocyclyl ring, (ii) phenyl ring, (iii) 5- to 6-membered monocyclic heteroaryl ring containing 1-4 heteroatoms independently selected from the group consisting of N, O, and S, or (iv) a 9- to 10-membered bicyclic ring comprising a phenyl ring, wherein the phenyl ring is fused to an aromatic or partially unsaturated 5- to 6-membered carbocyclic ring or 5- to 6-membered heterocyclic ring containing 1-4 heteroatoms independently selected from the group consisting of N, O, and S, and wherein Ring A is optionally substituted with one or more R^{a}. In some embodiments, Ring A is (i) a phenyl ring, (ii) 5- to 6-membered monocyclic heteroaryl ring containing 1-4 heteroatoms independently selected from the group consisting of N, O, and S, or (iii) a 9- to 10-membered bicyclic ring comprising a phenyl ring, wherein the phenyl ring is fused to an aromatic or partially unsaturated 5- to 6-membered carbocyclic ring or 5- to 6-membered heterocyclic ring containing 1-4 heteroatoms independently selected from the group consisting of N, O, and S, and wherein Ring A is optionally substituted with one or more R^{a}. In some embodiments, Ring A is (i) a phenyl ring or (ii) a 9- to 10-membered bicyclic ring comprising a phenyl ring, wherein the phenyl ring is fused to an aromatic or partially unsaturated 5- to 6-membered carbocyclic ring or 5- to 6-membered heterocyclic ring containing 1-4 heteroatoms independently selected from the group consisting of N, O, and S, and wherein Ring A is optionally substituted with one or more R^{a}. In some embodiments, Ring A is phenyl or naphthyl optionally substituted with one or more R^{a}. In some embodiments, Ring A is phenyl optionally substituted with one or more R^{a}. In some embodiments, Ring A is a phenyl ring substituted with at least one R^{a} in the para position.

In some embodiments of Formulas I, II, III, IV, IV-a, IV-b, and V, Ring A is selected from:

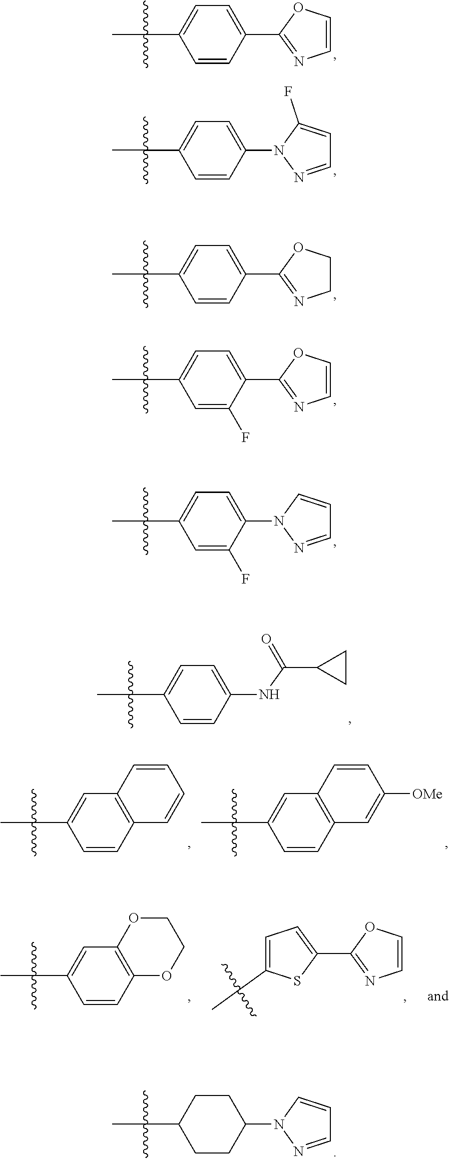

In some embodiments of Formulas I, II, III, IV, IV-a, IV-b, and V, Ring A is selected from:

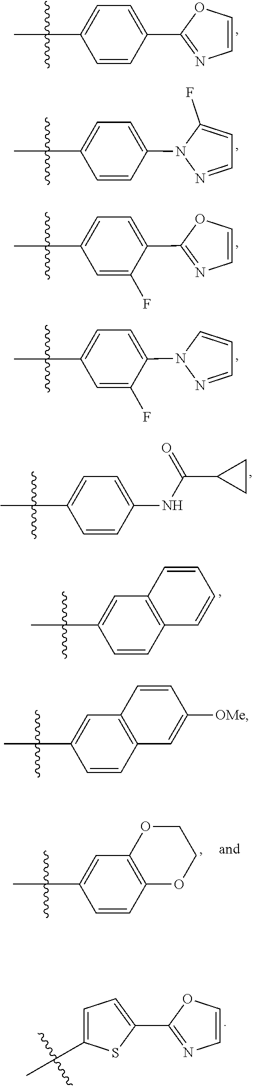

In some embodiments of Formulas I, II, III, IV, IV-a, IV-b, and V, Ring A is selected from:

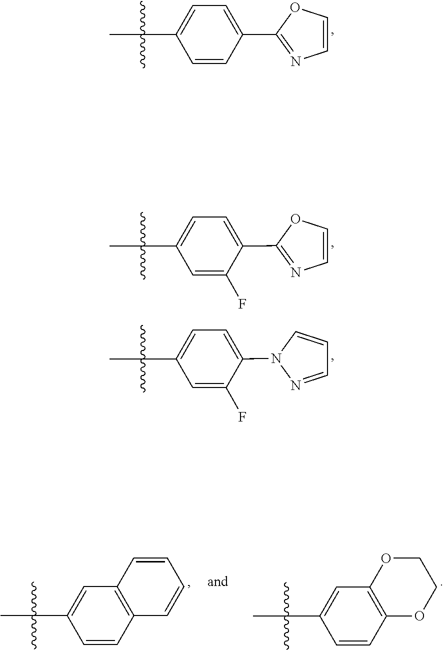

In some embodiments of Formulas I, II, III, IV, IV-a, IV-b, and V, Ring A is selected from:

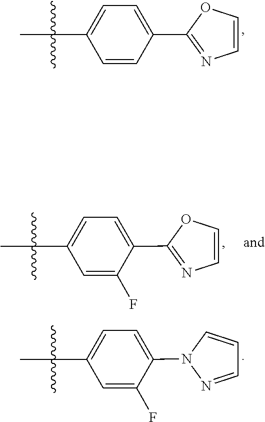

In some embodiments of Formulas I, II, III, IV, IV-a, IV-b, and V, Ring A is:

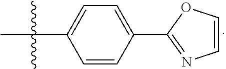 [SMILES: *c1ccc(-c2ncco2)cc1]

In some embodiments of Formulas I, II, III, IV, IV-a, IV-b, V, VI, VII, VIII, VIII-a, and VIII-b, each R^{a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-3 heteroatoms independently selected from N, O, and S, wherein an optionally substituted R^{a} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic. In some embodiments, each R^{a} is independently halogen, —OR, —NRC(O)R′, optionally substituted 3- to 10-membered heterocyclyl containing 1-4 heteroatoms independently selected from N, O, and S, or optionally substituted 5- to 10-membered heteroaryl containing 1-4 heteroatoms independently selected from N, O, and S, wherein an optionally substituted R^{a} group may be substituted with one or more halogen. In some embodiments, each R^{a} is independently halogen or optionally substituted 5- to 10-membered heteroaryl containing 1-4 heteroatoms independently selected from N, O, and S, wherein an optionally substituted R^{a} group may be substituted with one or more halogen. In some embodiments, each R^{a} is independently halogen or optionally substituted 5-membered heteroaryl containing 1-4 heteroatoms independently selected from N, O, and S, wherein an optionally substituted R^{a} group may be substituted with one or more halogen.

In some embodiments of Formulas I, II, III, IV, IV-a, IV-b, V, VI, VII, VIII, VIII-a, and VIII-b, each R^{a} is independently selected from the group consisting of fluoro, —OMe,

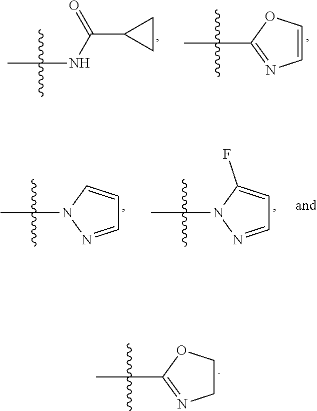 [SMILES: *NC(=O)C1CC1 ; *c1ncco1 ; *n1cccn1 ; *n1nccc1F ; *C1=NCCO1]

In some embodiments, each R^{a} is independently selected from the group consisting of fluoro, —OMe,

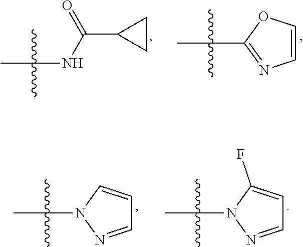 [SMILES: *NC(=O)C1CC1 ; *c1ncco1 ; *n1cccn1 ; *n1nccc1F]

In some embodiments, each R^{a} is independently selected from the group consisting of fluoro,

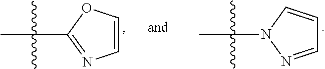 [SMILES: *c1ncco1 ; *n1cccn1]

In some embodiments of Formulas I, II, III, IV, IV-a, IV-b, and VI, Ring B is a monocyclic or bicyclic 3- to 12-membered ring,
wherein the ring is saturated, fully or partially unsaturated, or aromatic, and
wherein the ring contains 0-4 heteroatoms independently selected from the group consisting of N, O, and S, and
wherein Ring B is optionally substituted with one or more R^{b}.

In some embodiments of Formulas I, II, III, IV, IV-a, IV-b, and VI, Ring B is:
(i) a monocyclic ring selected from C₃-C₈carbocyclyl, 3- to 8-membered heterocyclyl, phenyl, or 5- to 8-membered heteroaryl,
   wherein the monocyclic ring contains 0-4 heteroatoms independently selected from the group consisting of N, O, and S, and
   wherein the monocyclic ring is optionally substituted with one or more R^{b}; or
(ii) a bicyclic 6- to 12-membered ring comprising a C₃-C₁₀carbocyclyl, 3- to 10-membered heterocyclyl, phenyl, or 5- to 8-membered heteroaryl ring,
   wherein the C₃-C₁₀carbocyclyl, 3- to 10-membered heterocyclyl, phenyl, or 5- to 8-membered heteroaryl ring is fused to an aromatic, saturated, or partially unsaturated 3- to 8-membered carbocyclic or heterocyclic ring,
   wherein the bicyclic ring contains 0-4 heteroatoms independently selected from the group consisting of N, O, and S, and
   wherein the bicyclic ring is optionally substituted with one or more R^{b}.

In some embodiments of Formulas I, II, III, IV, IV-a, IV-b, and VI, Ring B is:
(i) a monocyclic ring selected from C₃-C₈carbocyclyl or phenyl,
   wherein the monocyclic ring is optionally substituted with one or more R^{b}; or
(ii) a bicyclic 9- to 12-membered ring comprising a phenyl ring,
   wherein the phenyl ring is fused to an aromatic or partially unsaturated 3- to 8-membered carbocyclic or heterocyclic ring,
   wherein the bicyclic ring contains 0-4 heteroatoms independently selected from the group consisting of O, N, and S, and
   wherein the bicyclic ring is optionally substituted with one or more R^{b}.

In some embodiments of Formulas I, II, III, IV, IV-a, IV-b, and VI, Ring B is (i) monocyclic C₅-C₆carbocyclyl ring, (ii) phenyl ring, (iii) a 9- to 10-membered bicyclic ring comprising a phenyl ring, wherein the phenyl ring is fused to an aromatic or partially unsaturated 5- to 7-membered carbocyclic ring or 5- to 7-membered heterocyclic ring containing 1-4 heteroatoms independently selected from the group consisting of N, O, and S, and wherein Ring B is optionally substituted with one or more R^{b}. In some embodiments, Ring B is (i) phenyl ring or (ii) a 9- to 10-membered bicyclic ring comprising a phenyl ring, wherein the phenyl ring is fused to an aromatic or partially unsaturated 5- to 7-membered carbocyclic ring or 5- to 7-membered heterocyclic ring containing 1-4 heteroatoms independently selected from the group consisting of N, O, and S, and wherein Ring B is optionally substituted with one or more R^{b}. In some embodiments, Ring B is a phenyl ring optionally substituted with one or more R^{b}. In some embodiments, Ring B is a phenyl ring substituted with at least one R^{b} in the meta position.

In some embodiments of Formulas I, II, III, IV, IV-a, IV-b, and VI, Ring B is selected from:

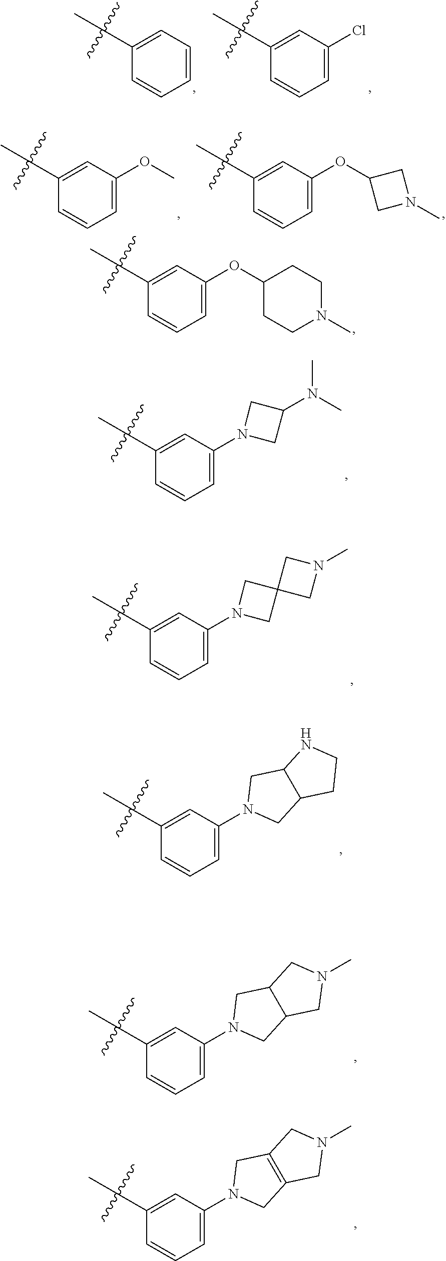

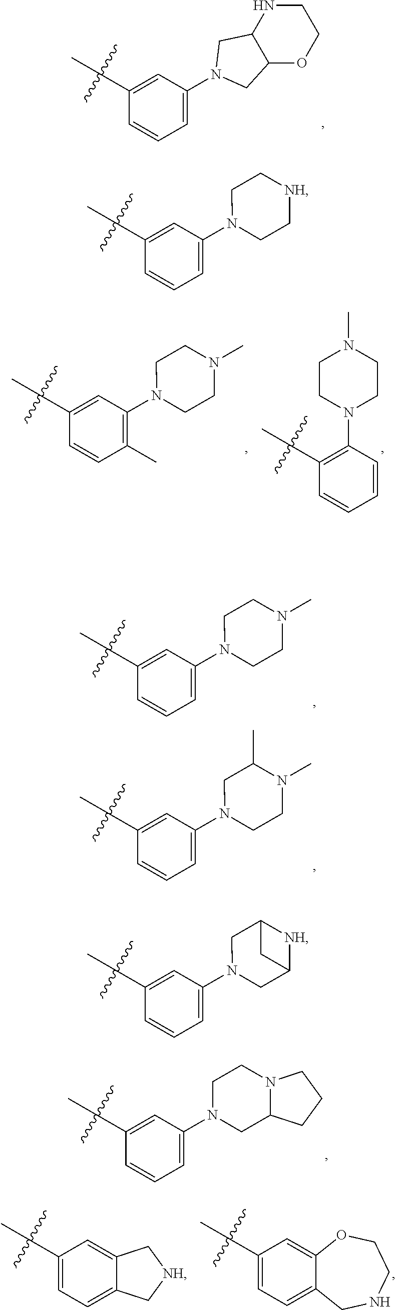

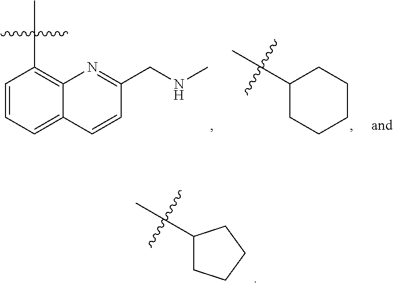 [SMILES: *c1cccc2ccc(CNC)nc12 ; *C1CCCCC1 ; *C1CCCC1]

In some embodiments of Formulas I, II, III, IV, IV-a, IV-b, and VI, Ring B is selected from:

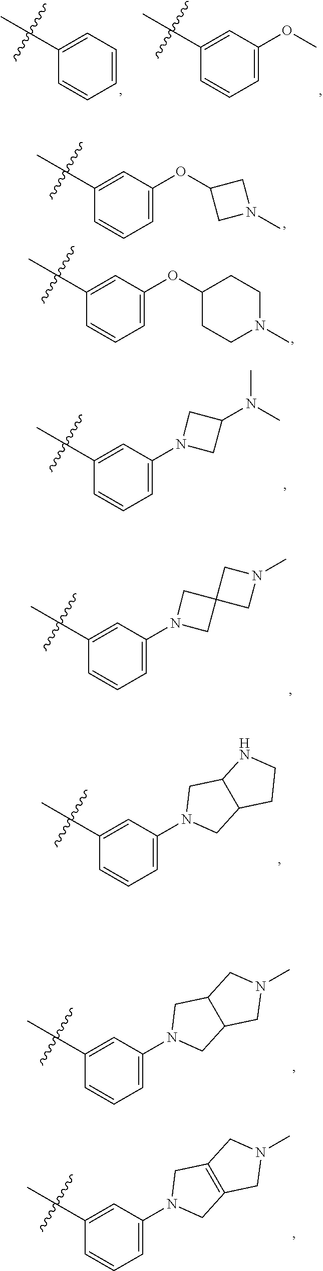

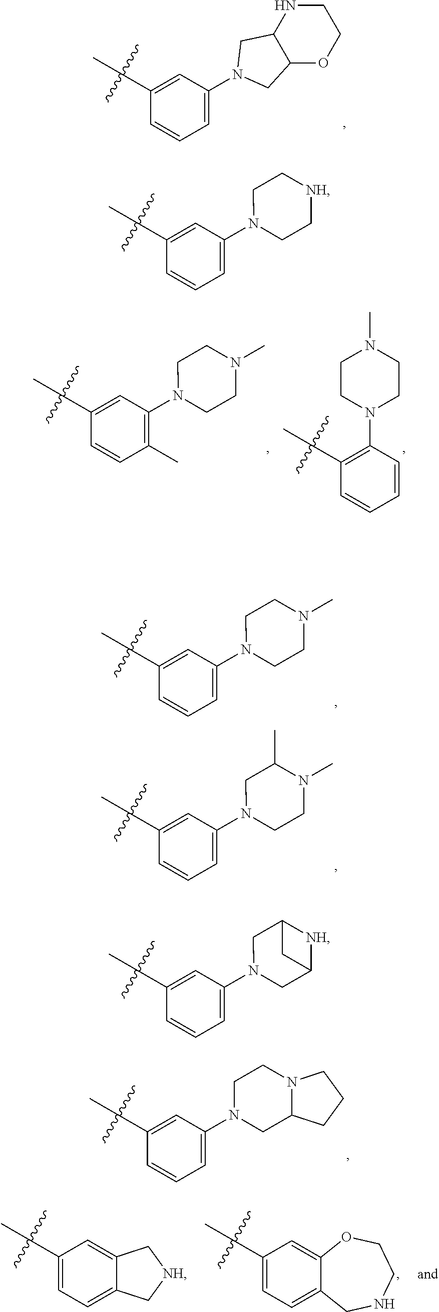

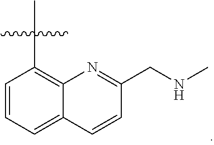 [SMILES: *c1cccc2ccc(CNC)nc12]

In some embodiments of Formulas I, II, III, IV, IV-a, IV-b, and VI, Ring B is selected from:

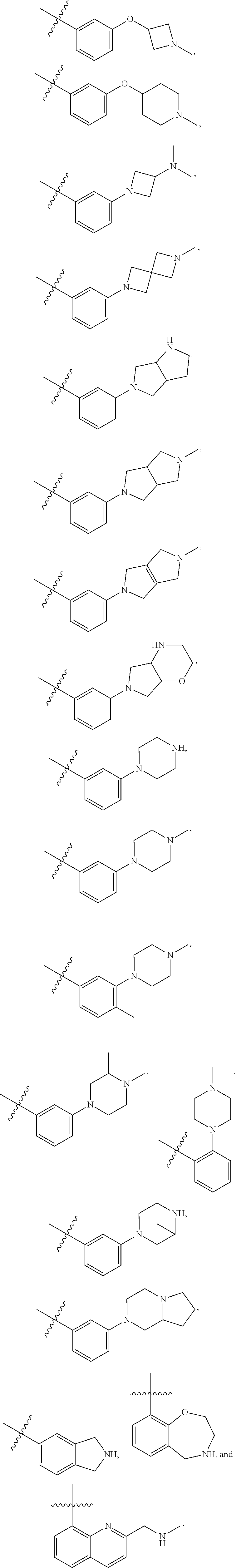

In some embodiments of Formulas I, II, III, IV, IV-a, IV-b, V, VI, VII, VIII, VIII-a, and VIII-b, each R^{b} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-3 heteroatoms independently selected from N, O, and S, wherein an optionally substituted R^{b} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic. In some embodiments, each R^{b} is independently selected from the group consisting of halogen, —OR, optionally substituted C₁-C₆ aliphatic, and optionally substituted 3- to 10-membered heterocyclyl containing 1-4 heteroatoms independently selected from N, O, and S, wherein an optionally substituted R^{b} group may be substituted with one or more substituents independently selected from the group consisting of —NR₂ and C₁-C₆ aliphatic. In some embodiments, each R^{b} is independently selected from the group consisting of —OR, optionally substituted C₁-C₆ aliphatic, and optionally substituted 3- to 10-membered heterocyclyl containing 1-4 heteroatoms independently selected from N, O, and S, wherein an optionally substituted R^{b} group may be substituted with one or more substituents independently selected from the group consisting of —NR₂ and C₁-C₆ aliphatic.

In some embodiments of Formulas I, II, III, IV, IV-a, IV-b, V, VI, VII, VIII, VIII-a, and VIII-b, each R^{b} is independently selected from the group consisting of chloro, —OMe, methyl, —CH₂NHMe,

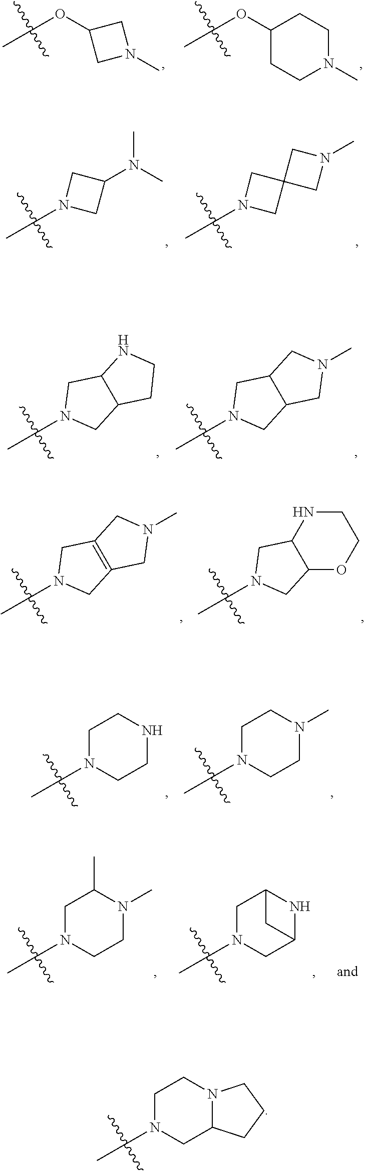

In some embodiments of Formulas I, II, III, IV, IV-a, IV-b, V, VI, VII, VIII, VIII-a, and VIII-b, each R^{b} is independently selected from the group consisting of —OMe, methyl, —CH₂NHMe,

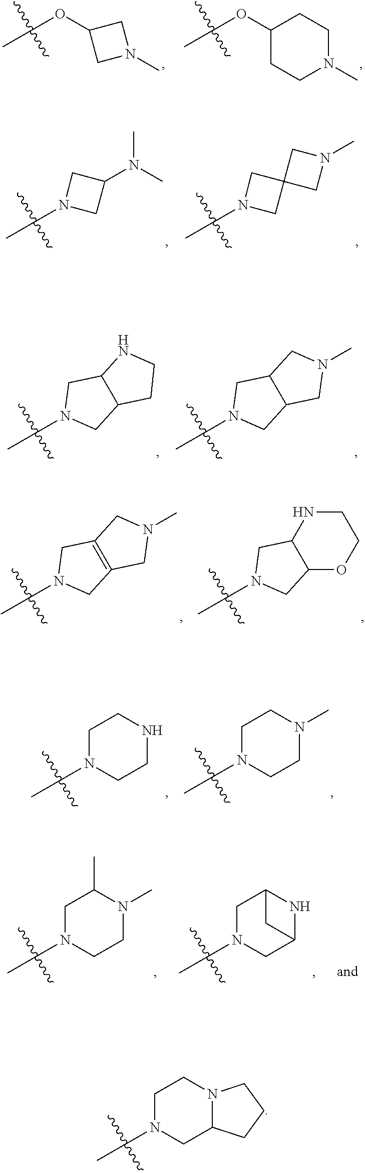

In some embodiments of Formulas I, II, III, IV, IV-a, IV-b, V, VI, VII, VIII, VIII-a, and VIII-b, each R^{b} is independently selected from the group consisting of methyl,

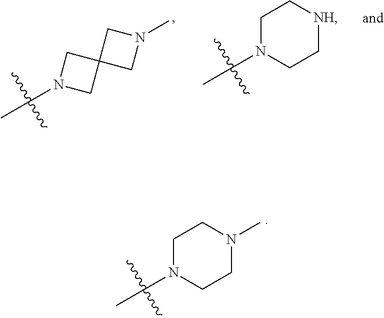

In some embodiments of Formulas I, II, and III, R¹ and R² are each independently selected from the group consisting of:
—H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-3 heteroatoms independently selected from N, O, and S,
or R¹ and R² combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
   wherein an optionally substituted R¹ and R² group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic.

In some embodiments of Formulas I, II, and III, R¹ and R² are each independently selected from the group consisting of —H, —OR, —NR₂, —CN, —C(O)NR₂, and C₁-C₆aliphatic. In some embodiments, R¹ and R² are each independently selected from the group consisting of —H, —OR, —CN, and C₁-C₆aliphatic. In some embodiments, R¹ and R² are each independently selected from the group consisting of —H and —OR. In some embodiments, R² is —H.

In some embodiments of Formulas I, II, and III, R¹ and R² are each independently selected from the group consisting of —H, —OH, —OMe, —NH₂, —NHMe, —CN, —C(O)NHMe, and methyl. In some embodiments, R¹ and R² are each independently selected from the group consisting of —H, —OH, —OMe, —CN, and methyl. In some embodiments, R¹ and R² are each independently selected from the group consisting of —H, —OH, and —OMe. In some embodiments, R² is —H. In some embodiments, R¹ is —OH, and R² is —H.

In some embodiments of Formulas IV, IV-a, IV-b, V, VI, and VII, R¹ is selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-3 heteroatoms independently selected from N, O, and S, wherein an optionally substituted R¹ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic.

In some embodiments of Formulas IV, IV-a, IV-b, V, VI, and VII, R¹ is selected from the group consisting of —OR, —NR₂, —CN, —C(O)NR₂, and C₁-C₆aliphatic. In some embodiments, R¹ is selected from the group consisting of —H, —OR, —CN, and C₁-C₆aliphatic. In some embodiments, R¹ is —OR. In some embodiments, R¹ is —OR, and m is 0.

In some embodiments of Formulas IV, IV-a, IV-b, V, VI, and VII, R¹ is selected from the group consisting of —OH, —OMe, —NH₂, —NHMe, —CN, —C(O)NHMe, and methyl. In some embodiments, R¹ is selected from the group consisting of —OH, —OMe, —CN, and methyl. In some embodiments, R¹ is selected from the group consisting of —OH and —OMe. In some embodiments, R¹ is —OH. In some embodiments, R¹ is —OH, and m is 0.

In some embodiments of Formulas I, II, and III, R³, R⁴, R⁵, and R⁶ are each independently selected from the group consisting of:
—H, optionally substituted C₁-C₆aliphatic, optionally substituted C₃-C₈cycloalkyl, and optionally substituted 3- to 8-membered heterocyclyl containing 1-4 heteroatoms independently selected from the group consisting of N, O, and S,
or R³ and R⁴, or R⁵ and R⁶, or a combination thereof, combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
   wherein an optionally substituted R³, R⁴, R⁵, and R⁶ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆ aliphatic.

In some embodiments of Formulas I, II, and III, R³, R⁴, R⁵, and R⁶ are each independently selected from the group consisting of —H and C₁-C₆aliphatic. In some embodiments, R³, R⁴, R⁵, and R⁶ are each independently selected from the group consisting of —H and methyl. In some embodiments, R³, R⁴, R⁵, and R⁶ are each —H. In some embodiments, R³ is methyl, and R⁴, R⁵, and R⁶ are each —H. In some embodiments, R⁵ is methyl, and R³, R⁴, and R⁶ are each —H.

In some embodiments of Formulas I, II, III, IV, IV-a, IV-b, V, VI, VII, VIII, VIII-a, and VIII-b, R⁷, R⁸, and R⁹ are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, and optionally substituted C₁-C₆aliphatic, wherein an optionally substituted R⁷, R⁸, and R⁹ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic. In some embodiments, R⁷, R⁸, and R⁹, if present, are each independently selected from the group consisting of —H, halogen, —OR, —NR₂, —CN, and C₁-C₆aliphatic optionally substituted with halogen. In some embodiments, R⁷, R⁸, and R⁹, if present, are each independently selected from the group consisting of —H, —OR, and C₁-C₆aliphatic. In some embodiments, R⁷, R⁸, and R⁹, if present, are each independently selected from the group consisting of —H, —OMe, and methyl. In some embodiments, R⁷, R⁸, and R⁹, if present, are each —H. In some embodiments, R⁷ is selected from the group consisting of —H, —OR, and C₁-C₆aliphatic. In some embodiments, R⁷ is selected from the group consisting of —H, —OMe, and methyl. In some embodiments, R⁷ is —H. In some embodiments, R⁸ is selected from the group consisting of —H, —OR, and C₁-C₆aliphatic. In some embodiments, R⁸ is selected from the group consisting of —H, —OMe, and methyl. In some embodiments, R⁸ is —H. In some embodiments, R⁹ is selected from the group consisting of —H, —OR, and C₁-C₆aliphatic. In some embodiments, R⁹ is selected from the group consisting of —H, —OMe, and methyl. In some embodiments, R⁹ is —H.

In some embodiments of Formulas I, II, III, IV, IV-a, IV-b, V, VI, VII, VIII, VIII-a, and VIII-b, each R is independently selected from the group consisting of —H, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-3 heteroatoms independently selected from N, O, and S, wherein an optionally substituted R group may be optionally substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic. In some embodiments, each R is independently selected from the group consisting of —H, optionally substituted C₁-C₆aliphatic, and optionally substituted 3- to 10-membered heterocyclyl containing 1-4 heteroatoms independently selected from N, O, and S, wherein an optionally substituted R group may be optionally substituted with one or more C₁-C₆aliphatic. In some embodiments, each R is independently selected from the group consisting of —H, methyl, and 4- to 6-membered heterocyclyl containing 1-2 heteroatoms independently selected from N, O, and S optionally substituted with methyl. In some embodiments, each R is —H.

In some embodiments, of Formulas I, II, III, IV, IV-a, IV-b, V, VI, VII, VIII, VIII-a, and VIII-b, each R′ is independently selected from the group consisting of optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-3 heteroatoms independently selected from N, O, and S, wherein an optionally substituted R′ group may be substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic. In some embodiments, each R′ is independently C₁-C₆aliphatic or C₃-C₁₀cycloalkyl. In some embodiments, each R′ is independently C₃-C₁₀cycloalkyl. In some embodiments, each R′ is cyclopropyl.

In some embodiments of Formulas I, II, III, IV, IV-a, IV-b, V, VI, and VII, m is 0, 1, or 2. In some embodiments, m is 0. In some embodiments, m is 0 or 1. In some embodiments, m is 0 or 2. In some embodiments, m is 1 or 2.

In some embodiments of Formulas I, II, III, IV, IV-a, IV-b, V, VI, and VII, n is 0, 1, or 2. In some embodiments, n is 0. In some embodiments, n is 0 or 1. In some embodiments, n is 0 or 2. In some embodiments, n is 1 or 2.

In some embodiments of Formulas IV, IV-a, IV-b, V, VI, and VII, m is 0, 1, or 2. In some embodiments, m is 0. In some embodiments, m is 1. In some embodiments, m is 2.

Another aspect of the present disclosure is a compound selected from Table 1, or a pharmaceutically acceptable salt thereof. It will be appreciated that in Table 1, where multiple Example numbers are indicated for a single chemical structure, each Example number refers to a stereoisomer of the compound. See Table 16 for additional information regarding such stereoisomers.

TABLE 1

| Ex. | Structure |
|---|---|
| 1 and 2 | 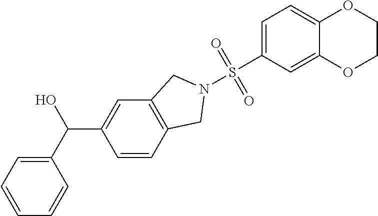 [SMILES: O=S(=O)(c1ccc2c(c1)OCCO2)N1Cc2ccc(C(O)c3ccccc3)cc2C1] |
| 3 and 4 | 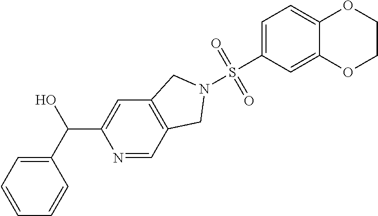 [SMILES: O=S(=O)(c1ccc2c(c1)OCCO2)N1Cc2cnc(C(O)c3ccccc3)cc2C1] |
| 5 and 6 | 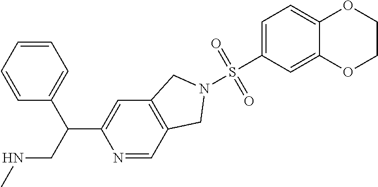 [SMILES: CNCC(c1ccccc1)c1cc2c(cn1)CN(S(=O)(=O)c1ccc3c(c1)OCCO3)C2] |
| 7 | 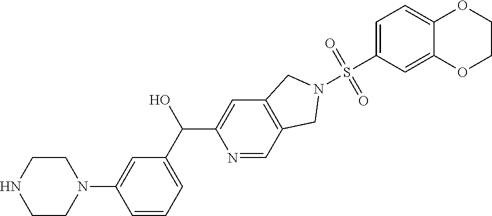 [SMILES: O=S(=O)(c1ccc2c(c1)OCCO2)N1Cc2cnc(C(O)c3cccc(N4CCNCC4)c3)cc2C1] |
| 8 | 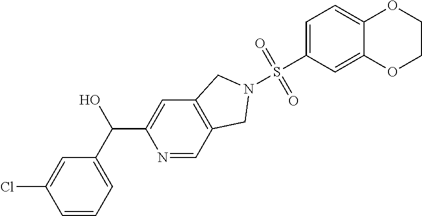 [SMILES: O=S(=O)(c1ccc2c(c1)OCCO2)N1Cc2cnc(C(O)c3cccc(Cl)c3)cc2C1] |
| 9 | 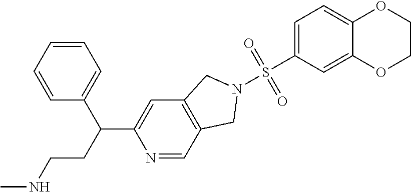 [SMILES: CNCCC(c1ccccc1)c1cc2c(cn1)CN(S(=O)(=O)c1ccc3c(c1)OCCO3)C2] |
| 10 | 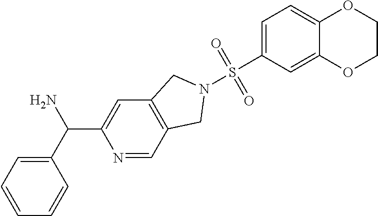 [SMILES: NC(c1ccccc1)c1cc2c(cn1)CN(S(=O)(=O)c1ccc3c(c1)OCCO3)C2] |
| 11 | 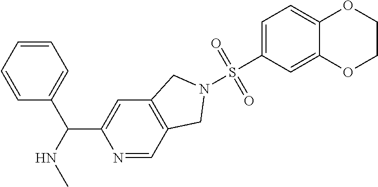 [SMILES: CNC(c1ccccc1)c1cc2c(cn1)CN(S(=O)(=O)c1ccc3c(c1)OCCO3)C2] |
| 12 | 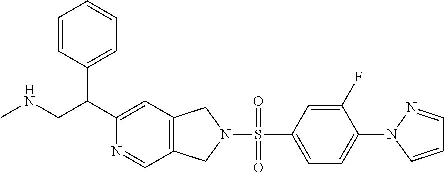 [SMILES: CNCC(c1ccccc1)c1cc2c(cn1)CN(S(=O)(=O)c1ccc(-n3cccn3)c(F)c1)C2] |
| 13 | 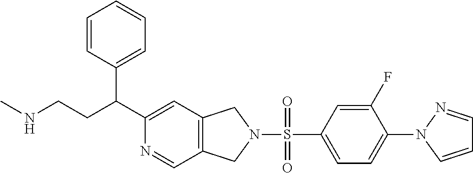 [SMILES: CNCCC(c1ccccc1)c1cc2c(cn1)CN(S(=O)(=O)c1ccc(-n3cccn3)c(F)c1)C2] |
| 14 and 15 | 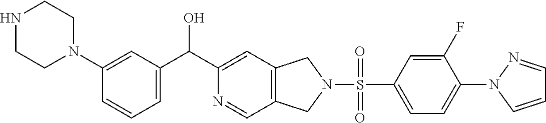 [SMILES: O=S(=O)(c1ccc(-n2cccn2)c(F)c1)N1Cc2cnc(C(O)c3cccc(N4CCNCC4)c3)cc2C1] |
| 16 | 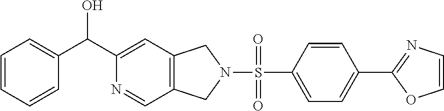 [SMILES: O=S(=O)(c1ccc(-c2ncco2)cc1)N1Cc2cnc(C(O)c3ccccc3)cc2C1] |
| 17 | 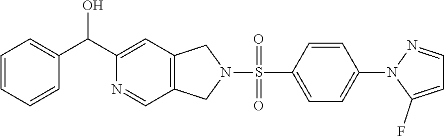 [SMILES: O=S(=O)(c1ccc(-n2nccc2F)cc1)N1Cc2cnc(C(O)c3ccccc3)cc2C1] |
| 18 | 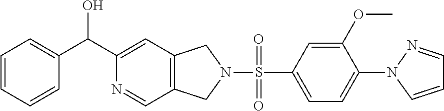 [SMILES: COc1cc(S(=O)(=O)N2Cc3cnc(C(O)c4ccccc4)cc3C2)ccc1-n1cccn1] |
| 19 | 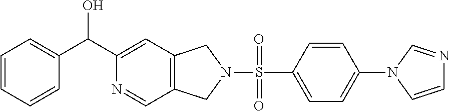 [SMILES: O=S(=O)(c1ccc(-n2ccnc2)cc1)N1Cc2cnc(C(O)c3ccccc3)cc2C1] |
| 20, 21, 22, and 23 | 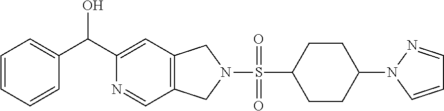 [SMILES: O=S(=O)(C1CCC(n2cccn2)CC1)N1Cc2cnc(C(O)c3ccccc3)cc2C1] |
| 24 | 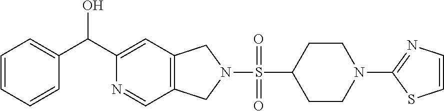 [SMILES: O=S(=O)(C1CCN(c2nccs2)CC1)N1Cc2cnc(C(O)c3ccccc3)cc2C1] |
| 25 | 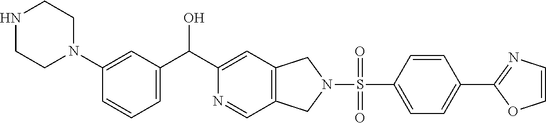 [SMILES: O=S(=O)(c1ccc(-c2ncco2)cc1)N1Cc2cnc(C(O)c3cccc(N4CCNCC4)c3)cc2C1] |
| 26 and 27 | 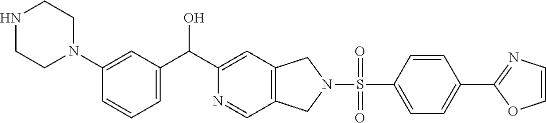 [SMILES: O=S(=O)(c1ccc(-c2ncco2)cc1)N1Cc2cnc(C(O)c3cccc(N4CCNCC4)c3)cc2C1] |
| 28 | 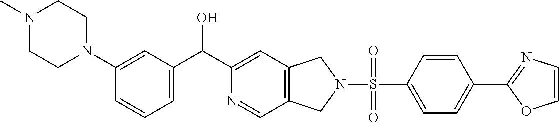 [SMILES: CN1CCN(c2cccc(C(O)c3cc4c(cn3)CN(S(=O)(=O)c3ccc(-c5ncco5)cc3)C4)c2)CC1] |
| 29 and 30 | 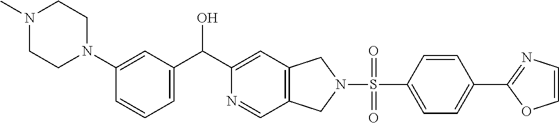 [SMILES: CN1CCN(c2cccc(C(O)c3cc4c(cn3)CN(S(=O)(=O)c3ccc(-c5ncco5)cc3)C4)c2)CC1] |
| 31 | 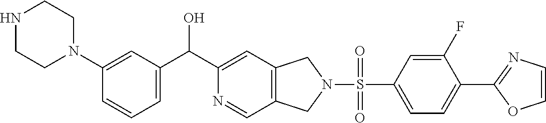 [SMILES: O=S(=O)(c1ccc(-c2ncco2)c(F)c1)N1Cc2cnc(C(O)c3cccc(N4CCNCC4)c3)cc2C1] |
| 32 | 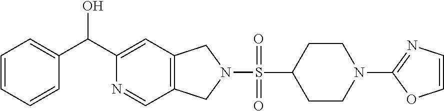 [SMILES: O=S(=O)(C1CCN(c2ncco2)CC1)N1Cc2cnc(C(O)c3ccccc3)cc2C1] |
| 33 | 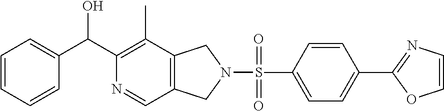 [SMILES: Cc1c(C(O)c2ccccc2)ncc2c1CN(S(=O)(=O)c1ccc(-c3ncco3)cc1)C2] |
| 34 | 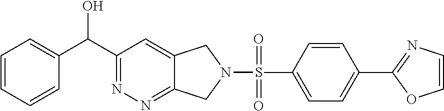 [SMILES: O=S(=O)(c1ccc(-c2ncco2)cc1)N1Cc2cc(C(O)c3ccccc3)nnc2C1] |
| 35 | 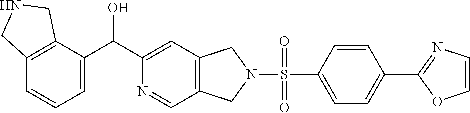 [SMILES: O=S(=O)(c1ccc(-c2ncco2)cc1)N1Cc2cnc(C(O)c3cccc4c3CNC4)cc2C1] |
| 36 | 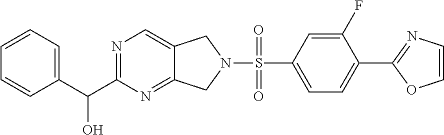 [SMILES: O=S(=O)(c1ccc(-c2ncco2)c(F)c1)N1Cc2cnc(C(O)c3ccccc3)nc2C1] |
| 37 | 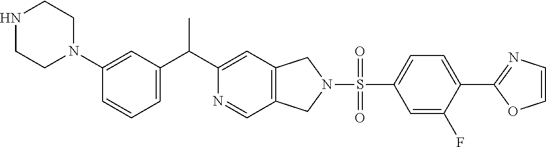 [SMILES: CC(c1cccc(N2CCNCC2)c1)c1cc2c(cn1)CN(S(=O)(=O)c1ccc(-c3ncco3)c(F)c1)C2] |
| 38 and 39 | 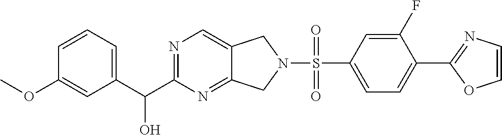 [SMILES: COc1cccc(C(O)c2ncc3c(n2)CN(S(=O)(=O)c2ccc(-c4ncco4)c(F)c2)C3)c1] |
| 40 and 41 | 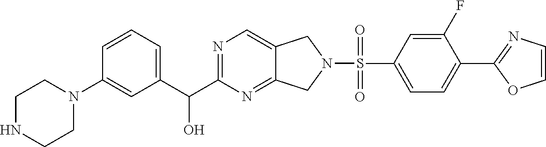 [SMILES: O=S(=O)(c1ccc(-c2ncco2)c(F)c1)N1Cc2cnc(C(O)c3cccc(N4CCNCC4)c3)nc2C1] |
| 42 and 43 | 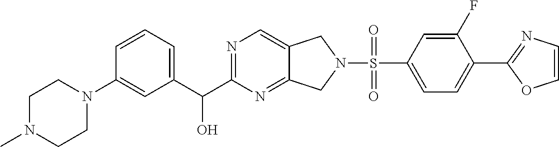 [SMILES: CN1CCN(c2cccc(C(O)c3ncc4c(n3)CN(S(=O)(=O)c3ccc(-c5ncco5)c(F)c3)C4)c2)CC1] |
| 44 | 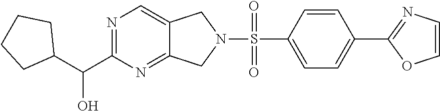 [SMILES: O=S(=O)(c1ccc(-c2ncco2)cc1)N1Cc2cnc(C(O)C3CCCC3)nc2C1] |
| 45 | 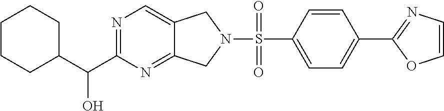 [SMILES: O=S(=O)(c1ccc(-c2ncco2)cc1)N1Cc2cnc(C(O)C3CCCCC3)nc2C1] |
| 46 and 47 | 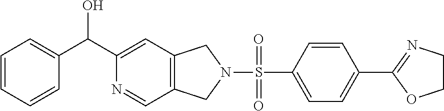 [SMILES: O=S(=O)(c1ccc(C2=NCCO2)cc1)N1Cc2cnc(C(O)c3ccccc3)cc2C1] |
| 48 and 49 | 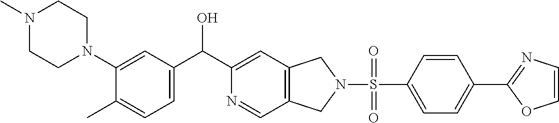 [SMILES: Cc1ccc(C(O)c2cc3c(cn2)CN(S(=O)(=O)c2ccc(-c4ncco4)cc2)C3)cc1N1CCN(C)CC1] |
| 50 and 51 | 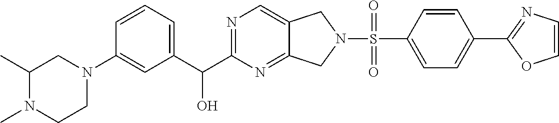 [SMILES: CC1CN(c2cccc(C(O)c3ncc4c(n3)CN(S(=O)(=O)c3ccc(-c5ncco5)cc3)C4)c2)CCN1C] |
| 52 and 53 | 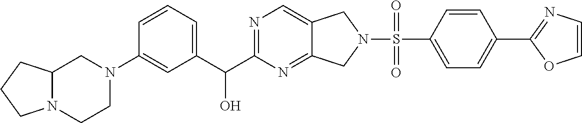 [SMILES: O=S(=O)(c1ccc(-c2ncco2)cc1)N1Cc2cnc(C(O)c3cccc(N4CCN5CCCC5C4)c3)nc2C1] |
| 54 | 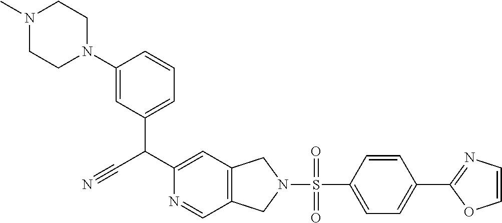 [SMILES: CN1CCN(c2cccc(C(C#N)c3cc4c(cn3)CN(S(=O)(=O)c3ccc(-c5ncco5)cc3)C4)c2)CC1] |
| 55 and 56 | 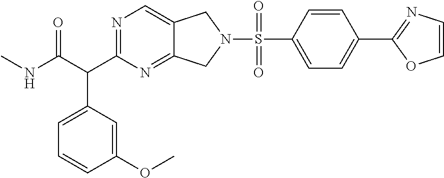 [SMILES: CNC(=O)C(c1cccc(OC)c1)c1ncc2c(n1)CN(S(=O)(=O)c1ccc(-c3ncco3)cc1)C2] |
| 57 and 58 | 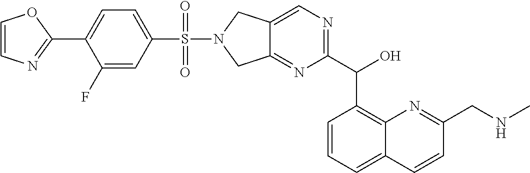 [SMILES: CNCc1ccc2cccc(C(O)c3ncc4c(n3)CN(S(=O)(=O)c3ccc(-c5ncco5)c(F)c3)C4)c2n1] |
| 59 and 60 | 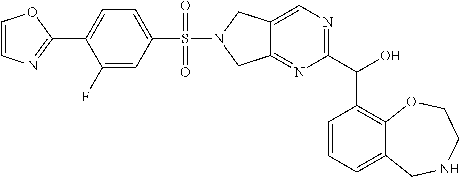 [SMILES: O=S(=O)(c1ccc(-c2ncco2)c(F)c1)N1Cc2cnc(C(O)c3cccc4c3OCCNC4)nc2C1] |
| 61, 62, 63, and 64 | 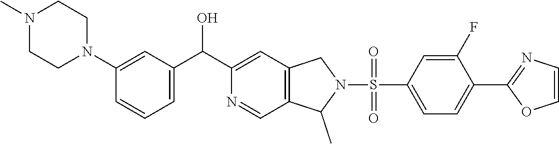 [SMILES: CC1c2cnc(C(O)c3cccc(N4CCN(C)CC4)c3)cc2CN1S(=O)(=O)c1ccc(-c2ncco2)c(F)c1] |
| 65, 66, 67, and 68 | 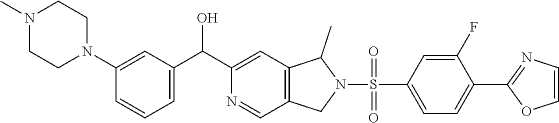 [SMILES: CC1c2cc(C(O)c3cccc(N4CCN(C)CC4)c3)ncc2CN1S(=O)(=O)c1ccc(-c2ncco2)c(F)c1] |
| 69 and 70 | 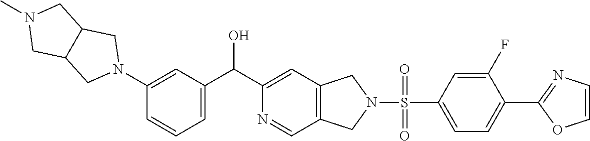 [SMILES: CN1CC2CN(c3cccc(C(O)c4cc5c(cn4)CN(S(=O)(=O)c4ccc(-c6ncco6)c(F)c4)C5)c3)CC2C1] |
| 71 and 72 | 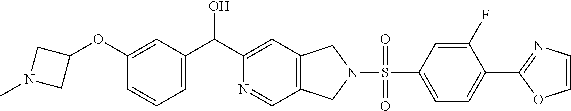 [SMILES: CN1CC(Oc2cccc(C(O)c3cc4c(cn3)CN(S(=O)(=O)c3ccc(-c5ncco5)c(F)c3)C4)c2)C1] |
| 73 and 74 | 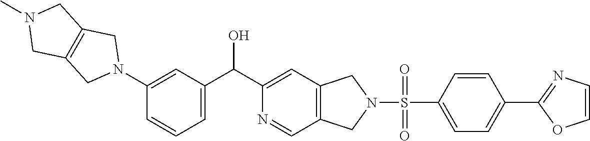 [SMILES: CN1CC2=C(C1)CN(c1cccc(C(O)c3cc4c(cn3)CN(S(=O)(=O)c3ccc(-c5ncco5)cc3)C4)c1)C2] |
| 75 | 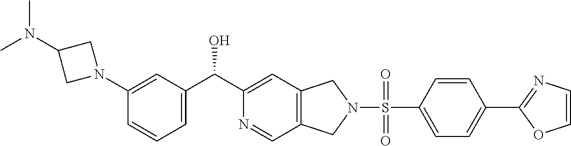 [SMILES: CN(C)C1CN(c2cccc([C@H](O)c3cc4c(cn3)CN(S(=O)(=O)c3ccc(-c5ncco5)cc3)C4)c2)C1] |
| 76 and 77 | 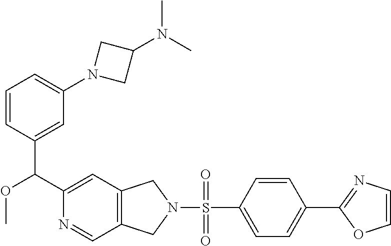 [SMILES: COC(c1cccc(N2CC(N(C)C)C2)c1)c1cc2c(cn1)CN(S(=O)(=O)c1ccc(-c3ncco3)cc1)C2] |
| 78 and 79 | 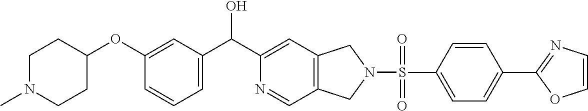 [SMILES: CN1CCC(Oc2cccc(C(O)c3cc4c(cn3)CN(S(=O)(=O)c3ccc(-c5ncco5)cc3)C4)c2)CC1] |
| 80 and 81 | 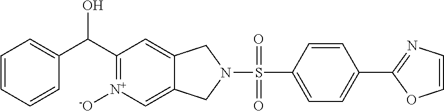 |
| 82 and 83 | 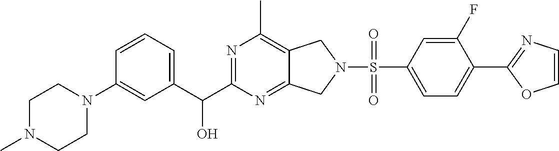 [SMILES: Cc1nc(C(O)c2cccc(N3CCN(C)CC3)c2)nc2c1CN(S(=O)(=O)c1ccc(-c3ncco3)c(F)c1)C2] |
| 84 and 85 | 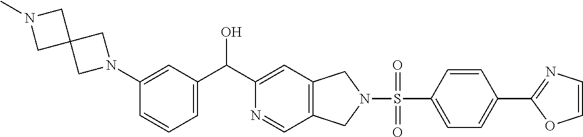 [SMILES: CN1CC2(C1)CN(c1cccc(C(O)c3cc4c(cn3)CN(S(=O)(=O)c3ccc(-c5ncco5)cc3)C4)c1)C2] |
| 86, 87, 88, and 89 | 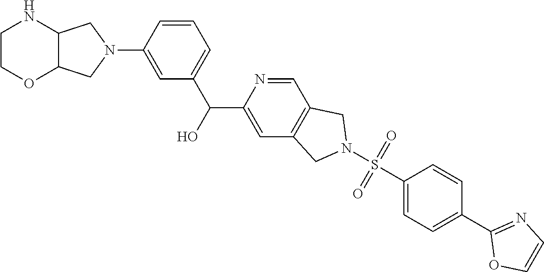 [SMILES: O=S(=O)(c1ccc(-c2ncco2)cc1)N1Cc2cnc(C(O)c3cccc(N4CC5NCCOC5C4)c3)cc2C1] |
| 90 and 91 | 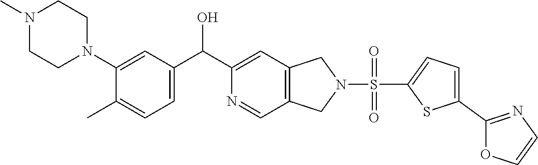 [SMILES: Cc1ccc(C(O)c2cc3c(cn2)CN(S(=O)(=O)c2ccc(-c4ncco4)s2)C3)cc1N1CCN(C)CC1] |
| 92, 93, 94, and 95 | 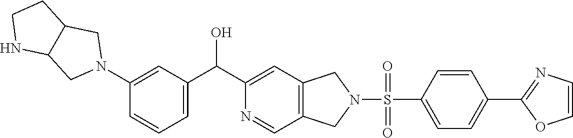 [SMILES: O=S(=O)(c1ccc(-c2ncco2)cc1)N1Cc2cnc(C(O)c3cccc(N4CC5CCNC5C4)c3)cc2C1] |
| 96 and 97 | 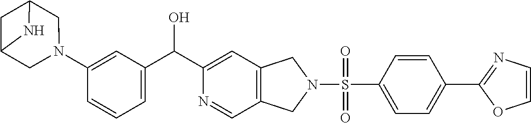 [SMILES: O=S(=O)(c1ccc(-c2ncco2)cc1)N1Cc2cnc(C(O)c3cccc(N4CC5CC(C4)N5)c3)cc2C1] |
| 98 and 99 | 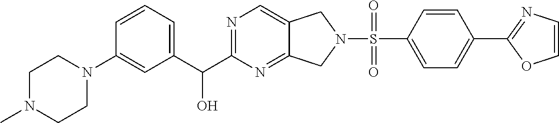 [SMILES: CN1CCN(c2cccc(C(O)c3ncc4c(n3)CN(S(=O)(=O)c3ccc(-c5ncco5)cc3)C4)c2)CC1] |
| 100 and 101 | 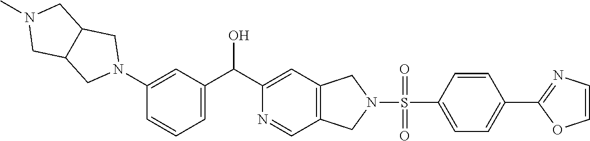 [SMILES: CN1CC2CN(c3cccc(C(O)c4cc5c(cn4)CN(S(=O)(=O)c4ccc(-c6ncco6)cc4)C5)c3)CC2C1] |
| 102 and 103 | 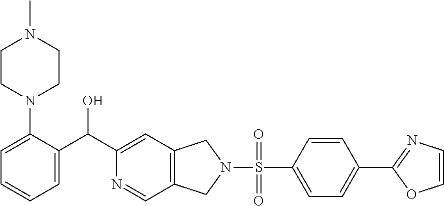 [SMILES: CN1CCN(c2ccccc2C(O)c2cc3c(cn2)CN(S(=O)(=O)c2ccc(-c4ncco4)cc2)C3)CC1] |
| 104 and 105 | 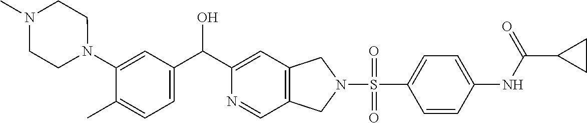 [SMILES: Cc1ccc(C(O)c2cc3c(cn2)CN(S(=O)(=O)c2ccc(NC(=O)C4CC4)cc2)C3)cc1N1CCN(C)CC1] |
| 106 and 107 | 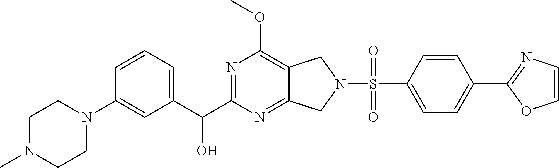 [SMILES: COc1nc(C(O)c2cccc(N3CCN(C)CC3)c2)nc2c1CN(S(=O)(=O)c1ccc(-c3ncco3)cc1)C2] |
| 108 and 109 | 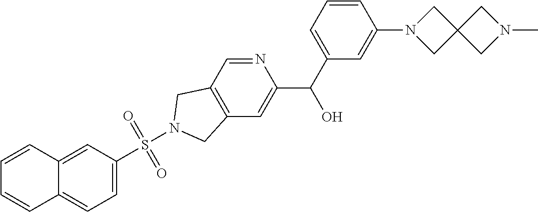 [SMILES: CN1CC2(C1)CN(c1cccc(C(O)c3cc4c(cn3)CN(S(=O)(=O)c3ccc5ccccc5c3)C4)c1)C2] |
| 110 | 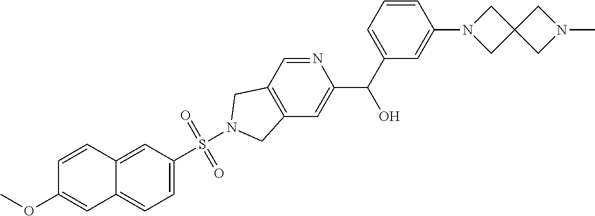 |

It will be appreciated that throughout the present disclosure, unless otherwise indicated, reference to a compound of Formula I is intended to also include II, III, IV, IV-a, IV-b, V, VI, VII, VIII, VIII-a, and VIII-b, and compound species of such formulas disclosed herein.

Unless otherwise stated, it will be appreciated that when “one or more” substituents are recited for a particular variable, it includes one, two, three, four, or more substituents as valency permits.

Unless otherwise stated, structures depicted herein are also meant to include all stereoisomeric (e.g., enantiomeric or diastereomeric) forms of the structure, as well as all geometric or conformational isomeric forms of the structure; for example, the R and S configurations for each stereocenter. Therefore, single stereochemical isomers, as well as enantiomeric, diastereomeric, and geometric (or conformational) mixtures of the present compounds are within the scope of the disclosure. For example, in some cases Table 1 shows one or more stereoisomers of a compound, and unless otherwise indicated, represents each stereoisomer alone and/or as a mixture. Unless otherwise stated, all tautomeric forms of the compounds of the disclosure are within the scope of the disclosure.

In some embodiments, a compound of Formula I is obtained by a process comprising a purification method in Table 15. In some embodiments, the compound is obtained by a process comprising a purification method in Table 15 and is the 1^{st} eluting isomer of the purification method. In some embodiments, the compound is obtained by a process comprising a purification method in Table 15 and is the 2^{nd} eluting isomer of the purification method. In some embodiments, the compound is obtained by a process comprising a purification method in Table 15 and is the 3^{rd} eluting isomer of the purification method. In some embodiments, the compound is obtained by a process comprising a purification method in Table 15 and is the 4^{th} eluting isomer of the purification method. In some embodiments, the compound is obtained by a process comprising a purification method in Table 15 and is the 5^{th}, 6^{th}, 7^{th}, or 8^{th} eluting isomer of the purification method.

In some embodiments, a USP9X Inhibitor is obtained by a process comprising a purification method in Table 15. In some embodiments, the USP9X Inhibitor is obtained by a process comprising a purification method in Table 15 and is the 1^{st} eluting isomer of the purification method. In some embodiments, the USP9X Inhibitor is obtained by a process comprising a purification method in Table 15 and is the 2^{nd} eluting isomer of the purification method. In some embodiments, the USP9X Inhibitor is obtained by a process comprising a purification method in Table 15 and is the 3^{rd} eluting isomer of the purification method. In some embodiments, the USP9X Inhibitor is obtained by a process comprising a purification method in Table 15 and is the 4^{th} eluting isomer of the purification method. In some embodiments, the USP9X Inhibitor is obtained by a process comprising a purification method in Table 15 and is the 5^{th}, 6^{th}, 7^{th}, or 8 eluting isomer of the purification method.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ³C- or ¹⁴C-enriched carbon are within the scope of this disclosure.

The disclosure also provides compounds of Formula I (e.g., compounds that are not USP9X Inhibitors) that are useful, for example, as analytical tools and/or control compounds in biological assays.

The compounds of Formula I may form salts which are also within the scope of this disclosure. Reference to a compound of the Formula I herein is understood to include reference to salts thereof, unless otherwise indicated. Pharmaceutically acceptable salts are well known in the art. For example, S. M. Berge, et al. describes pharmaceutically acceptable salts in detail in J. Pharmaceutical Sciences, 66: 1-19 (1977).

The disclosure also includes pharmaceutical compositions comprising one or more compounds as described herein, or a pharmaceutically acceptable salt thereof, and a pharmaceutically acceptable excipient. In some embodiments, pharmaceutical compositions reported herein can be provided in a unit dosage form (e.g., capsule, tablet or the like). In some embodiments, pharmaceutical compositions reported herein can be provided in an oral dosage form. In some embodiments, the pharmaceutical composition is orally administered in any orally acceptable dosage form. In some embodiments, an oral dosage form of a compound of Formula I can be a capsule. In some embodiments, an oral dosage form of a compound of Formula I is a tablet. In some embodiments, an oral dosage form comprises one or more fillers, disintegrants, lubricants, glidants, anti-adherents, and/or anti-statics. In some embodiments, an oral dosage form is prepared via dry blending. In some embodiments, an oral dosage form is a tablet and is prepared via dry granulation.

### Methods of Using the Disclosed Compounds

Another aspect of the present disclosure is the use of compounds of Formula I. Compounds of Formula I are useful in medicine. For example, compounds and compositions described herein are inhibitors of USP9X. Methods of treatment (e.g., by inhibiting USP9X) can comprise administering to a subject in need thereof a therapeutically effective amount of (i) a compound disclosed herein, or a pharmaceutically acceptable salt thereof, or (ii) a pharmaceutical composition comprising a compound disclosed herein, or a pharmaceutically acceptable salt thereof, and a pharmaceutically acceptable carrier. In some embodiments, a method of treating a disease associated with modulation of USP9X comprises administering a therapeutically effective amount of a compound disclosed herein. In some embodiments, a method of treating cancer comprises administering a therapeutically effective amount of a compound disclosed herein.

### Methods of Synthesizing the Disclosed Compounds

The compounds of the present disclosure may be made by a variety of methods, including standard chemistry. Suitable synthetic routes are depicted in the Schemes given below.

### EXAMPLES

### Analytical Methods, Materials, and Instrumentation

Unless otherwise noted, reagents and solvents were used as received from commercial suppliers. Unless otherwise noted, reactions were conducted under an inert atmosphere of nitrogen. NMR instrument: Bruker BBFO ASCEND™ 400 AVANCE III 400 MHz and Bruker BBFO ULTRASHIELD™ 300 AVANCE III 300 MHz. Internal standard: Tetramethylsilane (TMS). MassSpec instruments and ionization method: Shimadzu LC-2020, electrospray ionization, ESI. Chromatography instruments (Reverse phase chromatography: Agela Technologies MP200. Preparatory HPLC (Prep-HPLC): Waters. Supercritical fluid chromatography (SFC): Shimadzu).

### Abbreviations

Ac₂O Acetic anhydride
ACN Acetonitrile
AcOH Acetic acid
AIBN 2,2′-Azobis(2-methylpropionitrile)
atm atmosphere
BINAP 2,2′-Bis(diphenylphosphino)-1,1′-binaphthalene
BTMG 2-tert-Butyl-1,1,3,3-tetramethylguanidine
CbzCl Benzyl chloroformate
CDI 1,1′-Carbonyldiimidazole
Cp*Ru(cod)Cl Chloro(pentamethylcyclopentadienyl)(cyclooctadiene)ruthenium(II)
δ chemical shift
DCM Dichloromethane or methylene chloride
DCE 1,2-Dichloroethane
DEA Diethylamine
DEAD Diethyl azodicarboxylate
DIAD Diisopropyl azodicarboxylate
DIEA N,N-Diisopropylethylamine
DMA N,N-Dimethylacetamide
DME Dimethoxyethane
DMF N,N-Dimethylformamide
DMP Dess-Martin Periodinane
DMSO Dimethylsulfoxide
dppf 1.1-Bis(diphenylphosphino)ferrocene
EA Ethyl acetate
EDCI N-(3-Dimethylaminopropyl)-N′-ethylcarbodiimide hydrochloride
EDTA Ethylenediaminetetraacetic acid
ee enantiomeric excess
h hour
¹H NMR proton nuclear magnetic resonance
HATU 2-(3H-[1,2,3]Triazolo[4,5-b]pyridin-3-yl)-1,1,3,3-tetramethylisouronium hexafluorophosphate
HOBT 1H-Benzo[d][1,2,3]triazol-1-ol hydrate
HPLC high performance liquid chromatography
Hz Hertz
IPA Isopropyl alcohol
LAH lithium aluminum hydride
LCMS liquid chromatography/mass spectrometry
LiHMDS Lithium bis(trimethylsilyl)amide
m-CPBA m-Chloroperoxybenzoic acid
MeOH Methanol
min minutes
MS mass spectrometry
NMM 4-Methylmorpholine
NMP N-Methyl-2-pyrrolidone
Pd₂(dba)₃.CH₂Cl₂ Tris(dibenzylideneacetone)dipalladium(0) chloroform adduct
Pd(dppf)Cl₂ [1,1′-Bis(diphenylphosphino)ferrocene]dichloropalladium(II)
Pd(PPh₃)₄ Tetrakis(triphenylphosphine)palladium (0)
PE Petroleum ether
PTSA 4-methylbenzenesulfonic acid
rt room temperature
Rt retention time
RuCl₂[(R)-DM-BINAP] Dichloro[(S)-(−)-2,2′-bis[di(3,5-xylyl)phosphino]-1,1′-binaphthyl][(2S)—[(R)-DAIPEN] (+)-1,1-bis(4-methoxyphenyl)-3-methyl-1,2-butanediamine]ruthenium(II)
RuPhos 2-Dicyclohexylphosphino-2′,6′-diisopropoxybiphenyl
RuPhos 2G Chloro(2-dicyclohexylphosphino-2′,6′-diisopropoxy-1,1′-biphenyl)[2-(2′-amino-1,1′-biphenyl)]palladium(II)
TBDMS-Cl tert-butyl dimethylsilyl chloride
tBuXPhos 3G [(2-Di-tert-butylphosphino-2′,4′,6′-triisopropyl-1,1′-biphenyl)-2-(2′-amino-1,1′-biphenyl)] palladium(II) methanesulfonate
TEA Triethylamine
TFA Trifluoroacetic acid
THF Tetrahydrofuran
TLC thin layer chromatography
Xantphos 4,5-Bis(diphenylphosphino)-9,9-dimethylxanthene
XPhos 2-Dicyclohexylphosphino-2′,4′,6′-triisopropylbiphenyl
XPhos 2G Chloro(2-dicyclohexylphosphino-2′,4′,6′-triisopropyl-1,1′-biphenyl)[2-(2′-amino-1,1′-biphenyl)]palladium(II)
XPhos 3G Methanesulfonato(2-dicyclohexylphosphino-2′,4′,6′-tri-i-propyl-1,1′-biphenyl)(2′-amino-1,1′-biphenyl-2-yl)palladium(II) dichloromethane adduct
SelectFluor 1-Chloromethyl-4-fluoro-1,4-diazoniabicyclo[2.2.2]octane bis(tetrafluoroborate)
SPhos 2-Dicyclohexylphosphino-2′,6′-dimethoxybiphenyl
STAB Sodium triacetoxyborohydride
PFA Paraformaldehyde

### Example 1. Synthesis of Intermediates

### Intermediate 1-1. [(2S)-2-[2-(2,3-dihydro-1,4-benzodioxine-6-sulfonyl)-1H,2H,3H-pyrrolo[3,4-c]pyridin-6-yl]-2-phenylethyl] (methyl)amine; [(2R)-2-[2-(2,3-dihydro-1,4-benzodioxine-6-sulfonyl)-1H,2H,3H-pyrrolo[3,4-c]pyridin-6-yl]-2-phenylethyl](methyl) amine

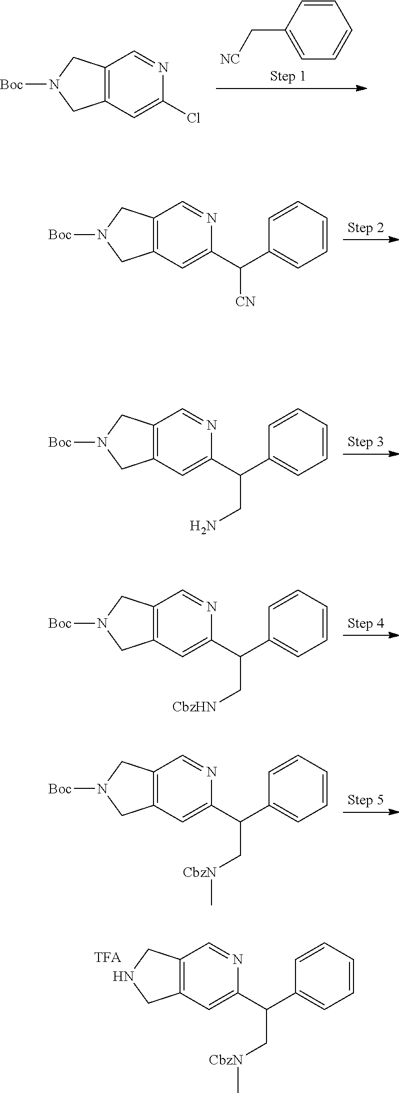

### Step 1. Tert-butyl 6-[cyano(phenyl)methyl]-1H,2H,3H-pyrrolo[3,4-c]pyridine-2-carboxylate

To a solution of tert-butyl 6-chloro-1H,2H,3H-pyrrolo[3,4-c]pyridine-2-carboxylate (300 mg, 1.18 mmol) in THF (3 mL) was added 2-phenylacetonitrile (152 mg, 1.30 mmol) and sodium amide (92 mg, 2.36 mmol). The resulting mixture was stirred for 30 min at 50° C. and then cooled to room temperature. The reaction mixture was poured into brine (5 mL) and then extracted with EA (3×5 mL). The combined organic layers were dried over anhydrous sodium sulfate, filtered and concentrated under vacuum. The resulting crude product was purified by silica gel chromatography (eluting with 1:3 EA/PE) to afford tert-butyl 6-[cyano(phenyl)methyl]-1H,2H,3H-pyrrolo[3,4-c]pyridine-2-carboxylate as light yellow oil (100 mg, 25%). LCMS (ES, m/z) 336 [M+H]⁺.

### Step 2. Tert-butyl 6-(2-amino-1-phenylethyl)-1H,2H,3H-pyrrolo[3,4-c]pyridine-2-carboxylate

To a solution of tert-butyl 6-[cyano(phenyl)methyl]-1H,2H,3H-pyrrolo[3,4-c]pyridine-2-carboxylate (100 mg, 0.30 mmol) in methanol (2 mL) was added Raney Ni (10 mg, 0.12 mmol). Then hydrogen was introduced with hydrogen balloon. The resulting mixture was stirred for 16 h at room temperature. The reaction mixture was filtered through a short pad of Celite and concentrated under vacuum to afford tert-butyl 6-(2-amino-1-phenylethyl)-1H,2H,3H-pyrrolo[3,4-c]pyridine-2-carboxylate as light yellow oil (90 mg, 89%). LCMS (ES, m/z) 340 [M+H]⁺.

### Step 3. Tert-butyl 6-(2-[[(benzyloxy)carbonyl]amino]-1-phenylethyl)-1H,2H,3H-pyrrolo[3,4-c]pyridine-2-carboxylate

To a solution of tert-butyl 6-(2-amino-1-phenylethyl)-1H,2H,3H-pyrrolo[3,4-c]pyridine-2-carboxylate (90 mg, 0.27 mmol) in DCM (1 mL) was added CbzCl (68 mg, 0.39 mmol) and TEA (54 mg, 0.53 mmol). The resulting mixture was stirred for 16 h at room temperature. The reaction mixture was poured into brine (3 mL) and then extracted with EA (3×3 mL). The combined organic layers were dried over anhydrous sodium sulfate, filtered and concentrated under vacuum. The resulting crude product was purified by silica gel chromatography (eluting with 1:1 EA/PE) to afford tert-butyl 6-(2-[[(benzyloxy)carbonyl]amino]-1-phenylethyl)-1H,2H,3H-pyrrolo[3,4-c]pyridine-2-carboxylate as light yellow oil (110 mg, 88%). LCMS (ES, m/z) 474 [M+H]⁺.

### Step 4. Tert-butyl 6-(2-[[(benzyloxy)carbonyl](methyl)amino]-1-phenylethyl)-1H,2H,3H-pyrrolo[3,4-c]pyridine-2-carboxylate

To a solution of tert-butyl 6-(2-[[(benzyloxy)carbonyl]amino]-1-phenylethyl)-1H,2H,3H-pyrrolo[3,4-c]pyridine-2-carboxylate (110 mg, 0.23 mmol) in THF (1 mL) was added sodium hydride (11 mg, 0.28 mmol, 60% dispersion in mineral oil) with stirring at 0° C. After stirring for 30 min at 0° C., iodomethane (66 mg, 0.46 mmol) was added in dropwise. The resulting mixture was stirred for 2 h at rt and then poured into brine (3 mL) and then extracted with EA (3×3 mL). The combined organic layers were dried over anhydrous sodium sulfate, filtered and concentrated under vacuum. The resulting crude product was purified by silica gel chromatography (eluting with 1:1 EA/PE) to afford tert-butyl 6-(2-[[(benzyloxy)carbonyl](methyl)amino]-1-phenylethyl)-1H,2H,3H-pyrrolo[3,4-c]pyridine-2-carboxylate as light yellow oil (100 mg, 88%). LCMS (ES, m/z) 488 [M+H]⁺.

### Step 5. Benzyl N-methyl-N-(2-phenyl-2-[1H,2H,3H-pyrrolo[3,4-c]pyridin-6-yl]ethyl)carbamate (TFA salt)

To a solution of tert-butyl 6-(2-[[(benzyloxy)carbonyl](methyl)amino]-1-phenylethyl)-1H,2H,3H-pyrrolo[3,4-c]pyridine-2-carboxylate (100 mg, 0.21 mmol) in dichloromethane (1 mL) was added TFA (0.2 mL). The resulting solution was stirred for 1 h at room temperature and concentrated under vacuum to afford benzyl N-methyl-N-(2-phenyl-2-[1H,2H,3H-pyrrolo[3,4-c]pyridin-6-yl]ethyl)carbamate (TFA salt) as light brown oil (100 mg, crude). LCMS (ES, m/z) 388 [M+H]⁺.

### Intermediate 2-1. (3-bromophenyl)([2-[3-fluoro-4-(1,3-oxazol-2-yl)benzenesulfonyl]-1H,2H,3H-pyrrolo[3,4-c]pyridin-6-yl])methanol

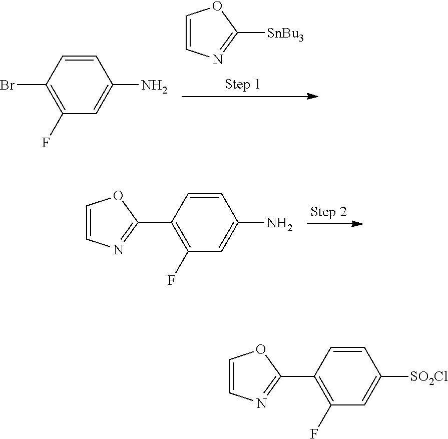

### Step 1. 3-fluoro-4-(1,3-oxazol-2-yl)aniline

To a solution of 4-bromo-3-fluoroaniline (474 mg, 2.51 mmol) in 1,4-dioxane (10 mL) was added Pd(dppf)Cl₂ (183 mg, 0.25 mmol) and 2-(tributylstannyl)-1,3-oxazole (900 mg, 2.52 mmol). The resulting mixture was stirred for 48 h at 100° C. and then cooled to room temperature. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 3-fluoro-4-(1,3-oxazol-2-yl)aniline (180 mg, 41%). LCMS (ES, m/z) 179 [M+H]⁺.

### Step 2. 3-fluoro-4-(1,3-oxazol-2-yl)benzene-1-sulfonyl chloride

Into glacial acetic acid (10 mL) was bubbled in SO₂ gas for 1 h at room temperature. Then CuCl₂ (34 mg, 0.25 mmol) was added and SO₂ gas was bubbled in for additional 2 h to afford solution A. To a pre-cooled solution of 3-fluoro-4-(1,3-oxazol-2-yl)aniline (180 mg, 1.01 mmol) in acetic acid (2 mL) and concentrated hydrochloric acid (6 mL) was added a solution of sodium nitrite (77 mg, 1.11 mmol) in distilled water (0.5 mL) dropwise with stirring at −10° C. After stirring for 15 min, solution A was added to this diazonium salt solution at −10° C. The resulting solution was allowed to warm to room temperature naturally and stirred for 16 h. The reaction mixture was treated with water (10 mL) and then extracted with EA (3×10 mL). The combined organic layers were dried over anhydrous sodium sulfate, filtered, and concentrated under vacuum. The resulting crude product was purified by silica gel chromatography (eluting with 0:100 to 12:88 ethyl acetate/petroleum ether) to afford 3-fluoro-4-(1,3-oxazol-2-yl)benzene-1-sulfonyl chloride (120 mg, 45%). LCMS (ES, m/z) 262, 264 [M+H]⁺.

The Intermediates in Table 2 were synthesized according to the procedure described for Intermediate 2-1 above.

TABLE 2

| | | LCMS: |
|---|---|---|
| | | (ESI) m/z |
| Intermediate | Compound | [M + H]⁺ |
| 2-2 | 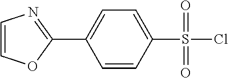 [SMILES: O=S(=O)(Cl)c1ccc(-c2ncco2)cc1] | 244 |
| | 4-(oxazol-2-yl)benzenesulfonyl chloride | |

### Intermediate 3-1. 1-tert-butyl-6-(3-[[2-(2,3-dihydro-1,4-benzodioxine-6-sulfonyl)-1H,2H,3H-pyrrolo[3,4-c]pyridin-6-yl]carbonyl]phenyl)-1lambda3,3,6-oxadiazocan-2-one

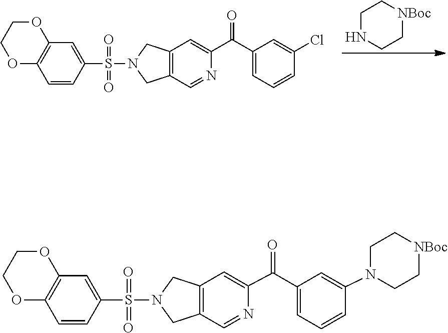 [SMILES: O=C(c1cccc(Cl)c1)c1cc2c(cn1)CN(S(=O)(=O)c1ccc3c(c1)OCCO3)C2 ; CC(C)(C)OC(=O)N1CCNCC1 ; CC(C)(C)OC(=O)N1CCN(c2cccc(C(=O)c3cc4c(cn3)CN(S(=O)(=O)c3ccc5c(c3)OCCO5)C4)c2)CC1]

To a solution of 6-[(3-chlorophenyl)carbonyl]-2-(2,3-dihydro-1,4-benzodioxine-6-sulfonyl)-1H,2H,3H-pyrrolo[3,4-c]pyridine (80 mg, 0.16 mmol) in toluene (8 mL) was added 1-tert-butyl-1lambda3,3,6-oxadiazocan-2-one (42 mg, 0.22 mmol), XPhos (19 mg, 0.04 mmol), Cs₂CO₃ (171 mg, 0.52 mmol) and Pd₂(dba)₃-CHCl₃ (18 mg, 0.02 mmol). The resulting mixture was stirred for 16 h at 100° C. and then cooled to room temperature. The reaction mixture was filtered and concentrated under vacuum. The resulting crude product was purified by silica gel chromatography (eluting with 1:10 EA/PE) to afford 1-tert-butyl-6-(3-[[2-(2,3-dihydro-1,4-benzodioxine-6-sulfonyl)-1H,2H,3H-pyrrolo[3,4-c]pyridin-6-yl]carbonyl]phenyl)-1lambda3,3,6-oxadiazocan-2-one as a yellow solid (80 mg, 84%). LCMS (ES, m/z) 607 [M+H]⁺.

The Intermediates in Table 3 were synthesized according to the procedure described for Intermediate 3-1 above.

TABLE 3

| | | LCMS: |
|---|---|---|
| | | (ESI) m/z |
| Intermediate | Compound | [M + H]⁺ |
| 3-2 | 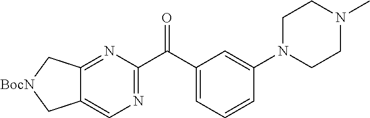 [SMILES: CN1CCN(c2cccc(C(=O)c3ncc4c(n3)CN(C(=O)OC(C)(C)C)C4)c2)CC1] | 424 |
| | tert-butyl 2-(3-(4-methylpiperazin-1-yl)benzoyl)-5,7- | |
| | dihydro-6H-pyrrolo[3,4-d]pyrimidine-6-carboxylate | |
| 3-3 | 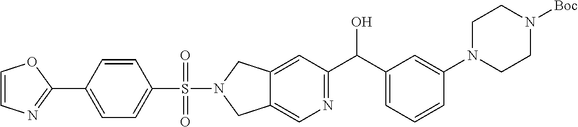 [SMILES: CC(C)(C)OC(=O)N1CCN(c2cccc(C(O)c3cc4c(cn3)CN(S(=O)(=O)c3ccc(-c5ncco5)cc3)C4)c2)CC1] | 618 |
| | tert-butyl 4-(3-(hydroxy(2-((4-(oxazol-2- | |
| | yl)phenyl)sulfonyl)-2,3-dihydro-1H-pyrrolo[3,4- | |
| | c]pyridin-6-yl)methyl)phenyl)piperazine-1-carboxylate | |
| 3-4^{a} | 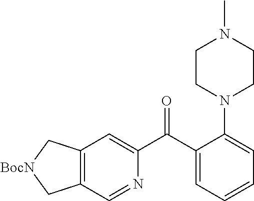 [SMILES: CN1CCN(c2ccccc2C(=O)c2cc3c(cn2)CN(C(=O)OC(C)(C)C)C3)CC1] | 423 |
| | tert-butyl 6-(2-(4-methylpiperazin-1-yl)benzoyl)-1,3- | |
| | dihydro-2H-pyrrolo[3,4-c]pyridine-2-carboxylate | |
| 3-5^{b} | 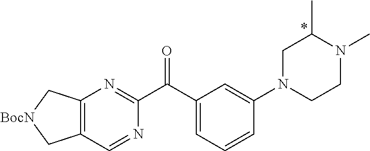 [SMILES: CC1CN(c2cccc(C(=O)c3ncc4c(n3)CN(C(=O)OC(C)(C)C)C4)c2)CCN1C] | 438 |
| | tert-butyl 2-(3-(3,4-dimethylpiperazin-1-yl)benzoyl)- | |
| | 5,7-dihydro-6H-pyrrolo[3,4-d]pyrimidine-6-carboxylate | |
| 3-6^{c} | 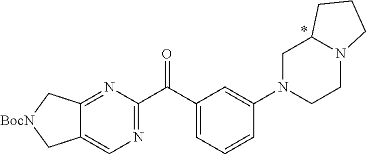 [SMILES: CC(C)(C)OC(=O)N1Cc2cnc(C(=O)c3cccc(N4CCN5CCCC5C4)c3)nc2C1] | 450 |
| | tert-butyl (S)-2-(3-(hexahydropyrrolo[1,2-a]pyrazin- | |
| | 2(1H)-yl)benzoyl)-5,7-dihydro-6H-pyrrolo[3,4- | |
| | d]pyrimidine-6-carboxylate | |
| 3-7^{b} | 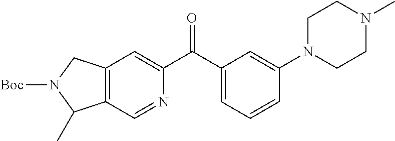 [SMILES: CC1c2cnc(C(=O)c3cccc(N4CCN(C)CC4)c3)cc2CN1C(=O)OC(C)(C)C] | 437 |
| | tert-butyl 3-methyl-6-(3-(4-methylpiperazin-1- | |
| | yl)benzoyl)-1,3-dihydro-2H-pyrrolo[3,4-c]pyridine-2- | |
| | carboxylate | |
| | 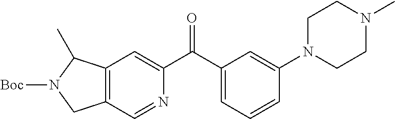 [SMILES: CC1c2cc(C(=O)c3cccc(N4CCN(C)CC4)c3)ncc2CN1C(=O)OC(C)(C)C] | |
| | tert-butyl 1-methyl-6-(3-(4-methylpiperazin-1- | |
| | yl)benzoyl)-1,3-dihydro-2H-pyrrolo[3,4-c]pyridine-2- | |
| | carboxylate | |
| 3-8^{d} | 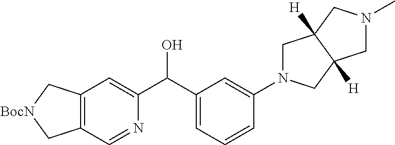 [SMILES: CN1C[C@@H]2CN(c3cccc(C(O)c4cc5c(cn4)CN(C(=O)OC(C)(C)C)C5)c3)C[C@@H]2C1] | 451 |
| | tert-butyl 6-(hydroxy(3-((3aR,6aS)-5- | |
| | methylhexahydropyrrolo[3,4-c]pyrrol-2(1H)- | |
| | yl)phenyl)methyl)-1,3-dihydro-2H-pyrrolo[3,4- | |
| | c]pyridine-2-carboxylate | |
| 3-9^{d} | 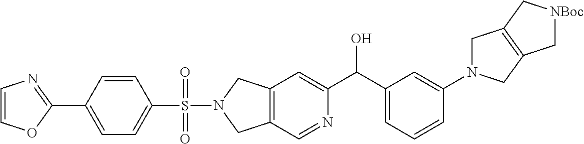 [SMILES: CC(C)(C)OC(=O)N1CC2=C(C1)CN(c1cccc(C(O)c3cc4c(cn3)CN(S(=O)(=O)c3ccc(-c5ncco5)cc3)C4)c1)C2] | 642 |
| | tert-butyl 5-(3-(hydroxy(2-((4-(oxazol-2- | |
| | yl)phenyl)sulfonyl)-2,3-dihydro-1H-pyrrolo[3,4- | |
| | c]pyridin-6-yl)methyl)phenyl)-3,4,5,6- | |
| | tetrahydropyrrolo[3,4-c]pyrrole-2(1H)-carboxylate | |

^{a}Pd₂(dba) ₃, BINAP, Cs₂CO₃, toluene, 100° C., 16 h;
^{b}Ruphos 3G, Ruphos, K₃PO₄, dioxane, 100° C., 16 h;
^{c}tBuXPhos 3G, BTMG, THF, 60° C.;
^{d}Ruphos 3G, Ruphos, Cs₂CO₃, dioxane, 100° C., 16 h;
*Absolute stereochemistry not determined.

### Intermediate 4-1. 1-(3-[[2-(2,3-dihydro-1,4-benzodioxine-6-sulfonyl)-1H,2H,3H-pyrrolo[3,4-c]pyridin-6-yl]carbonyl]phenyl) piperazine

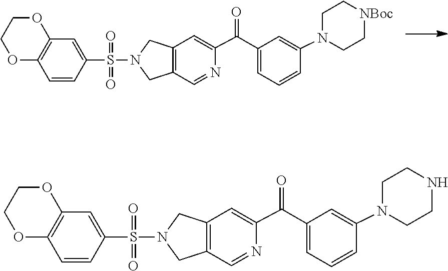 [SMILES: CC(C)(C)OC(=O)N1CCN(c2cccc(C(=O)c3cc4c(cn3)CN(S(=O)(=O)c3ccc5c(c3)OCCO5)C4)c2)CC1 ; O=C(c1cccc(N2CCNCC2)c1)c1cc2c(cn1)CN(S(=O)(=O)c1ccc3c(c1)OCCO3)C2]

To a solution of tert-butyl 4-(3-(2-((2,3-dihydrobenzo[b][1,4]dioxin-6-yl)sulfonyl)-2,3-dihydro-1H-pyrrolo[3,4-c]pyridine-6-carbonyl)phenyl)piperazine-1-carboxylate (80 mg, 0.12 mmol) in DCM (3 mL) was added TFA (1 mL). The resulting solution was stirred for 1 h at rt and then concentrated under vacuum. The resulting mixture was then basified to pH 8 with saturated aqueous potassium carbonate solution. The resulting mixture was extracted with DCM (3×5 mL). The combined organic layers were dried over anhydrous sodium sulfate, filtered and concentrated under vacuum to afford 1-(3-[[2-(2,3-dihydro-1,4-benzodioxine-6-sulfonyl)-1H,2H,3H-pyrrolo[3,4-c]pyridin-6-yl]carbonyl]phenyl)piperazine as a yellow solid (50 mg, 83%). LCMS (ES, m/z) 507 [M+H]⁺.

The Intermediates in Table 4 were synthesized according to the procedure described for Intermediate 4-1 above.

TABLE 4

| | | LCMS: |
|---|---|---|
| | | (ESI) m/z |
| Intermediate | Compound | [M + H]⁺ |
| 4-2 | 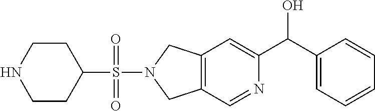 [SMILES: O=S(=O)(C1CCNCC1)N1Cc2cnc(C(O)c3ccccc3)cc2C1] | 374 |
| | phenyl(2-(piperidin-4-ylsulfonyl)-2,3-dihydro-1H- | |
| | pyrrolo[3,4-c]pyridin-6-yl)methanol- TFA salt | |
| 4-3 | 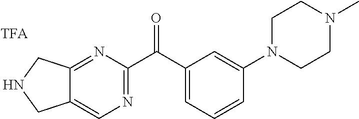 [SMILES: CN1CCN(c2cccc(C(=O)c3ncc4c(n3)CNC4)c2)CC1] | 324 |
| | (6,7-dihydro-5H-pyrrolo[3,4-d]pyrimidin-2-yl)(3-(4- | |
| | methylpiperazin-1-yl)phenyl)methanone TFA salt | |
| 4-4^{a} | 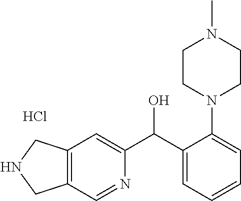 [SMILES: CN1CCN(c2ccccc2C(O)c2cc3c(cn2)CNC3)CC1 ; Cl] | 325 |
| | (2,3-dihydro-1H-pyrrolo[3,4-c]pyridin-6-yl)(2-(4- | |
| | methylpiperazin-1-yl)phenyl)methanol hydrochloride | |
| | HCl salt | |
| 4-5^{a} | 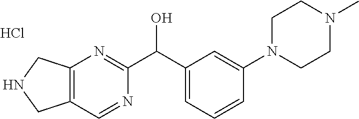 [SMILES: Cl ; CN1CCN(c2cccc(C(O)c3ncc4c(n3)CNC4)c2)CC1] | 326 |
| | (6,7-dihydro-5H-pyrrolo[3,4-d]pyrimidin-2-yl)(3-(4- | |
| | methylpiperazin-1-yl)phenyl)methanol HCl salt | |
| 4-6 | 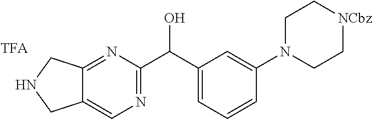 [SMILES: O=C(OCc1ccccc1)N1CCN(c2cccc(C(O)c3ncc4c(n3)CNC4)c2)CC1 ; "TFA"] | 446 |
| | benzyl 4-(3-((6,7-dihydro-5H-pyrrolo[3,4-d]pyrimidin- | |
| | 2-yl)(hydroxy)methyl)phenyl)piperazine-1-carboxylate | |
| | TFA salt | |
| 4-7 | 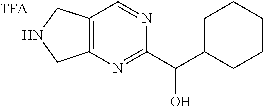 [SMILES: OC(c1ncc2c(n1)CNC2)C1CCCCC1] | 234 |
| | cyclohexyl(6,7-dihydro-5H-pyrrolo[3,4-d]pyrimidin-2- | |
| | yl)methanol TFA salt | |
| 4-9 | 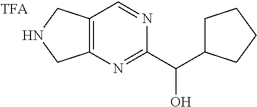 [SMILES: OC(c1ncc2c(n1)CNC2)C1CCCC1] | 220 |
| | cyclopentyl(6,7-dihydro-5H-pyrrolo[3,4-d]pyrimidin- | |
| | 2-yl)methanol TFA salt | |
| 4-11 | 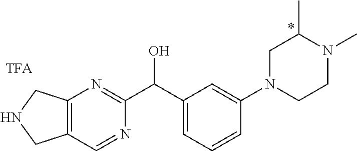 [SMILES: CC1CN(c2cccc(C(O)c3ncc4c(n3)CNC4)c2)CCN1C] | 340 |
| | (6,7-dihydro-5H-pyrrolo[3,4-d]pyrimidin-2-yl)(3-(3,4- | |
| | dimethylpiperazin-1-yl)phenyl)methanol TFA salt | |
| 4-12 | 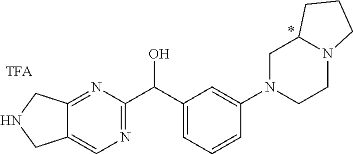 [SMILES: OC(c1cccc(N2CCN3CCCC3C2)c1)c1ncc2c(n1)CNC2] | 350 |
| | (6,7-dihydro-5H-pyrrolo[3,4-d]pyrimidin-2-yl)(3- | |
| | (hexahydropyrrolo[1,2-a]pyrazin-2(1H)- | |
| | yl)phenyl)methanone TFA salt | |
| 4-13 | 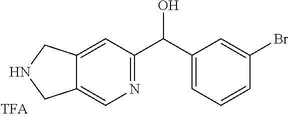 [SMILES: OC(c1cccc(Br)c1)c1cc2c(cn1)CNC2] | 305, 307 |
| | (3-bromophenyl)(2,3-dihydro-1H-pyrrolo[3,4- | |
| | c]pyridin-6-yl)methanol TFA salt | |
| 4-14^{a} | 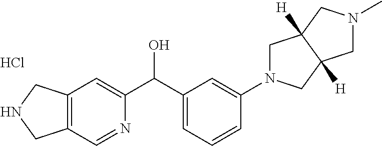 [SMILES: Cl ; CN1C[C@@H]2CN(c3cccc(C(O)c4cc5c(cn4)CNC5)c3)C[C@@H]2C1] | 351 |
| | (2,3-dihydro-1H-pyrrolo[3,4-c]pyridin-6-yl)(3- | |
| | ((3aR,6aS)-5-methylhexahydropyrrolo[3,4-c]pyrrol- | |
| | 2(1H)-yl)phenyl)methanol HCl salt | |
| 4-15 | 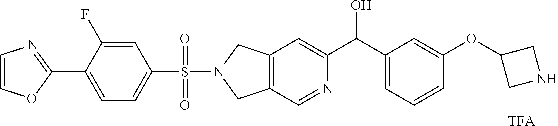 [SMILES: O=S(=O)(c1ccc(-c2ncco2)c(F)c1)N1Cc2cnc(C(O)c3cccc(OC4CNC4)c3)cc2C1 ; "TFA"] | 522 |
| | (3-(azetidin-3-yloxy)phenyl)(2-((3-fluoro-4-(oxazol-2- | |
| | yl)phenyl)sulfonyl)-2,3-dihydro-1H-pyrrolo[3,4- | |
| | c]pyridin-6-yl)methanol TFA salt | |
| 4-16 | 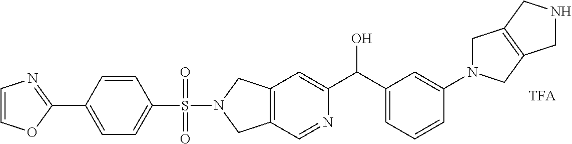 [SMILES: O=S(=O)(c1ccc(-c2ncco2)cc1)N1Cc2cnc(C(O)c3cccc(N4CC5=C(CNC5)C4)c3)cc2C1 ; "TFA"] | 542 |
| | (2-((4-(oxazol-2-yl)phenyl)sulfonyl)-2,3-dihydro-1H- | |
| | pyrrolo[3,4-c]pyridin-6-yl)(3-(3,4,5,6- | |
| | tetrahydropyrrolo[3,4-c]pyrrol-2(1H)- | |
| | yl)phenyl)methanol TFA salt | |

^{a}HCl salts were prepared from 4M HCl in dioxane in a stirred flask at room temperature;
*Absolute stereochemistry not determined.

### Intermediate 5-1. Methyl 3-methoxy-4-(1H-pyrazol-1-yl)benzene-1-sulfonyl chloride

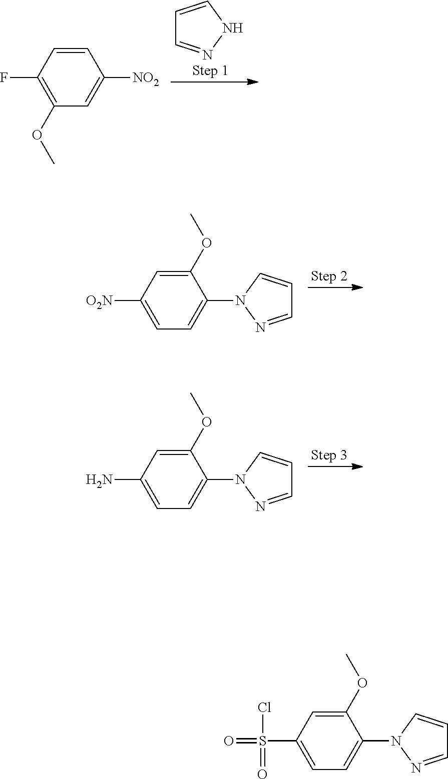 [SMILES: COc1cc([N+](=O)[O-])ccc1F ; c1cn[nH]c1 ; COc1cc([N+](=O)[O-])ccc1-n1cccn1 ; COc1cc(N)ccc1-n1cccn1 ; COc1cc(S(=O)(=O)Cl)ccc1-n1cccn1]

### Step 1. 1-(2-Methoxy-4-nitrophenyl)-1H-pyrazole

To a solution of 1H-pyrazole (2 g, 29.4 mmol) in DMF (30 mL) was added 1-fluoro-2-methoxy-4-nitrobenzene (5 g, 29.2 mmol), and potassium carbonate (12 g, 86.8 mmol). The resulting mixture was stirred for 14 h at 120° C. and then cooled to room temperature. The reaction mixture was poured into water (30 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 1-(2-methoxy-4-nitrophenyl)-1H-pyrazole as light yellow solid (5.0 g, 78%). LCMS (ES, m/z) 220 [M+H]⁺.

### Step 2. 3-Methoxy-4-(1H-pyrazol-1-yl)aniline

To a solution of 1-(2-methoxy-4-nitrophenyl)-1H-pyrazole (5 g, 22.8 mmol) in THF (30 mL) was added water (10 mL), EtOH (30 mL), iron powder (3.8 g, 68.1 mmol) and ammonium chloride (3.6 g, 67.3 mmol). The resulting mixture was stirred for 16 h at 80° C. and cooled to room temperature. The reaction mixture was filtered and extracted with EA (3×50 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 3-methoxy-4-(1H-pyrazol-1-yl) aniline as a black solid (4.9 g, crude). LCMS (ES, m/z) 190 [M+H]⁺.

### Step 3. Methyl 3-methoxy-4-(1H-pyrazol-1-yl)benzene-1-sulfonyl chloride

Into glacial acetic acid (20 mL) was bubbled in SO₂ gas for 1 h at room temperature. Then CuCl₂ (800 mg, 5.95 mmol) was added and S02 gas was bubbled in for additional 2 h to afford solution A. To a pre-cooled solution of 3-methoxy-4-(1H-pyrazol-1-yl)aniline (4.5 g, 23.8 mmol) in acetic acid (4 mL) and concentrated hydrochloric acid (12 mL) was added a solution of sodium nitrite (1.8 g, 26.1 mmol) in distilled water (5 mL) dropwise with stirring at −10° C. After stirring for 15 min, solution A was added to this diazonium salt solution at −10° C. The resulting solution was allowed to warm to room temperature naturally and stirred for 16 h. The reaction mixture was treated with water (100 mL) and then extracted with EA (3×100 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford methyl 3-methoxy-4-(1H-pyrazol-1-yl)benzene-1-sulfonyl chloride as a yellow solid (3.0 g, 46%). LCMS (EI, m/z): 273, 275 [M+H]⁺.

### Intermediate 6-1. 6-benzoyl-1H,2H,3H-pyrrolo[3,4-c]pyridine

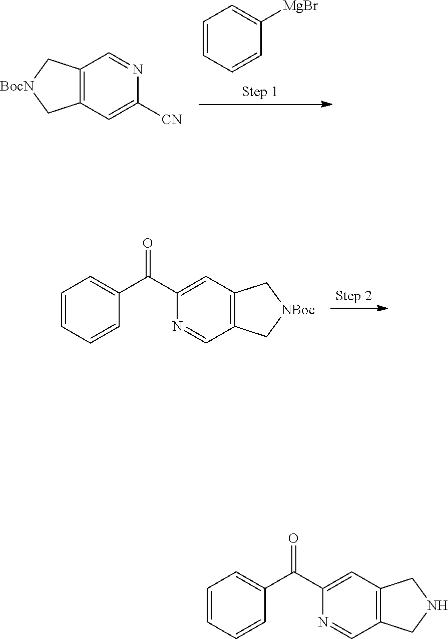

### Step 1. Tert-butyl 6-benzoyl-1H,2H,3H-pyrrolo[3,4-c]pyridine-2-carboxylate

To a solution of tert-butyl 6-cyano-1H,2H,3H-pyrrolo[3,4-c]pyridine-2-carboxylate (670 mg, 2.73 mmol) in THF (10 mL), was added a solution of bromo(phenyl)magnesium (5.5 mL, 1 M in THF) at 0° C. The resulting solution was stirred for 1 h at room temperature. The reaction mixture was treated with 1 N HCl (10 mL) for 30 min and then extracted with ethyl acetate (3×10 mL). The combined organic layers were dried over anhydrous sodium sulfate, filtered and concentrated under vacuum. The resulting crude product was purified by silica gel chromatography (eluting with 0:100 to 50:50 ethyl acetate/petroleum ether) to afford tert-butyl 6-benzoyl-1H,2H,3H-pyrrolo[3,4-c]pyridine-2-carboxylate as a white solid (600 mg, 68%). LCMS (ES, m/z) 325 [M+H]⁺.

### Step 2. 6-benzoyl-1H,2H,3H-pyrrolo[3,4-c]pyridine

To a solution of tert-butyl 6-benzoyl-1H,2H,3H-pyrrolo[3,4-c]pyridine-2-carboxylate (200 mg, 0.62 mmol) in dichloromethane (4 mL) was added TFA (1 mL). The resulting solution was stirred for 2 h at room temperature and concentrated under vacuum. The resulting mixture was basified to pH 8 with saturated potassium carbonate solution and extracted with dichloromethane (3×5 mL). The combined organic layers were dried over anhydrous sodium sulfate, filtered and concentrated under vacuum to afford 6-benzoyl-1H,2H,3H-pyrrolo[3,4-c]pyridine as brown oil (150 mg, crude). LCMS (ES, m/z) 225 [M+H]⁺.

### Intermediate 7-1. 4-(1H-imidazol-1-yl) benzene-1-sulfonyl chloride

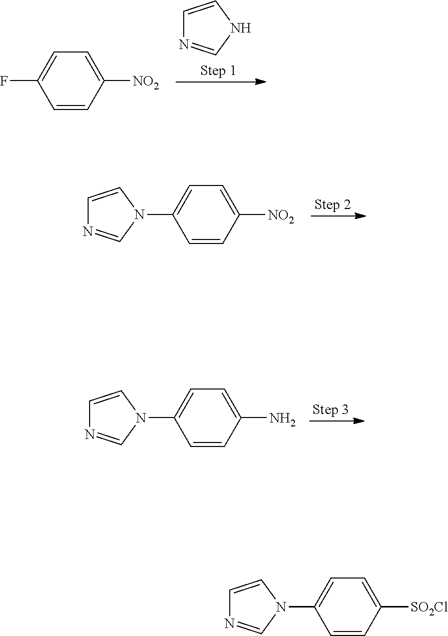 [SMILES: O=[N+]([O-])c1ccc(F)cc1 ; c1c[nH]cn1 ; O=[N+]([O-])c1ccc(-n2ccnc2)cc1 ; Nc1ccc(-n2ccnc2)cc1 ; O=S(=O)(Cl)c1ccc(-n2ccnc2)cc1]

### Step 1. 1-(4-nitrophenyl)-1H-imidazole

To a solution of 1-fluoro-4-nitrobenzene (3 g, 21.3 mmol), in N,N-dimethylformamide (30 mL), was added potassium carbonate (8.8 g, 63.7 mmol) and 1H-imidazole (1.4 g, 20.6 mmol). The resulting mixture was stirred for 18 h at 120° C. The reaction mixture was cooled and poured into water (30 mL). The resulting solution was extracted with ethyl acetate (3×100 mL). The combined organic layers were dried over anhydrous sodium sulfate, filtered, and concentrated under vacuum. The resulting crude product was purified by silica gel chromatography (eluting with 0:100 to 10:80 ethyl acetate/petroleum ether) to afford 1-(4-nitrophenyl)-1H-imidazole as a yellow solid (4.0 g, 99%). LCMS (ES, m/z) 190 [M+H]⁺.

### Step 2. 4-(1H-imidazol-1-yl)aniline

To a solution of 1-(4-nitrophenyl)-1H-imidazole (4 g, 21.14 mmol) in THF (30 mL), was added water (10 mL), EtOH (30 mL), iron powder (3.5 g, 62.7 mmol), and ammonium chloride (3.4 g, 63.6 mmol). The resulting mixture was stirred for 2 h at 80° C. and cooled to room temperature. The reaction mixture was filtered and extracted with EA (3×50 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 4-(1H-imidazol-1-yl) aniline as a black solid (2.0 g, 59%). LCMS (ES, m/z) 160 [M+H]⁺.

### Step 3. 4-(1H-imidazol-1-yl) benzene-1-sulfonyl chloride

Into glacial acetic acid (20 mL) was bubbled S02 gas for 1 h at room temperature. Then CuCl₂ (210 mg, 1.56 mmol) was added and SO₂ gas was bubbled in for additional 2 h to afford solution A. To a pre-cooled solution of 4-(1H-imidazol-1-yl) aniline (1 g, 6.28 mmol) in acetic acid (4 mL) and concentrated hydrochloric acid (12 mL), was added a solution of sodium nitrite (470 mg, 6.09 mmol) in distilled water (1 mL) dropwise with stirring at −10° C. After stirring for 15 min, solution A was added to this diazonium salt solution at −10° C. The resulting solution was allowed to warm to room temperature naturally and stirred for 16 h. The reaction mixture was treated with water (20 mL) and then extracted with EA (3×20 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 4-(1H-imidazol-1-yl) benzene-1-sulfonyl chloride as a white solid (400 mg, 26%). LCMS (EI, m/z): 243, 245 [M+H]⁺.

The Intermediates in Table 5 were synthesized according to the procedure described for Intermediate 7-1 above.

TABLE 5

| | | LCMS: (ESI) |
|---|---|---|
| Intermediate | Compound | m/z [M + H]⁺ |
| 7-2 | 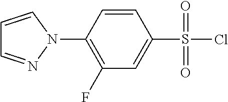 [SMILES: O=S(=O)(Cl)c1ccc(-n2cccn2)c(F)c1] | — |
| | 3-fluoro-4-(1H-pyrazol-1-yl) | |
| | benzenesulfonyl chloride | |

### Intermediate 8-1. 1-[4-({6-benzoyl-1H,2H,3H-pyrrolo[3,4-c]pyridinyl}sulfonyl)phenyl]-1H-imidazole

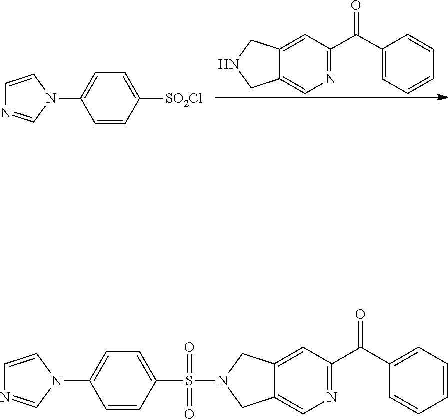 [SMILES: O=S(=O)(Cl)c1ccc(-n2ccnc2)cc1 ; O=C(c1ccccc1)c1cc2c(cn1)CNC2 ; O=C(c1ccccc1)c1cc2c(cn1)CN(S(=O)(=O)c1ccc(-n3ccnc3)cc1)C2]

To a solution of 4-(1H-imidazol-1-yl)benzene-1-sulfonyl chloride (200 mg, 0.82 mmol) in DCM (2 mL) was added 6-benzoyl-1H,2H,3H-pyrrolo[3,4-c]pyridine (124 mg, 0.55 mmol) and TEA (0.22 mL, 1.60 mmol). The resulting solution was stirred for 2 h at 25° C. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 1-[4-([6-benzoyl-1H,2H,3H-pyrrolo[3,4-c]pyridin-2-yl]sulfonyl)phenyl]-1H-imidazole as a white solid (95 mg, 22%). LCMS (ES, m/z) 431 [M+H]⁺.

The Intermediates in Table 6 were synthesized according to the procedure described for Intermediate 8-1 above.

TABLE 6

| | | LCMS: (ESI) |
|---|---|---|
| Intermediate | Compound | m/z [M + H]⁺ |
| 8-2 | 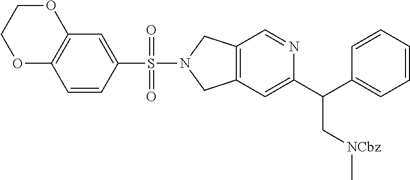 [SMILES: CN(CC(c1ccccc1)c1cc2c(cn1)CN(S(=O)(=O)c1ccc3c(c1)OCCO3)C2)C(=O)OCc1ccccc1] | 586 |
| | (3-[2-[2-(2,3-dihydro-1,4-benzodioxine-6-sulfonyl)- | |
| | 1H,2H,3H-pyrrolo[3,4-c]pyridin-6-yl]-2-phenylethyl] | |
| | phenyl)methyl N-methylcarbamate | |
| 8-3 | 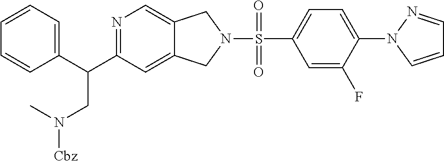 [SMILES: CN(CC(c1ccccc1)c1cc2c(cn1)CN(S(=O)(=O)c1ccc(-n3cccn3)c(F)c1)C2)C(=O)OCc1ccccc1] | 612 |
| | Benzyl N-(2-{2-[3-fluoro-4-(1H-pyrazol-1- | |
| | yl)benzenesulfonyl]-1H,2H,3H-pyrrolo[3,4-c] pyridine- | |
| | 6-yl}-2-phenylethyl)-N-methylcarbamate | |
| 8-4 | 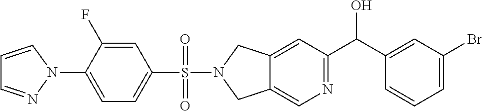 [SMILES: O=S(=O)(c1ccc(-n2cccn2)c(F)c1)N1Cc2cnc(C(O)c3cccc(Br)c3)cc2C1] | 529, 531 |
| | (3-bromophenyl)([2-[3-fluoro-4-(1H-pyrazol-1- | |
| | yl)benzenesulfonyl]-1H,2H,3H-pyrrolo[3,4-c]pyridine- | |
| | 6-yl])methanol | |
| 8-5 | 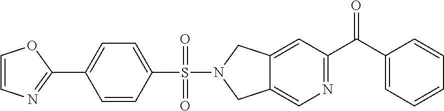 [SMILES: O=C(c1ccccc1)c1cc2c(cn1)CN(S(=O)(=O)c1ccc(-c3ncco3)cc1)C2] | 432 |
| | (2-((4-(oxazol-2-yl)phenyl)sulfonyl)-2,3-dihydro-1H- | |
| | pyrrolo[3,4-c]pyridin-6-yl)(phenyl)methanone | |
| 8-6 | 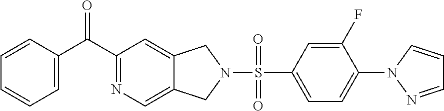 [SMILES: O=C(c1ccccc1)c1cc2c(cn1)CN(S(=O)(=O)c1ccc(-n3cccn3)c(F)c1)C2] | 449 |
| | (2-((3-fluoro-4-(1H-pyrazol-1-yl)phenyl)sulfonyl)-2,3- | |
| | dihydro-1H-pyrrolo[3,4-c]pyridin-6- | |
| | yl)(phenyl)methanone | |
| 8-7 | 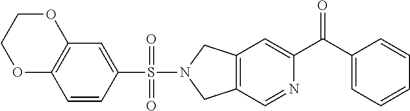 [SMILES: O=C(c1ccccc1)c1cc2c(cn1)CN(S(=O)(=O)c1ccc3c(c1)OCCO3)C2] | 423 |
| | (2-((2,3-dihydrobenzo[b][1,4]dioxin-6-yl)sulfonyl)-2,3- | |
| | dihydro-1H-pyrrolo[3,4-c]pyridin-6- | |
| | yl)(phenyl)methanone | |
| 8-8 | 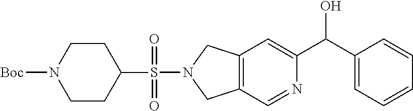 [SMILES: CC(C)(C)OC(=O)N1CCC(S(=O)(=O)N2Cc3cnc(C(O)c4ccccc4)cc3C2)CC1] | 474 |
| | tert-butyl 4-((6-(hydroxy(phenyl)methyl)-1,3-dihydro- | |
| | 2H-pyrrolo[3,4-c]pyridin-2-yl)sulfonyl)piperidine-1- | |
| | carboxylate | |
| 8-9 | 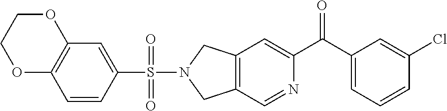 [SMILES: O=C(c1cccc(Cl)c1)c1cc2c(cn1)CN(S(=O)(=O)c1ccc3c(c1)OCCO3)C2] | 459 |
| | (3-chlorophenyl)(2-((2,3-dihydrobenzo[b][1,4]dioxin-6- | |
| | yl)sulfonyl)-2,3-dihydro-1H-pyrrolo[3,4-c]pyridin-6- | |
| | yl)methanone | |
| 8-10 | 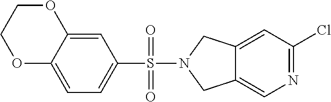 [SMILES: O=S(=O)(c1ccc2c(c1)OCCO2)N1Cc2cnc(Cl)cc2C1] | 353 |
| | 6-chloro-2-((2,3-dihydrobenzo[b][1,4]dioxin-6- | |
| | yl)sulfonyl)-2,3-dihydro-1H-pyrrolo[3,4-c]pyridine | |
| 8-11 | 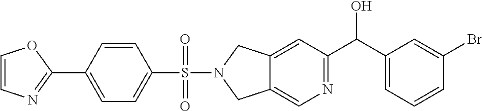 [SMILES: O=S(=O)(c1ccc(-c2ncco2)cc1)N1Cc2cnc(C(O)c3cccc(Br)c3)cc2C1] | 512, 514 |
| | (3-bromophenyl)(2-((4-(oxazol-2-yl)phenyl)sulfonyl)- | |
| | 2,3-dihydro-1H-pyrrolo[3,4-c]pyridin-6-yl)methanol | |
| 8-12 | 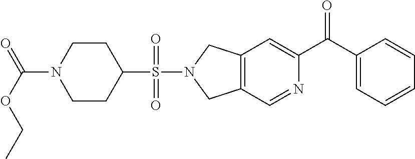 [SMILES: CCOC(=O)N1CCC(S(=O)(=O)N2Cc3cnc(C(=O)c4ccccc4)cc3C2)CC1] | 444 |
| | ethyl 4-((6-benzoyl-1,3-dihydro-2H-pyrrolo[3,4- | |
| | c]pyridin-2-yl)sulfonyl)piperidine-1-carboxylate | |
| 8-13 | 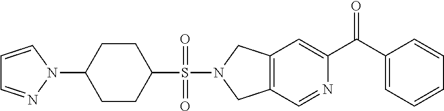 [SMILES: O=C(c1ccccc1)c1cc2c(cn1)CN(S(=O)(=O)C1CCC(n3cccn3)CC1)C2] | 437 |
| | (2-((4-(1H-pyrazol-1-yl)cyclohexyl)sulfonyl)-2,3- | |
| | dihydro-1H-pyrrolo[3,4-c]pyridin-6- | |
| | yl)(phenyl)methanone | |
| 8-14 | 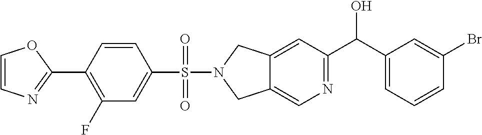 [SMILES: O=S(=O)(c1ccc(-c2ncco2)c(F)c1)N1Cc2cnc(C(O)c3cccc(Br)c3)cc2C1] | 530, 532 |
| | (3-bromophenyl)(2-((3-fluoro-4-(oxazol-2- | |
| | yl)phenyl)sulfonyl)-2,3-dihydro-1H-pyrrolo[3,4- | |
| | c]pyridin-6-yl)methanol | |
| 8-15 | 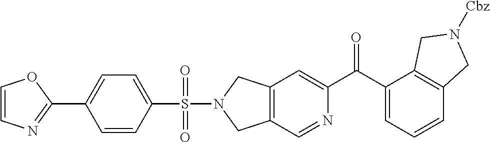 [SMILES: O=C(c1cc2c(cn1)CN(S(=O)(=O)c1ccc(-c3ncco3)cc1)C2)c1cccc2c1CN(C(=O)OCc1ccccc1)C2] | 607 |
| | benzyl 4-(2-((4-(oxazol-2-yl)phenyl)sulfonyl)-2,3- | |
| | dihydro-1H-pyrrolo[3,4-c]pyridine-6- | |
| | carbonyl)isoindoline-2-carboxylate | |
| 8-16 | 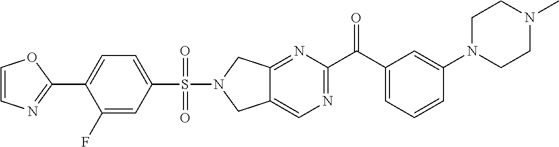 [SMILES: CN1CCN(c2cccc(C(=O)c3ncc4c(n3)CN(S(=O)(=O)c3ccc(-c5ncco5)c(F)c3)C4)c2)CC1] | 549 |
| | (6-((3-fluoro-4-(oxazol-2-yl)phenyl)sulfonyl)-6,7- | |
| | dihydro-5H-pyrrolo[3,4-d]pyrimidin-2-yl)(3-(4- | |
| | methylpiperazin-1-yl)phenyl)methanone | |
| 8-17 | 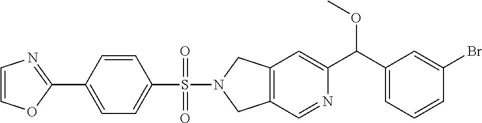 [SMILES: COC(c1cccc(Br)c1)c1cc2c(cn1)CN(S(=O)(=O)c1ccc(-c3ncco3)cc1)C2] | 526, 528 |
| | 2-(4-((6-((3-bromophenyl)(methoxy)methyl)-1,3- | |
| | dihydro-2H-pyrrolo[3,4-c]pyridin-2- | |
| | yl)sulfonyl)phenyl)oxazole | |
| 8-18 | 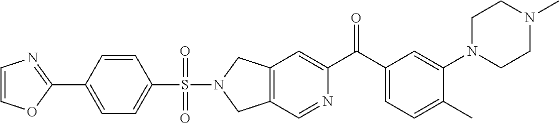 [SMILES: Cc1ccc(C(=O)c2cc3c(cn2)CN(S(=O)(=O)c2ccc(-c4ncco4)cc2)C3)cc1N1CCN(C)CC1] | 544 |
| | (4-methyl-3-(4-methylpiperazin-1-yl)phenyl)(2-((4- | |
| | (oxazol-2-yl)phenyl)sulfonyl)-2,3-dihydro-1H- | |
| | pyrrolo[3,4-c]pyridin-6-yl)methanone | |
| 8-19 | 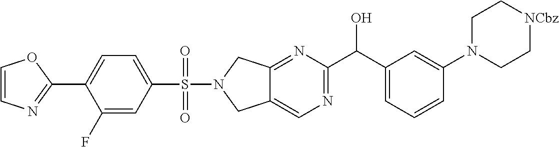 [SMILES: O=C(OCc1ccccc1)N1CCN(c2cccc(C(O)c3ncc4c(n3)CN(S(=O)(=O)c3ccc(-c5ncco5)c(F)c3)C4)c2)CC1] | 537 |
| | benzyl 4-(3-((6-((3-fluoro-4-(oxazol-2- | |
| | yl)phenyl)sulfonyl)-6,7-dihydro-5H-pyrrolo[3,4- | |
| | d]pyrimidin-2-yl)(hydroxy)methyl)phenyl)piperazine-1- | |
| | carboxylate | |
| 8-20 | 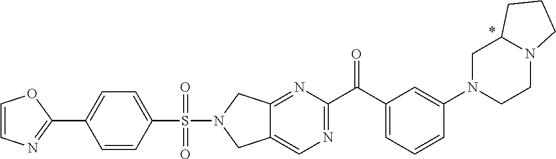 [SMILES: O=C(c1cccc(N2CCN3CCCC3C2)c1)c1ncc2c(n1)CN(S(=O)(=O)c1ccc(-c3ncco3)cc1)C2] | 557 |
| | (3-(hexahydropyrrolo[1,2-a]pyrazin-2(1H)- | |
| | yl)phenyl)(6-((4-(oxazol-2-yl)phenyl)sulfonyl)-6,7- | |
| | dihydro-5H-pyrrolo[3,4-d]pyrimidin-2-yl)methanone | |
| 8-21 | 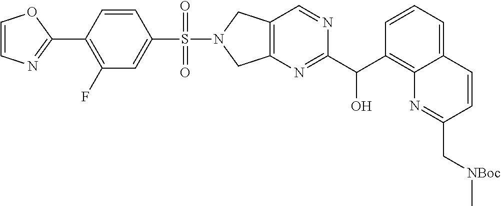 [SMILES: CN(Cc1ccc2cccc(C(O)c3ncc4c(n3)CN(S(=O)(=O)c3ccc(-c5ncco5)c(F)c3)C4)c2n1)C(=O)OC(C)(C)C] | 647 |
| | tert-butyl ((8-((6-((3-fluoro-4-(oxazol-2- | |
| | yl)phenyl)sulfonyl)-6,7-dihydro-5H-pyrrolo[3,4- | |
| | d]pyrimidin-2-yl)(hydroxy)methyl)quinolin-2- | |
| | yl)methyl)(methyl)carbamate | |
| 8-22 | 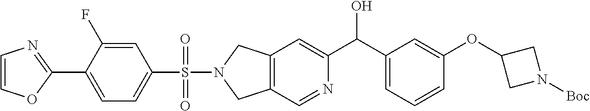 [SMILES: CC(C)(C)OC(=O)N1CC(Oc2cccc(C(O)c3cc4c(cn3)CN(S(=O)(=O)c3ccc(-c5ncco5)c(F)c3)C4)c2)C1] | 623 |
| | tert-butyl 3-(3-((2-((3-fluoro-4-(oxazol-2- | |
| | yl)phenyl)sulfonyl)-2,3-dihydro-1H-pyrrolo[3,4- | |
| | c]pyridin-6-yl)(hydroxy)methyl)phenoxy)azetidine-1- | |
| | carboxylate | |

*Absolute stereochemistry is not determined.

### Intermediate 9-1. Tert-butyl N-(3-{2-[3-fluoro-4-(1H-pyrazol-1-yl)benzenesulfonyl]-1H,2H,3H-pyrrolo[3,4-c]pyridine-6-yl}-3-phenylpropyl)-N-methylcarbamate

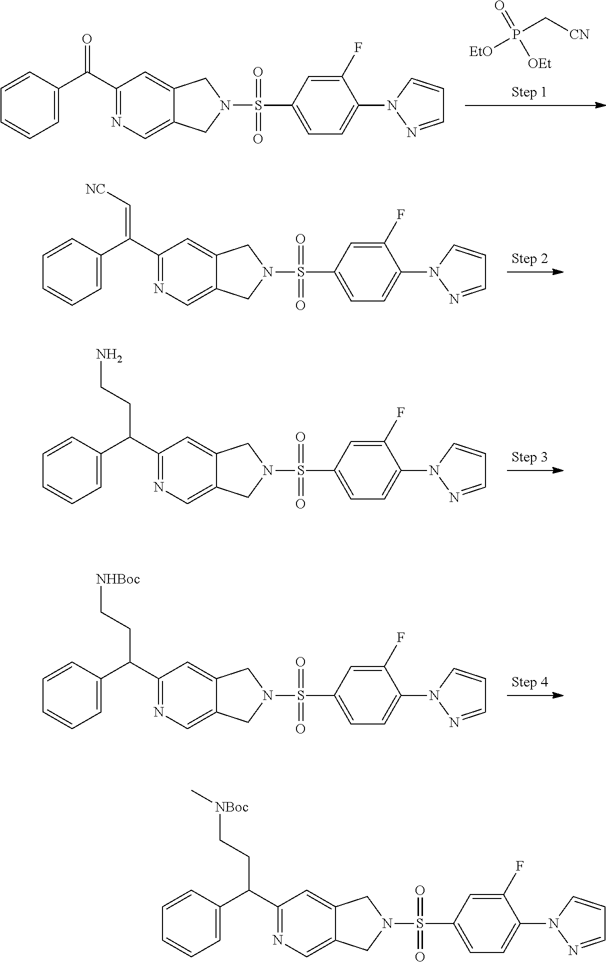 [SMILES: O=C(c1ccccc1)c1cc2c(cn1)CN(S(=O)(=O)c1ccc(-n3cccn3)c(F)c1)C2 ; CCOP(=O)(CC#N)OCC ; N#C/C=C(\c1ccccc1)c1cc2c(cn1)CN(S(=O)(=O)c1ccc(-n3cccn3)c(F)c1)C2 ; NCCC(c1ccccc1)c1cc2c(cn1)CN(S(=O)(=O)c1ccc(-n3cccn3)c(F)c1)C2 ; CC(C)(C)OC(=O)NCCC(c1ccccc1)c1cc2c(cn1)CN(S(=O)(=O)c1ccc(-n3cccn3)c(F)c1)C2 ; CN(CCC(c1ccccc1)c1cc2c(cn1)CN(S(=O)(=O)c1ccc(-n3cccn3)c(F)c1)C2)C(=O)OC(C)(C)C]

### Step 1. 3-{2-[3-fluoro-4-(1H-pyrazol-1-yl)benzenesulfonyl]-1H,2H,3H-pyrrolo[3,4-c]pyridine-6-yl}-3-phenylprop-2-enenitrile

To a solution of diethyl (cyanomethyl)phosphonate (65 mg, 0.37 mmol) in N,N-dimethylformamide (3 mL) was added sodium hydride (20 mg, 0.83 mmol, 60% dispersion in mineral oil) at 0° C. The resulting mixture was stirred for 30 min at 0° C. This was followed by addition of a solution of 1-[4-([6-benzoyl-1H,2H,3H-pyrrolo[3,4-c]pyridin-2-yl]sulfonyl)-2-fluorophenyl]-1H-pyrazole (110 mg, 0.25 mmol) in DMF (1 mL). The resulting solution was stirred for 1 h at room temperature. The reaction mixture was poured into saturated ammonium chloride solution (5 mL) and then extracted with ethyl acetate (3×5 mL). The combined organic layers were dried over anhydrous sodium sulfate, filtered and concentrated under vacuum. The resulting crude product was purified by prep-TLC (eluting with 1:1 ethyl acetate/petroleum ether) to afford 3-[2-[3-fluoro-4-(1H-pyrazol-1-yl)benzenesulfonyl]-1H,2H,3H-pyrrolo[3,4-c]pyridin-6-yl]-3-phenylprop-2-enenitrile as light yellow oil (90 mg, 78%). LCMS (ES, m/z) 472 [M+H]⁺.

### Step 2. 3-{2-[3-fluoro-4-(1H-pyrazol-1-yl)benzenesulfonyl]-1H,2H,3H-pyrrolo[3,4-c]pyridine-6-yl}-3-phenylpropan-1-amine

To a solution of 3-[2-[3-fluoro-4-(1H-pyrazol-1-yl)benzenesulfonyl]-1H,2H,3H-pyrrolo[3,4-c]pyridin-6-yl]-3-phenylprop-2-enenitrile (90 mg, 0.19 mmol) in methanol (2 mL) was added palladium carbon (10 mg, 10 wt % palladium on charcoal). Then hydrogen was introduced with hydrogen balloon. The resulting mixture was stirred for 16 h at room temperature. The reaction mixture was filtered and concentrated under vacuum to afford 3-[2-[3-fluoro-4-(1H-pyrazol-1-yl)benzenesulfonyl]-1H,2H,3H-pyrrolo[3,4-c]pyridin-6-yl]-3-phenylpropan-1-amine as yellow oil (50 mg, 55%). LCMS (ES, m/z) 478 [M+H]⁺.

### Step 3. Tert-butyl N-(3-{2-[3-fluoro-4-(1H-pyrazol-1-yl)benzenesulfonyl]-1H,2H,3H-pyrrolo [3,4-c]pyridine-6-yl}-3-phenylpropyl)carbamate

To a solution of 3-[2-[3-fluoro-4-(1H-pyrazol-1-yl)benzenesulfonyl]-1H,2H,3H-pyrrolo [3,4-c]pyridin-6-yl]-3-phenylpropan-1-amine (50 mg, 0.10 mmol) in dichloromethane (1 mL) was added TEA (32 mg, 0.32 mmol) and di-tert-butyl dicarbonate (46 mg, 0.21 mmol). The resulting solution was stirred for 2 h at room temperature and concentrated under vacuum. The resulting crude product was purified by prep-TLC (eluting with 2:1 ethyl acetate/petroleum ether) to afford tert-butyl N-(3-[2-[3-fluoro-4-(1H-pyrazol-1-yl)benzenesulfonyl]-1H,2H,3H-pyrrolo[3,4-c]pyridin-6-yl]-3-phenylpropyl)carbamate as light yellow oil (40 mg, 66%). LCMS (ES, m/z) 578 [M+H]⁺.

### Step 4. Tert-butyl N-(3-{2-[3-fluoro-4-(1H-pyrazol-1-yl)benzenesulfonyl]-1H,2H,3H-pyrrolo[3,4-c]pyridine-6-yl}-3-phenylpropyl)-N-methylcarbamate

To a solution of tert-butyl N-(3-[2-[3-fluoro-4-(1H-pyrazol-1-yl)benzenesulfonyl]-1H,2H,3H-pyrrolo [3,4-c] pyridin-6-yl]-3-phenylpropyl)carbamate (40 mg, 0.07 mmol) in tetrahydrofuran (1 mL) was added sodium hydride (6 mg, 0.15 mmol, 60% dispersion in mineral oil) at 0° C. The resulting mixture was stirred for 30 min at 0° C. This was followed by the addition of iodomethane (15 mg, 0.11 mmol). The resulting solution was stirred for 16 h at room temperature. The reaction mixture was poured into saturated NH₄Cl (2 mL) and then extracted with ethyl acetate (3×3 mL). The combined organic layers were dried over anhydrous sodium sulfate, filtered and concentrated under vacuum. The resulting crude product was purified by prep-TLC (eluting with 2:1 ethyl acetate/petroleum ether) to afford tert-butyl N-(3-[2-[3-fluoro-4-(1H-pyrazol-1-yl)benzenesulfonyl]-1H,2H,3H-pyrrolo[3,4-c] pyridin-6-yl]-3-phenylpropyl)-N-methylcarbamate as light yellow oil (30 mg, 73%). LCMS (ES, m/z) 592 [M+H]⁺.

The Intermediates in Table 7 were synthesized according to the procedure described for Intermediate 9-1 above.

TABLE 7

| | | LCMS: (ESI) |
|---|---|---|
| Intermediate | Compound | m/z [M + H]⁺ |
| 9-2 | 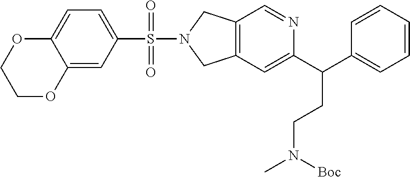 [SMILES: CN(CCC(c1ccccc1)c1cc2c(cn1)CN(S(=O)(=O)c1ccc3c(c1)OCCO3)C2)C(=O)OC(C)(C)C] | 566 |
| | tert-butyl (3-(2-((2,3-dihydrobenzo[b][1,4]dioxin-6- | |
| | yl)sulfonyl)-2,3-dihydro-1H-pyrrolo[3,4-c]pyridin-6-yl)-3- | |
| | phenylpropyl)(methyl)carbamate | |

### Intermediate 10-1. 6-[(3-chlorophenyl)carbonyl]-1H,2H,3H-pyrrolo[3,4-c]pyridine (TFA salt)

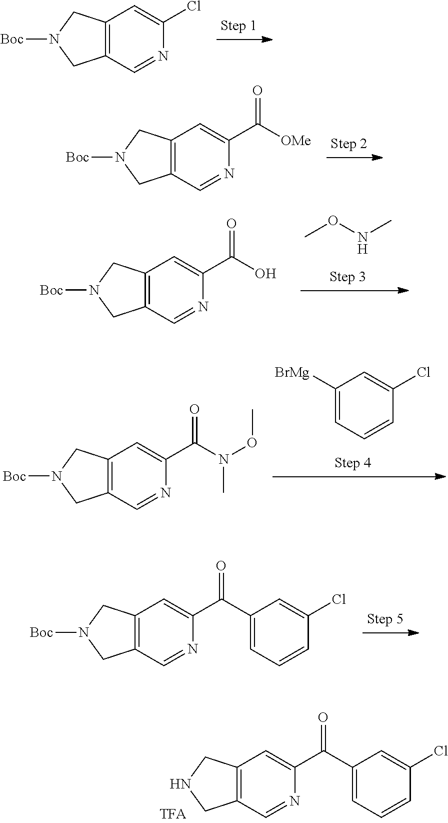

### Step 1. 2-tert-butyl 6-methyl 1H,2H,3H-pyrrolo[3,4-c]pyridine-2,6-dicarboxylate

Into a high pressure tank was placed a solution of tert-butyl 6-chloro-1H,2H,3H-pyrrolo[3,4-c]pyridine-2-carboxylate (2 g, 6.99 mmol) in MeOH (30 mL), Pd(dppf)Cl₂—CH2Cl2 (640 mg, 0.78 mmol) and TEA (3.28 mL, 23.7 mmol). Then CO (30 atm) was introduced. The resulting mixture was stirred for 16 h at 120° C. and cooled to room temperature. The reaction mixture was filtered and concentrated under vacuum. The resulting crude product was purified by silica gel chromatography (eluting with 1:5 EA/PE) to afford 2-tert-butyl 6-methyl 1H,2H,3H-pyrrolo[3,4-c]pyridine-2,6-dicarboxylate as a yellow solid (1.2 g, 56%). LCMS (ES, m/z) 279 [M+H]⁺.

### Step 2. 2-(tert-butoxycarbonyl)-2,3-dihydro-1H-pyrrolo[3,4-c]pyridine-6-carboxylic acid

To a solution of 2-tert-butyl 6-methyl 1H,2H,3H-pyrrolo[3,4-c]pyridine-2,6-dicarboxylate (2 g, 6.47 mmol) in THF (20 mL) was added water (15 mL) and LiOH (863 mg, 36.0 mmol). The resulting solution was stirred for 16 h at rt. The resulting mixture was washed with Et₂O (1×10 mL) and then acidified to pH 5 with hydrochloric acid solution (2 N). The resulting mixture was extracted with EA (3×25 mL). The combined organic layers were dried over anhydrous sodium sulfate, filtered and concentrated under vacuum. The resulting crude product was purified by reversed phase chromatography (eluting with 1:1 water/MeCN). The collected fractions were combined and concentrated under vacuum to afford 2-(tert-butoxycarbonyl)-2,3-dihydro-1H-pyrrolo[3,4-c]pyridine-6-carboxylic acid as yellow oil (1.0 g, 53%). LCMS (ES, m/z) 265 [M+H]⁺.

### Step 3. Tert-butyl 6-[methoxy(methyl)carbamoyl]-1H,2H,3H-pyrrolo[3,4-c]pyridine-2-carboxylate

To a solution of 2-[(tert-butoxy)carbonyl]-1H,2H,3H-pyrrolo[3,4-c]pyridine-6-carboxylic acid (1 g, 3.41 mmol) in DMF (15 mL) was added methoxy(methyl)amine hydrochloride (441 mg, 4.52 mmol), HATU (2.88 g, 7.57 mmol) and DIEA (1.98 mL, 11.37 mmol). The resulting solution was stirred for 1 h at rt. The reaction mixture was poured into water (15 mL) and then extracted with EA (3×15 mL). The combined organic layers were dried over anhydrous sodium sulfate, filtered and concentrate under vacuum. The resulting crude product was purified by silica gel chromatography (eluting with 1:10 EA/PE) to afford tert-butyl 6-[methoxy(methyl)carbamoyl]-1H,2H,3H-pyrrolo[3,4-c]pyridine-2-carboxylate as light yellow oil (700 mg, 67%). LCMS (ES, m/z) 308 [M+H]⁺.

### Step 4. Tert-butyl 6-[(3-chlorophenyl)carbonyl]-1H,2H,3H-pyrrolo[3,4-c]pyridine-2-carboxylate

To a solution of tert-butyl 6-[methoxy(methyl)carbamoyl]-1H,2H,3H-pyrrolo[3,4-c]pyridine-2-carboxylate (100 mg, 0.29 mmol) in THF (1 mL) was added a solution of bromo(3-chlorophenyl)magnesium (0.78 mL, 0.5 M in THF) dropwise with stirring at 0° C. The resulting solution was stirred for 1 h at rt and then poured into saturated ammonium chloride solution (5 mL). The resulting mixture was extracted with EA (3×5 mL). The combined organic layers were dried over anhydrous sodium sulfate, filtered and concentrated under vacuum. The resulting crude product was purified by silica gel chromatography (eluting with 1:3 EA/PE) to afford tert-butyl 6-[(3-chlorophenyl)carbonyl]-1H,2H,3H-pyrrolo[3,4-c]pyridine-2-carboxylate as yellow oil (80 mg, 76%). LCMS (ES, m/z) 359, 361 [M+H]⁺.

### Step 5. 6-[(3-chlorophenyl)carbonyl]-1H,2H,3H-pyrrolo[3,4-c]pyridine TFA salt

To a solution of tert-butyl 6-[(3-chlorophenyl)carbonyl]-1H,2H,3H-pyrrolo[3,4-c]pyridine-2-carboxylate (500 mg, 1.25 mmol) in dichloromethane (8 mL) was added TFA (2 mL). The resulting solution was stirred for 1 h at rt. The resulting mixture was concentrated under vacuum to afford 6-[(3-chlorophenyl)carbonyl]-1H,2H,3H-pyrrolo[3,4-c]pyridine (TFA salt) as brown oil (500 mg, crude). LCMS (ES, m/z) 259, 261 [M+H]⁺.

### Intermediate 11-1. 4-(5-fluoro-1H-pyrazol-1-yl) benzene-1-sulfonyl chloride

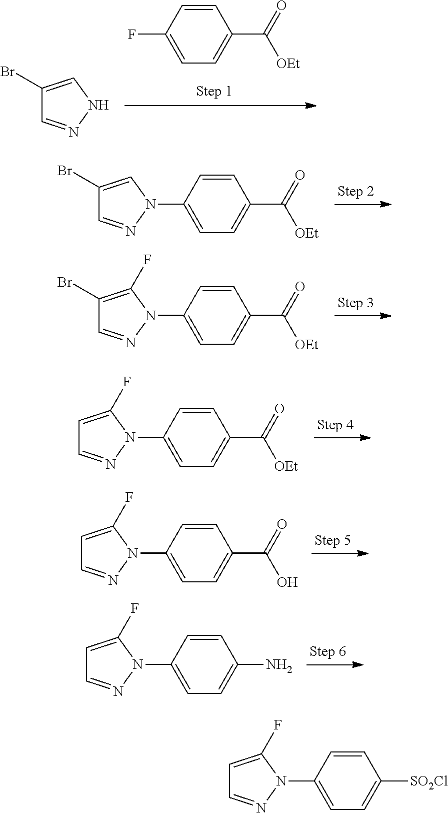

### Step 1. Ethyl 4-(4-bromo-1H-pyrazol-1-yl) benzoate

To a solution of 4-bromo-1H-pyrazole (10 g, 68.0 mmol) in N,N-dimethylformamide (100 mL) was added ethyl 4-fluorobenzoate (12 g, 85.1 mmol), and potassium carbonate (29 g, 210 mmol). The resulting mixture was stirred for 16 h at 120° C. and then cooled to room temperature. The reaction mixture was poured into water (80 mL) and then extracted with ethyl acetate (3×100 mL). The combined organic layers were washed with brine (3×100 mL), dried over anhydrous sodium sulfate, filtered, and concentrated under vacuum. The resulting crude product was purified by silica gel chromatography (eluting with 0:100 to 40:60 ethyl acetate/petroleum ether) to afford ethyl 4-(4-bromo-1H-pyrazol-1-yl) benzoate (7 g, 38%). LCMS (ES, m/z) 295, 297 [M+H]⁺.

### Step 2. Ethyl 4-(4-bromo-5-fluoro-1H-pyrazol-1-yl) benzoate

To a solution of ethyl 4-(4-bromo-1H-pyrazol-1-yl) benzoate (7 g, 23.7 mmol) in MeCN (70 mL) was added SelectFluor (26 g, 73.4 mmol). The resulting mixture was stirred for 48 h at 80° C. and then cooled to room temperature. The reaction mixture was poured into water (40 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 38:62 ethyl acetate/petroleum ether) to afford ethyl 4-(4-bromo-5-fluoro-1H-pyrazol-1-yl) benzoate (2.5 g, 34%). LCMS (ES, m/z) 313, 315 [M+H]⁺.

### Step 3. Ethyl 4-(5-fluoro-1H-pyrazol-1-yl) benzoate

To a solution of ethyl 4-(4-bromo-5-fluoro-1H-pyrazol-1-yl) benzoate (2.50 g, 7.98 mmol) in ethyl acetate (50 mL) was added palladium carbon (250 mg, 10 wt % palladium on charcoal). Then hydrogen was introduced in with hydrogen balloon. The resulting mixture was stirred for 1 h at room temperature, then filtered and concentrated under vacuum to afford ethyl 4-(5-fluoro-1H-pyrazol-1-yl)benzoate (800 mg, 43%). LCMS (ES, m/z) 235 [M+H]⁺.

### Step 4. 4-(5-fluoro-1H-pyrazol-1-yl) benzoic acid

To a solution of ethyl 4-(5-fluoro-1H-pyrazol-1-yl)benzoate (800 mg, 3.42 mmol) in THF (4 mL) was added water (4 mL) and lithium hydroxide (411 mg, 17.2 mmol). The resulting mixture was stirred for 2 h at room temperature. The reaction mixture was washed with diethyl ether (1×6 mL) and then acidified to pH 5 with hydrochloric acid (4 N). The resulting solution was extracted with ethyl acetate (2×6 mL). The combined organic layers were dried over anhydrous sodium sulfate, filtered, and concentrated under vacuum to afford 4-(5-fluoro-1H-pyrazol-1-yl)benzoic acid (600 mg, 85%). LCMS (ES, m/z) 207 [M+H]⁺.

### Step 5. 4-(5-fluoro-1H-pyrazol-1-yl) aniline

To a solution of 4-(5-fluoro-1H-pyrazol-1-yl) benzoic acid (500 mg, 2.43 mmol) in DMF (10 mL) was added TEA (1.0 mL, 7.28 mmol), and diphenylphosphoryl azide (1.0 g, 3.63 mmol). The resulting solution was stirred for 3 h at room temperature. To this was added a solution of sulfuric acid (3 mL, 1 M in water). The resulting solution was stirred for 1 h at 100° C. and then cooled to room temperature. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 4-(5-fluoro-1H-pyrazol-1-yl) aniline (400 mg, 93%). LCMS (ES, m/z) 178 [M+H]⁺.

### Step 6. 4-(5-fluoro-1H-pyrazol-1-yl) benzene-1-sulfonyl chloride

To glacial acetic acid (10 mL) was bubbled SO₂ gas for 1 h at room temperature. Then CuCl₂ (72 mg, 0.54 mmol) was added and SO₂ gas was bubbled in for additional 2 h to afford solution A. To a pre-cooled solution of 4-(5-fluoro-1H-pyrazol-1-yl)aniline (380 mg, 2.14 mmol) in acetic acid (2 mL) and concentrated hydrochloric acid (6 mL) was added a solution of sodium nitrite (163 mg, 2.36 mmol) in distilled water (0.5 mL) dropwise with stirring at −10° C. After stirring for 15 min, solution A was added to this diazonium salt solution at −10° C. The resulting solution was allowed to warm to room temperature naturally and stirred for 16 h. The reaction mixture was treated with water (10 mL) and then extracted with EA (3×10 mL). The combined organic layers were dried over anhydrous sodium sulfate, filtered, and concentrated under vacuum. The resulting crude product was purified by silica gel chromatography (eluting with 0:100 to 12:88 ethyl acetate/petroleum ether) to afford 4-(5-fluoro-1H-pyrazol-1-yl) benzene-1-sulfonyl chloride (350 mg, 63%). LCMS (ES, m/z) 261, 263 [M+H]⁺.

### Intermediate 12-1. Tert-butyl 6-[(3-bromophenyl)(hydroxy)methyl]-1H,2H,3H-pyrrolo[3,4-c]pyridine-2-carboxylate

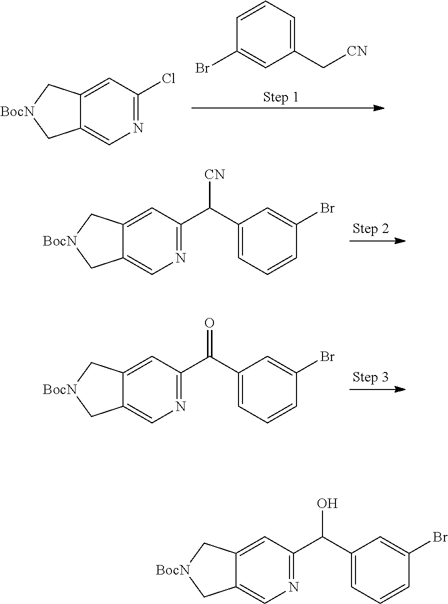

### Step 1. Tert-butyl 6-[(3-bromophenyl)(cyano)methyl]-1H,2H,3H-pyrrolo[3,4-c]pyridine-2-carboxylate

To a solution of tert-butyl 6-chloro-1H,2H,3H-pyrrolo[3,4-c]pyridine-2-carboxylate (2.0 g, 7.79 mmol) in tetrahydrofuran (20 mL) was added NaNH₂ (2.0 g, 7.87 mmol), and 2-(3-bromophenyl)acetonitrile (2.32 g, 11.8 mmol). The resulting mixture was stirred for 16 h at 50° C. and then cooled to room temperature. The reaction mixture was poured into water (20 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford tert-butyl 6-[(3-bromophenyl)(cyano)methyl]-1H,2H,3H-pyrrolo[3,4-c]pyridine-2-carboxylate (1.1 g, 34%). LCMS (ES, m/z) 414, 416 [M+H]⁺.

### Step 2. Tert-butyl 6-(3-bromobenzoyl)-1H,2H,3H-pyrrolo[3,4-c]pyridine-2-carboxylate

To a solution of sodium hydroxide (128 mg, 0.012 mmol) in water (0.13 mL) was added DMSO (8 mL), benzyltriethylammonium chloride (27 mg, 0.12 mmol) and a solution of tert-butyl 6-[(3-bromophenyl)(cyano)methyl]-1H,2H,3H-pyrrolo[3,4-c]pyridine-2-carboxylate (1.1 g, 2.66 mmol) in DMSO (10 mL). The resulting solution was stirred for 3 h at room temperature while oxygen was bubbling in. The reaction mixture was poured into water (20 mL) and then extracted with EA (3×20 mL). The combined organic layers were washed by brine, dried over anhydrous sodium sulfate, filtered and concentrated under vacuum. The resulting crude product was purified by silica gel chromatography (eluting with 0:100 to 20:80 ethyl acetate/petroleum ether) to afford tert-butyl 6-(3-bromobenzoyl)-1H,2H,3H-pyrrolo[3,4-c]pyridine-2-carboxylate (640 mg, 60%). LCMS (ES, m/z) 403, 405 [M+H]⁺.

### Step 3. Tert-butyl 6-[(3-bromophenyl)(hydroxy)methyl]-1H,2H,3H-pyrrolo[3,4-c]pyridine-2-carboxylate

To a solution of tert-butyl 6-(3-bromobenzoyl)-1H,2H,3H-pyrrolo[3,4-c]pyridine-2-carboxylate (500 mg, 1.24 mmol) in tetrahydrofuran (10 mL) was added NaBH₄ (95 mg, 2.49 mmol) at 0° C. The resulting mixture was stirred for 1 h at 0° C. The reaction mixture was poured into water (10 mL) and then extracted with EA (3×20 mL). The combined organic layers were washed with brine (3×20 mL), dried over anhydrous sodium sulfate, filtered, and concentrated under vacuum. The resulting crude product was purified by silica gel chromatography (eluting with 0:100 to 25:80 ethyl acetate/petroleum ether) to afford tert-butyl 6-[(3-bromophenyl)(hydroxy)methyl]-1H,2H,3H-pyrrolo[3,4-c]pyridine-2-carboxylate (400 mg, 80%). LCMS (ES, m/z) 405, 407 [M+H]⁺.

### Intermediate 13-1. 2-(2,3-dihydro-1,4-benzodioxine-6-sulfonyl)-1H,2H,3H-pyrrolo[3,4-c]pyridine-6-carbonitrile

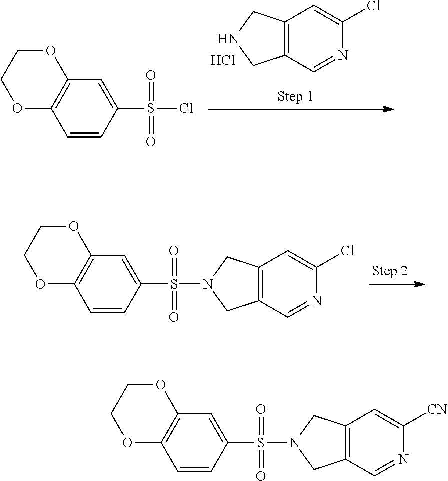 [SMILES: O=S(=O)(Cl)c1ccc2c(c1)OCCO2 ; Cl ; Clc1cc2c(cn1)CNC2 ; O=S(=O)(c1ccc2c(c1)OCCO2)N1Cc2cnc(Cl)cc2C1 ; N#Cc1cc2c(cn1)CN(S(=O)(=O)c1ccc3c(c1)OCCO3)C2]

### Step 1. 6-chloro-2-(2,3-dihydro-1,4-benzodioxine-6-sulfonyl)-1H,2H,3H-pyrrolo[3,4-c]pyridine

To a solution of 6-chloro-1H,2H,3H-pyrrolo[3,4-c]pyridine hydrochloride (810 mg, 4.24 mmol) in dichloromethane (10 mL) was added triethylamine (1.78 ml). This was followed by the addition of a solution of 2,3-dihydro-1,4-benzodioxine-6-sulfonyl chloride (1.00 g, 4.26 mmol) in dichloromethane (1 mL) dropwise with stirring at 0° C. The resulting mixture was stirred for 2 h at rt. The reaction mixture was poured into water (10 mL) and then extracted with dichloromethane (3×20 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 6-chloro-2-(2,3-dihydro-1,4-benzodioxine-6-sulfonyl)-1H,2H,3H-pyrrolo[3,4-c]pyridine (1 g, 60%). LCMS (ES, m/z) 353, 355 [M+H]⁺.

### Step 2. 2-(2,3-dihydro-1,4-benzodioxine-6-sulfonyl)-1H,2H,3H-pyrrolo[3,4-c]pyridine-6-carbonitrile

To a solution of 6-chloro-2-(2,3-dihydro-1,4-benzodioxine-6-sulfonyl)-1H,2H,3H-pyrrolo[3,4-c]pyridine (350 mg, 0.89 mmol) in N,N-dimethylformamide (3 mL) was added Zn(CN)₂ (173 mg, 1.47 mmol), and Pd(PPh₃)₄ (115 mg, 0.10 mmol). The resulting mixture was stirred for 1 h at 120° C. and then cooled to room temperature. The reaction mixture was poured into water (5 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 2-(2,3-dihydro-1,4-benzodioxine-6-sulfonyl)-1H,2H,3H-pyrrolo[3,4-c]pyridine-6-carbonitrile as a white solid (285 mg 84%) LCMS (ES, m/z) 344 [M+H]⁺.

### Intermediate 14-1. 4-(1,3-oxazol-2-yl)benzene-1-sulfonyl chloride

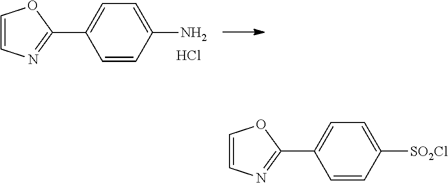 [SMILES: Nc1ccc(-c2ncco2)cc1 ; Cl ; O=S(=O)(Cl)c1ccc(-c2ncco2)cc1]

To glacial acetic acid (20 mL) was bubbled S02 gas for 1 h at room temperature. Then CuCl₂ (256 mg, 1.91 mmol) was added and S02 gas was bubbled in for additional 2 h to afford solution A. To a pre-cooled solution of 4-(1,3-oxazol-2-yl)aniline hydrochloride (1.5 g, 7.65 mmol) in acetic acid (5 mL) and concentrated hydrochloric acid (15 mL) was added a solution of sodium nitrite (581 mg, 8.42 mmol) in distilled water (2 mL) dropwise with stirring at −10° C. After stirring for 15 min, solution A was added to this diazonium salt solution at −10° C. The resulting solution was allowed to warm to room temperature naturally and stirred for 16 h. The reaction mixture was treated with water (20 mL) and then extracted with EA (3×30 mL). The combined organic layers were dried over anhydrous sodium sulfate, filtered, and concentrated under vacuum. The resulting crude product was purified by silica gel chromatography (eluting with 0:100 to 30:70 ethyl acetate/petroleum ether) to afford 4-(1,3-oxazol-2-yl)benzene-1-sulfonyl chloride as a white solid (1.3 g, 70%). LCMS (ES, m/z) 244, 246 [M+H]⁺.

### Intermediate 15-1. 2-(2,3-dihydro-1,4-benzodioxine-6-sulfonyl)-1H,2H,3H-pyrrolo[3,4-c]pyridine-6-carbaldehyde

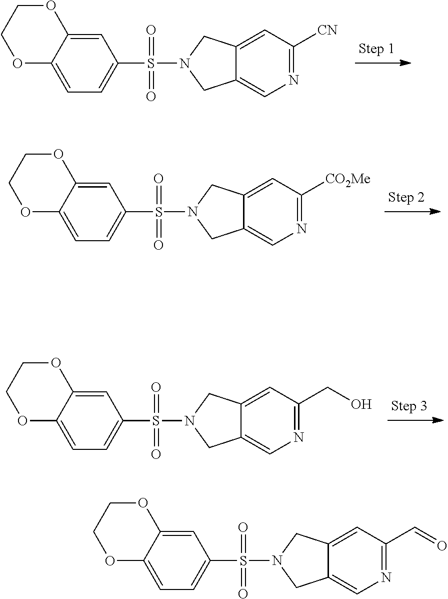 [SMILES: N#Cc1cc2c(cn1)CN(S(=O)(=O)c1ccc3c(c1)OCCO3)C2 ; COC(=O)c1cc2c(cn1)CN(S(=O)(=O)c1ccc3c(c1)OCCO3)C2 ; O=S(=O)(c1ccc2c(c1)OCCO2)N1Cc2cnc(CO)cc2C1 ; O=Cc1cc2c(cn1)CN(S(=O)(=O)c1ccc3c(c1)OCCO3)C2]

### Step 1. Methyl 2-(2,3-dihydro-1,4-benzodioxine-6-sulfonyl)-1H,2H,3H-pyrrolo[3,4-c]pyridine-6-carboxylate

Into a high pressure tank was placed a solution of 6-chloro-2-(2,3-dihydro-1,4-benzodioxine-6-sulfonyl)-1H,2H,3H-pyrrolo[3,4-c]pyridine (200 mg, 0.54 mmol) in MeOH (5 mL), Pd(dppf)Cl₂.CH2Cl2 (23 mg, 0.03 mmol) and TEA (0.24 mL, 1.70 mmol). Then CO (30 atm) was introduced. The resulting mixture was stirred for 5 h at 100° C. and cooled to room temperature. The reaction mixture was filtered and concentrated under vacuum. The resulting crude product was purified by silica gel chromatography (eluting with 10:1 DCM/MeOH) to afford methyl 2-(2,3-dihydro-1,4-benzodioxine-6-sulfonyl)-1H,2H,3H-pyrrolo[3,4-c]pyridine-6-carboxylate as a yellow solid (160 mg, 79%). LCMS (ES, m/z) 377 [M+H]⁺.

### Step 2. [2-(2,3-dihydro-1,4-benzodioxine-6-sulfonyl)-1H,2H,3H-pyrrolo[3,4-c]pyridin-6-yl]methanol

To a solution of methyl 2-(2,3-dihydro-1,4-benzodioxine-6-sulfonyl)-1H,2H,3H-pyrrolo[3,4-c]pyridine-6-carboxylate (200 mg, 0.50 mmol) in THF (5 mL) was added LiAlH₄ (10 mg, 0.26 mmol) carefully with stirring at 0° C. The resulting solution was stirred for 20 min at 0° C. The reaction was then quenched by addition of Na₂SO₄.10H₂O (100 mg). The reaction mixture was filtered and concentrated under vacuum. The resulting crude product was purified by silica gel chromatography (eluting with 10:1 DCM/MeOH) to afford [2-(2,3-dihydro-1,4-benzodioxine-6-sulfonyl)-1H,2H,3H-pyrrolo[3,4-c]pyridin-6-yl]methanol as a yellow solid (160 mg, 91%). LCMS (ES, m/z) 349 [M+H]⁺.

### Step 3. 2-(2,3-dihydro-1,4-benzodioxine-6-sulfonyl)-1H,2H,3H-pyrrolo[3,4-c]pyridine-6-carbaldehyde

To a solution of [2-(2,3-dihydro-1,4-benzodioxine-6-sulfonyl)-1H,2H,3H-pyrrolo[3,4-c]pyridin-6-yl]methanol (80 mg, 0.21 mmol) in 1,4-dioxane (3 mL) was added SeO₂ (13 mg, 0.12 mmol). The resulting mixture was stirred for 16 h at 80° C. and then cooled to room temperature. The reaction mixture was concentrated under vacuum. The resulting crude product was purified by silica gel chromatography (eluting with 1:1 EA/PE) to afford 2-(2,3-dihydro-1,4-benzodioxine-6-sulfonyl)-1H,2H,3H-pyrrolo[3,4-c]pyridine-6-carbaldehyde as yellow oil (40 mg, 56%). LCMS (ES, m/z) 347 [M+H]⁺.

### Intermediate 16-1. Phenyl[2-(piperidine-4-sulfonyl)-1H,2H,3H-pyrrolo[3,4-c]pyridine-6-yl]methanol

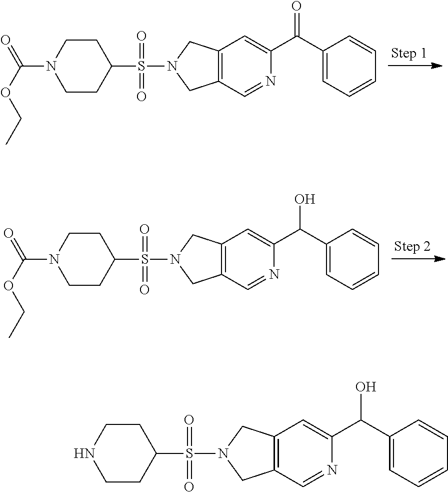 [SMILES: CCOC(=O)N1CCC(S(=O)(=O)N2Cc3cnc(C(=O)c4ccccc4)cc3C2)CC1 ; CCOC(=O)N1CCC(S(=O)(=O)N2Cc3cnc(C(O)c4ccccc4)cc3C2)CC1 ; O=S(=O)(C1CCNCC1)N1Cc2cnc(C(O)c3ccccc3)cc2C1]

### Step 1. Ethyl 4-({6-[hydroxy(phenyl)methyl]-1H,2H,3H-pyrrolo[3,4-c]pyridine-2-yl}sulfonyl) piperidine-1-carboxylate

To a solution of ethyl 4-([6-benzoyl-1H,2H,3H-pyrrolo[3,4-c]pyridin-2-yl]sulfonyl)piperidine-1-carboxylate (200 mg, 0.45 mmol) in methanol (4 mL), was added sodium borohydride (34 mg, 0.90 mmol). The resulting solution was stirred for 1 h at room temperature. The reaction mixture was poured into water (5 mL) and then extracted with ethyl acetate (2×5 mL). The combined organic layers were dried over anhydrous sodium sulfate, filtered, and concentrated under vacuum. The resulting crude product was purified by silica gel chromatography (eluting with 0:100 to 70:30 ethyl acetate/petroleum ether) to afford ethyl 4-([6-[hydroxy(phenyl)methyl]-1H,2H,3H-pyrrolo[3,4-c]pyridine-2-yl]sulfonyl)piperidine-1-carboxylate as light yellow oil (180 mg, 90%). LCMS (ES, m/z) 446 [M+H]⁺.

### Step 2. Phenyl[2-(piperidine-4-sulfonyl)-1H,2H,3H-pyrrolo[3,4-c]pyridine-6-yl]methanol

To a solution of ethyl 4-([6-[hydroxy(phenyl)methyl]-1H,2H,3H-pyrrolo[3,4-c]pyridin-2-yl] sulfonyl)piperidine-1-carboxylate (170 mg, 0.38 mmol) in tetrahydrofuran (2 mL) was added methanol (2 mL), water (2 mL) and sodium hydroxide (78 mg, 1.95 mmol). The resulting solution was stirred for 16 h at 65° C. The reaction mixture was poured into water (2 mL) and then extracted with ethyl acetate (2×5 mL). The combined organic layers were dried over anhydrous sodium sulfate, filtered, and concentrated under vacuum. The resulting crude product was purified by silica gel chromatography (eluting with 0:100 to 50:50 methanol/dichloromethane) to afford phenyl[2-(piperidine-4-sulfonyl)-1H,2H,3H-pyrrolo[3,4-c]pyridin-6-yl]methanol as yellow oil (90 mg, 63%). LCMS (ES, m/z) 374 [M+H]⁺.

### Intermediate 17-1. 4-(1H-pyrazol-1-yl)cyclohexane-1-sulfonyl chloride

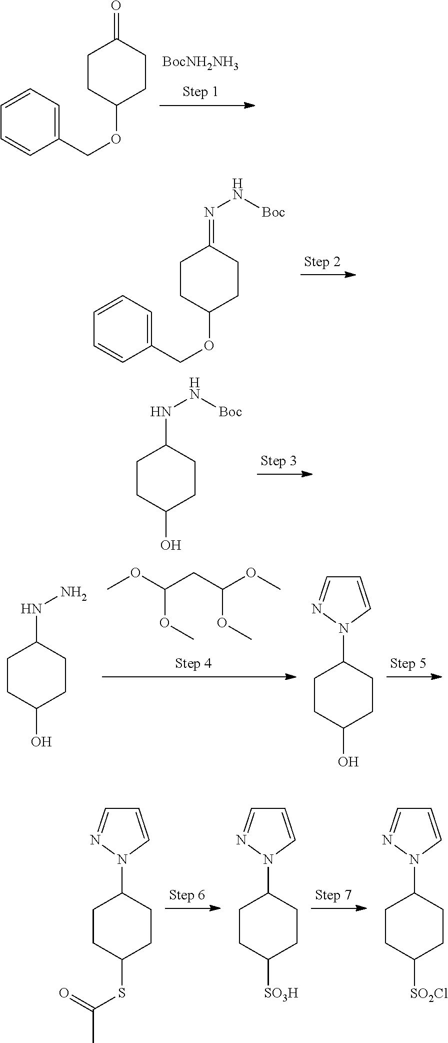

### Step 1. N′-[4-(benzyloxy)cyclohexylidene](tert-butoxy)carbohydrazide

To a solution of 4-(benzyloxy)cyclohexan-1-one (5 g, 0.025 mol) in methanol (25 mL) was added (tert-butoxy)carbohydrazide (3.2 g, 0.025 mol). The resulting solution was stirred for 1 h at rt. The resulting mixture was concentrated to minimum volume, filtered and dried under vacuum to afford N-4-(benzyloxy)cyclohexylidene](tert-butoxy)carbohydrazide as a yellow solid (3 g, 35%). 319 [M+H]⁺.

### Step 2. N′-(4-hydroxycyclohexyl)(tert-butoxy)carbohydrazide

To a solution of N-4-(benzyloxy)cyclohexylidene](tert-butoxy)carbohydrazide (3 g, 8.95 mmol) in methanol (300 mL) was added palladium carbon (300 mg, 10 wt % palladium on charcoal). Then hydrogen was introduced with hydrogen balloon. The resulting solution was stirred for 16 h at rt. The solids were filtered out. The filtrate was concentrated under vacuum to afford N′-(4-hydroxycyclohexyl)(tert-butoxy)carbohydrazide as yellow oil (2 g, 87%). LCMS (ES, m/z) 231 [M+H]⁺.

### Step 3. 4-hydrazinylcyclohexan-1-ol hydrochloride

To a solution of N′-(4-hydroxycyclohexyl)(tert-butoxy)carbohydrazide (2 g, 7.82 mmol) in dichloromethane (50 mL) was added a solution of hydrochloric acid (5 mL, 4 N in 1,4-dioxane). The resulting solution was stirred for 3 h at rt. The resulting mixture was concentrated under vacuum to afford 4-hydrazinylcyclohexan-1-ol hydrochloride as yellow oil (1.1 g, 85%). LCMS (ES, m/z) 131 [M+H]⁺.

### Step 4. 4-(1H-pyrazol-1-yl)cyclohexan-1-ol

To a solution of 4-hydrazinylcyclohexan-1-ol hydrochloride (1.8 g, 9.72 mmol) in ethanol (100 mL) was added 1,1,3,3-tetramethoxypropane (1.94 g, 11.8 mmol). The resulting mixture was stirred for 2 h at 90° C. and then cooled to room temperature. The resulting mixture was concentrated under vacuum. The resulting crude product was purified by silica gel chromatography (eluting with 0:100 to 20:80 ethyl acetate/petroleum ether) to afford 4-(1H-pyrazol-1-yl)cyclohexan-1-ol as yellow oil (1.2 g, 67%). LCMS (EI, m/z): 167 [M+H]⁺.

### Step 5. 1-{[4-(1H-pyrazol-1-yl)cyclohexyl]sulfanyl}ethan-1-one

To a solution of triphenylphosphine (4.1 g, 15.6 mmol) in tetrahydrofuran (10 mL) was added diisopropyl azodicarboxylate (3.2 g, 15.6 mmol) dropwise with stirring at −10° C. To this was added a solution of 4-(1H-pyrazol-1-yl)cyclohexan-1-ol (1.3 g, 7.8 mmol) in tetrahydrofuran (3 mL) dropwise with stirring at −10° C. and ethanethioic S-acid (1.2 g, 15.6 mmol). The resulting solution was stirred for 16 h at rt. The reaction mixture was poured into water (20 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 5:95 ethyl acetate/petroleum ether) to afford 1-{[4-(1H-pyrazol-1-yl)cyclohexyl]sulfanyl}ethan-1-one as yellow oil (700 mg, 36%). LCMS (ES, m/z) 225 [M+H]⁺.

### Step 6. 4-(1H-pyrazol-1-yl)cyclohexane-1-sulfonic acid

To a solution of 1-{[4-(1H-pyrazol-1-yl)cyclohexyl]sulfanyl}ethan-1-one (550 mg, 2.21 mmol) in formic acid (11 mL) and hydrogen peroxide (1.2 mL). The resulting solution was stirred for 2 h at room temperature. The resulting mixture was concentrated under vacuum to afford 4-(1H-pyrazol-1-yl)cyclohexane-1-sulfonic acid as a white solid (400 mg, 71%). LCMS (ES, m/z) 231 [M+H]⁺.

### Step 7. 4-(1H-pyrazol-1-yl)cyclohexane-1-sulfonyl chloride

To a solution of 4-(1H-pyrazol-1-yl)cyclohexane-1-sulfonic acid (400 mg, 1.74 mmol) in DCM (100 mL) was added oxalyl dichloride (0.74 mL, 8.69 mmol) dropwise with stirring at 0° C. The resulting mixture was stirred for 1 h at 0° C. and then concentrated under vacuum to afford 4-(1H-pyrazol-1-yl)cyclohexane-1-sulfonyl chloride as a yellow oil (400 mg, 93%). Intermediate 18-1. {7-methyl-1H,2H,3H-pyrrolo[3,4-c]pyridin-6-yl}(phenyl)methanol

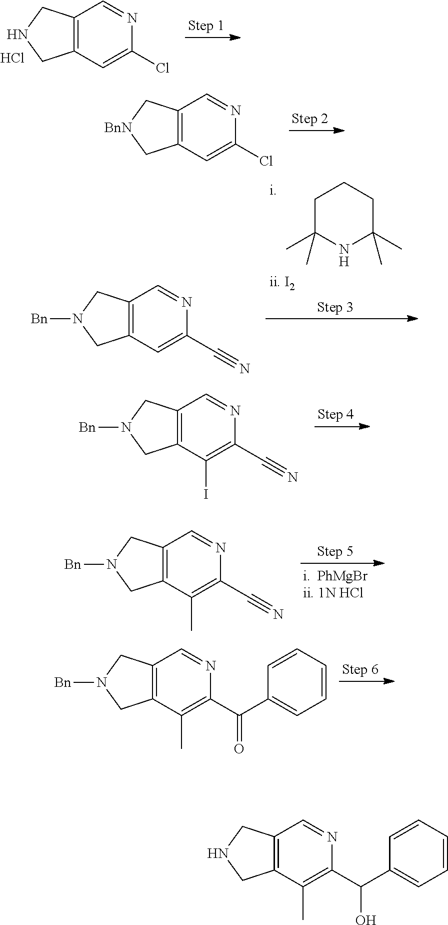

### Step 1. 2-Benzyl-6-chloro-1H,2H,3H-pyrrolo[3,4-c]pyridine

To a solution of 6-chloro-1H,2H,3H-pyrrolo[3,4-c]pyridine hydrochloride (1.20 g, 6.28 mmol) in MeOH (40 mL) was added triethylamine (0.87 mL, 6.29 mmol) and benzaldehyde (800 mg, 7.54 mmol). The resulting solution was stirred for 30 min at rt. Then sodium triacetoxyborohydride (3.90 g, 18.8 mmol) was added. The resulting solution was stirred for 16 h. The reaction mixture was poured into water (50 mL) and extracted with ethyl acetate (2×50 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 2-benzyl-6-chloro-1H,2H,3H-pyrrolo[3,4-c]pyridine as colorless oil (480 mg, 31%). LCMS (ES, m/z): 245, 247 [M+H]⁺.

### Step 2. 2-Benzyl-1H,2H,3H-pyrrolo[3,4-c]pyridine-6-carbonitrile

To a solution of 2-benzyl-6-chloro-1H,2H,3H-pyrrolo[3,4-c]pyridine (480 mg, 1.96 mmol) in DMF (8 mL) was added Zn(CN)₂ (461 mg, 3.93 mmol), and Pd(dppf)Cl2 (144 mg, 0.20 mmol). The resulting mixture was irradiated with microwave for 3 h at 140° C. After cooling to room temperature, the reaction mixture was poured into water (20 mL) and then extracted with ethyl acetate (2×20 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 2-benzyl-1H,2H,3H-pyrrolo[3,4-c]pyridine-6-carbonitrile as a white solid (300 mg, 65%). LCMS (ES, m/z): 236 [M+H]⁺.

### Step 3. 2-Benzyl-7-iodo-1H,2H,3H-pyrrolo[3,4-c]pyridine-6-carbonitrile

To a solution of 2,2,6,6-tetramethylpiperidine (461 mg, 3.26 mmol) in tetrahydrofuran (10 mL) was added n-BuLi (1.2 mL, 2.5 M in THF) at −78° C. The resulting solution was stirred for 30 min. Then a solution of 2-benzyl-1H,2H,3H-pyrrolo[3,4-c]pyridine-6-carbonitrile (480 mg, 2.04 mmol) in THF (5 mL) was added and stirred for 30 min at −78° C. After that a solution of diiodine (828 mg, 3.26 mmol) in THF (5 mL) was added. The resulting solution was stirred for 30 min at −78° C. and then allowed to warm to room temperature naturally. The reaction mixture was poured into saturated ammonium chloride (20 mL) and then extracted with ethyl acetate (2×20 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 2-benzyl-7-iodo-1H,2H,3H-pyrrolo[3,4-c]pyridine-6-carbonitrile as a yellow solid (611 mg. 83%). LCMS (ES, m/z): 362 [M+H]⁺.

### Step 4. 2-benzyl-7-methyl-1H,2H,3H-pyrrolo[3,4-c]pyridine-6-carbonitrile

To a solution of 2-benzyl-7-iodo-1H,2H,3H-pyrrolo[3,4-c]pyridine-6-carbonitrile (611 mg, 1.69 mmol) in 1,4-dioxane (20 mL) was added Sn(CH3)₄ (605 mg, 3.38 mmol), and Pd(dppf)Cl₂ (62 mg, 0.08 mmol). The resulting solution was stirred for overnight at 100° C. After cooling to room temperature, the reaction mixture was concentrated under vacuum. The crude product was purified by silica gel chromatography (eluting with 0:100 to 10:90 ethyl acetate/petroleum ether) to afford 2-benzyl-7-methyl-1H,2H,3H-pyrrolo[3,4-c]pyridine-6-carbonitrile as colorless oil (400 mg, 95%). LCMS (ES, m/z): 250 [M+H]⁺.

### Step 5. 6-benzoyl-2-benzyl-7-methyl-1H,2H,3H-pyrrolo[3,4-c]pyridine

To a solution of 2-benzyl-7-methyl-1H,2H,3H-pyrrolo[3,4-c]pyridine-6-carbonitrile (390 mg, 1.56 mmol) in tetrahydrofuran (15 mL) was added a solution of bromo(phenyl)magnesium (3.13 mL, 1 M in THF) at 0° C. The resulting mixture was stirred for 16 h and hydrochloric acid (10 mL, 1 N) was added. The resulting solution was stirred for 30 min and poured into water (50 mL) and then extracted with ethyl acetate (2×50 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 6-benzoyl-2-benzyl-7-methyl-1H,2H,3H-pyrrolo[3,4-c]pyridine as yellow oil (300 mg, 58%). LCMS (ES, m/z): 329 [M+H]⁺.

### Step 6. {7-methyl-1H,2H,3H-pyrrolo[3,4-c]pyridin-6-yl}(phenyl)methanol

To a solution of 6-benzoyl-2-benzyl-7-methyl-1H,2H,3H-pyrrolo[3,4-c]pyridine (150 mg, 0.46 mmol) in methanol (15 mL) was added Pd(OH)₂/C (150 mg, 20 wt % Pd). Then hydrogen was introduced in with hydrogen balloon. The resulting mixture was stirred for 2 h at rt. The reaction mixture was filtered and concentrated under vacuum to afford {7-methyl-1H,2H,3H-pyrrolo[3,4-c]pyridin-6-yl}(phenyl)methanol as yellow oil, which was used directly in the next step without further purification (150 mg, crude). LCMS (ES, m/z) 241 [M+H]⁺.

### Intermediate 19-1. Phenyl({5H,6H,7H-pyrrolo[3,4-c]pyridazin-3-yl})methanol

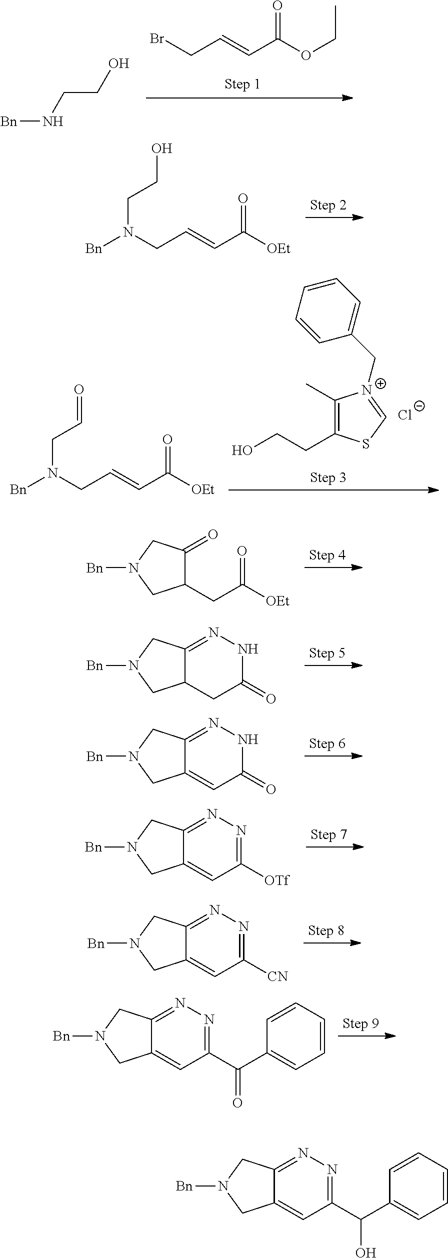

### Step 1. Ethyl 4-[benzyl(2-hydroxyethyl)amino]but-2-enoate

To a solution of 2-(benzylamino)ethan-1-ol (20.0 g, 133 mmol) in dichloromethane (150 mL), was added ethyl 4-bromobut-2-enoate (12.9 g, 66.8 mmol), potassium carbonate (18.5 g, 133 mmol). The resulting solution was stirred for 16 h at room temperature. The reaction mixture was filtered and concentrated under vacuum. The resulting crude product was purified by silica gel chromatography (eluting with 0:100 to 25:75 ethyl acetate/petroleum ether) to afford ethyl 4-[benzyl(2-hydroxyethyl)amino]but-2-enoate as colorless oil (13.0 g, 74%). LCMS (ES, m/z): 264 [M+H]⁺.

### Step 2. Ethyl 4-[benzyl(2-oxoethyl)amino]but-2-enoate

To a solution of oxalyl chloride (12.5 g, 98.7 mmol) in dichloromethane (150 mL) was added DMSO (10.5 mL, 148 mmol) dropwise at −78° C. After stirring for 20 min, to this solution was added a solution of ethyl 4-[benzyl(2-hydroxyethyl)amino]but-2-enoate (13.0 g, 49.4 mmol) in dichloromethane (20 mL) dropwise at −78° C. The resulting solution was stirred for 20 min and then triethylamine (41.0 mL, 0.29 mmol) was added. The resulting solution was allowed to warm to room temperature naturally and stirred for 12 h. The resulting solution was poured into saturated sodium bicarbonate solution (100 mL) and then extracted with dichloromethane (3×100 mL). The combined organic layers were dried over anhydrous sodium sulfate, filtered, and concentrated under vacuum. The crude product was purified by silica gel chromatography (eluting with 0:100 to 25:75 ethyl acetate/petroleum ether) to afford ethyl 4-[benzyl(2-oxoethyl)amino]but-2-enoate as light yellow oil (12.5 g, 99%). LCMS (ES, m/z): 262 [M+H]⁺.

### Step 3. Ethyl 2-(1-benzyl-4-oxopyrrolidin-3-yl) acetate

To a solution of ethyl (2E)-4-[benzyl(2-oxoethyl)amino]but-2-enoate (12.5 g, 47.8 mmol) in tetrahydrofuran (50 mL) was added potassium carbonate (1.30 g, 9.95 mmol) and 3-benzyl-5-(2-hydroxyethyl)-4-methylthiazol-3-ium chloride (2.70 g, 9.95 mmol). The resulting solution was stirred for 16 h at room temperature. The reaction mixture was poured into water (60 mL) and then extracted with ethyl acetate (3×60 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford ethyl 2-(1-benzyl-4-oxopyrrolidin-3-yl) acetate as light yellow oil (7.70 g, 62%). LCMS (ES, m/z): 262 [M+H]⁺.

### Step 4. 6-Benzyl-2H,3H,4H,4aH,5H,6H,7H-pyrrolo[3,4-c]pyridazin-3-one

To a solution of ethyl 2-(1-benzyl-4-oxopyrrolidin-3-yl) acetate (7.70 g, 29.5 mmol) in ethanol (50 mL), was added hydrazine monohydrate (1.30 mL, 26.5 mmol). The resulting solution was stirred for 2 h at 70° C. After cooling to room temperature, the reaction mixture was poured into water (50 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 10:90 methanol/dichloromethane) to afford 6-benzyl-2H,3H,4H,4aH,5H,6H,7H-pyrrolo[3,4-c]pyridazin-3-one as a white solid (2.50 g, 37%). LCMS (ES, m/z): 230 [M+H]⁺.

### Step 5. 6-Benzyl-2H,3H,5H,6H,7H-pyrrolo[3,4-c]pyridazin-3-one

To a solution of 6-benzyl-2H,3H,4H,4aH,5H,6H,7H-pyrrolo[3,4-c]pyridazin-3-one (2.50 g, 10.9 mmol) in MeCN (80 mL) was added CuCl₂ (2.9 g, 21.8 mmol). The resulting mixture was stirred for 2 h at 80° C. After cooling to room temperature, the reaction mixture was poured into water (100 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 10:90 methanol/dichloromethane) to afford 6-benzyl-2H,3H,5H,6H,7H-pyrrolo[3,4-c]pyridazin-3-one as a red solid (1.20 g, 48%). LCMS (ES, m/z): 228 [M+H]⁺.

### Step 6. 6-Benzyl-5H,6H,7H-pyrrolo[3,4-c]pyridazin-3-yl trifluoromethanesulfonate

To a solution of 6-benzyl-2H,3H,5H,6H,7H-pyrrolo [3,4-c]pyridazin-3-one (1.20 g, 5.28 mmol) in pyridine (15 mL) was added Tf₂O (1.9 g, 6.87 mmol) in a water/ice bath. Then the resulting solution was stirred for 2 h at 70° C. After cooling to room temperature, the reaction mixture was concentrated and treated with hydrochloric acid (60 mL, 1 N). The resulting solution was extracted with ethyl acetate (3×60 mL). The combined organic layers were dried over anhydrous sodium sulfate, filtered, and concentrated under vacuum. The resulting crude product was purified by silica gel chromatography (eluting with 0:100 to 30:70 ethyl acetate/petroleum ether) to afford 6-benzyl-5H,6H,7H-pyrrolo[3,4-c]pyridazin-3-yl trifluoromethanesulfonate as a green solid (1.00 g, 53%). LCMS (ES, m/z): 360 [M+H]⁺.

### Step 7. 6-Benzyl-5H,6H,7H-pyrrolo[3,4-c]pyridazine-3-carbonitrile

To a solution of 6-benzyl-5H,6H,7H-pyrrolo[3,4-c]pyridazin-3-yl trifluoromethanesulfonate (700 mg, 1.95 mmol) in N,N-dimethylformamide (15 mL) was added Zn(CN)₂ (252 mg, 2.15 mmol), Pd₂(dba)₃ (71 mg, 0.08 mmol) and dppf (108 mg, 0.20 mmol). The resulting mixture was stirred for 16 h at 100° C. After cooling to room temperature, the reaction mixture was poured into water (50 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 6-benzyl-5H,6H,7H-pyrrolo[3,4-c]pyridazine-3-carbonitrile as a light yellow solid (400 mg, 87%). LCMS (ES, m/z): 237 [M+H]⁺.

### Step 8. 3-Benzoyl-6-benzyl-5H,6H,7H-pyrrolo[3,4-c]pyridazine

To a solution of 6-benzyl-5H,6H,7H-pyrrolo[3,4-c]pyridazine-3-carbonitrile (100 mg, 0.42 mmol) in tetrahydrofuran (7 mL) was added bromo(phenyl)magnesium (0.21 mL, 3.0 M in THF) at 0° C. The resulting solution was stirred for 2 h at room temperature. The reaction mixture was treated with hydrochloric acid (10 mL, 1 N) and then extracted with ethyl acetate (3×15 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 3-benzoyl-6-benzyl-5H,6H,7H-pyrrolo[3,4-c]pyridazine as a white solid (100 mg, 75%). LCMS (ES, m/z): 316 [M+H]⁺.

### Step 9. Phenyl({5H,6H,7H-pyrrolo[3,4-c]pyridazin-3-yl})methanol

To a solution of 3-benzoyl-6-benzyl-5H,6H,7H-pyrrolo[3,4-c]pyridazine (50 mg, 0.16 mmol) in methanol (7 mL) was added Pd(OH)₂/C (8 mg, 20 wt % Pd) and several drops of hydrochloric acid (1 N). Then hydrogen was introduced with hydrogen balloon. The resulting mixture was stirred for 2 h at rt. The reaction mixture was filtered and concentrated under vacuum to afford phenyl({5H,6H,7H-pyrrolo[3,4-c]pyridazin-3-yl})methanol as a white solid (30 mg, 36%). LCMS (ES, m/z): 228 [M+H]⁺.

### Intermediate 20-1. Phenyl({5H,6H,7H-pyrrolo[3,4-d]pyrimidin-2-yl})methanol

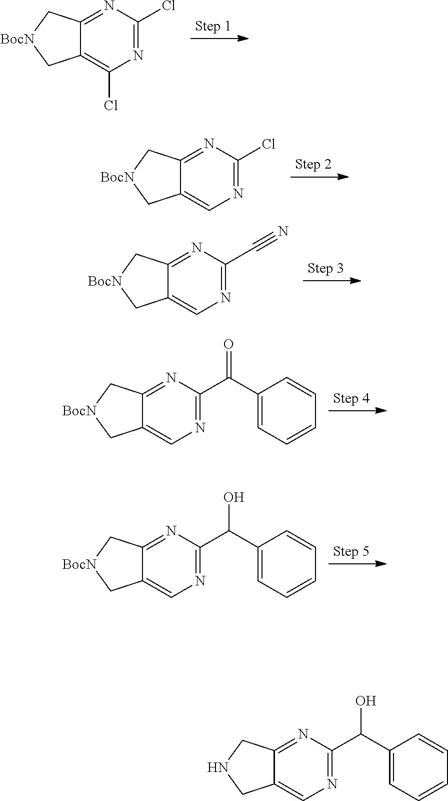

### Step 1. tert-Butyl 2-chloro-5H,6H,7H-pyrrolo[3,4-d]pyrimidine-6-carboxylate

To a solution of tert-butyl 2,4-dichloro-5H,6H,7H-pyrrolo[3,4-d]pyrimidine-6-carboxylate (6.00 g, 17.6 mmol) in methanol (48 mL) was added zinc powder (1.80 g, 26.4 mmol) and acetic acid (10.6 mL, 176 mmol). The resulting mixture was stirred for 16 h at 50° C. and cooled to room temperature. The resulting mixture was concentrated under vacuum. The resulting crude product was purified by silica gel chromatography (eluting with 1:1 ethyl acetate/petroleum ether) to afford tert-butyl 2-chloro-5H,6H,7H-pyrrolo[3,4-d]pyrimidine-6-carboxylate as a white solid (2.90 g, 54%). LCMS (ES, m/z): 256, 258 [M+H]⁺.

### Step 2. tert-Butyl 2-cyano-5H,6H,7H-pyrrolo[3,4-d]pyrimidine-6-carboxylate

To a solution of tert-butyl 2-chloro-5H,6H,7H-pyrrolo[3,4-d]pyrimidine-6-carboxylate (1.50 g, 4.99 mmol) in DMF (15 mL) was added Zn(CN)₂ (868 mg, 7.48 mmol) and Pd(dppf)Cl₂ (364 mg, 0.50 mmol). The resulting mixture was irradiated with microwave for 3 h at 140° C. After cooling to rt, the reaction mixture was poured into water (10 mL) and then extracted with ethyl acetate (3×15 mL). The combined organic layers were dried over anhydrous sodium sulfate, filtered, and concentrated under vacuum. The resulting crude product was purified by silica gel chromatography (eluting with 1:1 ethyl acetate/petroleum ether) to afford tert-butyl 2-cyano-5H,6H,7H-pyrrolo[3,4-d]pyrimidine-6-carboxylate as a yellow oil (500 mg, 34%). LCMS (ES, m/z): 247 [M+H]⁺.

### Step 3. tert-Butyl 2-benzoyl-5H,6H,7H-pyrrolo[3,4-d]pyrimidine-6-carboxylate

To a solution of tert-butyl 2-cyano-5H,6H,7H-pyrrolo[3,4-d]pyrimidine-6-carboxylate (200 mg, 0.69 mmol) in THF (2 mL) was added bromo(phenyl)magnesium (1.38 mL, 1 M in THF) dropwise at 0° C. The resulting mixture was stirred for 1 h at rt. Then 1 N hydrochloric acid (2 mL) was added. The resulting mixture was stirred for 30 min at rt and then extracted with EA (3×5 mL). The combined organic layers were dried over anhydrous sodium sulfate, filtered, and concentrated under vacuum. The resulting crude product was purified by silica gel chromatography (eluting with 4:5 ethyl acetate/petroleum ether) to afford tert-butyl 2-benzoyl-5H,6H,7H-pyrrolo[3,4-d]pyrimidine-6-carboxylate as a yellow oil (90 mg, 34%). LCMS (ES, m/z): 326 [M+H]⁺.

### Step 4. tert-Butyl 2-[hydroxy(phenyl)methyl]-5H,6H,7H-pyrrolo[3,4-d]pyrimidine-6-carboxylate

To a solution of tert-butyl 2-benzoyl-5H,6H,7H-pyrrolo[3,4-d]pyrimidine-6-carboxylate (90 mg, 0.24 mmol) in methanol (1 mL) was added sodium borohydride (18.7 mg, 0.47 mmol). The resulting mixture stirred for 1 h at rt. The reaction mixture was poured into water (5 mL) and then extracted with EA (3×5 mL). The combined organic layers were dried over anhydrous sodium sulfate, filtered, and concentrated under vacuum. The resulting crude product was purified by Prep-TLC (eluting with 2:5 ethyl acetate/petroleum ether) to afford tert-butyl 2-[hydroxy(phenyl)methyl]-5H,6H,7H-pyrrolo[3,4-d]pyrimidine-6-carboxylate as a yellow solid (60 mg, 77%). LCMS (ES, m/z): 328 [M+H]⁺.

### Step 5. Phenyl({5H,6H,7H-pyrrolo[3,4-d]pyrimidin-2-yl})methanol

To a solution of tert-butyl 2-[hydroxy(phenyl)methyl]-5H,6H,7H-pyrrolo[3,4-d]pyrimidine-6-carboxylate (60 mg, 0.18 mmol) in DCM (6 mL) was added trifluoroacetic acid (2 mL). The resulting mixture was stirred for 1 h at rt and concentrated under vacuum. The resulting mixture was basified to pH 8 with saturated potassium carbonate solution and extracted with DCM (3×5 mL). The combined organic layers were dried over anhydrous sodium sulfate, filtered, and concentrated under vacuum to afford phenyl({5H,6H,7H-pyrrolo[3,4-d]pyrimidin-2-yl})methanol as a yellow solid (35 mg, 85%). LCMS (ES, m/z): 228 [M+H]⁺.

The Intermediate in Table 8 were synthesized according to the procedure described for Intermediate 20-1 above.

TABLE 8

| | | LCMS: |
|---|---|---|
| Inter- | | (ESI) m/z |
| mediate | Compound | [M + H]⁺ |
| 20-2 | 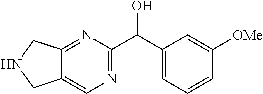 [SMILES: COc1cccc(C(O)c2ncc3c(n2)CNC3)c1] | 258 |
| | (6,7-dihydro-5H-pyrrolo[3,4-d]pyrimidin-2-yl)(3- | |
| | methoxyphenyl)methanol | |

### Intermediate 21-1. Benzyl 4-[1H,2H,3H-pyrrolo[3,4-c]pyridine-6-carbonyl]-2,3-dihydro-1H-isoindole-2-carboxylate

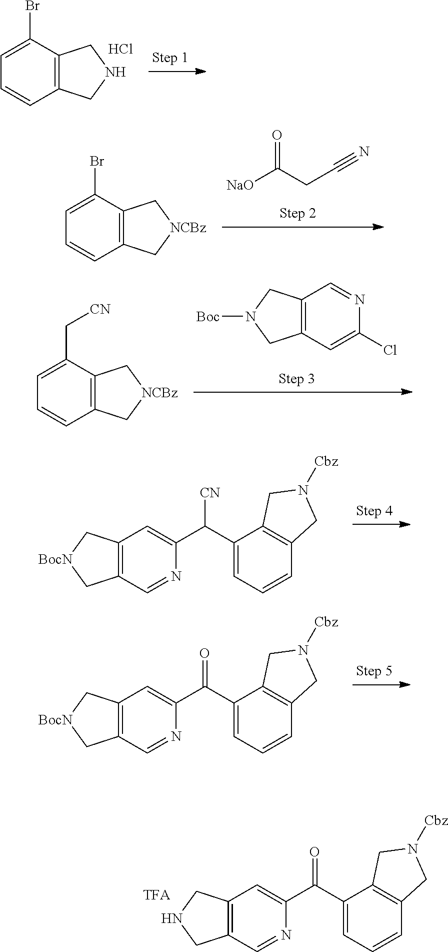

### Step 1. Benzyl 4-bromo-2,3-dihydro-1H-isoindole-2-carboxylate

To a solution of 4-bromo-2,3-dihydro-1H-isoindole hydrochloride (3.00 g, 12.2 mmol) and TEA (5.10 mL, 36.5 mmol) in dichloromethane (50 mL) was added CbzCl (4.10 g, 24.3 mmol) in portions at 0° C. The resulting solution was stirred for 5 h at room temperature. The reaction mixture was poured into water (50 mL) and then extracted with ethyl acetate (3×50 mL). The combined organic layers were dried over anhydrous sodium sulfate, filtered and concentrated under vacuum. The resulting crude product was purified by silica gel chromatography (eluting with 0:100 to 20:80 ethyl acetate/petroleum ether) to afford benzyl 4-bromo-2,3-dihydro-1H-isoindole-2-carboxylate as a pink solid (3.50 g, 87%). LCMS (ES, m/z): 332, 334[M+H]⁺.

### Step 2. tert-Butyl 4-(cyanomethyl)-2,3-dihydro-1H-isoindole-2-carboxylate

To a solution of tert-butyl 4-bromo-2,3-dihydro-1H-isoindole-2-carboxylate (1.50 g, 4.78 mmol) in mesitylene (20 mL) was added Pd(allyl)₂Cl2 (46 mg, 0.10 mmol), SPhos (118 mg, 0.29 mmol) and sodium 2-cyanoacetate (808 mg, 7.17 mmol). The resulting mixture was stirred for 5 h at 140° C. After cooling to room temperature, the reaction mixture was filtered and concentrated under vacuum. The resulting crude product was purified by silica gel chromatography (eluting with 0:100 to 50:50 ethyl acetate/petroleum ether) to afford tert-butyl 4-(cyanomethyl)-2,3-dihydro-1H-isoindole-2-carboxylate as a brown solid (1.00 g, 81%). LCMS (ES, m/z): 293[M+H]⁺.

### Step 3. Benzyl 4-([2-[(tert-butoxy)carbonyl]-1H,2H,3H-pyrrolo[3,4-c]pyridin-6-yl](cyano)methyl)-2,3-dihydro-1H-isoindole-2-carboxylate

To a solution of tert-butyl 6-chloro-1H,2H,3H-pyrrolo[3,4-c]pyridine-2-carboxylate (500 mg, 1.86 mmol) in THF (10 mL) was added benzyl 4-(cyanomethyl)-2,3-dihydro-1H-isoindole-2-carboxylate (861 mg, 2.80 mmol) and sodium amide (146 mg, 3.74 mmol). The resulting solution was stirred for 4 h at 50° C. After cooling to room temperature, the reaction mixture was poured into water (20 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 30:60 ethyl acetate/petroleum ether) to afford benzyl 4-([2-[(tert-butoxy)carbonyl]-1H,2H,3H-pyrrolo[3,4-c]pyridin-6-yl](cyano)methyl)-2,3-dihydro-1H-isoindole-2-carboxylate as yellow oil (300 mg, 32%). LCMS (ES, m/z): 511[M+H]⁺.

### Step 4. Benzyl 4-([2-[(tert-butoxy)carbonyl]-1H,2H,3H-pyrrolo[3,4-c]pyridin-6-yl](cyano)methyl)-2,3-dihydro-1H-isoindole-2-carboxylate

To a solution of benzyl 4-([2-[(tert-butoxy)carbonyl]-1H,2H,3H-pyrrolo[3,4-c]pyridin-6-yl](cyano)methyl)-2,3-dihydro-1H-isoindole-2-carboxylate (300 mg, 0.56 mmol) in DMSO (5 mL) was added benzyltriethylammonium chloride (6 mg, 0.03 mmol) and sodium hydroxide (0.2 mL, 4 N in water). Then oxygen was bubbled in. The resulting mixture was stirred for 2 h at room temperature. The reaction mixture was poured into water (20 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford benzyl 4-[2-[(tert-butoxy)carbonyl]-1H,2H,3H-pyrrolo[3,4-c]pyridine-6-carbonyl]-2,3-dihydro-1H-isoindole-2-carboxylate as yellow oil (200 mg, 72%). LCMS (ES, m/z): 500 [M+H]⁺.

### Step 5. Benzyl 4-[1H,2H,3H-pyrrolo[3,4-c]pyridine-6-carbonyl]-2,3-dihydro-1H-isoindole-2-carboxylate (TFA salt)

To a solution of benzyl 4-[2-[(tert-butoxy)carbonyl]-1H,2H,3H-pyrrolo[3,4-c]pyridine-6-carbonyl]-2,3-dihydro-1H-isoindole-2-carboxylate (200 mg, 0.38 mmol) in dichloromethane (5 mL) was added trifluoroacetic acid (1 mL). The resulting solution was stirred for 5 h at room temperature. The resulting mixture was concentrated under vacuum to afford benzyl 4-[1H,2H,3H-pyrrolo[3,4-c]pyridine-6-carbonyl]-2,3-dihydro-1H-isoindole-2-carboxylate (TFA salt) as yellow oil (180 mg, crude). LCMS (ES, m/z): 400[M+H]⁺.

### Intermediate 22-1. Tert-butyl 6-(3-bromobenzoyl)-1H,2H,3H-pyrrolo[3,4-c]pyridine-2-carboxylate

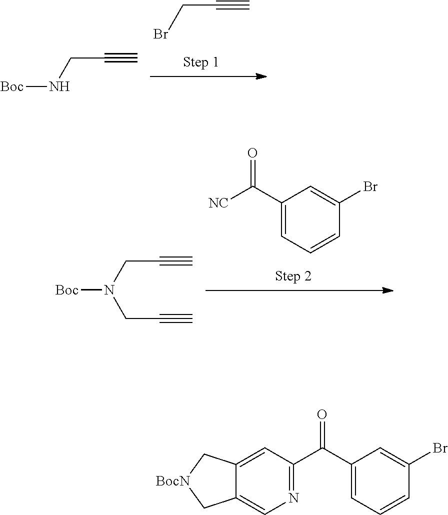

### Step 1. Tert-butyl N,N-bis(prop-2-yn-1-yl)carbamate

To a solution of tert-butyl N-(prop-2-yn-1-yl)carbamate (100 g, 612 mmol) in DMF (1.00 L), was added sodium hydride (29.4 g, 734 mmol, 60% dispersion in mineral oil) in portions with stirring at 0° C. The resulting mixture was stirred for 1 h at 0° C. Then to the above mixture was added propargyl bromide (82.6 mL, 918 mmol). The resulting mixture was stirred for 16 h at 50° C. After cooling to room temperature, the reaction mixture was poured into water (1 L) and then extracted with ethyl acetate (1.2 L×2). The organic layers were combined, dried over anhydrous sodium sulfate, filtered and concentrated under vacuum. The resulting crude product was purified by silica gel chromatography (eluting with 1:15 ethyl acetate/petroleum ether) to afford tert-butyl N,N-bis(prop-2-yn-1-yl)carbamate as yellow oil (90 g, 68%). LCMS (ES, m/z): 194 [M+H]⁺.

### Step 2. Tert-butyl 6-(3-bromobenzoyl)-1H,2H,3H-pyrrolo[3,4-c]pyridine-2-carboxylate

To a solution of 3-bromobenzoyl cyanide (5.00 g, 22.6 mmol) in 1,2-dichloroethane (50 mL) was added Cp*Ru(COD)Cl (172 mg, 0.45 mmol) and tert-butyl N,N-bis(prop-2-yn-1-yl)carbamate (4.60 g, 22.6 mmol) at 0° C. The resulting solution was stirred for 4 h at 60° C. The reaction mixture was cooled to room temperature and then concentrated under vacuum. The resulting crude product was purified by silica gel chromatography (eluting with 0:100 to 20:80 ethyl acetate/petroleum ether) to afford tert-butyl 6-(3-bromobenzoyl)-1H,2H,3H-pyrrolo[3,4-c]pyridine-2-carboxylate as a yellow solid (6.30 g, 63%). LCMS (ES, m/z): 403,405 [M+H]⁺.

The Intermediates in Table 9 were synthesized according to the procedure described for Intermediate 22-1 above.

TABLE 9

| | | LCMS: |
|---|---|---|
| Inter- | | (ESI) m/z |
| mediate | Compound | [M + H]⁺ |
| 22-2 | 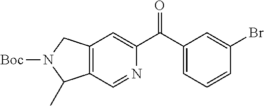 [SMILES: CC1c2cnc(C(=O)c3cccc(Br)c3)cc2CN1C(=O)OC(C)(C)C] | 417, 419 |
| | tert-butyl 6-(3-bromobenzoyl)-3-methyl-1,3-dihydro- | |
| | 2H-pyrrolo [3,4-c]pyridine-2-carboxylate | |
| | 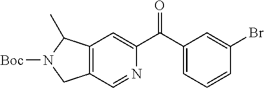 [SMILES: CC1c2cc(C(=O)c3cccc(Br)c3)ncc2CN1C(=O)OC(C)(C)C] | |
| | tert-butyl 6-(3-bromobenzoyl)-1-methyl-1,3-dihydro- | |
| | 2H-pyrrolo[3,4-c]pyridine-2-carboxylate | |

### Intermediate 23-1. 6-[(3-bromophenyl)(methoxy)methyl]-1H,2H,3H-pyrrolo[3,4-c]pyridine

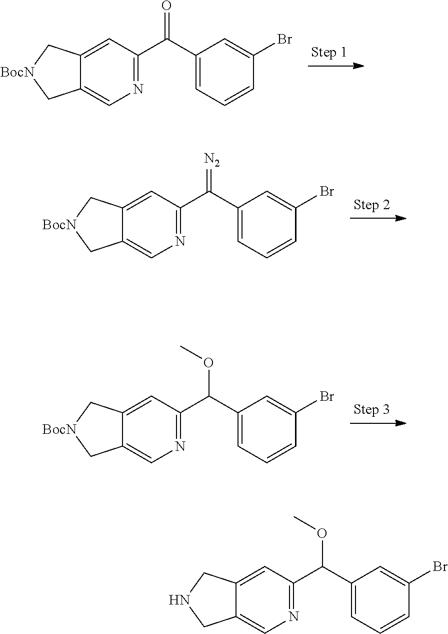

### Step 3. Tert-butyl 6-[(3-bromophenyl)(diazo)methyl]-1H,2H,3H-pyrrolo[3,4-c]pyridine-2-carboxylate

To a solution of tert-butyl 6-(3-bromobenzoyl)-1H,2H,3H-pyrrolo[3,4-c]pyridine-2-carboxylate (2.20 g, 4.63 mmol) in MeOH (50 mL) was added 4-methylbenzene-1-sulfonohydrazide (1.36 g, 6.95 mmol). The resulting mixture was stirred for 16 h at 60° C. After cooling to room temperature, the reaction mixture was concentrated. The solids were collected by filtration and dried under vacuum to afford tert-butyl 6-[(3-bromophenyl)(diazo) methyl]-1H,2H,3H-pyrrolo[3,4-c]pyridine-2-carboxylate as an off-white solid (1.90 g, 60%). LCMS (EI, m/z): 415, 417 [M+H]⁺.

### Step 4. Tert-butyl 6-[(3-bromophenyl)(methoxy)methyl]-1H,2H,3H-pyrrolo[3,4-c]pyridine-2-carboxylate

To a solution of tert-butyl 6-[(3-bromophenyl)(diazo)methyl]-1H,2H,3H-pyrrolo[3,4-c]pyridine-2-carboxylate (1.20 g, 2.45 mmol) in MeOH (20 mL) and 1,4-dioxane (20 mL) was added dirhodium tetraacetate (46 mg, 0.10 mmol). The resulting mixture was stirred for 16 h at 110° C. After cooling to room temperature, the reaction mixture was poured into water (30 mL) and then extracted with ethyl acetate (3×30 mL). The organic layers were combined, dried over anhydrous sodium sulfate, filtered, and concentrated under vacuum. The resulting crude product was purified by silica gel chromatography (eluting with 0:100 to 20:80 ethyl acetate/petroleum ether) to afford tert-butyl 6-[(3-bromophenyl)(methoxy)methyl]-1H,2H,3H-pyrrolo[3,4-c]pyridine-2-carboxylate as a yellow solid (900 mg, 72%). LCMS (ES, m/z): 419,421 [M+H]⁺.

### Step 5. 6-[(3-bromophenyl)(methoxy)methyl]-1H,2H,3H-pyrrolo[3,4-c]pyridine

To a solution of tert-butyl 6-[(3-bromophenyl)(methoxy)methyl]-1H,2H,3H-pyrrolo[3,4-c]pyridine-2-carboxylate (900 mg, 1.82 mmol) in DCM (5 mL) was added hydrochloric acid (5 mL, 4 N in 1,4-dioxane). The resulting solution was stirred for 2 h at room temperature. The reaction mixture was concentrated under vacuum to afford 6-[(3-bromophenyl)(methoxy)methyl]-1H,2H,3H-pyrrolo[3,4-c]pyridine as a black solid (700 mg, 90%). LCMS (ES, m/z): 319, 321 [M+H]⁺.

### Intermediate 24-1. Tert-butyl 6-(2-bromobenzoyl)-1H,2H,3H-pyrrolo[3,4-c]pyridine-2-carboxylate

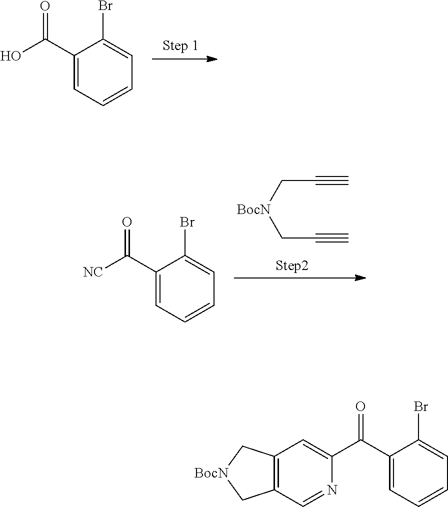 [SMILES: O=C(O)c1ccccc1Br ; N#CC(=O)c1ccccc1Br ; C#CCN(CC#C)C(=O)OC(C)(C)C ; CC(C)(C)OC(=O)N1Cc2cnc(C(=O)c3ccccc3Br)cc2C1]

### Step 1. 2-bromobenzoyl cyanide

To 2-bromobenzoic acid (4.00 g, 18.9 mmol) was added oxalic dichloride (8.46 mL, 94.3 mmol). The resulting solution was stirred for 2 h at 25° C. Then the reaction mixture was concentrated under vacuum. The resulting mixture was dissolved with MeCN (20 mL). To the above solution was added copper (I) cyanide (3.60 g, 37.8 mmol). The resulting mixture was stirred for 2 h at 85° C. After cooling to room temperature, the reaction mixture was concentrated under vacuum. The resulting mixture was washed with diethyl ether (2×100 mL). The solids were filtered out. The filtrate was concentrated under vacuum. The resulting mixture was washed with hexane (2×100 mL). The solids were filtered out. The filtrate was concentrated under vacuum to afford 2-bromobenzoyl cyanide as a yellow solid (2.71 g, 62%).

### Step 2. Tert-butyl 6-(2-bromobenzoyl)-1H,2H,3H-pyrrolo[3,4-c]pyridine-2-carboxylate

To a solution of 2-bromobenzoyl cyanide (2.20 g, 9.07 mmol) in DCE (25 mL) was added tert-butyl N,N-bis(prop-2-yn-1-yl)carbamate (2.50 g, 12.2 mmol), and Cp*Ru(COD)Cl (73 mg, 0.18 mmol). The resulting mixture was stirred for 1 h at 60° C. After cooling to room temperature, the reaction mixture was concentrated under vacuum. The resulting crude product was purified by silica gel chromatography (eluting with 1:2 ethyl acetate/petroleum ether) to afford tert-butyl 6-(2-bromobenzoyl)-1H,2H,3H-pyrrolo[3,4-c]pyridine-2-carboxylate as a brown solid (962 mg, 22%). LCMS (ES, m/z): 403, 405 [M+H]⁺.

The Intermediates in Table 10 were synthesized according to the procedure described for Intermediate 24-1 above.

TABLE 10

| | | LCMS: |
|---|---|---|
| Inter- | | (ESI) m/z |
| mediate | Compound | [M + H]⁺ |
| 24-2 | 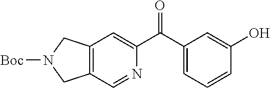 [SMILES: CC(C)(C)OC(=O)N1Cc2cnc(C(=O)c3cccc(O)c3)cc2C1] | 341 |
| | tert-butyl 6-(3-hydroxybenzoyl)-1,3-dihydro-2H- | |
| | pyrrolo[3,4-c]pyridine-2-carboxylate | |
| 24-3 | 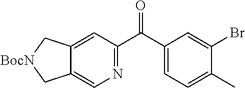 [SMILES: Cc1ccc(C(=O)c2cc3c(cn2)CN(C(=O)OC(C)(C)C)C3)cc1Br] | 417, 419 |
| | tert-butyl 6-(3-bromo-4-methylbenzoyl)-1,3-dihydro- | |
| | 2H-pyrrolo[3,4-c]pyridine-2-carboxylate | |
| 24-4 | 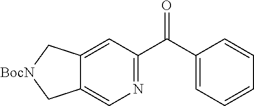 [SMILES: CC(C)(C)OC(=O)N1Cc2cnc(C(=O)c3ccccc3)cc2C1] | 325 |
| | tert-butyl 6-benzoyl-1,3-dihydro-2H-pyrrolo[3,4- | |
| | c]pyridine-2-carboxylate | |

### Intermediate 25-1. Tert-butyl 6-[hydroxy[2-(4-methylpiperazin-1-yl)phenyl]methyl]-1H, 2H, 3H-pyrrolo [3,4-c] pyridine-2-carboxylate

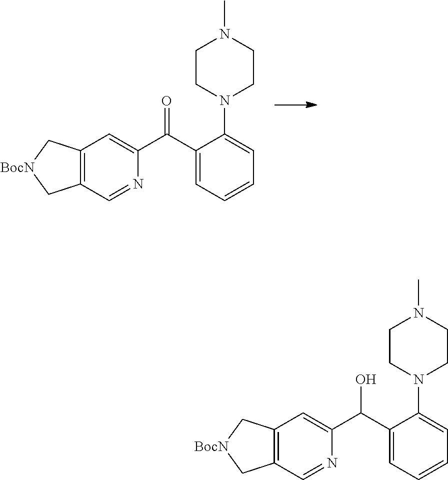 [SMILES: CN1CCN(c2ccccc2C(=O)c2cc3c(cn2)CN(C(=O)OC(C)(C)C)C3)CC1 ; CN1CCN(c2ccccc2C(O)c2cc3c(cn2)CN(C(=O)OC(C)(C)C)C3)CC1]

To a solution of tert-butyl 6-[2-(4-methylpiperazin-1-yl)benzoyl]-1H, 2H, 3H-pyrrolo [3,4-c] pyridine-2-carboxylate (200 mg, 0.43 mmol) in MeOH (10 mL) was added sodium borohydride (8 mg, 0.21 mmol). The resulting mixture was stirred for 1 h at 25° C. The reaction mixture was concentrated under vacuum. The resulting crude product was purified by silica gel chromatography (eluting with 1:10 MeOH/DCM) to afford tert-butyl 6-[hydroxy[2-(4-methylpiperazin-1-yl)phenyl]methyl]-1H,2H,3H-pyrrolo[3,4-c]pyridine-2-carboxylate as yellow oil (170 mg, 85%). LCMS (ES, m/z): 425 [M+H]⁺.

The Intermediates in Table 11 were synthesized according to the procedure described for Intermediate 25-1 above.

TABLE 11

| | | LCMS: (ESI) |
|---|---|---|
| Intermediate | Compound | m/z [M + H]⁺ |
| 25-2 | 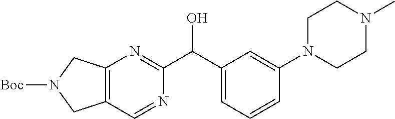 [SMILES: CN1CCN(c2cccc(C(O)c3ncc4c(n3)CN(C(=O)OC(C)(C)C)C4)c2)CC1] | 426 |
| | tert-butyl 2-(hydroxy(3-(4-methylpiperazin-1- | |
| | yl)phenyl)methyl)-5,7-dihydro-6H-pyrrolo[3,4- | |
| | d]pyrimidine-6-carboxylate | |
| 25-3 | 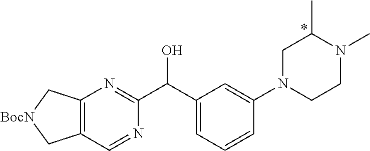 [SMILES: CC1CN(c2cccc(C(O)c3ncc4c(n3)CN(C(=O)OC(C)(C)C)C4)c2)CCN1C] | 440 |
| | tert-butyl 2-((3-(3,4-dimethylpiperazin-1- | |
| | yl)phenyl)(hydroxy)methyl)-5,7-dihydro-6H- | |
| | pyrrolo[3,4-d]pyrimidine-6-carboxylate | |

*Absolute stereochemistry not determined.

### Intermediate 26-1. 1-methyl-4-(3-{2-[4-(1,3-oxazol-2-yl)benzenesulfonyl]-1H,2H,3H-pyrrolo[3,4-c]pyridine-6-carbonyl} phenoxy)piperidine

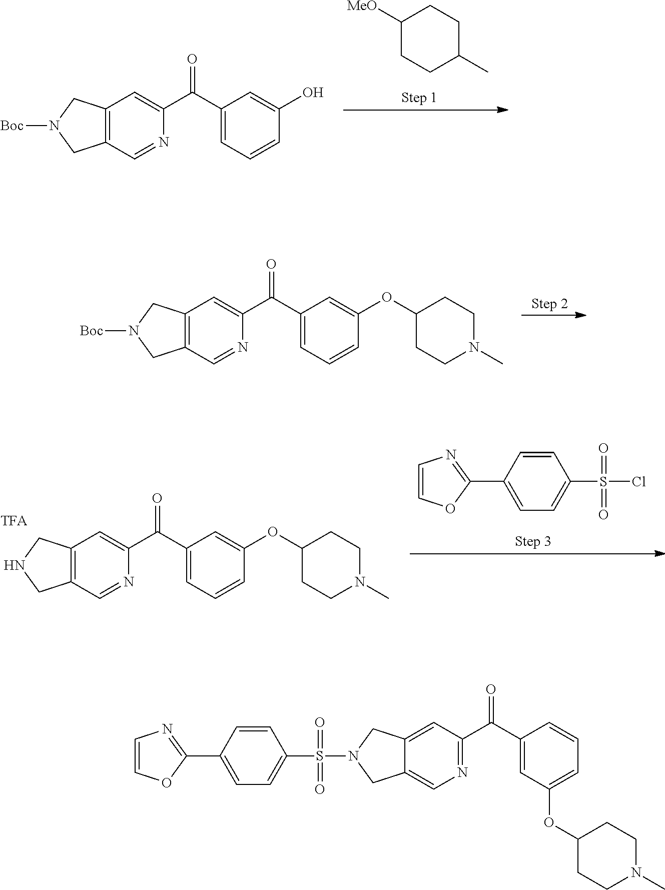

### Step 1. Tert-butyl 6-{3-[(1-methylpiperidin-4-yl)oxy]benzoyl}-1H,2H,3H-pyrrolo[3,4-c]pyridine-2-carboxylate

To a solution of tert-butyl 6-(3-hydroxybenzoyl)-1H,2H,3H-pyrrolo[3,4-c]pyridine-2-carboxylate (500 mg, 1.47 mmol) in DMF (10 mL), was added 1-methylpiperidin-4-yl methanesulfonate (1.42 g, 7.34 mmol), cesium carbonate (1.44 g, 4.40 mmol) and potassium iodide (24 mg, 0.15 mmol). The resulting mixture was stirred for 16 h at 100° C. After cooling to room temperature, the reaction mixture was poured into water (30 mL) and then extracted with ethyl acetate (3×30 mL). The organic layers were combined, dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 6-[3-[(1-methylpiperidin-4-yl)oxy]benzoyl]-1H,2H,3H-pyrrolo [3,4-c]pyridine-2-carboxylate as light yellow oil (400 mg, 62%). LCMS (ES, m/z): 438 [M+H]⁺.

### Step 2. 1-methyl-4-(3-{1H,2H,3H-pyrrolo[3,4-c]pyridine-6-carbonyl}phenoxy)piperidine (TFA salt)

To a solution of tert-butyl 6-[3-[(1-methylpiperidin-4-yl)oxy]benzoyl]-1H,2H,3H-pyrrolo [3,4-c]pyridine-2-carboxylate (300 mg, 0.68 mmol) in dichloromethane (4 mL), was added trifluoroacetic acid (1 mL). The resulting solution was stirred for 2 h at 25° C. The reaction mixture was concentrated under vacuum to afford 1-methyl-4-(3-[1H,2H,3H-pyrrolo[3,4-c]pyridine-6-carbonyl]phenoxy)piperidine (TFA salt) as light yellow oil (200 mg, 86%). LCMS (ES, m/z): 338 [M+H]⁺.

### Step 3. 1-methyl-4-(3-{2-[4-(1,3-oxazol-2-yl)benzenesulfonyl]-1H,2H,3H-pyrrolo[3,4-c]pyridine-6-carbonyl} phenoxy)piperidine

To a solution of 1-methyl-4-(3-[1H,2H,3H-pyrrolo[3,4-c]pyridine-6-carbonyl]phenoxy)piperidine (TFA salt) (200 mg, 0.59 mmol) in DCM (8 mL) was added TEA (0.49 mL, 3.55 mmol) and 4-(1,3-oxazol-2-yl)benzene-1-sulfonyl chloride (144 mg, 0.59 mmol). The resulting mixture was stirred for 2 h at 25° C. and then concentrated under vacuum. The resulting crude product was purified by silica gel chromatography (eluting with 100:0 to 90:10 dichloromethane/methanol) to afford 1-methyl-4-(3-[2-[4-(1,3-oxazol-2-yl)benzenesulfonyl]-1H,2H,3H-pyrrolo [3,4-c]pyridine-6-carbonyl] phenoxy)piperidine as light yellow oil (100 mg, 31%). LCMS (ES, m/z): 545 [M+H]⁺.

### Intermediate 27-1. Tert-butyl 2-(3-bromobenzoyl)-5H,6H,7H-pyrrolo[3,4-d]pyrimidine-6-carboxylate

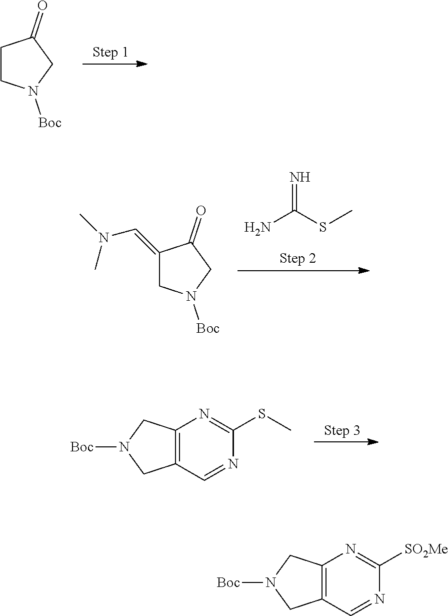

### Step 1. Tert-butyl (3E)-3-[(dimethylamino)methylidene]-4-oxopyrrolidine-1-carboxylate

To tert-butyl 3-oxopyrrolidine-1-carboxylate (20 g, 102 mmol) was added dimethylformamide dimethyl acetal (200 mL). The resulting solution was stirred for 12 h at 140° C. After cooling to room temperature, the resulting mixture was concentrated. The residue was dissolved with a minimum amount of DCM and then treated with hexane (100 mL). The resulting solids were collected by filtration and dried under vacuum to afford tert-butyl (3E)-3-[(dimethylamino)methylidene]-4-oxopyrrolidine-1-carboxylate as a yellow solid (15 g, 58%). LCMS (ES, m/z): 241 [M+H]⁺.

### Step 2. Tert-butyl 2-(methylsulfanyl)-5H,6H,7H-pyrrolo[3,4-d]pyrimidine-6-carboxylate

To a solution of (methylsulfanyl)methanimidamide (17 g, 183 mmol) in EtOH (200 mL) was added sodium ethoxide (13 g, 183 mmol) at 0° C. After stirring for 10 min, to the above solution was added tert-butyl (3E)-3-[(dimethylamino)methylidene]-4-oxopyrrolidine-1-carboxylate (15 g, 61.2 mmol). The resulting mixture was stirred for 4 h at 80° C. After cooling to room temperature, the reaction mixture was concentrated under vacuum. The residue was dissolved with water (100 mL) and then extracted with ethyl acetate (3×100 mL). The organic layers were combined, dried over anhydrous sodium sulfate, filtered and concentrated under vacuum. The resulting crude product was purified by silica gel chromatography (eluting with 1:2 ethyl acetate/petroleum ether) to afford tert-butyl 2-(methylsulfanyl)-5H,6H,7H-pyrrolo[3,4-d]pyrimidine-6-carboxylate as a yellow solid (4 g, 41%). LCMS (ES, m/z): 268 [M+H]⁺.

### Step 3. Tert-butyl 2-methanesulfonyl-5H,6H,7H-pyrrolo[3,4-d]pyrimidine-6-carboxylate

To a solution of tert-butyl 2-(methylsulfanyl)-5H,6H,7H-pyrrolo[3,4-d]pyrimidine-6-carboxylate (4 g, 14.1 mmol) in DCM (80 mL) was added m-CPBA (7.5 g, 42.6 mmol). The resulting mixture was stirred for 5 h at 0° C. The resulting mixture was washed with saturated sodium bicarbonate solution (5×100 mL), dried over anhydrous sodium sulfate, filtered and concentrated. The resulting crude product was purified by silica gel chromatography (eluting with 2:1 ethyl acetate/petroleum ether) to afford tert-butyl 2-methanesulfonyl-5H,6H,7H-pyrrolo[3,4-d]pyrimidine-6-carboxylate as a yellow solid (4 g, 89%). LCMS (ES, m/z): 300 [M+H]⁺.

### Intermediate 28-1. {4-methyl-5H,6H,7H-pyrrolo[3,4-d]pyrimidin-2-yl}[3-(4-methylpiperazin-1-yl)phenyl]methanol (HCl salt)

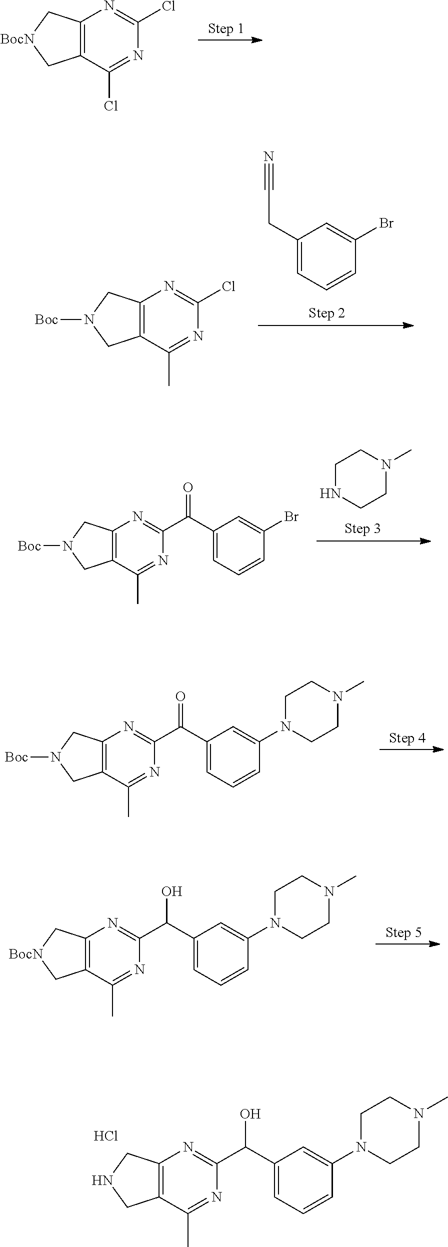

### Step 1. Tert-butyl 2-chloro-4-methyl-5H,6H,7H-pyrrolo[3,4-d]pyrimidine-6-carboxylate

To a solution of tert-butyl 2,4-dichloro-5H,6H,7H-pyrrolo[3,4-d]pyrimidine-6-carboxylate (1.10 g, 3.79 mmol) in tetrahydrofuran (20 mL) was added ferric acetylacetonate (416 mg, 1.18 mmol), and methylmagnesium chloride (2 mL, 3 M in THF) at 0° C. The resulting mixture was stirred for 18 h at 0° C. The reaction mixture was poured into water (10 mL) and then extracted with ethyl acetate (3×20 mL). The organic layers were combined, dried over anhydrous sodium sulfate, filtered, and concentrated under vacuum. The resulting crude product was purified by silica gel chromatography (eluting with 0:100 to 30:70 ethyl acetate/petroleum ether) to afford tert-butyl 2-chloro-4-methyl-5H,6H,7H-pyrrolo[3,4-d]pyrimidine-6-carboxylate as a yellow solid (550 mg, 48%). LCMS (ES, m/z): 270, 272 [M+H]⁺.

### Step 2. Tert-butyl 2-(3-bromobenzoyl)-4-methyl-5H,6H,7H-pyrrolo[3,4-d]pyrimidine-6-carboxylate

To a solution of 2-(3-bromophenyl)acetonitrile (600 mg, 3.06 mmol) in THF (10 mL), was added lithium bis(trimethylsilyl)amide (3 mL, 1 M in THF), and tert-butyl 2-chloro-4-methyl-5H,6H,7H-pyrrolo[3,4-d] pyrimidine-6-carboxylate (550 mg, 2.04 mmol). The resulting mixture was stirred for 18 h at room temperature while oxygen was kept bubbling in. The reaction mixture was poured into water (10 mL) and then extracted with ethyl acetate (3×20 mL). The organic layers were combined, dried over anhydrous sodium sulfate, filtered, and concentrated under vacuum. The resulting crude product was purified by silica gel chromatography (eluting with 0:100 to 50:50 ethyl acetate/petroleum ether) to afford tert-butyl 2-(3-bromobenzoyl)-4-methyl-5H,6H,7H-pyrrolo[3,4-d]pyrimidine-6-carboxylate as a light yellow solid (380 mg, 40%). LCMS (ES, m/z): 418, 420 [M+H]⁺.

### Step 3. Tert-butyl 4-methyl-2-[3-(4-methylpiperazin-1-yl)benzoyl]-5H,6H,7H-pyrrolo [3,4-d]pyrimidine-6-carboxylate

To a solution of tert-butyl 2-(3-bromobenzoyl)-4-methyl-5H,6H,7H-pyrrolo[3,4-d]pyrimidine-6-carboxylate (370 mg, 0.88 mmol) in 1,4-dioxane (10 mL) was added cesium carbonate (865 mg, 2.66 mmol), 1-methylpiperazine (266 mg, 2.66 mmol), RuPhos (42 mg, 0.09 mmol), and RuPhos 3G (74 mg, 0.09 mmol). The resulting mixture was stirred for 18 h at 100° C. After cooling to room temperature, the reaction mixture was poured into water (10 mL) and then extracted with ethyl acetate (3×20 mL). The organic layers were combined, dried over anhydrous sodium sulfate, filtered, and concentrated under vacuum. The resulting crude product was purified by silica gel chromatography (eluting with 0:100 to 80:20 ethyl acetate/petroleum ether) to afford tert-butyl 4-methyl-2-[3-(4-methylpiperazin-1-yl)benzoyl]-5H,6H,7H-pyrrolo[3,4-d]pyrimidine-6-carboxylate as a yellow solid (200 mg, 46%). LCMS (ES, m/z) 438 [M+H]⁺.

### Step 4. Tert-butyl 2-{hydroxy[3-(4-methylpiperazin-1-yl)phenyl]methyl}-4-methyl-5H,6H,7H-pyrrolo [3,4-d]pyrimidine-6-carboxylate

To a solution of tert-butyl 4-methyl-2-[3-(4-methylpiperazin-1-yl)benzoyl]-5H,6H,7H-pyrrolo [3,4-d]pyrimidine-6-carboxylate (200 mg, 0.45 mmol) in methanol (5 mL), was added sodium borohydride (35 mg, 0.92 mmol). The resulting mixture was stirred for 1 h at room temperature. The reaction mixture was poured into water (5 mL) and then extracted with ethyl acetate (3×10 mL). The organic layers were combined, dried over anhydrous sodium sulfate, filtered, and concentrated under vacuum. The resulting crude product was purified by prep-TLC (eluting with 1:10 methanol/dichloromethane) to afford tert-butyl 2-[hydroxy[3-(4-methylpiperazin-1-yl)phenyl]methyl]-4-methyl-5H,6H,7H-pyrrolo[3,4-d]pyrimidine-6-carboxylate as yellow oil (180 mg, 80%). LCMS (ES, m/z): 440 [M+H]⁺.

### Step 5. {4-methyl-5H,6H,7H-pyrrolo[3,4-d]pyrimidin-2-yl}[3-(4-methylpiperazin-1-yl)phenyl]methanol (HCl salt)

To a solution of tert-butyl 2-[hydroxy[3-(4-methylpiperazin-1-yl)phenyl]methyl]-4-methyl-5H,6H,7H-pyrrolo [3,4-d]pyrimidine-6-carboxylate (180 mg, 0.41 mmol) in dichloromethane (3 mL) was added hydrochloric acid (3 mL, 4 N in 1,4-dioxane). The resulting mixture was stirred for 1 h at room temperature. The reaction mixture was concentrated under vacuum to afford [4-methyl-5H,6H,7H-pyrrolo[3,4-d]pyrimidin-2-yl] [3-(4-methylpiperazin-1-yl)phenyl]methanol (HCl salt) as a yellow solid (130 mg, 84%). LCMS (ES, m/z): 340 [M+H]⁺.

### Intermediate 29-1. {4-methoxy-5H,6H,7H-pyrrolo[3,4-d]pyrimidin-2-yl}[3-(4-methylpiperazin-1-yl)phenyl]methanol (HCl salt)

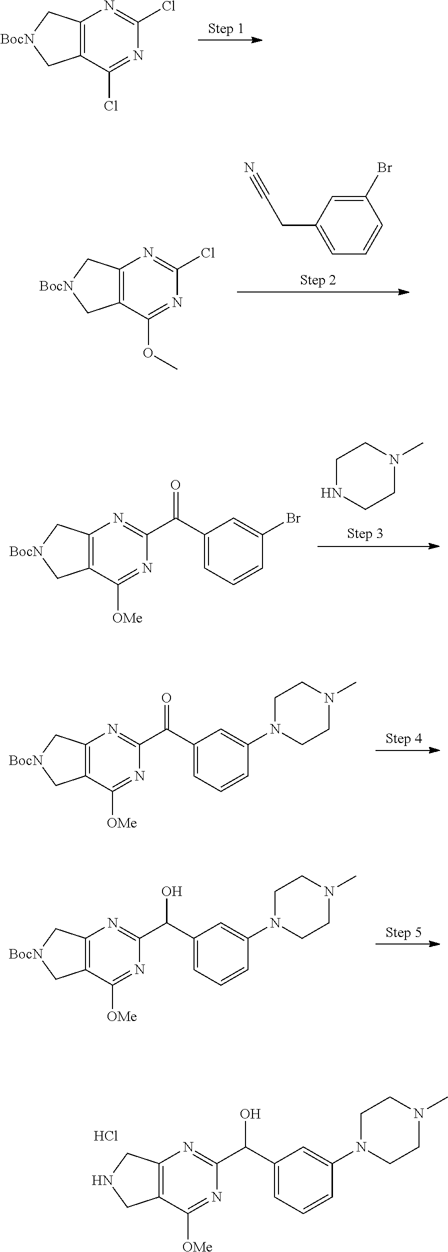

### Step 1. Tert-butyl 2-chloro-4-methoxy-5H,6H,7H-pyrrolo[3,4-d]pyrimidine-6-carboxylate

To a solution of tert-butyl 2,4-dichloro-5H,6H,7H-pyrrolo[3,4-d]pyrimidine-6-carboxylate (2.00 g, 6.55 mmol) in methanol (15 mL) was added a solution of sodium methoxide (447 mg, 7.86 mmol) in MeOH (5 mL) dropwise with stirring at 0° C. The resulting mixture was stirred for 14 h at room temperature. The reaction mixture was poured into water (10 mL) and then extracted with ethyl acetate (3×20 mL). The organic layers were combined, dried over anhydrous sodium sulfate, filtered, and concentrated under vacuum. The resulting crude product was purified by silica gel chromatography (eluting with 0:100 to 40:60 ethyl acetate/petroleum ether) to afford tert-butyl 2-chloro-4-methoxy-5H,6H,7H-pyrrolo[3,4-d]pyrimidine-6-carboxylate as yellow oil (1.8 g, 82%). LCMS (ES, m/z): 286, 288 [M+H]⁺.

### Step 2. Tert-butyl 2-(3-bromobenzoyl)-4-methoxy-5H,6H,7H-pyrrolo[3,4-d]pyrimidine-6-carboxylate

To a solution of tert-butyl 2-chloro-4-methoxy-5H,6H,7H-pyrrolo[3,4-d]pyrimidine-6-carboxylate (1.60 g, 5.60 mmol) in THF (20 mL) was added 2-(3-bromophenyl)acetonitrile (1.65 g, 8.40 mmol), and lithium hexamethyldisilazide solution (11.2 mL, 1.0 M in THF). The resulting mixture was stirred for 14 h at room temperature while oxygen was kept bubbling in. The reaction mixture was poured into water (10 mL) and then extracted with ethyl acetate (3×20 mL). The organic layers were combined, dried over anhydrous sodium sulfate, filtered, and concentrated under vacuum. The resulting crude product was purified by silica gel chromatography (eluting with 0:100 to 40:60 ethyl acetate/petroleum ether) to afford tert-butyl 2-(3-bromobenzoyl)-4-methoxy-5H,6H,7H-pyrrolo[3,4-d]pyrimidine-6-carboxylate as a yellow solid (1.2 g, 49%). LCMS (ES, m/z): 434, 436 [M+H]⁺.

### Step 3. Tert-butyl 4-methoxy-2-[3-(4-methylpiperazin-1-yl)benzoyl]-5H,6H,7H-pyrrolo[3,4-d]pyrimidine-6-carboxylate

To a solution of tert-butyl 2-(3-bromobenzoyl)-4-methoxy-5H,6H,7H-pyrrolo[3,4-d]pyrimidine-6-carboxylate (500 mg, 1.15 mmol) in 1,4-dioxane (5 mL) was added 1-methylpiperazine (0.79 mL, 11.5 mmol), cesium carbonate (1.13 g, 3.45 mmol), RuPhos (54 mg, 0.12 mmol), and RuPhos 3G (96 mg, 0.12 mmol). The resulting mixture was stirred for 3 h at 100° C. After cooling to room temperature, the reaction mixture was poured into water (5 mL) and then extracted with ethyl acetate (3×10 mL). The organic layers were combined, dried over anhydrous sodium sulfate, filtered, and concentrated under vacuum. The resulting crude product was purified by silica gel chromatography (eluting with 100:1 to 10:1 dichloromethane/methanol) to afford tert-butyl 4-methoxy-2-[3-(4-methylpiperazin-1-yl)benzoyl]-5H,6H,7H-pyrrolo[3,4-d]pyrimidine-6-carboxylate as yellow oil (350 mg, 57%). LCMS (ES, m/z): 454 [M+H]⁺.

### Step 4. Tert-butyl 2-{hydroxy[3-(4-methylpiperazin-1-yl)phenyl]methyl}-4-methoxy-5H,6H,7H-pyrrolo[3,4-d]pyrimidine-6-carboxylate

To a solution of tert-butyl 4-methoxy-2-[3-(4-methylpiperazin-1-yl)benzoyl]-5H,6H,7H-pyrrolo[3,4-d]pyrimidine-6-carboxylate (350 mg, 0.77 mmol) in methanol (5 mL) was added sodium borohydride (44 mg, 1.16 mmol) at 0° C. The resulting mixture was stirred for 1 h at room temperature. The resulting mixture was concentrated and diluted with water (2 mL). The reaction mixture was extracted with ethyl acetate (4×5 mL). The organic layers were combined, dried over anhydrous sodium sulfate, filtered, and concentrated under vacuum. The resulting crude product was purified by prep-TLC (eluting with 15:1 dichloromethane/methanol) to afford tert-butyl 2-{hydroxy[3-(4-methylpiperazin-1-yl)phenyl]methyl}-4-methoxy-5H,6H,7H-pyrrolo[3,4-d]pyrimidine-6-carboxylate as yellow oil (200 mg, 57%). LCMS (ES, m/z): 456 [M+H]⁺.

### Step 5. {4-methoxy-5H,6H,7H-pyrrolo[3,4-d]pyrimidin-2-yl}[3-(4-methylpiperazin-1-yl)phenyl]methanol (HCl salt)

To a solution of tert-butyl 2-{hydroxy[3-(4-methylpiperazin-1-yl)phenyl]methyl}-4-methoxy-5H,6H,7H-pyrrolo[3,4-d]pyrimidine-6-carboxylate (200 mg, 0.44 mmol) in dichloromethane (4 mL) was added hydrochloric acid (1 mL, 4 N in 1,4-dioxane). The resulting solution was stirred for 2 h at room temperature and concentrated under vacuum to afford {4-methoxy-5H,6H,7H-pyrrolo[3,4-d]pyrimidin-2-yl}[3-(4-methylpiperazin-1-yl)phenyl]methanol (HCl salt) as a yellow solid (60 mg, crude). LCMS (ES, m/z): 356 [M+H]⁺.

### Intermediate 30-1. Benzyl 4-[3-({6-[(tert-butoxy)carbonyl]-5H,6H,7H-pyrrolo[3,4-d]pyrimidin-2-yl} (hydroxy)methyl)phenyl]piperazine-1-carboxylate

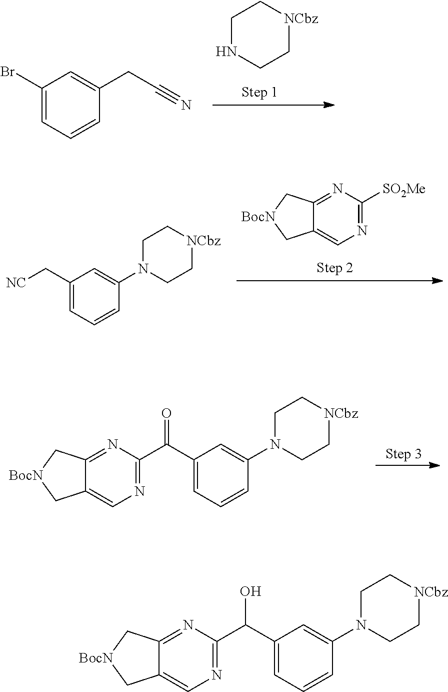 [SMILES: O=C(OCc1ccccc1)N1CCNCC1 ; N#CCc1cccc(Br)c1 ; N#CCc1cccc(N2CCN(C(=O)OCc3ccccc3)CC2)c1 ; CC(C)(C)OC(=O)N1Cc2cnc(S(C)(=O)=O)nc2C1 ; CC(C)(C)OC(=O)N1Cc2cnc(C(=O)c3cccc(N4CCN(C(=O)OCc5ccccc5)CC4)c3)nc2C1 ; CC(C)(C)OC(=O)N1Cc2cnc(C(O)c3cccc(N4CCN(C(=O)OCc5ccccc5)CC4)c3)nc2C1]

### Step 1. Benzyl 4-[3-(cyanomethyl)phenyl]piperazine-1-carboxylate

To a solution of 2-(3-bromophenyl)acetonitrile (3 g, 15.3 mmol) in 1,4-dioxane (50 mL), was added benzyl piperazine-1-carboxylate (6.74 g, 30.6 mmol), RuPhos (714 mg, 1.53 mmol), potassium phosphate (9.74 g, 45.9 mmol) and RuPhos 3G (1.28 g, 1.53 mmol). The resulting mixture was stirred for 16 h at 85° C. After cooling to room temperature, the reaction mixture was poured into water (100 mL) and then extracted with ethyl acetate (3×100 mL). The organic layers were combined, dried over anhydrous sodium sulfate, filtered, and concentrated under vacuum. The resulting crude product was purified by silica gel chromatography (eluting with 0:100 to 10:90 ethyl acetate/petroleum ether) to afford benzyl 4-[3-(cyanomethyl)phenyl]piperazine-1-carboxylate as a light yellow oil (1.0 g, 19.5%). LCMS (ES, m/z): 336 [M+H]⁺.

### Step 2. Benzyl 4-(3-{6-[(tert-butoxy)carbonyl]-5H,6H,7H-pyrrolo[3,4-d]pyrimidine-2-carbonyl}phenyl)piperazine-1-carboxylate

To a solution of benzyl 4-[3-(cyanomethyl)phenyl]piperazine-1-carboxylate (600 mg, 1.78 mmol) in THF (30 mL) was added tert-butyl 2-methanesulfonyl-5H,6H,7H-pyrrolo[3,4-d]pyrimidine-6-carboxylate (642 mg, 2.15 mmol) and potassium hexamethyldisilazide (3.58 mL, 1 M in THF). The resulting mixture was stirred for 4 h at 25° C. while oxygen was kept bubbling in. The reaction mixture was concentrated under vacuum. The resulting crude product was purified by silica gel chromatography (eluting with 0:100 to 10:90 ethyl acetate/petroleum ether) to afford benzyl 4-(3-{6-[(tert-butoxy)carbonyl]-5H,6H,7H-pyrrolo[3,4-d]pyrimidine-2-carbonyl}phenyl)piperazine-1-carboxylate as light yellow oil (350 mg, 48%). LCMS (ES, m/z): 544 [M+H]⁺.

### Step 3. Benzyl 4-[3-({6-[(tert-butoxy)carbonyl]-5H,6H,7H-pyrrolo[3,4-d]pyrimidin-2-yl}(hydroxy)methyl)phenyl]piperazine-1-carboxylate

To a solution of benzyl 4-(3-[6-[(tert-butoxy)carbonyl]-5H,6H,7H-pyrrolo[3,4-d]pyrimidine-2-carbonyl] phenyl)piperazine-1-carboxylate (390 mg, 0.72 mmol) in MeOH (10 mL) was added sodium borohydride (8 mg, 0.21 mmol). The resulting mixture was stirred for 2 h at 25° C. The reaction was quenched by addition of water (0.5 mL). The resulting mixture was concentrated under vacuum. The resulting crude product was purified by silica gel chromatography (eluting with 0:100 to 10:90 ethyl acetate/petroleum ether) to afford benzyl 4-[3-({6-[(tert-butoxy)carbonyl]-5H,6H,7H-pyrrolo[3,4-d]pyrimidin-2-yl}(hydroxy)methyl)phenyl]piperazine-1-carboxylate as light yellow oil (290 mg, 74%). LCMS (ES, m/z): 546 [M+H]⁺.

### Intermediate 31-1. Tert-butyl 2-[cyclohexyl(hydroxy)methyl]-5H,6H,7H-pyrrolo[3,4-d]pyrimidine-6-carboxylate

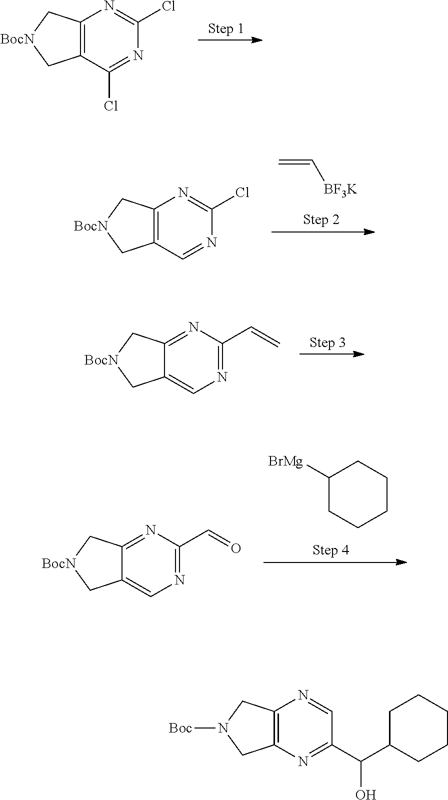

### Step 1. Tert-butyl 2-chloro-5H,6H,7H-pyrrolo[3,4-d]pyrimidine-6-carboxylate

To a solution of tert-butyl 2,4-dichloro-5H,6H,7H-pyrrolo[3,4-d]pyrimidine-6-carboxylate (3.0 g, 10.1 mmol) in MeOH (24 mL) was added zinc powder (1.3 g, 17.9 mmol) and acetic acid (5.8 mL, 101 mmol). The resulting mixture was stirred for 16 h at 50° C. After cooling to room temperature, the reaction mixture was filtered and concentrated under vacuum. The resulting crude product was purified by silica gel chromatography (eluting with 1:8 EA/PE) to afford tert-butyl 2-chloro-5H,6H,7H-pyrrolo[3,4-d]pyrimidine-6-carboxylate as a white solid (1.5 g, 52%). LCMS (ES, m/z): 256, 258 [M+H]⁺.

### Step 2. Tert-butyl 2-ethenyl-5H,6H,7H-pyrrolo[3,4-d]pyrimidine-6-carboxylate

To a solution of tert-butyl 2-chloro-5H,6H,7H-pyrrolo[3,4-d]pyrimidine-6-carboxylate (690 mg, 2.70 mmol) in 1,4-dioxane (20 mL) and water (4 mL) was added sodium carbonate (572 mg, 5.40 mmol), ethenyltrifluoro-λ⁴-borane potassium (542 mg, 4.05 mmol) and Pd(dppf)Cl₂ (197 mg, 0.27 mmol). The resulting mixture was stirred for 16 h at 100° C. After cooling to room temperature, the reaction mixture was poured into water (50 mL) and then extracted with ethyl acetate (3×50 mL). The organic layers were combined, dried over anhydrous sodium sulfate, filtered, and concentrated under vacuum. The resulting crude product was purified by silica gel chromatography (eluting with 10:90 ethyl acetate/petroleum ether) to afford tert-butyl 2-ethenyl-5H,6H,7H-pyrrolo[3,4-d]pyrimidine-6-carboxylate as a light yellow solid (430 mg, 64%). LCMS (ES, m/z): 248 [M+H]⁺.

### Step 3. Tert-butyl 2-formyl-5H,6H,7H-pyrrolo[3,4-d]pyrimidine-6-carboxylate

To a solution of tert-butyl 2-ethenyl-5H,6H,7H-pyrrolo[3,4-d]pyrimidine-6-carboxylate (450 mg, 1.73 mmol) in THF (10 mL) and water (5 mL) was added sodium periodate (739 mg, 3.45 mmol) and osmium tetraoxide (44 mg, 0.17 mmol). The resulting mixture was stirred for 2 h at 25° C. The reaction mixture was poured into water (15 mL) and then extracted with ethyl acetate (3×15 mL). The organic layers were combined, dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 2-formyl-5H,6H,7H-pyrrolo[3,4-d]pyrimidine-6-carboxylate as a light yellow solid (300 mg, 70%). LCMS (ES, m/z): 250 [M+H]⁺.

### Step 4. Tert-butyl 2-[cyclohexyl(hydroxy)methyl]-5H,6H,7H-pyrrolo[3,4-d]pyrimidine-6-carboxylate

To a solution of tert-butyl 2-formyl-5H,6H,7H-pyrrolo[3,4-d]pyrimidine-6-carboxylate (150 mg, 0.57 mmol) in THF (8 mL), was added bromo(cyclohexyl)magnesium (0.9 mL, 1.0 M in THF) at 0° C. The resulting solution was stirred for 2 h at 25° C. The reaction mixture was poured into saturated ammonium chloride (10 mL) and then extracted with ethyl acetate (3×10 mL). The organic layers were combined, dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 2-[cyclohexyl(hydroxy)methyl]-5H,6H,7H-pyrrolo[3,4-d]pyrimidine-6-carboxylate as a light yellow solid (30 mg, 16%). LCMS (ES, m/z): 334 [M+H]⁺.

The Intermediate in Table 12 were synthesized according to the procedure described for Intermediate 31-1 above.

TABLE 12

| | | LCMS: |
|---|---|---|
| Inter- | | (ESI) m/z |
| mediate | Compound | [M + H]⁺ |
| 31-2 | 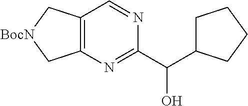 [SMILES: CC(C)(C)OC(=O)N1Cc2cnc(C(O)C3CCCC3)nc2C1] | 320 |
| | tert-butyl 2-(cyclopentyl(hydroxy)methyl)-5,7-dihydro- | |
| | 6H-pyrrolo[3,4-d]pyrimidine-6-carboxylate | |

### Intermediate 32-1. 4-({6-[hydroxy(phenyl)methyl]-1H,2H,3H-pyrrolo[3,4-c]pyridin-2-yl}sulfonyl)benzonitrile

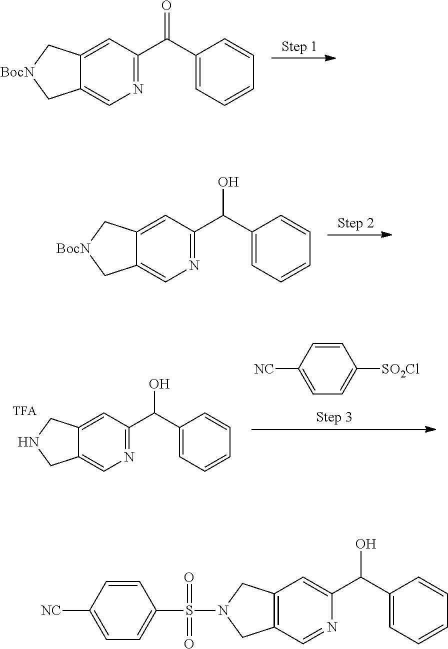 [SMILES: CC(C)(C)OC(=O)N1Cc2cnc(C(=O)c3ccccc3)cc2C1 ; CC(C)(C)OC(=O)N1Cc2cnc(C(O)c3ccccc3)cc2C1 ; OC(c1ccccc1)c1cc2c(cn1)CNC2 ; N#Cc1ccc(S(=O)(=O)Cl)cc1 ; N#Cc1ccc(S(=O)(=O)N2Cc3cnc(C(O)c4ccccc4)cc3C2)cc1]

### Step 1. Tert-butyl 6-[hydroxy(phenyl)methyl]-1H,2H,3H-pyrrolo[3,4-c]pyridine-2-carboxylate

To a solution of tert-butyl 6-benzoyl-1H,2H,3H-pyrrolo[3,4-c]pyridine-2-carboxylate (340 mg, 1.05 mmol) in MeOH (10 mL), was added sodium borohydride (12 mg, 0.32 mmol). The resulting mixture was stirred for 1 h at 25° C. The reaction mixture was poured into water (15 mL) and then extracted with ethyl acetate (3×10 mL). The organic layers were combined, dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 6-[hydroxy(phenyl)methyl]-1H,2H,3H-pyrrolo[3,4-c]pyridine-2-carboxylate as a yellow solid (300 mg, 88%). LCMS (ES, m/z): 327 [M+H]⁺.

### Step 2. Phenyl({1H,2H,3H-pyrrolo[3,4-c]pyridin-6-yl})methanol (TFA salt)

To a solution of tert-butyl 6-[hydroxy(phenyl)methyl]-1H,2H,3H-pyrrolo[3,4-c]pyridine-2-carboxylate (280 mg, 0.86 mmol) in dichloromethane (4 mL) was added TFA (1 mL). The resulting mixture was stirred for 1 h at 25° C. The reaction mixture was concentrated under vacuum to afford phenyl({1H,2H,3H-pyrrolo[3,4-c]pyridin-6-yl})methanol (TFA salt) as light yellow oil (200 mg, crude). LCMS (ES, m/z): 227 [M+H]⁺.

### Step 3. 4-({6-[hydroxy(phenyl)methyl]-1H,2H,3H-pyrrolo[3,4-c]pyridin-2-yl}sulfonyl)benzonitrile

To a solution of phenyl({1H,2H,3H-pyrrolo[3,4-c]pyridin-6-yl})methanol (TFA salt) (225 mg, 0.99 mmol) in dichloromethane (7 mL) was added TEA (0.55 mL, 3.98 mmol) and 4-cyanobenzene-1-sulfonyl chloride (200 mg, 0.99 mmol). The resulting mixture was stirred for 2 h at 25° C. and concentrated under vacuum. The resulting crude product was purified by silica gel chromatography (eluting with 0:100 to 10:90 ethyl acetate/petroleum ether) to afford 4-({6-[hydroxy(phenyl)methyl]-1H,2H,3H-pyrrolo[3,4-c]pyridin-2-yl}sulfonyl)benzonitrile as a white solid (200 mg, 51%). LCMS (ES, m/z): 392 [M+H]⁺.

The Intermediate in Table 13 were synthesized according to the procedure described for Intermediate 32-1 above.

TABLE 13

| | | LCMS: (ESI) |
|---|---|---|
| Intermediate | Compound | m/z [M + H]⁺ |
| 32-2 | 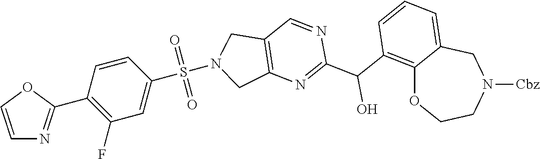 [SMILES: O=C(OCc1ccccc1)N1CCOc2c(cccc2C(O)c2ncc3c(n2)CN(S(=O)(=O)c2ccc(-c4ncco4)c(F)c2)C3)C1] | 658 |
| | benzyl 9-((6-((3-fluoro-4-(oxazol-2- | |
| | yl)phenyl)sulfonyl)-6,7-dihydro-5H-pyrrolo[3,4- | |
| | d]pyrimidin-2-yl)(hydroxy)methyl)-2,3- | |
| | dihydrobenzo[f][1,4]oxazepine-4(5H)-carboxylate | |

### Intermediate 33-1. Tert-butyl 2-(3-bromobenzoyl)-5H,6H,7H-pyrrolo[3,4-d]pyrimidine-6-carboxylate

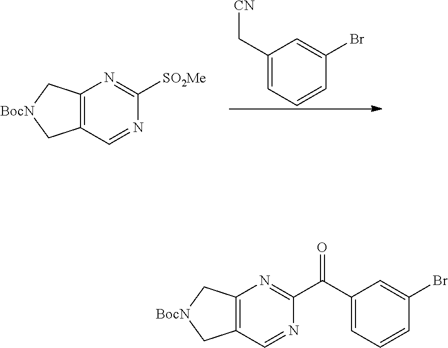 [SMILES: CC(C)(C)OC(=O)N1Cc2cnc(S(C)(=O)=O)nc2C1 ; N#CCc1cccc(Br)c1 ; CC(C)(C)OC(=O)N1Cc2cnc(C(=O)c3cccc(Br)c3)nc2C1]

To a solution of 2-(3-bromophenyl)acetonitrile (1.96 g, 9.52 mmol) in THF (50 mL) was added tert-butyl 2-methanesulfonyl-5H,6H,7H-pyrrolo[3,4-d]pyrimidine-6-carboxylate (2 g, 6.35 mmol) and potassium bis(trimethylsilyl)amide solution (10 mL, 1 M in THF). The resulting mixture was stirred for 12 h at room temperature while oxygen was kept bubbling in. The reaction mixture was poured into water (50 mL) and then extracted with ethyl acetate (3×50 mL). The organic layers were combined, dried over anhydrous sodium sulfate, filtered, and concentrated under vacuum. The resulting crude product was purified by silica gel chromatography (eluting with 0:100 to 30:70 ethyl acetate/petroleum ether) to afford tert-butyl 2-(3-bromobenzoyl)-5H,6H,7H-pyrrolo[3,4-d]pyrimidine-6-carboxylate as a yellow solid (1.20 g, 44%). LCMS (ES, m/z): 404, 406 [M+H]⁺.

The Intermediate in Table 14 were synthesized according to the procedure described for Intermediate 33-1 above.

TABLE 14

| | | LCMS: (ESI) |
|---|---|---|
| Intermediate | Compound | m/z [M + H]⁺ |
| 33-2 | 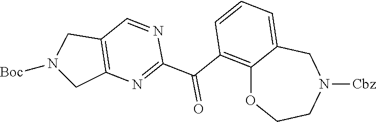 [SMILES: CC(C)(C)OC(=O)N1Cc2cnc(C(=O)c3cccc4c3OCCN(C(=O)OCc3ccccc3)C4)nc2C1] | 531 |
| | benzyl 9-(6-(tert-butoxycarbonyl)-6,7-dihydro-5H- | |
| | pyrrolo[3,4-d]pyrimidine-2-carbonyl)-2,3- | |
| | dihydrobenzo[f][1,4]oxazepine-4(5H)-carboxylate | |

### Intermediate 34-1. Benzyl 2-methanesulfonyl-5H,6H,7H-pyrrolo[3,4-d]pyrimidine-6-carboxylate

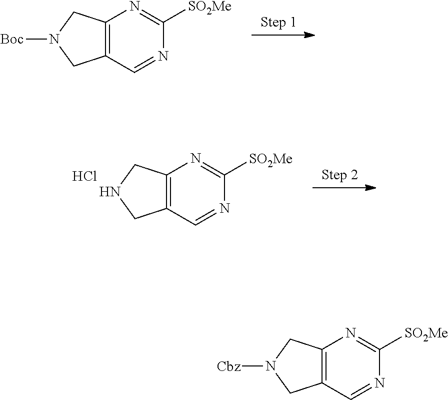 [SMILES: CC(C)(C)OC(=O)N1Cc2cnc(S(C)(=O)=O)nc2C1 ; Cl ; CS(=O)(=O)c1ncc2c(n1)CNC2 ; CS(=O)(=O)c1ncc2c(n1)CN(C(=O)OCc1ccccc1)C2]

### Step 1. 2-methanesulfonyl-5H,6H,7H-pyrrolo[3,4-d]pyrimidine (HCl salt)

To tert-butyl 2-methanesulfonyl-5H,6H,7H-pyrrolo[3,4-d]pyrimidine-6-carboxylate (2 g, 6.35 mmol) was added hydrochloric acid (20 mL, 4 N in 1,4-dioxane). The resulting solution was stirred for 2 h at room temperature. The reaction mixture was concentrated to afford 2-methanesulfonyl-5H,6H,7H-pyrrolo[3,4-d]pyrimidine HCl salt as yellow oil (1.4 g, crude). LCMS (ES, m/z): 200 [M+H]⁺.

### Step 2. Benzyl 2-methanesulfonyl-5H,6H,7H-pyrrolo[3,4-d]pyrimidine-6-carboxylate

To a solution of 2-methanesulfonyl-5H,6H,7H-pyrrolo[3,4-d]pyrimidine (HCl salt) (1.4 g, 6.33 mmol) in DCM (20 mL) was added TEA (2.77 mL, 19.0 mmol), and CbzCl (1.90 mL, 12.6 mmol) at 0° C. The resulting solution was stirred for 5 h at room temperature. The reaction mixture was poured into water (50 mL) and then extracted with ethyl acetate (3×50 mL). The organic layers were combined, dried over anhydrous sodium sulfate, filtered and concentrated under vacuum. The resulting crude product was purified by silica gel chromatography (eluting with 2:1 ethyl acetate/petroleum ether) to afford benzyl 2-methanesulfonyl-5H,6H,7H-pyrrolo[3,4-d]pyrimidine-6-carboxylate as a yellow solid (800 mg, 36%). LCMS (ES, m/z): 334 [M+H]⁺.

### Intermediate 35-1. [(Tert-butoxy)(hydroxy)methyl](methyl)[(8-[5H,6H,7H-pyrrolo[3,4-d]pyrimidine-2-carbonyl]quinolin-2-yl)methyl]amine

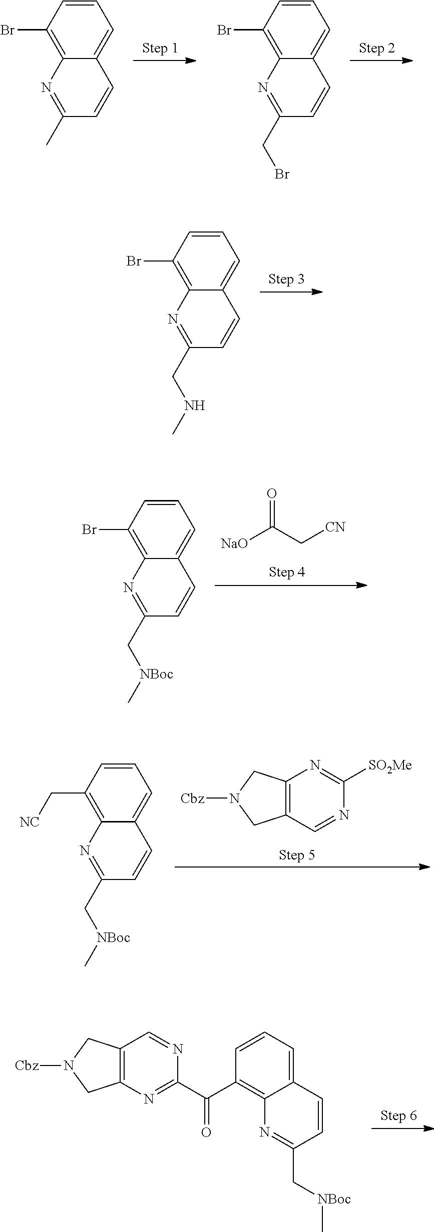

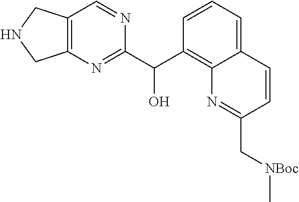 [SMILES: CN(Cc1ccc2cccc(C(O)c3ncc4c(n3)CNC4)c2n1)C(=O)OC(C)(C)C]

### Step 1. 8-bromo-2-(bromomethyl)quinoline

To a solution of 8-bromo-2-methylquinoline (5 g, 21.4 mmol) in tetrachloride carbon (50 mL) was added NBS (4.4 g, 23.5 mmol) and AIBN (37 mg, 0.21 mmol). The resulting mixture was stirred overnight at 80° C. After cooling to room temperature, the reaction mixture was filtered and concentrated under vacuum. The resulting crude product was purified by silica gel chromatography (eluting with 0:100 to 20:80 ethyl acetate/petroleum ether) to afford 8-bromo-2-(bromomethyl)quinoline as a white solid (2 g, 28%). LCMS (ES, m/z): 300, 302, 304 [M+H]⁺.

### Step 2. [(8-bromoquinolin-2-yl)methyl](methyl)amine

To a solution of 8-bromo-2-(bromomethyl)quinoline (2 g, 5.32 mmol) in THF (20 mL) was added methylamine (20 mL, 2 M in THF). The resulting solution was stirred overnight at room temperature. The reaction mixture was filtered and concentrated under vacuum to afford [(8-bromoquinolin-2-yl)methyl](methyl)amine as yellow oil (800 mg, 48%). LCMS (ES, m/z): 251, 253 [M+H]⁺.

### Step 3. Tert-butyl N-[(8-bromoquinolin-2-yl)methyl]-N-methylcarbamate

To a solution of [(8-bromoquinolin-2-yl)methyl](methyl)amine (800 mg, 2.55 mmol) in DCM (10 mL) was added Boc₂O (2.0 mL, 8.71 mmol). The resulting solution was stirred for 5 h at room temperature. The resulting mixture was concentrated. The resulting crude product was purified by silica gel chromatography (eluting with 0:100 to 30:60 ethyl acetate/petroleum ether) to afford tert-butyl N-[(8-bromoquinolin-2-yl)methyl]-N-methylcarbamate as yellow oil (600 mg, 60%). LCMS (ES, m/z): 351, 353 [M+H]⁺.

### Step 4. Tert-butyl N-[[8-(cyanomethyl)quinolin-2-yl]methyl]-N-methylcarbamate

To a solution of tert-butyl N-[(8-bromoquinolin-2-yl)methyl]-N-methylcarbamate (600 mg, 1.54 mmol) in mesitylene (10 mL) was added Pd₂(allyl)₂Cl₂ (12 mg, 0.03 mmol), S-Phos (40 mg, 0.09 mmol), and sodium 2-cyanoacetate (260 mg, 2.31 mmol). The resulting mixture was stirred for 5 h at 140° C. After cooling to room temperature, the reaction mixture was poured into water (20 mL) and then extracted with ethyl acetate (3×20 mL). The organic layers were combined, dried over anhydrous sodium sulfate, filtered, and concentrated under vacuum. The resulting crude product was purified by Prep-TLC (eluting with 1:2 ethyl acetate/petroleum ether) to afford tert-butyl N-[[8-(cyanomethyl)quinolin-2-yl]methyl]-N-methylcarbamate as yellow solid (500 mg, 94%). LCMS (ES, m/z): 312 [M+H]⁺.

### Step 5. Benzyl 2-[2-([[(tert-butoxy)carbonyl](methyl)amino]methyl)quinoline-8-carbonyl]-5H,6H,7H-pyrrolo[3,4-d]pyrimidine-6-carboxylate

To a solution of tert-butyl N-[[8-(cyanomethyl)quinolin-2-yl]methyl]-N-methylcarbamate (500 mg, 1.45 mmol) in THF (10 mL) was added benzyl 2-methanesulfonyl-5H,6H,7H-pyrrolo[3,4-d]pyrimidine-6-carboxylate (535 mg, 1.45 mmol), and LiHMDS (2.2 mL, 1 M in THF). The resulting mixture was stirred for 2 days at room temperature while oxygen was kept bubbling in. The reaction mixture was poured into water (20 mL) and then extracted with ethyl acetate (3×20 mL). The organic layers were combined, dried over anhydrous sodium sulfate, filtered, and concentrated under vacuum. The resulting crude product was purified by Prep-TLC (eluting with 2:1 ethyl acetate/petroleum ether) to afford benzyl 2-[2-([[(tert-butoxy)carbonyl](methyl)amino]methyl)quinoline-8-carbonyl]-5H,6H,7H-pyrrolo[3,4-d]pyrimidine-6-carboxylate as yellow solid (150 mg, 18%). LCMS (ES, m/z): 554[M+H]⁺.

### Step 6. [(Tert-butoxy)(hydroxy)methyl](methyl)[(8-[5H,6H,7H-pyrrolo[3,4-d]pyrimidine-2-carbonyl]quinolin-2-yl)methyl]amine

To a solution of benzyl 2-[2-([[(tert-butoxy)carbonyl](methyl)amino]methyl)quinoline-8-carbonyl]-5H,6H,7H-pyrrolo[3,4-d]pyrimidine-6-carboxylate (150 mg, 0.26 mmol) in MeOH (5 mL) was added Pd/C (15 mg, 10% Pd). The resulting mixture was stirred for 24 h at room temperature under hydrogen atmosphere (2-3 atm). The reaction mixture was filtered and concentrated under vacuum to afford [(tert-butoxy)(hydroxy)methyl](methyl)[(8-[5H,6H,7H-pyrrolo[3,4-d]pyrimidine-2-carbonyl]quinolin-2-yl)methyl]amine as yellow oil (80 mg, 49%). LCMS (ES, m/z): 422 [M+H]⁺.

### Intermediate 36-1. {3-methyl-1H,2H,3H-pyrrolo[3,4-c]pyridin-6-yl}[3-(4-methylpiperazin-1-yl)phenyl]methanol (HCl salt) and {1-methyl-1H,2H,3H-pyrrolo[3,4-c]pyridin-6-yl}[3-(4-methylpiperazin-1-yl)phenyl]methanol (HCl salt)

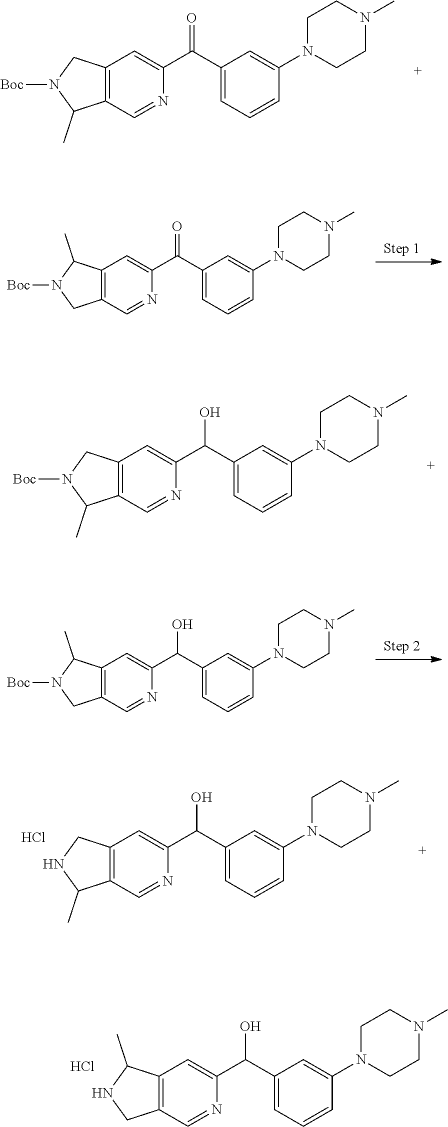

### Step 1. Tert-butyl 6-{hydroxy[3-(4-methylpiperazin-1-yl)phenyl]methyl}-3-methyl-1H,2H,3H-pyrrolo [3,4-c]pyridine-2-carboxylate and tert-butyl 6-{hydroxy[3-(4-methylpiperazin-1-yl)phenyl]methyl}-1-methyl-1H,2H,3H-pyrrolo[3,4-c]pyridine-2-carboxylate

To a solution of tert-butyl 3-methyl-6-[3-(4-methylpiperazin-1-yl)benzoyl]-1H,2H,3H-pyrrolo[3,4-c]pyridine-2-carboxylate and tert-butyl 1-methyl-6-[3-(4-methylpiperazin-1-yl)benzoyl]-1H,2H,3H-pyrrolo[3,4-c]pyridine-2-carboxylate (1 g, 2.29 mmol) in tert-butanol (8 mL) was added potassium tert-butoxide (12 mg, 0.11 mmol) and RuCl₂[(R)-DM-BINAP][(R)-DAIPEN] (27 mg, 0.02 mmol). The resulting mixture was stirred for 16 h at 25° C. under hydrogen atmosphere (8 atm). The reaction mixture was concentrated under vacuum. The resulting crude product was purified by silica gel chromatography (eluting with 0:100 to 10:90 ethyl acetate/petroleum ether) to afford tert-butyl 6-[hydroxy[3-(4-methylpiperazin-1-yl)phenyl]methyl]-3-methyl-1H,3H-pyrrolo[3,4-c]pyridine-2-carboxylate and tert-butyl 6-[hydroxy[3-(4-methylpiperazin-1-yl)phenyl]methyl]-1-methyl-1H,3H-pyrrolo[3,4-c]pyridine-2-carboxylate as light yellow oil (700 mg, 70%). LCMS (ES, m/z): 439 [M+H]⁺.

### Step 2. {3-methyl-1H,2H,3H-pyrrolo[3,4-c]pyridin-6-yl}[3-(4-methylpiperazin-1-yl)phenyl]methanol (HCl salt) and {1-methyl-1H,2H,3H-pyrrolo[3,4-c]pyridin-6-yl}[3-(4-methylpiperazin-1-yl)phenyl]methanol (HCl salt)

To a solution of tert-butyl 6-[hydroxy[3-(4-methylpiperazin-1-yl)phenyl]methyl]-3-methyl-1H,3H-pyrrolo[3,4-c]pyridine-2-carboxylate and tert-butyl 6-[hydroxy[3-(4-methylpiperazin-1-yl)phenyl]methyl]-1-methyl-1H,3H-pyrrolo[3,4-c]pyridine-2-carboxylate (600 mg, 1.36 mmol) in DCM (5 mL), was added hydrochloric acid (15 mL, 4 N in 1,4-dioxane). The resulting mixture was stirred for 16 h at room temperature. The reaction mixture was concentrated under vacuum to afford {3-methyl-1H,2H,3H-pyrrolo[3,4-c]pyridin-6-yl}[3-(4-methylpiperazin-1-yl)phenyl]methanol (HCl salt) and {1-methyl-1H,2H,3H-pyrrolo[3,4-c]pyridin-6-yl}[3-(4-methylpiperazin-1-yl)phenyl]methanol (HCl salt) as a light yellow solid (350 mg, 68%). LCMS (ES, m/z): 339 [M+H]⁺.

### Intermediate 37-1. Tert-butyl 3-[3-[hydroxy([1H,2H,3H-pyrrolo[3,4-c]pyridin-6-yl])methyl]phenoxy]azetidine-1-carboxylate

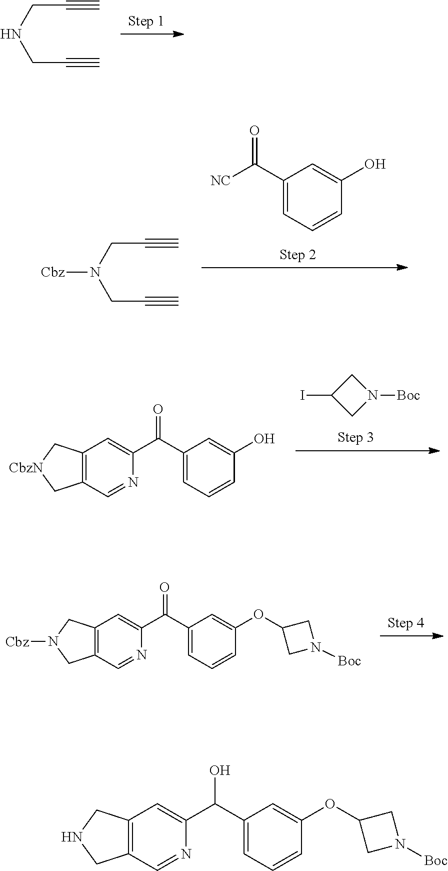

### Step 1. Benzyl N,N-bis(prop-2-yn-1-yl)carbamate

To a solution of bis(prop-2-yn-1-yl)amine (5 g, 51.0 mmol) in DCM (50 mL) was added TEA (21.7 mL, 153 mmol) and CbzCl (15.1 mL, 102 mmol). The resulting solution was stirred for 12 h at room temperature. The reaction mixture was poured into water (50 mL) and then extracted with ethyl acetate (3×50 mL). The organic layers were combined, dried over anhydrous sodium sulfate, filtered, and concentrated under vacuum. The resulting crude product was purified by silica gel chromatography (eluting with 0:100 to 20:80 ethyl acetate/petroleum ether) to afford benzyl N,N-bis(prop-2-yn-1-yl)carbamate as yellow oil (8 g, 66%). LCMS (ES, m/z): 228 [M+H]⁺.

### Step 2. Benzyl 6-(3-hydroxybenzoyl)-1H,2H,3H-pyrrolo[3,4-c]pyridine-2-carboxylate

To a solution of 3-hydroxybenzoyl cyanide (1.6 g, 9.50 mmol) in DCE (20 mL) was added Cp*Ru(COD)Cl (61 mg, 0.16 mmol), and benzyl N,N-bis(prop-2-yn-1-yl)carbamate (2 g, 7.92 mmol) at 0° C. The resulting solution was stirred for 1 h at 60° C. After cooling to room temperature, the reaction mixture was concentrated under vacuum. The resulting crude product was purified by Prep-TLC (eluting with 1:1 ethyl acetate/petroleum ether) to afford benzyl 6-(3-hydroxybenzoyl)-1H,2H,3H-pyrrolo[3,4-c]pyridine-2-carboxylate as a yellow solid (500 mg, 16%). LCMS (ES, m/z): 375 [M+H]⁺.

### Step 3. Tert-butyl 3-(3-[2-[(benzyloxy)carbonyl]-1H,2H,3H-pyrrolo[3,4-c]pyridine-6-carbonyl]phenoxy)azetidine-1-carboxylate

To a solution of benzyl 6-(3-hydroxybenzoyl)-1H,2H,3H-pyrrolo[3,4-c]pyridine-2-carboxylate (450 mg, 1.14 mmol) in DMF (5 mL) was added cesium carbonate (1.2 g, 3.43 mmol) and tert-butyl 3-iodoazetidine-1-carboxylate (1.0 g, 3.42 mmol). The resulting mixture was stirred for 5 h at 85° C. After cooling to room temperature, the reaction mixture was poured into water (20 mL) and then extracted with ethyl acetate (3×20 mL). The organic layers were combined, dried over anhydrous sodium sulfate, filtered, and concentrated under vacuum. The resulting crude product was purified by Prep-TLC (eluting with 1:1 ethyl acetate/petroleum ether) to afford benzyl N,N-bis(prop-2-yn-1-yl)carbamate as yellow oil (300 mg, 47%). LCMS (ES, m/z): 530 [M+H]⁺.

### Step 4. Tert-butyl 3-[3-[hydroxy([1H,2H,3H-pyrrolo[3,4-c]pyridin-6-yl])methyl]phenoxy]azetidine-1-carboxylate

To a solution of tert-butyl 3-(3-[2-[(benzyloxy)carbonyl]-1H,2H,3H-pyrrolo[3,4-c]pyridine-6-carbonyl]phenoxy) azetidine-1-carboxylate (250 mg, 0.45 mmol) in MeOH (10 mL) was added Pd/C (50 mg, 10% Pd). The resulting mixture was stirred for 5 h at room temperature under hydrogen atmosphere (2-3 atm). The reaction mixture was filtered and concentrated under vacuum to afford tert-butyl 3-[3-[hydroxy([1H,2H,3H-pyrrolo[3,4-c]pyridin-6-yl])methyl]phenoxy]azetidine-1-carboxylate as yellow oil (180 mg, 81%). LCMS (ES, m/z): 398 [M+H]⁺.

### Intermediate 38-1. Tert-butyl 4,5-dihydropyrrolo[3,4-c]pyrrole-2(1H,3H,4H)-carboxylate (p-toluenesulfonate salt)

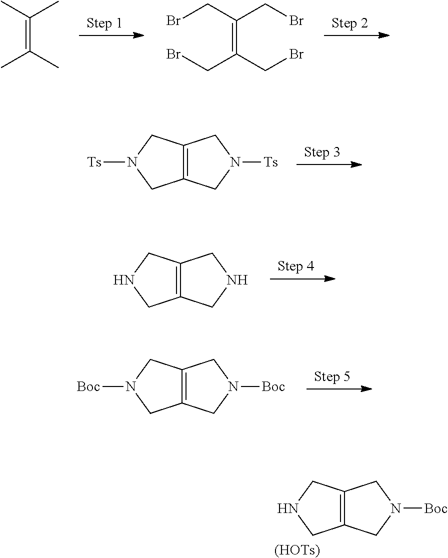

### Step 1. 1,4-dibromo-2,3-bis(bromomethyl)but-2-ene

To a solution of 2,3-dimethylbut-2-ene (1000 g, 11.9 mol) in DCM (1000 mL) in 4 L 4-necked round bottom flask was added aqueous hydrobromide solution (150 mL, 48%). This was followed by the slow addition of bromine (3.71 L, 62.0 mol) with stirring at 10-15° C. The resulting mixture was stirred for 2 days at 45° C. in an oil bath. After cooling to room temperature, the reaction mixture was carefully poured into saturated sodium hydrogen sulfite solution (10 L). The precipitation was collected by filtration and dried in oven to afford 1,4-dibromo-2,3-bis(bromomethyl)but-2-ene as a light yellow solid (3000 g, 44%). GCMS: (EI, m/z): 398, 400, 402 [M]⁺.

### Step 2. 2,5-ditosyl-1,2,3,4,5,6-hexahydropyrrolo[3,4-c]pyrrole

To a solution of 1,4-dibromo-2,3-bis(bromomethyl)but-2-ene (2000 g, 3.50 mol) in DMF (20 L) was added 4-methylbenzene-1-sulfonamide (2137 g, 12.5 mol) and potassium carbonate (5175 g, 37.4 mol). The resulting mixture was stirred for 2 days at room temperature. The reaction mixture was then slowly poured into water/ice (20 L). The precipitation was collected by filtration, washed with ethanol and dried in oven to afford 2,5-ditosyl-1,2,3,4,5,6-hexahydropyrrolo[3,4-c]pyrrole as a light yellow solid (1345 g, 78%). LCMS (ES, m/z): 419 [M+H]⁺.

### Step 3. 1,2,3,4,5,6-hexahydropyrrolo[3,4-c]pyrrole hydrobromide salt

To a solution of 2,5-ditosyl-1,2,3,4,5,6-hexahydropyrrolo[3,4-c]pyrrole (1345 g, 2.73 mol) in aqueous hydrobromide solution (4500 mL, 48%) in 10 L 4-necked round-bottom flask, was added phenol (1270 g, 13.5 mol). The resulting mixture was stirred for 2 days at 120° C. After cooling to room temperature, the aqueous layer was collected and concentrated under vacuum. The resulting solids were washed with DCM/MeOH (v:v=10:1, 3×300 mL) and dried in oven to afford 1,2,3,4,5,6-hexahydropyrrolo[3,4-c]pyrrole hydro bromide salt as a yellow solid (480 g, 61%). LCMS (ES, m/z): 111 [M+H]⁺.

### Step 4. Di-tert-butyl pyrrolo[3,4-c]pyrrole-2,5(1H,3H,4H,6H)-dicarboxylate

To a suspension of 1,2,3,4,5,6-hexahydropyrrolo[3,4-c]pyrrole hydrobromide salt (458 g, 1.52 mol) in water (4 L) was added sodium bicarbonate (424 g, 5.05 mol). This was followed by the dropwise addition of a solution of di-tert-butyl dicarbonate (807 g, 3.70 mol) in methanol (500 mL) with stirring at 0° C. The resulting solution was stirred for 16 h at 25° C. The precipitation was collected by filtration and dried in oven to afford di-tert-butyl pyrrolo[3,4-c]pyrrole-2,5(1H,3H,4H,6H)-dicarboxylate as a white solid (300 g, 61%). LCMS (ES, m/z): 311[M+H]⁺.

### Step 5. Tert-butyl 4,5-dihydropyrrolo[3,4-c]pyrrole-2(1H,3H,4H)-carboxylate (p-toluenesulfonate salt)

To a solution of di-tert-butyl pyrrolo[3,4-c]pyrrole-2,5(1H,3H,4H,6H)-dicarboxylate (200 g, 612 mmol) in propan-2-yl acetate (5 L) was added 4-methylbenzene-1-sulfonic acid (123 g, 647 mmol) in portions at 0° C. The resulting mixture was stirred for 16 h at 55° C. in an oil bath. After cooling to room temperature, the precipitation were collected by filtration and dried in oven to afford tert-butyl 4,5-dihydropyrrolo[3,4-c]pyrrole-2(1H,3H,4H)-carboxylate 4-methylbenzene-1-sulfonic acid salt as a yellow solid (197 g, 80%). LCMS (ES, m/z): 211[M+H]⁺.

### Example 2. Synthesis of Disclosed Compounds

### Method A

### [(2S)-2-[2-(2,3-dihydro-1,4-benzodioxine-6-sulfonyl)-1H,2H,3H-pyrrolo[3,4-c]pyridin-6-yl]-2-phenylethyl] (methyl)amine; [(2R)-2-[2-(2,3-dihydro-1,4-benzodioxine-6-sulfonyl)-1H,2H,3H-pyrrolo[3,4-c]pyridin-6-yl]-2-phenylethyl](methyl) amine

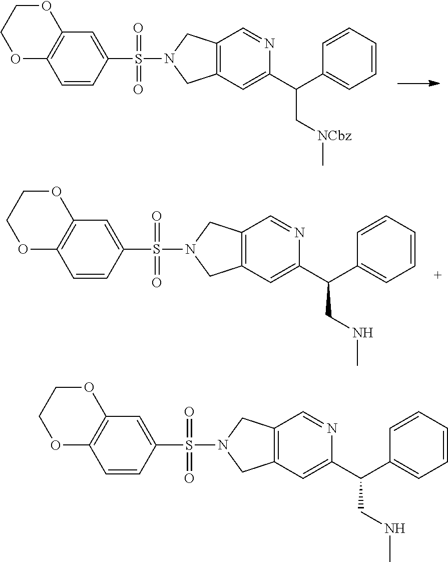 [SMILES: CN(CC(c1ccccc1)c1cc2c(cn1)CN(S(=O)(=O)c1ccc3c(c1)OCCO3)C2)C(=O)OCc1ccccc1 ; CNC[C@H](c1ccccc1)c1cc2c(cn1)CN(S(=O)(=O)c1ccc3c(c1)OCCO3)C2 ; CNC[C@@H](c1ccccc1)c1cc2c(cn1)CN(S(=O)(=O)c1ccc3c(c1)OCCO3)C2]

To a solution of (3-[2-[2-(2,3-dihydro-1,4-benzodioxine-6-sulfonyl)-1H,2H,3H-pyrrolo[3,4-c]pyridin-6-yl]-2-phenylethyl]phenyl)methyl N-methylcarbamate (100 mg, 0.17 mmol) in methanol (2 mL) was added palladium carbon (10 mg, 10 wt % palladium on charcoal). Then hydrogen was introduced with hydrogen balloon. The resulting mixture was stirred for 16 h at room temperature. The reaction mixture was filtered and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (5% to 30% over 25 min); Flow rate: 20 mL/min; Detector: UV 254 nm). The two enantiomers were further separated by Chiral-Pre-HPLC (Column: CHIRALPAK IG, 5 μm, 20×250 mm; Mobile Phase, A: methanol (containing 0.1% DEA) and B: DCM (hold 50% B over 10 min); Detector: UV 254/220 nm; Retention time: 1⁴ eluting isomer, 3.965 min; 2^{nd} eluting isomer, 5.955 min). The product fractions of 1^{st} eluting isomer were concentrated and lyophilized to afford a white solid (10.1 mg, 26%). ¹H-NMR (Methanol-d₄, 400 MHz,) δ (ppm): 8.40 (s, 1H), 7.36-7.16 (m, 7H), 7.17 (s, 1H), 6.99-6.92 (m, 1H), 4.60 (s, 2H), 4.54 (s, 2H), 4.35-4.33 (m, 1H), 4.26-4.22 (m, 4H), 3.53-3.50 (m, 1H), 3.19-3.14 (m, 1H), 2.44 (s, 3H). LCMS (ES, m/z) 452 [M+H]⁺. The product fractions of 2^{nd} eluting isomer were concentrated and lyophilized to a white solid (11.5 mg, 30%). ¹H-NMR (Methanol-d₄, 400 MHz,) δ (ppm): 8.40 (s, 1H), 7.36-7.16 (m, 7H), 7.17 (s, 1H), 6.99-6.92 (m, 1H), 4.61 (s, 2H), 4.56 (s, 2H), 4.42-4.39 (m, 1H), 4.26-4.22 (m, 4H), 3.66-3.59 (m, 1H), 3.19-3.14 (m, 1H), 2.55 (s, 3H). LCMS (ES, m/z) 452 [M+H]⁺.

### Method B

### [2-[3-fluoro-4-(1,3-oxazol-2-yl)benzenesulfonyl]-1H,2H,3H-pyrrolo[3,4-c]pyridin-6-yl][3-(piperazin-1-yl)phenyl]methanol

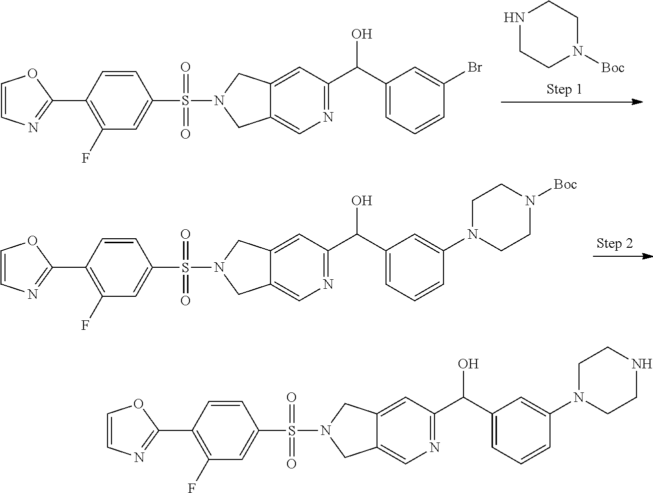 [SMILES: O=S(=O)(c1ccc(-c2ncco2)c(F)c1)N1Cc2cnc(C(O)c3cccc(Br)c3)cc2C1 ; CC(C)(C)OC(=O)N1CCNCC1 ; CC(C)(C)OC(=O)N1CCN(c2cccc(C(O)c3cc4c(cn3)CN(S(=O)(=O)c3ccc(-c5ncco5)c(F)c3)C4)c2)CC1 ; O=S(=O)(c1ccc(-c2ncco2)c(F)c1)N1Cc2cnc(C(O)c3cccc(N4CCNCC4)c3)cc2C1]

### Step 1. Tert-butyl 4-[3-[[2-[3-fluoro-4-(1,3-oxazol-2-yl)benzenesulfonyl]-1H,2H,3H-pyrrolo[3,4-c]pyridin-6-yl](hydroxy)methyl]phenyl]piperazine-1-carboxylate

To a solution of (3-bromophenyl)([2-[3-fluoro-4-(1,3-oxazol-2-yl)benzenesulfonyl]-1H,2H,3H-pyrrolo[3,4-c]pyridin-6-yl])methanol (50 mg, 0.09 mmol) in 1,4-dioxane (2 mL) was added K₃PO₄ (61 mg, 0.29 mmol), tert-butyl piperazine-1-carboxylate (88 mg, 0.47 mmol), RuPhos 3G (16 mg, 0.02 mmol), and RuPhos (9 mg, 0.02 mmol). The resulting mixture was stirred for 2 h at 100° C. and then cooled to room temperature. The reaction mixture was poured into water (3 mL) and then extracted with ethyl acetate (3×10 mL). The combined organic layers were dried over anhydrous sodium sulfate, filtered, and concentrated under vacuum. The resulting crude product was purified by prep-TLC (eluting with 1:1 ethyl acetate/petroleum ether) to afford tert-butyl 4-[3-[[2-[3-fluoro-4-(1,3-oxazol-2-yl)benzenesulfonyl]-1H,2H,3H-pyrrolo [3,4-c]pyridin-6-yl](hydroxy)methyl]phenyl}piperazine-1-carboxylate (40 mg, 67%). LCMS (ES, m/z) 636 [M+H]⁺.

### Step 2. [2-[3-fluoro-4-(1,3-oxazol-2-yl)benzenesulfonyl]-1H,2H,3H-pyrrolo[3,4-c]pyridin-6-yl][3-(piperazin-1-yl)phenyl]methanol

To a solution of tert-butyl 4-[3-[[2-[3-fluoro-4-(1,3-oxazol-2-yl)benzenesulfonyl]-1H,2H,3H-pyrrolo[3,4-c]pyridin-6-yl](hydroxy)methyl]phenyl}piperazine-1-carboxylate (40 mg, 0.06 mmol) in dichloromethane (4 mL) was added TFA (1 mL). The resulting solution was stirred for 2 h at room temperature and concentrated under vacuum. The resulting mixture was basified to pH 8 with saturated potassium carbonate solution and then extracted with dichloromethane (2×5 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (25% to 45% over 7 min); Flow rate: 20 mL/min; Detector: UV 254 nm). The product fractions were concentrated and lyophilized to afford [2-[3-fluoro-4-(1,3-oxazol-2-yl)benzenesulfonyl]-1H,2H,3H-pyrrolo[3,4-c]pyridin-6-yl][3-(piperazin-1-yl)phenyl]methanol (13.1 mg, 37%). ¹H-NMR (DMSO-d₆, 400 MHz) δ (ppm) 8.35 (d, J=16.8 Hz, 2H), 8.24-8.22 (m, 1H), 7.90-7.83 (m, 2H), 7.52 (s, 1H), 7.47 (s, 1H), 7.06-7.02 (m, 1H), 6.95 (s, 1H), 6.76-6.67 (m, 2H), 5.98 (d, J=4.0 Hz, 1H), 5.60 (d, J=4.0 Hz, 1H), 4.73-4.62 (m, 4H), 2.98-2.81 (m, 4H), 2.80-2.70 (m, 4H), 2.57-2.54 (m, 1H). LCMS (ES, m/z) 536 [M+H]⁺.

### Method C

### [2-(2,3-dihydro-1,4-benzodioxine-6-sulfonyl)-1H,2H,3H-pyrrolo[3,4-c]pyridin-6-yl][3-(piperazin-1-yl)phenyl]methanol

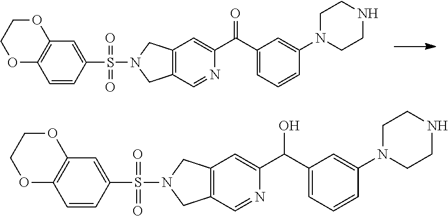 [SMILES: O=C(c1cccc(N2CCNCC2)c1)c1cc2c(cn1)CN(S(=O)(=O)c1ccc3c(c1)OCCO3)C2 ; O=S(=O)(c1ccc2c(c1)OCCO2)N1Cc2cnc(C(O)c3cccc(N4CCNCC4)c3)cc2C1]

To a solution of 1-(3-[[2-(2,3-dihydro-1,4-benzodioxine-6-sulfonyl)-1H,2H,3H-pyrrolo[3,4-c]pyridin-6-yl]carbonyl]phenyl)piperazine (30 mg, 0.05 mmol) in THF (0.5 mL) was added NaBH₄ (2 mg, 0.05 mmol) at 0° C. The resulting solution was stirred for 30 min at 0° C. The reaction mixture was poured into water (3 mL) and then extracted with DCM (3×3 mL). The combined organic layers were dried over anhydrous sodium sulfate, filtered and concentrated under vacuum. The resulting crude product was purified by silica gel chromatography (eluting with 1:10 MeOH/DCM), and further purified by Prep-HPLC (Column: XBridge Shield C18 OBD Column, 5 μm, 19×150 mm; Mobile Phase, A: water (containing 10 mmol/L NH₄HCO₃) and B: CH₃CN (20% to 42% over 7 min); Flow rate: 20 mL/min; Detector: UV 254/220 nm). The product fractions were concentrated and lyophilized to afford [2-(2,3-dihydro-1,4-benzodioxine-6-sulfonyl)-1H,2H,3H-pyrrolo[3,4-c]pyridin-6-yl][3-(piperazin-1-yl)phenyl]methanol as a white solid (3.6 mg, 13%). ¹H-NMR (Methanol-d₄, 400 MHz) δ (ppm): 8.30 (s, 1H), 7.49 (s, 1H), 7.40-7.27 (m, 2H), 7.21-7.10 (m, 1H), 7.08-7.01 (m, 1H), 6.99-6.94 (m, 1H), 6.88-6.79 (m, 2H), 5.71 (s, 1H), 4.59 (s, 4H), 4.36-4.20 (m, 4H), 3.16-3.07 (m, 4H), 3.01-2.91 (m, 4H). LCMS (ES, m/z) 509 [M+H]⁺.

### Method D-1

### [2-[4-(1,3-oxazol-2-yl)benzenesulfonyl]-1H,2H,3H-pyrrolo[3,4-c]pyridin-6-yl][3-(piperazin-1-yl)phenyl] methanol (HCl salt)

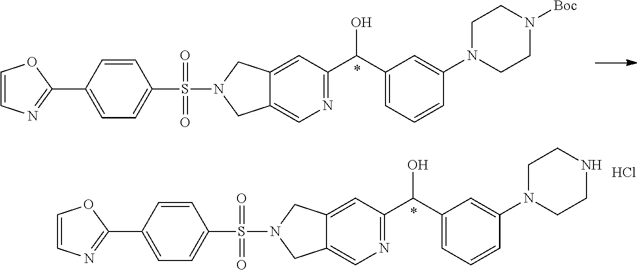

To a solution of HCl in 1,4-dioxane (15 mL, 4 N) was added tert-butyl 4-[3-[(S)-hydroxy([2-[4-(1,3-oxazol-2-yl)benzenesulfonyl]-1H,2H,3H-pyrrolo[3,4-c]pyridin-6-yl])methyl]phenyl]piperazine-1-carboxylate (1.30 g, 2.10 mmol) and water (5 mL). The resulting mixture was stirred for 4 h at room temperature. The reaction mixture was concentrated and lyophilized. The resulting crude product was purified by reverse phase chromatography (eluting with 10% to 50% MeCN/water). The product fractions were concentrated and lyophilized to afford (S)-[2-[4-(1,3-oxazol-2-yl)benzenesulfonyl]-1H,2H,3H-pyrrolo[3,4-c]pyridin-6-yl][3-(piperazin-1-yl)phenyl methanol hydrochloride salt as a white solid (857 mg, 79%). ¹H-NMR (DMSO-d₆, 400 MHz) δ (ppm): 9.09 (br s, 2H), 8.33 (d, J=2.8 Hz, 2H), 8.16 (d, J=8.4 Hz, 2H), 8.01 (d, J=8.4 Hz, 2H), 7.48 (d, J=3.2 Hz, 2H), 7.12-7.08 (m, 1H), 7.03-7.01 (m, 1H), 6.80-6.77 (m, 2H), 6.07 (br s, 1H), 5.62 (s, 1H), 4.69-4.58 (m, 4H), 3.52-3.35 (m, 4H), 3.30-3.19 (m, 4H). LCMS (ES, m/z) 518 [M+H]⁺. *Absolute stereochemistry not determined.

### Method D-2

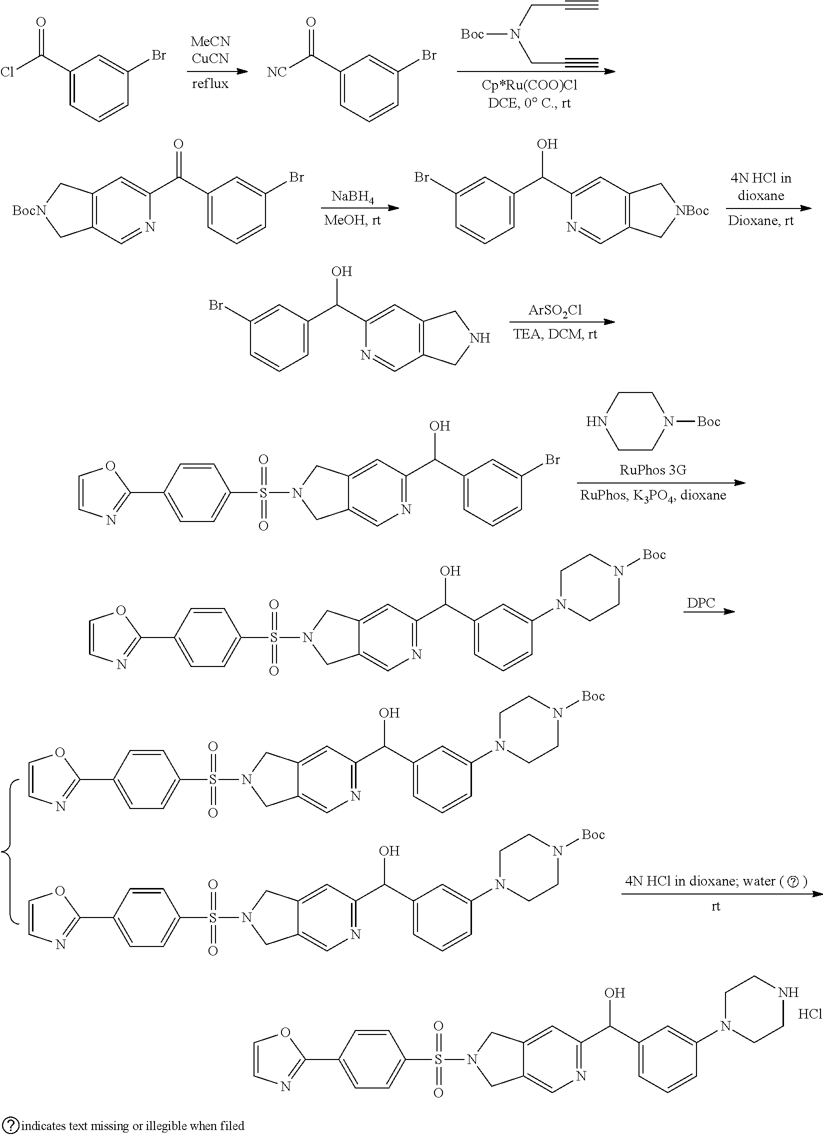

### Step 1. 3-bromobenzoyl cyanide

Into a 5-L 4-necked round-bottom flask, was placed 3-bromobenzoyl chloride (500 g, 2.27 mol, 1.00 equiv) and coppercarbonitrile (612.1 g, 6.83 mol, 3.00 equiv) in acetonitrile (2.5 L). The resulting solution was stirred for 2 hours at 80° C. and cooled to room temperature. Filtered and the filtrate was concentrated under vacuum. The resulting solution was extracted with 2×500 mL of ether and organic was concentrated under vacuum. The resulting solution was extracted with 3×500 mL of n-hexane, and organic was concentrated under vacuum. This resulted of 3-bromobenzoyl cyanide as a white solid (336 g, 71%).

### Step 2. tert-butyl 6-(3-bromobenzoyl)-1H,2H,3H-pyrrolo[3,4-c]pyridine-2-carboxylate

Into a 3-L 4-necked round-bottom flask purged and maintained with an inert atmosphere of nitrogen was added a solution of 3-bromobenzoyl cyanide (130 g, 0.62 mol, 1.00 equiv) in 1,2-dichloroethane (1.3 L). Then chloro(1,5-cyclooctadiene)(pentamethylcyclopentadienyl)ruthenium (II) (23.5 g, 0.06 mol, 0.10 equiv) and tert-butyl N,N-bis(prop-2-yn-1-yl)carbamate (179.4 g, 0.93 mol, 1.50 equiv) was added at 0° C. The resulting mixture was stirred for 4 h at room temperature. The resulting mixture was concentrated under vacuum. The resulting crude product was purified by silica gel chromatography (eluting with 0:100 to 20:80 ethyl acetate/petroleum ether) to afford tert-butyl 6-(3-bromobenzoyl)-1H,2H,3H-pyrrolo[3,4-c]pyridine-2-carboxylate as a yellow solid (150 g, 60%).

### Step 3. tert-butyl 6-[(3-bromophenyl) (hydroxy)methyl]-1H,2H,3H-pyrrolo[3,4-c]pyridine-2-carboxylate

Into a 3-L 4-necked round-bottom flask, was placed tert-butyl 6-(3-bromobenzoyl)-1H,2H,3H-pyrrolo[3,4-c]pyridine-2-carboxylate (150 g, 370 mmol, 1.00 equiv) in methanol (1.5 L). Then, sodium borohydride (7.05 g, 180 mmol, 0.50 equiv) was added at 0° C. The resulting mixture was stirred for 1 h at room temperature. The reaction mixture was poured into water (500 mL) and then extracted with ethyl acetate (3×1 L). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford tert-butyl 6-[(3-bromophenyl) (hydroxy)methyl]-1H,2H,3H-pyrrolo[3,4-c]pyridine-2-carboxylate as a yellow solid (140 g, 93%).

### Step 4. (3-bromophenyl)([1H,2H,3H-pyrrolo[3,4-c]pyridin-6-yl])methanol

Into a 2-L 3-necked round-bottom flask, was placed tert-butyl 6-[(3-bromophenyl)(hydroxy)methyl]-1H,2H,3H-pyrrolo[3,4-c]pyridine-2-carboxylate (120 g, 0.296 mol, 1.00 equiv) and 4N HCl (gas) in 1,4-dioxane (738.4 mL) in dioxane (1.2 L). The resulting solution was stirred for 4 hours at room temperature. The solids were collected by filtration. The filter cake was concentrated under vacuum. This resulted of (3-bromophenyl)([1H,2H,3H-pyrrolo[3,4-c]pyridin-6-yl])methanol as a green solid (96 g, crude).

### Step 5. (3-bromophenyl)([2-[4-(1,3-oxazol-2-yl)benzenesulfonyl]-1H,2H,3H-pyrrolo[3,4-c]pyridin-6-yl])methanol

To a solution of 6-(3-bromobenzoyl)-1H,2H,3H-pyrrolo[3,4-c]pyridine HCl salt (100 g, 0.328 mol, 1.00 equiv) in DCM (1 L) was added triethylamine (99.8 g, 0. 986 mol, 3.00 equiv) at 0° C. The resulting mixture was stirred for 30 min. Then a solution of 4-(1,3-oxazol-2-yl)benzene-1-sulfonyl chloride (79.9 g, 0.328 mol, 1.00 equiv) in DCM (500 mL) was added slowly. The resulting mixture was stirred for 2 hours at room temperature. The resulting mixture was concentrated under vacuum. The solids were washed with water and were an oven. This resulted in (3-bromophenyl)([2-[4-(1,3-oxazol-2-yl)benzenesulfonyl]-1H,2H,3H-pyrrolo[3,4-c]pyridin-6-yl])methanol as a yellow solid (129 g, 80% in two steps).

### Step 6. tert-butyl 4-[3-[hydroxy([2-[4-(1,3-oxazol-2-yl)benzenesulfonyl]-1H,2H,3H-pyrrolo[3,4-c]pyridin-6-yl])methyl]phenyl]piperazine-]-carboxylate

Into a 3-L 4-necked round-bottom flask purged and maintained with an inert atmosphere of nitrogen was added a solution of (3-bromophenyl)([2-[4-(1,3-oxazol-2-yl)benzenesulfonyl]-1H,2H,3H-pyrrolo [3,4-c]pyridin-6-yl])methanol (130 g, 0.253 mol, 1.00 equiv) in 1,4-dioxane (1.3 L) and maintained with an inert atmosphere of nitrogen 30 min. Then tert-butyl piperazine-1-carboxylate (141.2 g, 0.759 mol, 3.00 equiv), potassium phosphate (161.1 g, 0.759 mol, 3.00 equiv), RuPhos 3G (21.16 g, 25.3 mmol, 0.1 equiv) and RuPhos (11.8 g, 25.3 mmol, 0.1 equiv) was added. The resulting mixture was stirred for 16 h at 100° C. and cooled to room temperature. The reaction mixture was poured into water (500 mL) and then extracted with ethyl acetate (3×500 mL). The combined organic layers were dried over anhydrous sodium sulfate, filtered, and concentrated under vacuum. The resulting crude product was purified by silica gel chromatography (eluting with 0:100 to 70:30 ethyl acetate/petroleum ether). This resulted in 80 g (51%) of tert-butyl 4-[3-[hydroxy([2-[4-(1,3-oxazol-2-yl)benzenesulfonyl]-1H,2H,3H-pyrrolo[3,4-c]pyridin-6-yl])methyl]phenyl]piperazine-1-carboxylate as a white solid (80 g, 51%).

### Step 7. tert-butyl 4-[3-[(R)-hydroxy([2-[4-(1,3-oxazol-2-yl)benzenesulfonyl]-1H,2H,3H-pyrrolo[3,4-c]pyridin-6-yl])methyl]phenyl]piperazine-1-carboxylate and tert-butyl 4-[3-[(S)-hydroxy([2-[4-(1,3-oxazol-2-yl)benzenesulfonyl]-1H,2H,3H-pyrrolo[3,4-c]pyridin-6-yl])methyl]phenyl]piperazine-1-carboxylate

The two enantiomers were separated by Prep-SFC (Column: CHIRALPAK,IF, 5 μm, 2×25 cm; Mobile Phase A: CO₂, Mobile Phase B: IPA:DCM=1:1 (0.1% NH₃.H₂O) (keep 60% B over 12 min); Flow rate: 180 mL/min; Detector: 220 nm; Retention time, 7 A: 6.1 min; 7B: 8.1 min). The product fractions were concentrated and lyophilized to afford tert-butyl 4-[3-[(R)-hydroxy([2-[4-(1,3-oxazol-2-yl)benzenesulfonyl]-1H,2H,3H-pyrrolo[3,4-c]pyridin-6-yl])methyl]phenyl]piperazine-1-carboxylate as a yellow solid (31 g, 38%) and tert-butyl 4-[3-[(S)-hydroxy([2-[4-(1,3-oxazol-2-yl)benzenesulfonyl]-1H,2H,3H-pyrrolo[3,4-c]pyridin-6-yl])methyl]phenyl]piperazine-1-carboxylate (37 g, 46%) as a yellow solid.

### Step 8. (S)-[2-[4-(1,3-oxazol-2-yl)benzenesulfonyl]-1H,2H,3H-pyrrolo[3,4-c]pyridin-6-yl][3-(piperazin-1-yl)phenyl]methanol hydrochloride salt

To a solution of tert-butyl 4-[3-[(S)-hydroxy([2-[4-(1,3-oxazol-2-yl)benzenesulfonyl]-1H,2H,3H-pyrrolo[3,4-c]pyridin-6-yl])methyl]phenyl]piperazine-1-carboxylate (37 g, 59 mmol, 1.00 equiv) and water (148 mL) was added HCl in 1,4-dioxane (444 mL, 4 mol/L). The resulting mixture was stirred for 4 hours at room temperature. The reaction mixture was concentrated under vacuum. The resulting crude product was purified by reverse phase chromatography (eluting with 10% to 50% MeCN/water). The product fractions were concentrated and lyophilized to afford (S)-[2-[4-(1,3-oxazol-2-yl)benzenesulfonyl]-1H,2H,3H-pyrrolo[3,4-c]pyridin-6-yl][3-(piperazin-1-yl)phenyl]methanol hydrochloride salt as a white solid (16.7 g, 48%).

LCMS: (ES, m/z) 518 [M+H]⁺;

H-NMR: (400 MHz, DMSO-d6, ppm) δ 9.29 (br s, 2H), 8.33 (d, J=2.8 Hz, 2H), 8.16 (d, J=8.4 Hz, 2H), 8.01 (d, J=8.4 Hz, 2H), 7.48 (d, J=3.2 Hz, 2H), 7.12-7.08 (m, 1H), 7.03-7.01 (m, 1H), 6.80-6.77 (m, 2H), 6.07 (br s, 1H), 5.62 (s, 1H), 4.69-4.58 (m, 4H), 3.52-3.35 (m, 4H), 3.30-3.19 (m, 4H).

### Method D-3

### [2-[4-(1,3-oxazol-2-yl)benzenesulfonyl]-1H,2H,3H-pyrrolo[3,4-c]pyridin-6-yl][3-(piperazin-1-yl)phenyl] methanol (free base)

To a solution of tert-butyl 4-[3-[hydroxy([2-[4-(1,3-oxazol-2-yl)benzenesulfonyl]-1H,2H,3H-pyrrolo[3,4-c]pyridin-6-yl])methyl]phenyl]piperazine-1-carboxylate (300 mg, 0.49 mmol) in dichloromethane (5 mL) was added trifluoroacetic acid (1 mL). The resulting solution was stirred for 2 hours at room temperature and concentrated under vacuum to remove volatile components. The resulting aqueous mixture was brought to pH 8 with saturated potassium carbonate solution and then extracted with dichloromethane (2×5 mL). The combined organic layers were combined, dried over anhydrous sodium sulfate, filtered, and concentrated under vacuum. The crude product was purified by silica gel chromatography (eluting with methanol/dichloromethane (1/9)) to afford the racemic product. The racemate was separated by Chiral Prep-HPLC with the following conditions: Column: Chiralpak IC-3 0.46×5 cm, 3 um; mobile phase A: MTBE (0.1% IPA), B: EtOH (50B over 6 min); Flow rate 1 mL/min, 220/254 nm, Retention time: OA, 2.35 min; OB, 4.16 min). The product fractions were concentrated and lyophilized to afford (R)-(2-((4-(oxazol-2-yl)phenyl)sulfonyl)-2,3-dihydro-1H-pyrrolo[3,4-c]pyridin-6-yl)(3-(piperazin-1-yl)phenyl)methanol (34.8 mg, 14%).

LCMS (ES, m/z) 518 [M+H]⁺. ¹H-NMR (DMSO-d₆, 400 MHz) δ (ppm): 8.32 (d, J=4.4 Hz, 2H), 8.16 (d, J=8.4 Hz, 2H), 8.00 (d, J=8.4 Hz, 2H), 7.47 (s, 2H), 7.05-7.01 (m, 1H), 6.95 (s, 1H), 6.71-6.66 (m, 2H), 5.97 (d. J=3.2 Hz, 1H), 5.59 (s. 1H), 4.69-4.58 (m, 4H), 2.98-2.96 (m, 4H), 2.85-2.78 (m 4H). And (S)-(2-((4-(oxazol-2-yl)phenyl)sulfonyl)-2,3-dihydro-1H-pyrrolo[3,4-c]pyridin-6-yl)(3-(piperazin-1-yl)phenyl)methanol (34.1 mg, 13%). LCMS (ES, m/z) 518 [M+H]⁺.

¹H-NMR (DMSO-d₆, 400 MHz) δ (ppm): 8.32 (d, J=4.0 Hz, 2H), 8.16 (d, J=8.4 Hz, 2H), 8.00 (d, J=8.4 Hz, 2H), 7.47 (s, 2H), 7.05-7.01 (m, 1H), 6.94 (s, 1H), 6.71-6.66 (m, 2H), 5.97 (d. J=3.2 Hz, 1H), 5.59 (s. 1H), 4.65-4.59 (m, 4H), 2.96 (d. J=4.8 Hz, 4H), 2.85-2.79 (m 4H).

### Method E

### [2-(2,3-dihydro-1,4-benzodioxine-6-sulfonyl)-1H,2H,3H-pyrrolo[3,4-c]pyridin-6-yl](phenyl)methyl] (methyl)amine

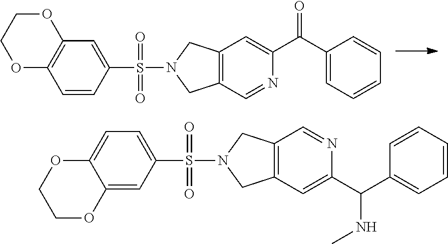

To a solution of 6-benzoyl-2-(2,3-dihydro-1,4-benzodioxine-6-sulfonyl)-1H,2H,3H-pyrrolo[3,4-c]pyridine (80 mg, 0.19 mmol) in methanol (1 mL) was added a solution of methylamine (0.5 mL, 2 M in THF). This was followed by the addition of titanium isopropoxide (108 mg, 0.38 mmol) with stirring at room temperature. The resulting solution was stirred for 16 h at room temperature. Then sodium borohydride (14 mg, 0.37 mmol) was added and the resulting mixture was stirred for an additional 2 h at room temperature. The reaction mixture was poured into water (2 mL) and then extracted with ethyl acetate (3×3 mL). The combined organic layers were dried over anhydrous sodium sulfate, filtered and concentrated under vacuum. The resulting crude product was purified by Prep-HPLC (Column: XBridge Shield C18 OBD Column, 5 μm, 19×150 mm; Mobile Phase, A: water (containing 10 mmol/L NH₄HCO₃) and B: CH₃CN (30% to 65% over 7 min); Flow rate: 20 mL/min; Detector: UV 254/220 nm). The product fractions were concentrated and lyophilized to afford [2-(2,3-dihydro-1,4-benzodioxine-6-sulfonyl)-1H,2H,3H-pyrrolo[3,4-c]pyridin-6-yl](phenyl)methyl](methyl)amine as a white solid (37 mg, 45%). ¹H-NMR (Methanol-d₄, 400 MHz) δ (ppm): 8.37 (s, 1H), 7.40-7.18 (m, 8H), 7.00-6.93 (m, 1H), 4.79 (s, 1H), 4.62-4.53 (m, 4H), 4.31-4.21 (m, 4H), 2.31 (s, 3H). LCMS (ES, m/z) 438 [M+H]⁺.

### Method F

### [2-[4-(5-fluoro-1H-pyrazol-1-yl) benzenesulfonyl]-1H,2H,3H-pyrrolo[3,4-c]pyridine-6-yl](phenyl) methanol

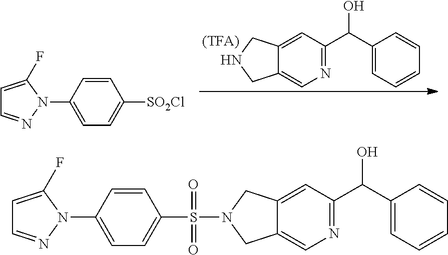

To a solution of phenyl([1H,2H,3H-pyrrolo[3,4-c]pyridine-6-yl])methanol (TFA salt) (100 mg, 0.29 mmol) in dichloromethane (10 mL) and N,N-dimethylformamide (2 mL) was added potassium carbonate (122 mg, 0.88 mmol). The resulting mixture was stirred for 30 min at room temperature. To this was added 4-(5-fluoro-1H-pyrazol-1-yl) benzene-1-sulfonyl chloride (77 mg, 0.30 mmol). The resulting solution was stirred for 1 h at room temperature. The reaction mixture was poured into water (3 mL) and then extracted with ethyl acetate (3×10 mL). The combined organic layers were dried over anhydrous sodium sulfate, filtered, and concentrated under vacuum. The resulting crude product was purified by prep-TLC (eluting with 99:1 ethyl acetate/petroleum ether), and further purified by prep-HPLC (Column: XBridge Shield RP18 OBD Column, 5 μm, 19×150 mm; Mobile Phase, A: water (containing 10 mmol/L NH₄HCO₃) and B: CH₃CN (30% B to 62% B over 7 min); Flow rate: 20 mL/min; Detector: UV 254 nm). The product fractions were concentrated and lyophilized to afford [2-[4-(5-fluoro-1H-pyrazol-1-yl)benzenesulfonyl]-1H,2H,3H-pyrrolo[3,4-c]pyridine-6-yl](phenyl)methanol (13.6 mg, 10%). ¹H-NMR (DMSO-d₆, 400 MHz) δ (ppm): 8.33 (s, 1H), 8.07-7.98 (m, 2H), 7.88-7.85 (m, 2H), 7.74-7.73 (m, 1H), 7.49 (s, 1H), 7.31 (d, J=6.8 Hz, 2H), 7.24-7.21 (m, 2H), 7.17-7.14 (m, 1H). 6.30-6.29 (m, 1H), 6.08-6.06 (m, 1H), 5.64 (d, J=4.0 Hz, 1H), 4.69-4.57 (m, 4H). LCMS (ES, m/z) 451 [M+H]⁺.

### Method G

### [2-(2,3-dihydro-1,4-benzodioxine-6-sulfonyl)-1H,2H,3H-pyrrolo[3,4-c]pyridine-6-yl](phenyl) methanamine

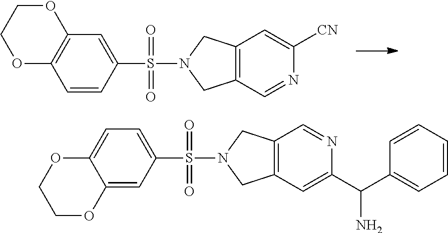

To a solution of 2-(2,3-dihydro-1,4-benzodioxine-6-sulfonyl)-1H,2H,3H-pyrrolo[3,4-c]pyridine-6-carbonitrile (300 mg, 0.79 mmol) in toluene (10 mL) was added a solution of bromo(phenyl)magnesium (1.3 mL, 1M in tetrahydrofuran) dropwise with stirring at 0° C. The resulting solution was stirred for 1 h at room temperature. The reaction was quenched by addition of 2-methylpropan-1-ol (71 mg, 0.96 mmol). The resulting solution was stirred for 2 h at room temperature. This was followed by addition of NaBH₄ (100 mg, 2.64 mmol). The resulting solution was stirred for overnight at rt. The reaction mixture was poured into water (10 mL) and extracted with ethyl acetate (3×20 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (30% to 58% over 7 min); Flow rate: 20 mL/min; Detector: UV 254 nm). The product fractions were concentrated and lyophilized to afford [2-(2,3-dihydro-1,4-benzodioxine-6-sulfonyl)-1H,2H,3H-pyrrolo[3,4-c]pyridine-6-yl](phenyl)methanamine as a white solid (250 mg 74%). ¹H-NMR (DMSO-d₆, 300 MHz) δ (ppm): 8.34 (s, 1H), 7.41 (s, 1H), 7.35-7.21 (m, 6H), 7.17-7.12 (m, 1H), 7.04-7.01 (m, 1H), 5.08 (s, 1H), 4.53 (s, 4H), 4.29-4.25 (m, 4H), 2.41 (s, 2H). LCMS (ES, m/z): 424 [M+H]⁺.

### Method H

### (2-{[1-(1,3-oxazol-2-yl)piperidin-4-yl]sulfonyl}-1H,2H,3H-pyrrolo[3,4-c]pyridine-6-yl)(phenyl)methanol

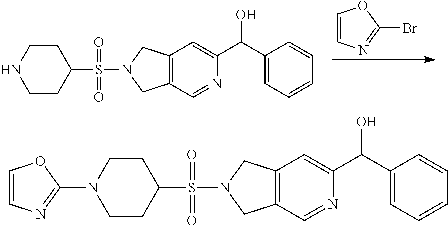

To a solution of phenyl[2-(piperidine-4-sulfonyl)-1H,2H,3H-pyrrolo[3,4-c]pyridin-6-yl]methanol (80 mg, 0.21 mmol) in N,N-dimethylformamide (2 mL) was added DIEA (83 mg, 0.64 mmol). This was followed by the addition of 2-bromo-1,3-oxazole (64 mg, 0.43 mmol) at 70° C. with stirring. The resulting solution was stirred for 2 h at 70° C. and then cooled to room temperature. The reaction mixture was poured into water (5 mL) and then extracted with ethyl acetate (3×5 mL). The combined organic layers were dried over anhydrous sodium sulfate, filtered, and concentrated under vacuum. The resulting crude product was purified by silica gel chromatography (eluting with 0:100 to 10:90 methanol/dichloromethane), and further purified by Prep-HPLC (Column: XBridge Shield C₁₈ OBD Column, 5 μm, 19×150 mm; Mobile Phase, A: water (containing 10 mmol/L NH₄HCO₃) and B: CH₃CN (25% to 50% over 7 min); Flow rate: 20 mL/min; Detector: UV 254/220 nm). The product fractions were concentrated and lyophilized to afford (2-[[1-(1,3-oxazol-2-yl)piperidin-4-yl]sulfonyl]-1H,2H,3H-pyrrolo[3,4-c]pyridin-6-yl)(phenyl)methanol as a white solid (13.0 mg, 14%). ¹H-NMR (Methanol-d₄, 400 MHz) δ (ppm): 8.43 (s, 1H), 7.63 (s, 1H), 7.43-7.41 (m, 3H), 7.33-7.30 (m, 2H), 7.26-7.23 (m, 1H), 6.84 (s, 1H), 5.85 (s, 1H), 4.92-4.83 (s, 4H), 4.15-4.11 (m, 2H), 3.59-3.52 (m, 1H), 3.08-3.01 (m, 2H), 2.16-2.12 (m, 2H), 1.91-1.85 (m, 2H). LCMS (ES, m/z) 441 [M+H]⁺.

### Method I

### (S)-[2-(2,3-dihydro-1,4-benzodioxine-6-sulfonyl)-1H,2H,3H-pyrrolo[3,4-c]pyridin-6-yl](phenyl)-methanol; (R)-[2-(2,3-dihydro-1,4-benzodioxine-6-sulfonyl)-1H,2H,3H-pyrrolo[3,4-c]pyridin-6-yl](phenyl)-methanol

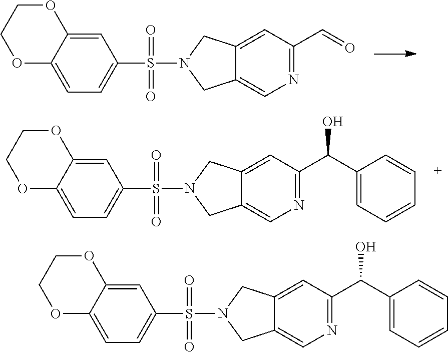

To a solution of 2-(2,3-dihydro-1,4-benzodioxine-6-sulfonyl)-1H,2H,3H-pyrrolo[3,4-c]pyridine-6-carbaldehyde (40 mg, 0.10 mmol) in THF (1 mL) was added a solution of phenylmagnesium bromide (0.1 mL, 1.0 M in THF) dropwise with stirring at 0° C. The resulting solution was stirred for 2 h at 0° C. The reaction mixture was poured into saturated ammonium chloride solution (10 mL) and then extracted with EA (3×8 mL). The combined organic layers were dried over anhydrous sodium sulfate, filtered and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (10% to 30% over 20 min); Flow rate: 20 mL/min; Detector: UV 254 nm). The two enantiomers were further separated by Chiral Prep-HPLC (Column: CHIRALPAK IA, 5 μm, 20×250 mm; Mobile Phase, A: methanol and B: DCM (hold 50% B over 25 min); Detector: UV 254/220 nm; Retention time: 1^{st} eluting isomer, 7.62 min; 2^{nd} eluting isomer, 9.89 min). The product fractions of 1^{st} eluting isomer were concentrated and lyophilized to afford a white solid (2.1 mg, 5%). 1H-NMR (Methanol-d₄, 300 MHz) δ (ppm): 8.34-8.29 (m, 1H), 7.53-7.49 (m, 1H), 7.42-7.17 (m, 7H), 7.04-6.95 (m, 1H), 5.79 (s, 1H), 4.64 (s, 4H), 4.35-4.22 (m, 4H). LCMS (ES, m/z) 425 [M+H]⁺. The product fractions of 2^{nd} eluting isome were concentrated and lyophilized to afford a white solid (1.4 mg, 3%). 1H-NMR (Methanol-d₄, 300 MHz) δ (ppm): 8.34-8.29 (m, 1H), 7.53-7.49 (m, 1H), 7.42-7.17 (m, 7H), 7.04-6.95 (m, 1H), 5.79 (s, 1H), 4.64 (s, 4H), 4.35-4.22 (m, 4H). LCMS (ES, m/z) 425 [M+H]⁺.

### Method J

### Phenyl(2-{[1-(1,3-thiazol-2-yl)piperidin-4-yl]sulfonyl}-1H,2H,3H-pyrrolo[3,4-c]pyridine-6-yl)methanol

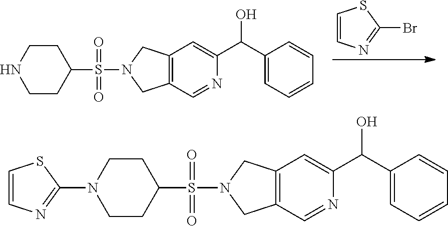

To a solution of phenyl[2-(piperidine-4-sulfonyl)-1H,2H,3H-pyrrolo[3,4-c]pyridin-6-yl]methanol (40 mg, 0.11 mmol) in 1,4-dioxane (1 mL), was added 2-bromo-1,3-thiazole (18 mg, 0.11 mmol), Cs₂CO₃ (105 mg, 0.32 mmol) and RuPhos 3G (10 mg, 0.01 mmol). The resulting mixture was stirred for 16 h at 100° C. and then cooled to room temperature. The reaction mixture was poured into water (2 mL) and then extracted with ethyl acetate (2×5 mL). The combined organic layers were dried over anhydrous sodium sulfate, filtered, and concentrated under vacuum. The resulting crude product was purified by silica gel chromatography (eluting with 0:100 to 10:90 methanol/dichloromethane) and further purified by Prep-HPLC (Column: XBridge Shield C18 OBD Column, 5 μm, 19×150 mm; Mobile Phase, A: water (containing 10 mmol/L NH₄HCO₃) and B: CH3CN (30% to 65% over 7 min); Flow rate: 20 mL/min; Detector: UV 254/220 nm). The product fractions were concentrated and lyophilized to afford phenyl(2-[[1-(1,3-thiazol-2-yl)piperidin-4-yl]sulfonyl]-1H,2H,3H-pyrrolo[3,4-c]pyridin-6-yl)methanol as a white solid (2.0 mg, 4%). ¹H-NMR (CDCl3, 400 MHz,) δ (ppm): 8.51 (s, 1H), 7.47-7.35 (m, 4H), 7.32-7.31 (m, 1H), 7.19 (s, 1H), 7.10 (s, 1H), 6.62-6.60 (s, 1H), 5.79 (s, 1H), 4.98 (s, 1H), 4.87-4.83 (m, 2H), 4.79-4.69 (m, 2H), 4.19-4.16 (m, 2H), 3.29-3.22 (m, 1H), 3.07-3.01 (m, 2H), 2.23-2.20 (m, 2H), 2.06-1.96 (m, 2H). LCMS (ES, m/z) 457 [M+H]⁺.

### Method K

### (S)-{2-[4-(4,5-dihydro-1,3-oxazol-2-yl)benzenesulfonyl]-1H,2H,3H-pyrrolo[3,4-c]pyridin-6-yl}(phenyl) methanol; (R)-{2-[4-(4,5-dihydro-1,3-oxazol-2-yl)benzenesulfonyl]-1H,2H,3H-pyrrolo[3,4-c]pyridin-6-yl}(phenyl)methanol

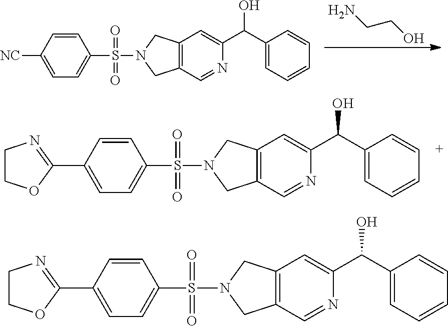

To a solution of 4-({6-[hydroxy(phenyl)methyl]-1H,2H,3H-pyrrolo[3,4-c]pyridin-2-yl}sulfonyl)benzonitrile (150 mg, 0.38 mmol) in 2-aminoethan-1-ol (1 mL), was added indium chloride (8 mg, 0.04 mmol). The resulting mixture was irradiated with microwave for 40 min at 80° C. After cooling to room temperature, the reaction mixture was concentrated under vacuum. The resulting crude product was purified by silica gel chromatography (eluting with 100:0 to 80:20 dichloromethane/methanol). The product fractions were concentrated under vacuum. The two enantiomers were further separated by Chiral Prep-HPLC (Column: CHIRALPAK IC, 5 μm, 20×250 mm; Mobile Phase, A: hexane:DCM=3:1 (containing 0.1% DEA), B: EtOH; Gradient: keep 50% B over 24 min; Flow rate: 18 mL/min; Detector: UV 254/220 nm; Retention time: 1^{st} eluting isomer, 12.618 min; 2^{nd} eluting isomer, 19.401 min). The product fractions of the 1^{st} eluting isomer were concentrated and lyophilized to afford a white solid (13.0 mg, 8%). ¹H-NMR (DMSO-d6, 400 MHz) δ (ppm): 8.32 (s, 1H), 8.03 (d, J=8.0 Hz, 2H), 7.95 (d, J=8.0 Hz, 2H), 7.49 (s, 1H), 7.31 (d, J=8.0 Hz, 2H), 7.25 (t, J=8.0 Hz, 2H), 7.19-7.16 (m, 1H), 6.08 (d, J=4.0 Hz, 1H), 5.67 (d, J=4.0 Hz, 1H), 4.71-4.54 (m, 4H), 4.43 (t, J=8.0 Hz, 2H), 3.98 (t, J=8.0 Hz, 2H). LCMS (ES, m/z): 436 [M+H]⁺. The product fractions of the 2^{nd} eluting isomer were concentrated and lyophilized to afford a white solid (13.5 mg, 8%). ¹H-NMR (DMSO-d6, 400 MHz) δ (ppm): 8.32 (s, 1H), 8.03 (d, J=8.0 Hz, 2H), 7.95 (d, J=8.0 Hz, 2H), 7.49 (s, 1H), 7.31 (d, J=8.0 Hz, 2H), 7.25 (t, J=8.0 Hz, 2H), 7.19-7.16 (m, 1H), 6.08 (d, J=4.0 Hz, 1H), 5.67 (d, J=4.0 Hz, 1H), 4.71-4.54 (m, 4H), 4.43 (t, J=8.0 Hz, 2H), 3.98 (t, J=8.0 Hz, 2H). LCMS (ES, m/z): 436 [M+H]⁺.

### Method L

### (S)-[2-[3-fluoro-4-(1,3-oxazol-2-yl)benzenesulfonyl]-1H,2H,3H-pyrrolo[3,4-c]pyridin-6-yl]([3-[(1-methylazetidin-3-yl)oxy]phenyl])methanol; (R)-[2-[3-fluoro-4-(1,3-oxazol-2-yl)benzenesulfonyl]-1H,2H,3H-pyrrolo[3,4-c]pyridin-6-yl]([3-[(1-methylazetidin-3-yl)oxy]phenyl])methanol

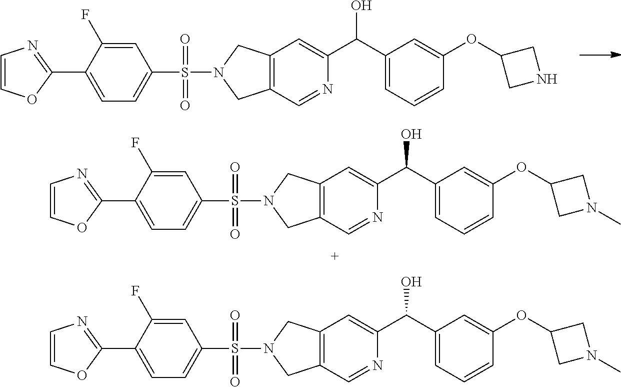

To a solution of [3-(azetidin-3-yloxy)phenyl]([2-[3-fluoro-4-(1,3-oxazol-2-yl)benzenesulfonyl]-1H,2H,3H-pyrrolo[3,4-c]pyridin-6-yl])methanol (TFA salt) (80 mg, 0.14 mmol) in MeOH (2 mL) was added formaldehyde (1 mL, 30% in water). The resulting solution was stirred for 30 min at room temperature. This was followed by the addition of STAB (92 mg, 0.41 mmol). The resulting solution was stirred for 12 h at room temperature. The reaction mixture was poured into water (10 mL) and then extracted with ethyl acetate (3×10 mL). The organic layers were combined, dried over anhydrous sodium sulfate, filtered, and concentrated under vacuum. The resulting crude product was purified by Prep-HPLC (Column: XBridge Shield RP18 OBD Column, 5 μm, 30×150 mm; Mobile Phase, A: water (containing 10 mmol/L NH₄HCO₃) and B: CH₃CN (25% to 50% in 7 min); Flow rate: 60 mL/min; Detector: UV 220 nm). The product fractions were concentrated under vacuum. The two enantiomers were further separated by Chiral Prep-HPLC (Column: CHIRALPAK IC, 5 μm, 20×250 mm; Mobile Phase, A: MeOH (containing 0.1% DEA) and B: DCM (keep 40% B in 15 min); Flow rate: 20 mL/min; Detector: UV 254/220 nm; Retention time: 1^{st} eluting isomer, 10.772 min; 2^{nd} eluting isomer, 13.314 min). The product fractions were concentrated and lyophilized to afford 1^{st} eluting isomer as a white solid (12.4 mg, 16%). ¹H-NMR (DMSO-d₆, 400 MHz) δ (ppm): 8.38 (s, 1H), 8.34 (s, 1H), 8.24-8.20 (m, 1H), 7.90-7.83 (m, 2H), 7.53 (s, 1H), 7.47 (s, 1H), 7.14-7.10 (m, 1H), 6.88 (d, J=7.6 Hz, 1H), 6.81 (s, 1H), 6.60-6.57 (m, 1H), 6.10 (d, J=4.0 Hz, 1H), 5.63 (d, J=4.4 Hz, 1H), 4.70-4.63 (m, 5H), 3.71-3.68 (m, 2H), 2.92-2.89 (m, 2H), 2.27 (s, 3H). LCMS (ES, m/z): 537 [M+H]⁺. The product fractions were concentrated and lyophilized to afford 2^{nd} eluting isomer as a white solid (13.3 mg, 18%). ¹H-NMR (DMSO-d₆, 400 MHz) δ (ppm): 8.38 (s, 1H), 8.34 (s, 1H), 8.24-8.20 (m, 1H), 7.90-7.83 (m, 2H), 7.53 (s, 1H), 7.47 (s, 1H), 7.14-7.10 (m, 1H), 6.88 (d, J=7.6 Hz, 1H), 6.81 (s, 1H), 6.60-6.58 (m, 1H), 6.10 (d, J=4.4 Hz, 1H), 5.63 (d, J=4.0 Hz, 1H), 4.69-4.65 (m, 5H), 3.74-3.71 (m, 2H), 2.92-2.89 (m, 2H), 2.29 (s, 3H). LCMS (ES, m/z): 537 [M+H]⁺.

### Method M

### (S)-[4-methyl-3-(4-methylpiperazin-1-yl)phenyl]({2-[4-(1,3-oxazol-2-yl)benzenesulfonyl]-1H,2H,3H-pyrrolo[3,4-c]pyridine-6-yl})methanol (first eluting isomer); (R)-[4-methyl-3-(4-methylpiperazin-1-yl)phenyl]({2-[4-(1,3-oxazol-2-yl)benzenesulfonyl]-1H,2H,3H-pyrrolo[3,4-c]pyridine-6-yl})methanol (second eluting isomer)

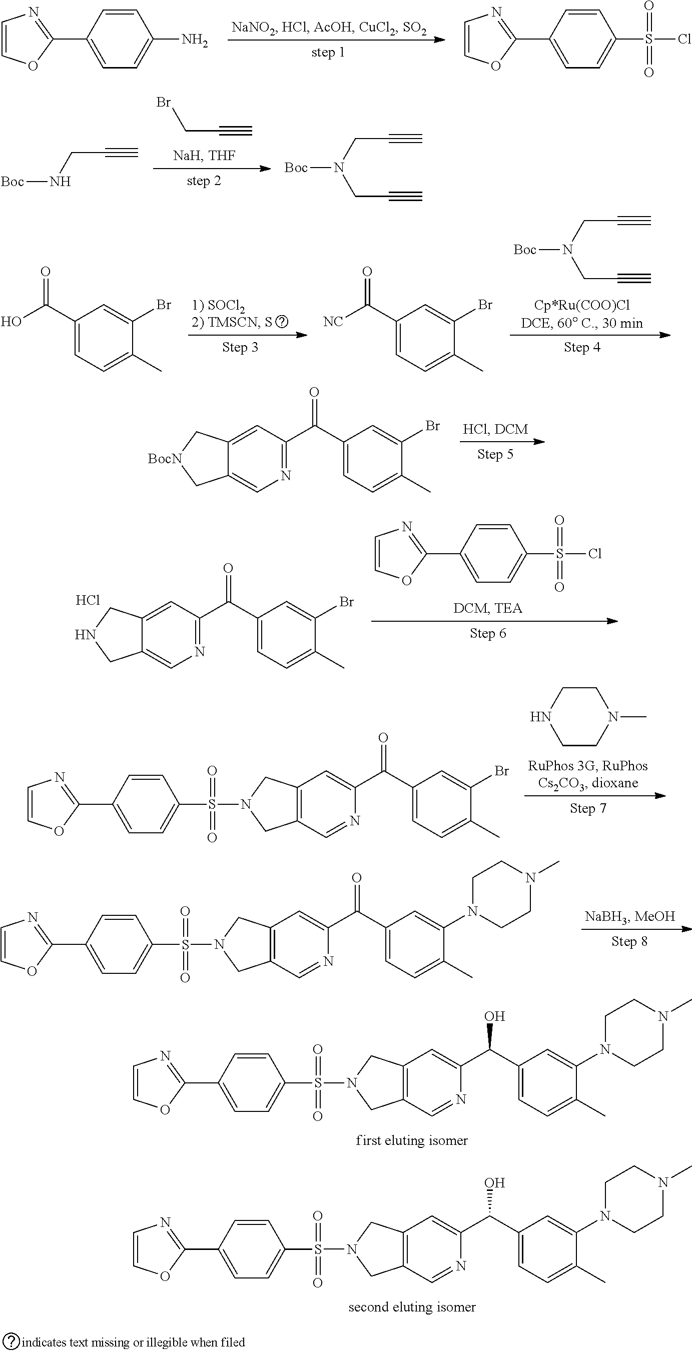

### Step 1. 4-(1,3-oxazol-2-yl)benzenesulfonyl chloride

To a stirred mixture of 4-(1,3-oxazol-2-yl) aniline (1.00 g, 6.24 mmol) in conc. HCl (12 mL) and acetic acid (4 mL) was added NaNO₂ (517 mg, 7.49 mmol) in water (0.5 mL) dropwise at −10′° C. The mixture was stirred for 1 h at 0° C. to afford the fresh prepared diazonium salt (mixture A). In a separated 3-necked round-bottom flask, SO₂ was pumped into the stirred mixture of CuCl₂ (235 mg, 1.75 mmol) in acetic acid (20 mL) for 2 h at 20° C. to afford mixture B. The mixture A was then added into the mixture B at 20° C. The resulting mixture was stirred for additional overnight at 25° C. The reaction mixture was diluted with water (40 mL), the precipitated solids were collected by filtration, washed with water (2×20 mL) and dried under vacuum to afford 4-(1,3-oxazol-2-yl)benzenesulfonyl chloride (1.20 g, 67%) as an off-white solid. LCMS (ES, m/z): 244, 246 [M+H]⁺.

### Step 2. tert-butyl N,N-bis(prop-2-yn-1-yl)carbamate

To a stirred mixture of tert-butyl N-(prop-2-yn-1-yl)carbamate (5.00 g, 30.6 mmol) in DMF (50 mL) was added NaH (1.35 g, 33.7 mmol, 60%) in portions at 0° C. The resulting mixture was stirred for 30 min at 0° C. under nitrogen atmosphere. This was followed by the additional of 3-bromoprop-1-yne (4.60 g, 36.7 mmol). The resulting mixture was stirred for 3 h at 26° C. under nitrogen atmosphere. The reaction mixture was poured into water/ice (100 mL) and extracted with ethyl acetate (2×100 mL). The combined organic layers were washed with brine (3×100 mL), dried over anhydrous sodium sulfate, and concentrated under vacuum. The residue was purified by silica gel column chromatography, eluted with PE/EtOAc (1:1) to afford tert-butyl N,N-bis(prop-2-yn-1-yl)carbamate (2.60 g, 42%) as a yellow oil. LCMS (ES, m/z): 194 [M+H]⁺.

### Step 3. 3-bromo-4-methylbenzoyl cyanide

A mixture of 3-bromo-4-methylbenzoic acid (80 g, 316 mmol, 85%) in SOCl₂ (70 mL) was stirred for 2 h at 80° C. The mixture was cooled to room temperature and concentrated under vacuum. The fresh prepared acyl chloride was dissolved in DCM (900 mL), to the above mixture was added SnCl₄ (43.4 g, 158 mmol) and TMSCN (34.6 g, 332 mmol). The resulting mixture was stirred for 2 h at 25° C. The reaction mixture was poured into water (200 mL) and extracted with DCM (3×500 mL). The combined organic layers were dried over anhydrous sodium sulfate and concentrated under vacuum. The crude product was purified by silica gel chromatography (eluting with ethyl acetate/petroleum ether (1/10)) to afford 3-bromo-4-methylbenzoyl cyanide as yellow oil (35.2 g, 49%). LCMS (ES, m/z): 224, 226 [M+H]⁺.

### Step 4. Tert-butyl 6-(3-bromo-4-methylbenzoyl)-1H,2H,3H-pyrrolo[3,4-c]pyridine-2-carboxylate

To a solution of 3-bromo-4-methylbenzoyl cyanide (2.50 g, 0.01 mol) in DCE (250 mL) was added tert-butyl N,N-bis(prop-2-yn-1-yl)carbamate (1.21 g, 0.01 mol), chloro(1,5-cyclooctadiene)(pentamethylcyclopentadienyl)ruthenium(II) (0.35 g, 0.92 mmol). The resulting mixture was stirred for 1 h at 60° C. The mixture was cooled to room temperature and concentrated under vacuum. The crude product was purified by silica gel chromatography (eluting with ethyl acetate/petroleum ether (1/9)) to afford tert-butyl 6-(3-bromo-4-methylbenzoyl)-1H,2H,3H-pyrrolo[3,4-c]pyridine-2-carboxylate as yellow solid (2.50 g, 53%). LCMS (ES, m/z): 417, 419 [M+H]⁺.

### Step 5. 6-(3-bromo-4-methylbenzoyl)-1H,2H,3H-pyrrolo[3,4-c]pyridine hydrochloride

To a solution of tert-butyl 6-(3-bromo-4-methylbenzoyl)-1H,2H,3H-pyrrolo[3,4-c]pyridine-2-carboxylate (2.50 g, 6.01 mmol) in DCM (30 mL) was added HCl in dioxane (10 mL, 4 M). The resulting solution was stirred for 2 h at room temperature. The mixture was concentrated under vacuum to afford 6-(3-bromo-4-methylbenzoyl)-1H,2H,3H-pyrrolo[3,4-c]pyridine hydrochloride as yellow solid (1.90 g, 90%). LCMS (ES, m/z): 317, 319 [M−HCl+H]⁺.

### Step 6. 2-{4-[[6-(3-bromo-4-methylbenzoyl)-]H,2H,3H-pyrrolo[3,4-c]pyridine-2-yl]sulfonyl}phenyl)-1,3-oxazole

To a mixture of 6-(3-bromo-4-methylbenzoyl)-1H,2H,3H-pyrrolo[3,4-c]pyridine hydrochloride (800 mg, 2.27 mmol) in DCM (30 mL) was added triethylamine (0.87 mL, 6.73 mmol) and 4-(1,3-oxazol-2-yl)benzene-1-sulfonyl chloride (660 mg, 2.71 mmol) dropwise at 0° C. The resulting mixture was stirred for 2 h at 25° C. The mixture was concentrated under vacuum. The crude product was purified by silica gel chromatography (eluting with ethyl acetate/petroleum ether (1/10)) to afford 2-(4-{[6-(3-bromo-4-methylbenzoyl)-1H,2H,3H-pyrrolo[3,4-c]pyridine-2-yl]sulfonyl}phenyl)-1,3-oxazole as a white solid (750 mg, 63%). LCMS (ES, m/z): 524, 526 [M+H]⁺.

### Step 7. 1-methyl-4-(2-methyl-5-{2-[4-(1,3-oxazol-2-yl)benzenesulfonyl]-1H,2H,3H-pyrrolo[3,4-c]pyridine-6-carbonyl}phenyl)piperazine

To a mixture of 2-(4-{[6-(3-bromo-4-methylbenzoyl)-1H,2H,3H-pyrrolo[3,4-c]pyridine-2-yl]sulfonyl}phenyl)-1,3-oxazole (600 mg, 1.15 mmol) in 1,4-dioxane (70 mL) was added 1-methylpiperazine (1.20 g, 12.0 mmol), RuPhos-Pd 3G (100 mg, 0.12 mmol), RuPhos (600 mg, 1.28 mmol) and Cs₂CO₃ (1.10 g, 3.34 mmol). The resulting mixture was stirred for 5 h at 100° C. The mixture was cooled to room temperature, poured into water (50 mL) and extracted with ethyl acetate (3×120 mL). The combined organic layers were dried over anhydrous sodium sulfate and concentrated under vacuum. The crude product was purified by silica gel chromatography (eluting with methanol/dichloromethane (1/10)) to afford 1-methyl-4-(2-methyl-5-{2-[4-(1,3-oxazol-2-yl)benzenesulfonyl]-1H,2H,3H-pyrrolo[3,4-c]pyridine-6-carbonyl}phenyl)piperazine as a white solid (400 mg, 64%). LCMS (ES, m/z): 544 [M+H]⁺.

### Step 8. (S)-[4-methyl-3-(4-methylpiperazin-1-yl)phenyl]({2-[4-(1,3-oxazol-2-yl)benzenesulfonyl]-1H,2H,3H-pyrrolo[3,4-c]pyridine-6-yl})methanol (PH-FMA-PJ00135-640-OA, first eluting isomer) and (R)-[4-methyl-3-(4-methylpiperazin-1-yl)phenyl]({2-[4-(1,3-oxazol-2-yl)benzenesulfonyl]-1H,2H,3H-pyrrolo[3,4-c]pyridine-6-yl})methanol (PH-FMA-PJ00135-640-OB, second eluting isomer)

To a mixture of 1-methyl-4-(2-methyl-5-{2-[4-(1,3-oxazol-2-yl)benzenesulfonyl]-1H,2H,3H-pyrrolo[3,4-c]pyridine-6-carbonyl}phenyl)piperazine (250 mg, 0.46 mmol) in methanol (3 mL) was added NaBH4 (16 mg, 0.42 mmol) in portions at 0° C. The resulting mixture was stirred for 1 h at 25° C. The mixture was poured into water (25 mL) and extracted with ethyl acetate (3×50 mL). The combined organic layers were dried over anhydrous sodium sulfate and concentrated under vacuum. The crude product was purified by silica gel chromatography (eluting with methanol/dichloromethane (1/9)) to afford the racemic product. The racemate was separated by Chiral Prep-HPLC with the following conditions: Column: Chiralpak IC 2×25 cm, 5 um; mobile phase A: MTBE (0.2% IPA), B: MeOH (50B to 50B in 18 min); Flow rate 20 mL/min, 220/254 nm, RT1: 15.54, RT2: 19.22. The product fractions were concentrated and lyophilized to afford (S)-[4-methyl-3-(4-methylpiperazin-1-yl)phenyl]({2-[4-(1,3-oxazol-2-yl)benzenesulfonyl]-1H,2H,3H-pyrrolo[3,4-c]pyridine-6-yl})methanol (first eluting isomer, RT1: 15.54) (34.4 mg, 15.97%) as a white solid. And (R)-[4-methyl-3-(4-methylpiperazin-1-yl)phenyl]({2-[4-(1,3-oxazol-2-yl)benzenesulfonyl]-1H,2H,3H-pyrrolo[3,4-c]pyridine-6-yl})methanol (second eluting isomer, RT2: 19.22) (32.6 mg, 15.13%) as a white solid.

First eluting isomer: 1H-NMR (DMSO, 400 MHz) δ (ppm): 8.32 (s, 2H), 8.16 (d, J=8.4 Hz, 2H), 8.01 (d, J=8.4 Hz, 2H), 7.46 (d, J=8.4 Hz, 2H), 7.05 (s, 1H), 6.96 (d, J=7.6 Hz, 1H), 6.79 (d, J=7.6 Hz, 1H), 5.99 (d, J=4.0 Hz, 1H), 5.60 (d, J=4.0 Hz, 1H), 4.65-4.59 (m, 4H), 2.76 (br s, 4H), 2.43 (br s, 4H), 2.21 (s, 3H), 2.13 (s, 3H). LCMS (ES, m/z): 546 [M+H]⁺.

Second eluting isomer: 1H-NMR (DMSO, 400 MHz) δ (ppm): 8.32 (s, 2H), 8.16 (d, J=8.8 Hz, 2H), 8.01 (d, J=8.4 Hz, 2H), 7.47-7.45 (m, 2H), 7.04 (s, 1H), 6.96 (d, J=8.0 Hz, 1H), 6.79-6.77 (m, 1H), 5.99 (d, J=4.4 Hz, 1H), 5.60 (d, J=4.0 Hz, 1H), 4.69-4.59 (m, 4H), 2.76 (br s, 4H), 2.45 (br s, 4H), 2.23 (s, 3H), 2.13 (s, 3H). LCMS (ES, m/z): 546 [M+H]⁺.

### Method N

### (S)-(3-[6-methyl-2,6-diazaspiro[3.3]heptan-2-yl]phenyl)([2-[4-(1,3-oxazol-2-yl)benzenesulfonyl]-1H,2H,3H-pyrrolo[3,4-c]pyridin-6-yl])methanol (first eluting isomer); (R)-(3-[6-methyl-2,6-diazaspiro[3.3]heptan-2-yl]phenyl)([2-[4-(1,3-oxazol-2-yl)benzenesulfonyl]-1H,2H,3H-pyrrolo[3,4-c]pyridin-6-yl])methanol (second eluting isomer)

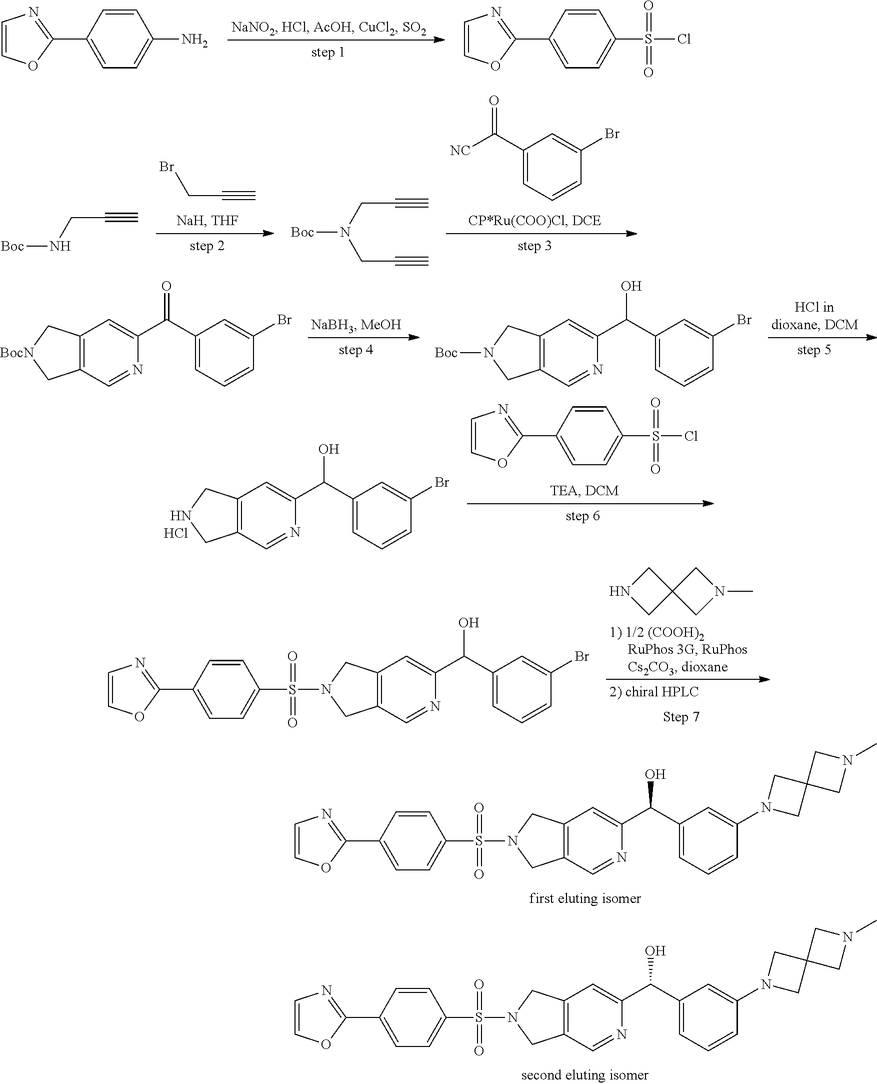

### Step 1. 4-(1,3-oxazol-2-yl)benzenesulfonyl chloride

To a stirred mixture of 4-(1,3-oxazol-2-yl) aniline (1.00 g, 6.24 mmol) in conc. HCl (12 mL) and acetic acid (4 mL) was added NaNO₂ (517 mg, 7.49 mmol) in water (0.5 mL) dropwise at −10′° C. The mixture was stirred for 1 h at 0° C. to afford the fresh prepared diazonium salt (mixture A). In a separated 3-necked round-bottom flask, SO₂ was pumped into the stirred mixture of CuCl₂ (235 mg, 1.75 mmol) in acetic acid (20 mL) for 2 h at 20° C. to afford mixture B. The mixture A was then added into the mixture B at 20° C. The resulting mixture was stirred for additional overnight at 25° C. The reaction mixture was diluted with water (40 mL), the precipitated solids were collected by filtration, washed with water (2×20 mL) and dried under vacuum to afford 4-(1,3-oxazol-2-yl)benzenesulfonyl chloride (1.20 g, 67%) as an off-white solid. LCMS (ES, m/z): 244, 246 [M+H]⁺.

### Step 2. tert-butyl N,N-bis(prop-2-yn-1-yl)carbamate

To a stirred mixture of tert-butyl N-(prop-2-yn-1-yl)carbamate (5.00 g, 30.6 mmol) in DMF (50 mL) was added NaH (1.35 g, 33.7 mmol, 60%) in portions at 0° C. The resulting mixture was stirred for 30 min at 0° C. under nitrogen atmosphere. This was followed by the additional of 3-bromoprop-1-yne (4.60 g, 36.7 mmol). The resulting mixture was stirred for 3 h at 26° C. under nitrogen atmosphere. The reaction mixture was poured into water/ice (100 mL) and extracted with ethyl acetate (2×100 mL). The combined organic layers were washed with brine (3×100 mL), dried over anhydrous sodium sulfate, and concentrated under vacuum. The residue was purified by silica gel column chromatography, eluted with PE/EtOAc (1:1) to afford tert-butyl N,N-bis(prop-2-yn-1-yl)carbamate (2.60 g, 42%) as a yellow oil. LCMS (ES, m/z): 194 [M+H]⁺.

### Step 3. tert-butyl 6-(3-bromobenzoyl)-1H,3H-pyrrolo[3,4-c]pyridine-2-carboxylate

To a stirred mixture of tert-butyl N,N-bis(prop-2-yn-1-yl)carbamate (1.50 g, 7.76 mmol) and 3-bromobenzoyl cyanide (1.63 g, 7.76 mmol) in DCE (20 mL) was added chloro(1,5-cyclooctadiene)(pentamethylcyclopentadienyl)ruthenium(II) (236 mg, 0.62 mmol). The resulting mixture was stirred for 1 h at 60° C. under nitrogen atmosphere. The mixture was cooled to room temperature, poured into water/ice (40 mL) and extracted with ethyl acetate (2×40 mL). The combined organic layers were washed with brine (40 mL), dried over anhydrous sodium sulfate, and concentrated under vacuum. The residue was purified by silica gel column chromatography, eluted with PE/EtOAc (1:1) to afford tert-butyl 6-(3-bromobenzoyl)-1H,3H-pyrrolo[3,4-c]pyridine-2-carboxylate (1.80 g, 55%) as a brown solid. LCMS (ES, m/z): 403, 405 [M+H]⁺.

### Step 4. Tert-butyl 6-[(3-bromophenyl)(hydroxy)methyl]-1H,2H,3H-pyrrolo[3,4-c]pyridine-2-carboxylate

To a solution of tert-butyl 6-(3-bromobenzoyl)-1H,3H-pyrrolo[3,4-c]pyridine-2-carboxylate (2.40 g, 5.95 mmol) in methanol (15 mL) and dichloromethane (15 mL) was added NaBH₄ (450 mg, 11.9 mmol) in portions at 0° C. The resulting mixture was stirred for 1 h at room temperature. The mixture was concentrated and diluted with water (10 mL). The resulting mixture was extracted with dichloromethane (3×20 mL), dried over anhydrous sodium sulfate, and concentrated under vacuum. The crude product was purified by silica gel chromatography (eluting with ethyl acetate/petroleum ether (1/1)) to afford tert-butyl 6-[(3-bromophenyl)(hydroxy)methyl]-1H,2H,3H-pyrrolo[3,4-c]pyridine-2-carboxylate as a yellow oil (2.20 g, 91%). LCMS (ES, m/z): 405, 407 [M+H]⁺.

### Step 5. (3-bromophenyl)({1H,2H,3H-pyrrolo[3,4-c]pyridin-6-yl})methanol hydrochloride

To a solution of tert-butyl 6-[(3-bromophenyl)(hydroxy)methyl]-1H,2H,3H-pyrrolo[3,4-c]pyridine-2-carboxylate (2.20 g, 5.43 mmol) in dichloromethane (20 mL) was added HCl (5 mL, 4 M in dioxane). The resulting solution was stirred for 3 h at room temperature. The mixture was concentrated under vacuum to afford (3-bromophenyl)({1H,2H,3H-pyrrolo[3,4-c]pyridin-6-yl})methanol hydrochloride as yellow solid (1.70 g, 91%). LCMS (ES, m/z): 305, 307 [M−HCl+H]⁺.

### Step 6. (3-bromophenyl)({2-[4-(1,3-oxazol-2-yl)benzenesulfonyl]-1H,2H,3H-pyrrolo[3,4-c]pyridin-6-yl})methanol

To a stirred mixture of (3-bromophenyl)({1H,2H,3H-pyrrolo[3,4-c]pyridin-6-yl})methanol hydrochloride (1.68 g, 4.92 mmol) in dichloromethane (20 mL) was added TEA (6.85 mL, 67.7 mmol) and 4-(1,3-oxazol-2-yl)benzene-1-sulfonyl chloride (1.20 g, 4.93 mmol) dropwise at 0° C. The resulting mixture was stirred for 2 h at room temperature. The mixture was concentrated under vacuum and the residue was diluted with of methanol (30 mL). The solids were collected by filtration and dried under vacuum to afford (3-bromophenyl)({2-[4-(1,3-oxazol-2-yl)benzenesulfonyl]-1H,2H,3H-pyrrolo[3,4-c]pyridin-6-yl})methanol as pink solid (2.00 g, 79%). LCMS (ES, m/z): 512, 514 [M+H]⁺.

### Step 7. (S)-(3-[6-methyl-2,6-diazaspiro[3.3]heptan-2-yl]phenyl)([2-[4-(1,3-oxazol-2-yl)benzenesulfonyl]-1H,2H,3H-pyrrolo[3,4-c]pyridin-6-yl])methanol (first eluting isomer) and (R)-(3-[6-methyl-2,6-diazaspiro[3.3]heptan-2-yl]phenyl)([2-[4-(1,3-oxazol-2-yl)benzenesulfonyl]-1H,2H,3H-pyrrolo[3,4-c]pyridin-6-yl])methanol (second eluting isomer)

To a solution of (3-bromophenyl)([2-[4-(1,3-oxazol-2-yl)benzenesulfonyl]-1H,2H,3H-pyrrolo[3,4-c]pyridin-6-yl])methanol (100 mg, 0.19 mmol) in dioxane (5 mL) was added RuPhos 3 G (16 mg, 0.02 mmol), RuPhos (9 mg, 0.02 mmol), Cs₂CO₃ (191 mg, 0.56 mmol) and bis(2-methyl-2,6-diazaspiro[3.3]heptane); oxalic acid (66 mg, 0.21 mmol). The resulting mixture was stirred for 4 h at 110° C. The reaction mixture was cooled to room temperature, poured into water (30 mL) and extracted with ethyl acetate (3×30 mL). The combined organic layers were dried over anhydrous sodium sulfate concentrated under vacuum. The crude product was purified by Prep-HPLC (Column: XBridge Shield RP18 OBD Column, 5 μm, 30×150 mm; Mobile Phase, A: water (containing 10 mmol/L NH₄HCO₃) and B: CH3CN (10% to 40% in 7 min); Flow rate: 60 mL/min; Detector: UV 254 nm). The product fractions were concentrated under vacuum to afford the racemic product. The racemate was separated by Chiral Prep-HPLC with the following condition: Column: CHIRALPAK IC, 5 μm, 20×250 mm; Mobile Phase, A: methanol (containing 0.1% DEA) and B: DCM (keep 50% B in 18 min); Flow rate: 20 mL/min; Detector: UV 254/220 nm; RT1: 10.692 min; RT2: 14.71 min. The product fractions were concentrated and lyophilized to afford (S)-(3-[6-methyl-2,6-diazaspiro[3.3]heptan-2-yl]phenyl)([2-[4-(1,3-oxazol-2-yl)benzenesulfonyl]-1H,2H,3H-pyrrolo [3,4-c]pyridin-6-yl])methanol (first eluting isomer, RT1: 10.692 men) as a white solid (16.9 mg, 16%). And (R)-(3-[6-methyl-2,6-diazaspiro[3.3]heptan-2-yl]phenyl)([2-[4-(1,3-oxazol-2-yl)benzenesulfonyl]-1H,2H,3H-pyrrolo[3,4-c]pyridin-6-yl])methanol (second eluting isomer, RT2: 14.71 mm;) as a white solid (15.2 mg, 15%).

First eluting isomer: H-NMR (DMSO-d6, 400 MHz) δ (ppm): 8.32 (s, 2H), 8.16 (d, J 8.8 Hz, 2H), 8.01 (d, J=8.4 Hz, 2H), 7.47-7.44 (m, 2H), 7.00-6.96 (m, 1H), 6.58 (d, J=8.0 Hz, 1H), 6.44 (s, 1H), 6.20 (d, J=8.4 Hz, 1H), 5.96 (d, J=4.0 Hz, 1H), 5.56 (d, J=423.6 Hz, 1H), 4.64-4.58 (i, 4H), 3.78 (s, 4H), 3.33-3.27 (m, 4H), 2.20 (s, 3H). LCMS (ES, m5z): 544 [M+H]⁺.

Second eluting isomer: 1H-NMR (DMSO-d6, 400 MHz) δ (ppm): 8.32 (s, 2H), 8.16 (d, J=8.4 Hz, 2H), 8.01 (d, J=8.4 Hz, 2H), 7.47-7.44 (m, 2H), 7.00-6.96 (m, 1H), 6.58 (d, J=7.6 Hz, 1H), 6.44 (s, 1H), 6.20 (d, J=7.6 Hz, 1H), 5.96 (d, J=4.0 Hz, 1H), 5.56 (d, J=4.0 Hz, 1H), 4.65-4.58 (m, 4H), 3.79-3.75 (m, 4H), 3.25 (s, 4H), 2.19 (s, 3H). LCMS (ES, m/z): 544 [M+H]⁺.

TABLE 15

| Cmpd. No. | Purification Methods |
|---|---|
| 3 and 4 | Prep-HPLC (Column: XBridge Shield RP18 OBD Column, 5 μm, 19 × 150 mm; Mobile Phase, A: |
| | water (containing 10 mmol/L NH4HCO3) and B: CH3CN (10% to 30% over 20 min); Flow rate: 20 |
| | mL/min; Detector: UV 254 nm). The two enantiomers were further separated by Chiral Prep-HPLC |
| | (Column: CHIRALPAK IA, 5 μm, 20 × 250 mm; Mobile Phase, A: methanol and B: DCM (hold |
| | 50% B over 25 min); Detector: UV 254/220 nm; Retention time: 1^{st}, 7.62 min; 2^{nd}, 9.89 min) |
| 5 and 6 | CHIRALPAK IG, 20 × 250 mm, 5 mm; Mobile Phase A: MeOH (0.1% DEA), Mobile Phase B: |
| | DCM; Flow rate: 20 mL/min; Gradient: 50 B to 50 B in 10 min; 220/254 nm; Retention time: 1^{st}, |
| | 3.97; Retention time: 2^{nd}, 5.96 |
| 7 | Prep-HPLC (Column: XBridge Shield C18 OBD Column, 5 μm, 19 × 150 mm; Mobile Phase, A: |
| | water (containing 10 mmol/L NH4HCO3) and B: CH3CN (20% to 42% over 7 min); Flow rate: 20 |
| | mL/min; Detector: UV 254/220 nm) |
| 8 | Prep-HPLC (Column: XBridge Shield C18 OBD Column, 5 μm, 19 × 150 mm; Mobile Phase, A: |
| | water (containing 10 mmol/L NH4HCO3) and B: CH3CN (40% to 57% over 7 min); Flow rate: 20 |
| | mL/min; Detector: UV 254/220 nm) |
| 9 | Prep-HPLC (Column: XBridge Shield RP18 OBD Column, 5 μm, 19 × 150 mm; Mobile Phase, A: |
| | water (containing 10 mmol/L NH4HCO3) and B: CH3CN (25% to 60% over 5.8 min); Flow rate: |
| | 20 mL/min; Detector: UV 254 nm) |
| 10 | Prep-HPLC (Column: XBridge Shield RP18 OBD Column, 5 μm, 19 × 150 mm; Mobile Phase, A: |
| | water (containing 10 mmol/L NH4HCO3) and B: CH3CN (30% to 58% over 7 min) |
| 11 | Prep-HPLC (Column: XBridge Shield C18 OBD Column, 5 μm, 19 × 150 mm; Mobile Phase, A: |
| | water (containing 10 mmol/L NH4HCO3) and B: CH3CN (30% to 65% over 7 min); Flow rate: 20 |
| | mL/min; Detector: UV 254/220 nm) |
| 12 | Prep-HPLC (Column: XBridge Shield RP18 OBD Column, 5 μm, 19 × 150 mm; Mobile Phase, A: |
| | water (containing 10 mmol/L NH4HCO3) and B: MeCN (25% to 38% over 8 min); Flow rate: 60 |
| | mL/min; Detector: UV 254 nm) |
| 13 | Prep-HPLC (Column: XBridge Shield RP18 OBD Column, 5 μm, 19 × 150 mm; Mobile Phase, A: |
| | water (containing 10 mmol/L NH4HCO3) and B: MeCN (25% to 65% over 8 min); Flow rate: 20 |
| | mL/min; Detector: UV 254 nm) |
| 14 and 15 | Prep HPLC (CHIRALPAK IG, 5 μm, 20 × 250 mm; Mobile Phase, A: hexane: DCM (3:1, |
| | containing 0.1% isopropyl amine) and B: MeOH: DCM (1:1); Gradient: keep 30% B over 17 min; |
| | Flow rate: 20 mL/min; Detector: UV 254/220 nm; Retention time: 1^{st}, 8.426 min; 2^{nd}, 10.756 min) |
| 17 | prep-HPLC (Column: XBridge Shield RP18 OBD Column, 5 μm, 19 × 150 mm; Mobile Phase, A: |
| | water (containing 10 mmol/L NH4HCO3) and B: CH3CN (30% B to 62% B over 7 min); Flow rate: |
| | 20 mL/min; Detector: UV 254 nm) |
| 18 | Prep-HPLC (Column: XBridge Shield C18 OBD Column, 5 μm, 19 × 150 mm; Mobile Phase, A: |
| | water (containing 10 mmol/L NH4HCO3) and B: CH3CN (40% to 62% over 7 min); Flow rate: 20 |
| | mL/min; Detector: UV 254/220 nm) |
| 19 | Prep-HPLC (Column: XBridge Shield C18 OBD Column, 5 μm, 19 × 150 mm; Mobile Phase, A: |
| | water (containing 10 mmol/L NH4HCO3) and B: CH3CN (30% to 65% over 7 min); Flow rate: 20 |
| | mL/min; Detector: UV 254/220 nm) |
| 20, 21, 22, | Prep-HPLC (Column: XBridge Shield RP18 OBD Column, 5 μm, 19 × 150 mm; Mobile Phase, A: |
| and 23 | water (containing 10 mmol/L NH4HCO3) and B: CH3CN (5% to 5% over 3 min); Flow rate: 20 |
| | mL/min; Detector: UV 254 nm). The four enantiomers were further separated by Chiral-Prep-HPLC |
| | (Column: CHIRALPAK IC, 5 μm, 20 × 250 mm; Mobile Phase, A: methanol (containing 0.2% |
| | isopropyl amine) and B: isopropyl alcohol (hold 50% B over 25 min); Detector: UV 254/220 nm; |
| | Retention time: 1^{st}, 1.583 min; 2^{nd}, 2.189 min; 3^{rd}, 1.698 min; 4^{th}, 3.285 min) |
| 25 | XBridge Shield RP18 OBD Column, 5 μm, 19 × 150 mm; Mobile Phase, A: water (containing 10 |
| | mmol/L NH4HCO3) and B: CH3CN (20% to 40% over 7 min); Flow rate: 20 mL/min; Detector: |
| | UV 254 nm). |
| 26 and 27 | CHIRALPAK IF, 5 μm, 20 × 250 mm; Mobile Phase, A: MTBE (containing 0.2% IPA) and B: EtOH |
| | (keep 50% B over 25 min); Detector: UV 254/220 nm; Retention time: 1^{st}, 10.759 min; 2^{nd}, 17.742 |
| | min). |
| 28 | XBridge Shield RP18 OBD Column, 5 μm, 19 × 150 mm; Mobile Phase, A: water (containing 10 |
| | mmol/L NH4HCO3) and B: CH3CN (20% to 40% over 7 min); Flow rate: 20 mL/min; Detector: |
| | UV 254 nm). |
| 29 and 30 | Prep-HPLC (Column: XBridge Shield RP18 OBD Column, 5 μm, 19 × 150 mm; Mobile Phase, A: |
| | water (containing 10 mmol/L NH4HCO3) and B: CH3CN (25% to 45% over 8 min); Flow rate: 20 |
| | mL/min; Detector: UV 254 nm). The product fractions were concentrated under vacuum. The two |
| | enantiomers were separated by Prep-Chiral-HPLC (Column: CHIRALPAK IF, 5 μm, 20 × 250 mm; |
| | Mobile Phase, A: MTBE (containing 0.2% IPA) and B: EtOH (keep 50% B over 25 min); Detector: |
| | UV 254/220 nm; Retention time: 1^{st}, 8.583 min; 2^{nd}, 12.347 min |
| 31 | Prep-HPLC (Column: XBridge Shield RP18 OBD Column, 5 μm, 19 × 150 mm; Mobile Phase, A: |
| | water (containing 10 mmol/L NH4HCO3) and B: CH3CN (25% to 45% over 7 min); Flow rate: 20 |
| | mL/min; Detector: UV 254 nm) |
| 32 | Prep-HPLC (Column: XBridge Shield C18 OBD Column, 5 μm, 19 × 150 mm; Mobile Phase, A: |
| | water (containing 10 mmol/L NH4HCO3) and B: CH3CN (25% to 50% over 7 min); Flow rate: 20 |
| | mL/min; Detector: UV 254/220 nm) |
| 33 | Prep-HPLC (Column: XBridge Prep OBD C18 Column, 5 μm, 19 × 150 mm; Mobile Phase, A: |
| | water (containing 10 mmol/L NH4HCO3) and B: CH3CN (37% to 62% over 7 min); Flow rate: 20 |
| | mL/min; Detector: UV 254/220 nm) |
| 34 | Prep-HPLC (Column: XBridge Shield RP18 OBD Column, 5 μm, 30 × 150 mm; Mobile Phase, A: |
| | water (containing 10 mmol/L NH4HCO3) and B: CH3CN (32% to 42% over 7 min); Flow rate: 60 |
| | mL/min; Detector: UV 254/220 nm) |
| 35 | Prep-HPLC (Column: XBridge Shield RP18 OBD Column, 5 μm, 19 × 150 mm; Mobile Phase, A: |
| | water (containing 10 mmol/L NH4HCO3) and B: CH3CN (15% to 45% over 7 min) |
| 36 | Prep-HPLC (Column: XBridge Shield RP18 OBD Column, 5 μm, 30 × 150 mm; Mobile Phase, A: |
| | water (containing 10 mmol/L NH4HCO3) and B: CH3CN (25% to 55% over 7 min); Flow rate: 20 |
| | mL/min; Detector: UV 254/220 nm) |
| 37 | XBridge Shield RP18 OBD Column, 5 μm, 19 × 150 mm; Mobile Phase, A: water (containing 10 |
| | mmol/L NH4HCO3) and B: CH3CN (30% to 50% over 7 min); Flow rate: 20 mL/min; Detector: |
| | UV 254 nm). |
| 38 and 39 | Chiral Prep-HPLC (Column: CHIRALPAK IE, 5 μm, 20 × 250 mm; Mobile Phase, A: MTBE |
| | (containing 0.1% TFA) and B: EtOH; Gradient: keep 5% B over 22 min; Flow rate: 20 mL/min; |
| | Detector: UV 254/220 nm; Retention time: 1^{st}, 15.705 min; 2^{nd}, 19.957 min) |
| 40 and 41 | Chiral Prep-HPLC (Column: CHIRALPAK IE, 5 μm, 20 × 250 mm; Mobile Phase, A: MTBE |
| | (containing 0.2% IPA) and B: MeOH (keep 30% B over 14 min); Flow rate: 18 mL/min; Detector: |
| | UV 254/220 nm; Retention time: 1^{st}, 8.184 min; 2^{nd}, 10.819 min) |
| 42 and 43 | Prep-SFC (Column: CHIRALPAK IG, 5 μm, 20 × 250 mm; Mobile Phase A: CO2, Mobile Phase B: |
| | EtOH:DCM = 1:1 (keep 50% B over 12 min); Flow rate: 40 mL/min; Detector, UV 220 nm; |
| | Retention time: 1^{st}, 8.33 min; 2^{nd}, 10.81 min) |
| 44 | Prep-HPLC (Column: XBridge Shield RP18 OBD Column, 5 μm, 30 × 150 mm; Mobile Phase, A: |
| | water (containing 10 mmol/L NH4HCO3) and B: MeCN (30% to 55% over 7 min); Flow rate: 60 |
| | mL/min; Detector: UV 254 nm) |
| 45 | Prep-HPLC (Column: XBridge Shield RP18 OBD Column, 5 μm, 30 × 150 mm; Mobile Phase, A: |
| | water (containing 10 mmol/L NH4HCO3) and B: MeCN (35% to 55% over 7 min); Flow rate: 60 |
| | mL/min; Detector: UV 254 nm) |
| 46 and 47 | Chiral Prep-HPLC (Column: CHIRALPAK IC, 5 μm, 20 × 250 mm; Mobile Phase, A: hexane: |
| | DCM = 3:1 (containing 0.1% DEA), B: EtOH; Gradient: keep 50% B over 24 min; Flow rate: 18 |
| | mL/min; Detector: UV 254/220 nm; Retention time: 1^{st}, 12.618 min; 2^{nd}, 19.401 min) |
| 48 and 49 | Chiral Prep-HPLC (Column: CHIRALPAK IC, 5 μm, 20 × 250 mm; Mobile Phase, A: MTBE |
| | (containing 0.2% DEA) and B: MeOH; Gradient: keep 50% B over 18 min; Flow rate: 20 mL/min; |
| | Detector: UV 220/254 nm; Retention time: 1^{st}, 9.22 min; 2^{nd}, 15.54 min) |
| 50 and 51 | Prep-HPLC (Column: XBridge Shield RP18 OBD Column, 5 μm, 30 × 150 mm; Mobile Phase, A: |
| | water (containing 10 mmol/L NH4HCO3) and B: CH3CN (25% to 40% over 8 min); Flow rate: 60 |
| | mL/min; Detector: UV 254 nm) |
| 52 and 53 | Chiral Prep-HPLC (Column: CHIRAL ART Cellulose-SB, 5 μm, 20 × 250 mm; Mobile Phase, A: |
| | MTBE (0.2% IPA) and B: MeOH (keep 20% B over 16 min); Flow rate: 20 mL/min; Detector: UV |
| | 254/220 nm; Retention time: 1^{st}, 10.406 min; 2^{nd}, 13.941 min) |
| 54 | XBridge Shield RP18 OBD Column, 5 μm, 19 × 150 mm; Mobile Phase, A: water (containing 10 |
| | mmol/L NH4HCO3) and B: CH3CN (22% to 37% over 7 min); Flow rate: 20 mL/min; Detector: |
| | UV 254 nm). |
| 55 and 56 | CHIRALPAK IC, 5 μm, 20 × 150 mm; Mobile Phase, A: MeCN and B: water (containing 10 |
| | mmol/L NH4HCO3) (keep 20% B over 12 min); Flow rate: 20 mL/min; Detector: UV 254/220 nm; |
| | Retention time: 1^{st}, 5.931 min; 2^{nd}, 10.037 min). |
| 57 and 58 | Chiral Prep-HPLC (Column: CHIRALPAK IG, 5 μm, 20 × 250 mm; Mobile Phase, A: methanol |
| | (containing 0.1% DEA) and B: DCM (keep 50% B in 35 min); Flow rate: 16 mL/min; Detector: UV |
| | 254/220 nm; Retention time: 1^{st}, 19.689 min; 2^{nd}, 29.857 min) |
| 59 and 60 | Chiral Prep-HPLC (Column: CHIRALPAK IG, 5 μm, 20 × 250 mm; Mobile Phase, A: MTBE |
| | (0.2% IPA) and B: EtOH (keep 50% B over 25 min); Flow rate: 20 mL/min; Detector: UV 254/220 |
| | nm; Retention time: 1^{st}, 10.086 min; 2^{nd}, 17.541 min) |
| 61, 62, 63, | Chiral Prep-HPLC (CHIRALPAK IC, 2 × 25 cm, 5 μm; Mobile Phase A: Hexane: DCM = 3:1 |
| and 64 | (containing 0.1% DEA), Mobile Phase B: isopropanol; Flow rate: 20 mL/min; Gradient: keep 30% |
| | B over 30 min; Detector: 220/254 nm; Rt: 1^{st}: 17.624 min; 2^{nd}: 23.93 min) |
| 65, 66, 67 | Chiral Prep-HPLC (CHIRALPAK IC, 2 × 25 cm, 5 μm; Mobile Phase A: MTBE (containing 0.1% |
| and 68 | IPA), Mobile Phase B: MeOH; Flow rate: 20 mL/min; Gradient: keep 10% B over 20 min; Detector: |
| | 220/254 nm; Rt: 1^{st}: 7.451 min; 2^{nd}: 10.399 min) |
| 69 and 70 | Chiral Prep-HPLC (Column: CHIRALPAK IC, 2 × 25 cm, 5 μm; Mobile Phase A: MTBE |
| | (containing 0.1% DEA), Mobile Phase B: MeOH; Flow rate: 20 mL/min; Gradient: 50 B to 50 B in |
| | 18 min; Detector: 220/254 nm; RT: 1^{st}: 8.833 min; 2^{nd}: 13.602 min) |
| 71 and 72 | Chiral Prep-HPLC (Column: CHIRALPAK IC, 5 μm, 20 × 250 mm; Mobile Phase, A: MeOH |
| | (containing 0.1% DEA) and B: DCM (keep 40% B in 15 min); Flow rate: 20 mL/min; Detector: UV |
| | 254/220 nm; Retention time: 1^{st}, 10.772 min; 2^{nd}, 13.314 min) |
| 73 and 74 | Chiral Prep-HPLC (Column: CHIRALPAK IC, 2 × 25 cm, 5 μm; Mobile Phase A: MeOH, Mobile |
| | Phase B: DCM; Flow rate: 15 mL/min; Gradient: keep 30% B over 30 min; Detector: UV 254/220 |
| | nm; Rt: 1^{st}, 13.818 min; 2^{nd}, 25.13 min) |
| 75 | Chiral Prep-HPLC (Column: CHIRALPAK IF, 5 μm, 20 × 250 mm; Mobile Phase, A: MeOH |
| | (containing 0.1% DEA) and B: DCM (keep 50% B over 28 min); Detector: UV 254/220 nm; |
| | Retention time: 1^{st}, 12.7 min; 2^{nd}, 22.3 min) |
| 76 and 77 | Chiral Prep-HPLC (Column: CHIRALPAK IF, 5 μm, 20 × 250 mm; Mobile Phase, A: methanol |
| | (containing 0.1% DEA) and B: DCM; Gradient: keep 30% B over 18 min; Detector: UV 254/220 |
| | nm; Retention time: 1^{st}, 8.2 min; 2^{nd}, 10.215 min) |
| 78 and 79 | Chiral Prep-HPLC (Column: CHIRALPAK IC, 5 μm, 20 × 250 mm; Mobile Phase, A: MeOH |
| | (containing 0.1% ammonia) and B: DCM (keep 50% B over 11 min); Flow rate: 20 mL/min; |
| | Detector: UV 254/220 nm; Retention time: 1^{st}, 6.831 min; 2^{nd}, 8.288 min) |
| 82 and 83 | Chiral Prep-HPLC (Column: CHIRALPAK IC, 2 × 25cm, 5 μm; Mobile Phase, A: MTBE |
| | (containing 0.2% IPA) and B: MeOH; Flow rate: 20 mL/min; Gradient: keep 50% B over 13 min; |
| | Detector: UV 254/220 nm; Retention time: 1^{st}, 8.541 min; 2^{nd}, 9.831 min) |
| 84 and 85 | Chiral Prep-HPLC (Column: CHIRALPAK IC, 5 μm, 20 × 250 mm; Mobile Phase, A: methanol |
| | (containing 0.1% DEA) and B: DCM (keep 50% B over 18 min); Flow rate: 20 mL/min; Detector: |
| | UV 254/220 nm; Retention time: 1^{st}, 10.692 min; 2^{nd}, 14.71 min) |
| 86, 87, 88, | Chiral Prep-HPLC (Column: CHIRALPAK IA, 5 μm, 20 × 250 mm; Mobile Phase, A: MTBE |
| and 89 | (containing 0.1% IPA) and B: ethanol (keep 50% B over 60 min); Flow rate: 13 mL/min; Detector: |
| | UV 254/220 nm; Retention time: 1^{st}, 18.353 min; 2^{nd}, 22.077 min; 3^{rd}, 30.765 min; 4^{th}, 47.141 min) |
| 90 and 91 | CHIRALPAK IC, 5 μm, 20 × 250 mm; Mobile Phase, A: MTBE(0.2% IPA)--HPLC and B: EtOH-- |
| | HPLC (keep 40% B over 14 min); Flow rate: 17 mL/min; Detector: UV 220/254 nm; Retention |
| | time: 1^{st}, 7.122 min; 2^{nd}, 11.298 min). |
| 92, 93, 94 | Chiral Prep-HPLC (Column: CHIRALPAK IG, 5 μm, 20 × 250 mm; Mobile Phase, A: hexane: |
| and 95 | DCM = 3:1 (containing 0.1% IPA) and B: MeOH: DCM = 1:1; Gradient: keep 70% B over 14 min; |
| | Flow rate: 20 mL/min; Detector: UV 220/254 nm; Retention time: 3^{rd}, 17.159 min; 4^{th}, 10.963 min) |
| 96 and 97 | Chiral Prep-HPLC (Column: CHIRALPAK IG, 5 μm, 20 × 250 mm; Mobile Phase, A: hexane: |
| | DCM = 3:1 (containing 0.1% IPA) and B: MeOH; Gradient: keep 40% B over 22 min; Flow rate: 20 |
| | mL/min; Detector: UV 254/220 nm; Retention time: 1^{st}, 15.522 min; 2^{nd}, 19.584 min) |
| 98 and 99 | Chiral Prep-HPLC (Column: CHIRAL ART Cellulose-SB, 5 μm, 20 × 250 mm; Mobile Phase, A: |
| | MeOH (containing 0.1% DEA) and B: DCM; Gradient: keep 15% B over 22 min; Flow rate: 20 |
| | mL/min; Detector: UV 254/220 nm; Retention time: 1^{st}, 11.342 min; 2^{nd}, 17.438 min) |
| 100 and | Chiral Prep-HPLC (Column: CHIRALPAK IC, 5 μm, 20 × 250 mm; Mobile Phase, A: hexane: |
| 101 | DCM = 3:1 (containing 0.1% DEA) and B: MeOH: DCM = 1:1 (keep 50% B over 20 min); Flow |
| | rate: 20 mL/min; Detector: UV 220/254 nm; Retention time: 1^{st}, 11.313 min; 2^{nd}, 15.462 min) |
| 102 and | Chiral Prep-HPLC (Column: CHIRALPAK IC, 5 μm, 20 × 250 mm; Mobile Phase, A: MeOH and |
| 103 | B: DCM; Gradient: keep 50% B over 12 min; Flow rate: 20 mL/min; Detector: UV 220/254 nm; |
| | Retention time: 1^{st}, 6.744 min; 2^{nd}, 9.757 min) |
| 104 and | CHIRALPAK IF, 5 μm, 20 × 250 mm; Mobile Phase A:HEX:DCM = 3:1(0.2% IPA), Mobile Phase |
| 105 | B: EtOH (keep 50% B over 17 min); Detector: UV 254/220 nm; Retention time: 1^{st},6.956 min; 2^{nd}, |
| | 13.145 min). |
| 106 and | Chiral Prep-HPLC (Column: CHIRALPAK IC, 5 μm, 20 × 250 mm; Mobile Phase, A: hexane: |
| 107 | DCM = 3:1 (containing 0.1% DEA) and B: MeOH: DCM = 1:1; Gradient: keep 60% B over 10 min; |
| | Flow rate: 20 mL/min; Detector: UV 220/254 nm; Retention time: 1^{st}, 6.446 min; 2^{nd}, 8.034 min) |

### Example 3. Biochemical Assay for USP9X Inhibition

The assay was performed in a final volume of 6 μL assay buffer containing 20 mM Tris-HCl (pH 8.0, (1M Tris-HCl, pH 8.0 solution; Corning 46-031-CM)), L-Glutathione (GSH) reducing agent (1 mM, Sigma-Aldrich, G4251-100G), 0.03% Bovine Gamma Globulin (BGG) (0.22 μM filtered, Sigma, G7516-25G), and 0.01% Triton X-100 (Sigma, T9284-10L). DMSO solutions of the compounds in nanoliter quantities (10-point, 3-fold serial dilutions) were dispensed into 1536 assay plates (Corning, #3724BC) for final test concentrations of 25 μM to 1.3 nM, top to lowest dose, respectively. Concentration and incubation times were optimized for the maximal signal-to-background while maintaining initial velocity conditions at a fixed substrate concentration (<<Kₘ). The final concentration of USP9X (Enzyme, E) was 0.025 nM, and the final concentration of Ubiquitin-Rhoadmine 110 (Ub-Rh110, UbiQ-126) (Substrate, S) was 25 nM. To assay plates (pre-stamped with compound) was added 3 μL 2× Enzyme. The enzyme was preincubated for 30 minutes and then treated with 3 μL of 2× Substrate. Plates were incubated for 11 min (continuous kinetic read) at room temperature before the fluorescence was read on the Envision plate reader (Perkin Elmer) or PheraSTAR plate reader (BMG), with excitation at 485 nm and emission at 535 nm. The slope (best fit linear regression) of the five reads was used to normalize for inhibition. For all assays, data are reported as percent inhibition compared with control wells based on the following equation: % inh=100*((FLU−AveLow)/(AveHigh−AveLow)), wherein FLU is measured Fluorescence, AveLow is average Fluorescence of no enzyme control (n=64), and AveHigh is average Fluorescence of DMSO control (n=64). IC₅₀ values are determined by curve fitting of the standard 4 parameter logistic fitting algorithm included in the Activity Base software package: IDBS XE Designer Model205. Data are fitted using the Levenburg Marquardt algorithm.

As set forth in Tables 16 and 17, IC₅₀ values are defined as follows: >25 μM (−−); ≤25 μM and >10 μM (+); ≤10 μM and >1 μM (++); ≤1 μM and >0.1 μM (+++); ≤0.1 μM and >0.001 μM (++++).

In Tables 16 and 17, absolute stereochemistry has not been determined for some Examples. Accordingly, assignment of any Examples as the “R” or “S” stereoisomer is arbitrary, unless otherwise noted. In some cases, Examples are labeled with “1^{st} eluting isomer”, “2^{nd} eluting isomer”, etc. based on the purification method used to separate the stereoisomers (see Table 15).

TABLE 16

| Cmpd. | IC₅₀ | Inter- | Synth. | | MS m/z | ¹H |
|---|---|---|---|---|---|---|
| No. | (μM) | mediates | Method | Chemical Name | [M + H]⁺ | NMR |
| 1 | ++ | | | (S)-[2-(2,3-dihydro-1,4- | 424.0 | (CD₃OD, 400 MHz) δ |
| (1^{st} | | | | benzodioxine-6-sulfonyl)-2,3- | | (ppm): 7.34-7.18 (m, 9H), |
| eluting | | | | dihydro-1H-isoindol-5- | | 7.15-7.13 (m, 1H), 6.98-6.96 |
| isomer) | | | | yl](phenyl)methanol; | | (m, 1H), 5.72 (s, 1H), 4.53 |
| 2 | +++ | | | (R)-[2-(2,3-dihydro-1,4- | | (s, 4H), 4.29-4.24 (m, 4H). |
| (2^{nd} | | | | benzodioxine-6-sulfonyl)-2,3- | | |
| eluting | | | | dihydro-1H-isoindol-5- | | |
| isomer) | | | | yl](phenyl)methanol | | |
| 3 | +++ | 8-10, | I | (S)-[2-(2,3-dihydro-1,4- | 425.0 | (Methanol-d4, 300 MHz) δ |
| (1^{st} | | 15-1 | | benzodioxine-6-sulfonyl)- | | (ppm): 8.34-8.29 (m, 1H), |
| eluting | | | | 1H,2H,3H-pyrrolo[3,4- | | 7.53-7.49 (m, 1H), 7.42-7.17 |
| isomer) | | | | c]pyridin-6- | | (m, 7H), 7.04-6.95 (m, 1H), |
| 4 | +++ | | | yl](phenyl)methanol; | | 5.79 (s, 1H), 4.64 (s, 4H), |
| (2^{nd} | | | | (R)-[2-(2,3-dihydro-1,4- | | 4.35-4.22 (m, 4H). |
| eluting | | | | benzodioxine-6-sulfonyl)- | | |
| isomer) | | | | 1H,2H,3H-pyrrolo[3,4- | | |
| | | | | c]pyridin-6- | | |
| | | | | yl](phenyl)methanol | | |
| 5 | +++ | 8-2, 1-1 | A | [(2S)-2-[2-(2,3-dihydro-1,4- | 452.0 | (400 MHz, Methanol-d4) δ |
| (1^{st} | | | | benzodioxine-6-sulfonyl)- | | 8.40 (d, J = 1.1 Hz, 1H), 7.36- |
| eluting | | | | 1H,2H,3H-pyrrolo[3,4- | | 7.16 (m, 7H), 7.17 (s, 1H), |
| isomer) | | | | c]pyridin-6-yl]-2- | | 6.99-6.92 (m, 1H), 4.60 (d, |
| 6 | | | | phenylethyl](methyl)amine; | | J = 2.0 Hz, 2H), 4.56-4.47 |
| (2^{nd} | +++ | | | [(2R)-2-[2-(2,3-dihydro-1,4- | | (m, 2H), 4.35 (dd, J = 8.8, |
| eluting | | | | benzodioxine-6-sulfonyl)- | | 6.3 Hz, 1H), 4.26 (tt, J = 5.0, |
| isomer) | | | | 1H,2H,3H-pyrrolo[3,4- | | 2.6 Hz, 4H), 3.51 (dd, J = |
| | | | | c]pyridin-6-yl]-2- | | 12.2, 8.8 Hz, 1H), 3.17 (dd, |
| | | | | phenylethyl](methyl)amine | | J = 12.1, 6.3 Hz, 1H), 2.44 |
| | | | | | | (s, 3H). |
| 7 | ++++ | 3-1, 4-1, | C | [2-(2,3-dihydro-1,4- | 509.0 | (Methanol-d4, 400 MHz) δ |
| | | 10-1 | | benzodioxine-6-sulfonyl)- | | (ppm): 8.30 (s, 1H), 7.49 (s, |
| | | | | 1H,2H,3H-pyrrolo[3,4- | | 1H), 7.40-7.27 (m, 2H), |
| | | | | c]pyridin-6-yl][3-(piperazin-1- | | 7.21-7.10 (m, 1H), 7.08-7.01 |
| | | | | yl)phenyl]methanol | | (m, 1H), 6.99-6.94 (m, 1H), |
| | | | | | | 6.88-6.79 (m, 2H), 5.71 (s, |
| | | | | | | 1H), 4.59 (s, 4H), 4.36-4.20 |
| | | | | | | (m, 4H), 3.16-3.07 (m, 4H), |
| | | | | | | 3.01-2.91 (m, 4H). |
| 8 | +++ | 10-1, 8-9 | C | (3-chlorophenyl)[2-(2,3- | 459.0 | (DMSO-d6, 400 MHz) δ |
| | | | | dihydro-1,4-benzodioxine-6- | | (ppm): 8.33 (s, 1H), 7.48 (s, |
| | | | | sulfonyl)-1H,2H,3H- | | 1H), 7.46-7.37 (m, 1H), |
| | | | | pyrrolo[3,4-c]pyridin-6- | | 7.36-7.17 (m, 5H), 7.01 (d, |
| | | | | yl]methanol | | J = 8.4 Hz, 1H), 6.31-6.18 (m, |
| | | | | | | 1H), 5.75-5.62 (m, 1H), |
| | | | | | | 4.62-4.45 (m, 4H), 4.34-4.16 |
| | | | | | | (m, 4H). |
| 9 | +++ | 6-1, 9-2, | D | {3-[2-(2,3-dihydro-1,4- | 466.0 | (Methanol-d4, 400 MHz) δ |
| | | 8-7 | | benzodioxine-6-sulfonyl)- | | (ppm): 8.35 (s, 1H), 7.38- |
| | | | | 1H,2H,3H-pyrrolo[3,4- | | 7.22 (m, 7H), 7.22-7.13 (m, |
| | | | | c]pyridin-6-yl]-3- | | 1H), 7.01-6.94 (m, 1H), 4.57 |
| | | | | phenylpropyl}(methyl)amine | | (s, 2H), 4.52 (s, 2H), 4.32- |
| | | | | | | 4.22 (m, 4H), 4.13 (t, J = 7.6 |
| | | | | | | Hz, 1H), 2.53-2.43 (m, 2H), |
| | | | | | | 2.47-2.33 (m, 1H), 2.33 (s, |
| | | | | | | 3H), 2.28-2.21 (m, 1H). |
| 10 | +++ | 13-1 | G | [2-(2,3-dihydro-1,4- | 424.0 | (DMSO-d6, 300 MHz) δ |
| | | | | benzodioxine-6-sulfonyl)- | | (ppm): 8.34 (s, 1H), 7.41 (s, |
| | | | | 1H,2H,3H-pyrrolo[3,4- | | 1H), 7.35-7.21 (m, 6H), |
| | | | | c]pyridin-6- | | 7.17-7.12 (m, 1H), 7.04-7.01 |
| | | | | yl](phenyl)methanamine | | (m, 1H), 5.08 (s, 1H), 4.53 |
| | | | | | | (s, 4H), 4.29-4.25 (m, 4H), |
| | | | | | | 2.41 (s, 2H). |
| 11 | +++ | 6-1 | E | {[2-(2,3-dihydro-1,4- | 438.0 | (Methanol-d4, 400 MHz) δ |
| | | | | benzodioxine-6-sulfonyl)- | | (ppm): 8.37 (s, 1H), 7.40- |
| | | | | 1H,2H,3H-pyrrolo[3,4- | | 7.18 (m, 8H), 7.00-6.93 (m, |
| | | | | c]pyridin-6- | | 1H), 4.79 (s, 1H), 4.62-4.53 |
| | | | | yl](phenyl)methyl}(methyl)amine | | (m, 4H), 4.31-4.21 (m, 4H), |
| | | | | | | 2.31 (s, 3H). |
| 12 | +++ | 1-1, 8- | A | (2-{2-[3-fluoro-4-(1H-pyrazol- | 478.0 | (CD₃OD, 400 MHz) δ |
| | | 3, 7-2 | | 1-yl)benzenesulfonyl]- | | (ppm): 8.45 (s, 1H), 8.20 (m, |
| | | | | 1H,2H,3H-pyrrolo[3,4- | | 1H), 8.19-8.06 (m, 1H), |
| | | | | c]pyridin-6-yl}-2- | | 7.92-7.89 (m, 1H), 7.86-7.81 |
| | | | | phenylethyl)(methyl)amine | | (m, 2H), 7.32-7.16 (m, 6H), |
| | | | | | | 6.62-6.60 (m, 1H), 4.74 (s, |
| | | | | | | 2H), 4.65 (s, 2H), 4.39-4.35 |
| | | | | | | (m, 1H), 3.54-3.49 (m, 1H), |
| | | | | | | 3.20-3.15 (m, 1H), 2.42 (s, |
| | | | | | | 3H). |
| 13 | +++ | 6-1, 7- | D | (3-{2-[3-fluoro-4-(1H-pyrazol- | 492.0 | (Methanol-d4, 400 MHz) δ |
| | | 2, 8-6, | | 1-yl)benzenesulfonyl]- | | (ppm): 8.40 (s, 1H), 8.20 (s, |
| | | 9-1 | | 1H,2H,3H-pyrrolo[3,4- | | 1H), 8.11-8.07 (m, 1H), |
| | | | | c]pyridin-6-yl}-3- | | 7.92-7.89 (m, 1H), 7.85-7.81 |
| | | | | phenylpropyl)(methyl)amine | | (m, 2H), 7.33-7.22 (m, 5H), |
| | | | | | | 7.18-7.14 (m, 1H), 6.61 (s, |
| | | | | | | 1H), 4.72-4.68 (d, J = 16.0 |
| | | | | | | Hz, 4H), 4.16-4.12 (m, 1H), |
| | | | | | | 2.53-2.49 (m, 2H), 2.45-2.31 |
| | | | | | | (m, 4H), 2.29-2.22 (m, 1H). |
| 14 | ++++ | 7-2, 8- | B | (R)-{2-[3-fluoro-4-(1H- | 535.0 | (DMSO-d6, 400 MHz) δ |
| (1^{st} | | 4, 4-13 | | pyrazol-1-yl)benzenesulfonyl]- | | (ppm): 8.33-8.31 (m, 1H), |
| eluting | | | | 1H,2H,3H-pyrrolo[3,4- | | 8.29-8.28 (m, 1H), 8.10-8.08 |
| isomer) | | | | c]pyridin-6-yl}[3-(piperazin-1- | | (m, 1H), 8.06-8.00 (m, 1H), |
| 15 | ++++ | | | yl)phenyl]methanol; | | 7.87-7.83 (m, 2H), 7.47 (s, |
| (2^{nd} | | | | (S)-{2-[3-fluoro-4-(1H- | | 1H), 7.06-7.04 (m, 1H), |
| eluting | | | | pyrazol-1-yl)benzenesulfonyl]- | | 6.96-6.93 (m, 1H), 6.72-6.70 |
| isomer) | | | | 1H,2H,3H-pyrrolo[3,4- | | (m, 2H), 6.63-6.62 (m, 1H), |
| | | | | c]pyridin-6-yl}[3-(piperazin-1 - | | 6.01-5.99 (m, 1H), 5.61-5.99 |
| | | | | yl)phenyl]methanol | | (m, 1H), 4.72-4.64 (m, 4H), |
| | | | | | | 2.99-2.94 (m, 4H), 2.97-2.82 |
| | | | | | | (m, 4H), 2.33-2.18 (m, 1H). |
| 16 | +++ | 6-1, 8- | C | {2-[4-(1,3-oxazol-2- | | |
| | | 5, 14-1 | | yl)benzenesulfonyl]- | | |
| | | | | 1H,2H,3H-pyrrolo[3,4- | | |
| | | | | c]pyridin-6- | | |
| | | | | yl}(phenyl)methanol | | |
| 17 | +++ | 11-1, 6-1 | F | {2-[4-(5-fluoro-1H-pyrazol-1 - | 451.0 | (DMSO-d6, 400 MHz) δ |
| | | | | yl)benzenesulfonyl]- | | (ppm): 8.33 (s, 1H), 8.07- |
| | | | | 1H,2H,3H-pyrrolo[3,4- | | 7.98 (m, 2H), 7.88-7.85 (m, |
| | | | | c]pyridin-6- | | 2H), 7.74-7.73 (m, 1H), 7.49 |
| | | | | yl}(phenyl)methanol | | (s, 1H), 7.31 (d, J = 6.8 Hz, |
| | | | | | | 2H), 7.24-7.21 (m, 2H), |
| | | | | | | 7.17-7.14 (m, 1H). 6.30-6.29 |
| | | | | | | (m, 1H), 6.08-6.06 (m, 1H), |
| | | | | | | 5.64 (d, J = 4.0 Hz, 1H), |
| | | | | | | 4.69-4.57 (m, 4H). |
| 18 | + | 6-1, 5-1 | C | {2-[3-methoxy-4-(1H-pyrazol- | 463.0 | (DMSO-d6, 400 MHz) δ |
| | | | | 1-yl)benzenesulfonyl]- | | (ppm): 8.34 (s, 1H), 8.30 (d, |
| | | | | 1H,2H,3H-pyrrolo[3,4- | | J = 2.4 Hz, 1H), 7.93-7.77 |
| | | | | c]pyridin-6- | | (m, 1H), 7.76 (s, 1H), 7.59- |
| | | | | yl}(phenyl)methanol | | 7.56 (m, 1H), 7.53-7.50 (m, |
| | | | | | | 2H), 7.33 (d, J = 7.2 Hz, |
| | | | | | | 2H), 7.25-7.21 (m, 2H), |
| | | | | | | 7.18-7.14 (m, 1H), 6.53-6.52 |
| | | | | | | (m, 1H), 6.09-6.07 (m, 1H), |
| | | | | | | 5.69-5.67 (m, 1H),4.73-4.61 |
| | | | | | | (m, 4H), 3.97 (s, 3H). |
| 19 | −− | 6-1, 7-1 | C | {2-[4-(1H-imidazol-1- | 433.0 | (DMSO-d6, 400 MHz) δ |
| | | | | yl)benzenesulfonyl]- | | (ppm): 8.38 (s, 1H), 8.34 (s, |
| | | | | 1H,2H,3H-pyrrolo{3,4- | | 1H), 7.99-7.91 (m, 2H), |
| | | | | c]pyridin-6- | | 7.88-7.84 (m, 3H), 7.49 (s, |
| | | | | yl}(phenyl)methanol | | 1H), 7.33-7.31 (m, 2H), |
| | | | | | | 7.25-7.21 (m, 2H), 7.18-7.13 |
| | | | | | | (m, 2H), 6.08 (d, J = 4.4 Hz, |
| | | | | | | 1H), 5.67 (d, J = 4.0 Hz, |
| | | | | | | 1H), 4.66-4.58 (m, 4H). |
| 20 | −− | 6-1, 17- | C | (S)-phenyl(2-{[(1r,4r)-4-(1H- | 439.0 | 1^{st}:(DMSO-d6, 400 MHz) δ |
| (1^{st} | | 1, 8-13 | | pyrazol-1- | | (ppm): 8.42 (s, 1H), 7.79 (s, |
| eluting | | | | yl)cyclohexyl]sulfonyl]- | | 1H), 7.57 (s, 1H), 7.45 (s, |
| isomer) | | | | 1H,2H,3H-pyrrolo{3,4- | | 1H), 7.40-7.38 (m, 2H), |
| 21 | −− | | | c]pyridin-6-yl)methanol; | | 7.31-7.27 (m, 2H),7.22-7.18 |
| (2^{nd} | | | | (S)-phenyl(2-{[(1s,4s)-4-(1H- | | (m, 1H), 6.26 (s, 1H), 6.13 |
| eluting | | | | pyrazol-1- | | (s, 1H), 5.74 (s, 1H), 4.70- |
| isomer) | | | | yl)cyclohexyl]sulfonyl}- | | 4.65 (m, 4H), 4.32 (s, 1H), |
| 22 | ++ | | | 1H,2H,3H-pyrrolo{3,4- | | 3.56-3.53 (m, 1H), 2.42-2.33 |
| (3^{rd} | | | | c]pyridin-6-yl)methanol; | | (m, 2H), 1.85-1.84 (m, 6H). |
| eluting | | | | (R)-phenyl(2-{[(1r,4r)-4-(1H- | | 2^{nd}: (DMSO-d6, 400 MHz) δ |
| isomer) | | | | pyrazol-1- | | (ppm): 8.43 (s, 1H), 7.78 (s, |
| 23 | −− | | | yl)cyclohexyl]sulfonyl}- | | 1H), 7.68 (s, 1H), 7.58-7.41 |
| (4^{th} | | | | 1H,2H,3H-pyrrolo[3,4- | | (m, 3H), 7.30-7.27 (m, 2H), |
| eluting | | | | c]pyridin-6-yl)methanol; | | 7.22-7.18 (m, 1H), 6.26-6.21 |
| isomer) | | | | (R)-phenyl(2-{[(1s,4s)-4-(1H- | | (m, 1H), 6.14-6.13 (m, 1H), |
| | | | | pyrazol-1- | | 5.75 (s, 1H), 4.74 (s, 4H), |
| | | | | yl)cyclohexyl]sulfonyl}- | | 4.23-4.17 (m, 1H), 3.49-3.44 |
| | | | | 1H,2H,3H-pyrrolo{3,4- | | (m, 1H), 2.15-1.99 (m, 4H), |
| | | | | c]pyridin-6-yl)methanol | | 1.84-1.67 (m, 4H). |
| | | | | | | 3^{rd}: (DMSO-d6, 400 MHz) δ |
| | | | | | | (ppm): 8.42 (s, 1H), 7.78 (s, |
| | | | | | | 1H), 7.57 (s, 1H), 7.45-7.38 |
| | | | | | | (m, 3H), 7.31-7.27 (m, 2H), |
| | | | | | | 7.22-7.18 (m, 1H), 6.26 (s, |
| | | | | | | 1H), 6.13-6.12 (m, 1H), |
| | | | | | | 5.74-5.73 (m, 1H), 4.70 (s, |
| | | | | | | 4H), 4.32 (s, 1H), 3.55 (s, |
| | | | | | | 1H), 2.39-2.33 (m, 2H), |
| | | | | | | 1.85-1.84 (m, 6H). |
| | | | | | | 4^{th}: (DMSO-d6, 400 MHz) δ |
| | | | | | | (ppm): 8.43 (s, 1H), 7.68 (s, |
| | | | | | | 1H), 7.58 (s, 1H), 7.47-7.39 |
| | | | | | | (m, 3H), 7.30-7.27 (m, 2H), |
| | | | | | | 7.22-7.18 (m, 1H), 6.21 (s, |
| | | | | | | 1H), 6.14-6.13 (m, 1H), |
| | | | | | | 5.75-5.74 (m, 1H), 4.74 (s, |
| | | | | | | 4H), 4.23-4.17 (m, 1H), |
| | | | | | | 3.50-3.44 (m, 1H), 2.15-2.00 |
| | | | | | | (m, 4H), 1.84-1.67 (m, 4H). |
| 24 | −− | 8-12, | J | phenyl(2-{[1-(1,3-thiazol-2- | 457.3 | |
| | | 16-1 | | yl)piperidin-4-yl]sulfonyl}- | | |
| | | | | 1H,2H,3H-pyrrolo[3,4- | | |
| | | | | c]pyridin-6-yl)methanol | | |
| 25 | ++++ | 4-13, 8- | B | {2-[4-(1,3-oxazol-2- | 518.0 | (DMSO-d6, 300 MHz) δ |
| | | 11 | | yl)benzenesulfonyl}- | | (ppm): 8.36 (s, 1H), 8.32 (s, |
| | | | | 1H,2H,3H-pyrrolo[3,4- | | 1H), 8.26 (s, 1H), 8.16 (d, J = |
| | | | | c]pyridin-6-yl}[3-(piperazin-1- | | 8.4 Hz, 2H), 8.07 (d, J = |
| | | | | yl)phenyl]methanol | | 8.4 Hz, 2H), 7.46 (d, J = 14.0 |
| | | | | | | Hz, 2H), 7.10-7.06 (m, |
| | | | | | | 1H), 6.99 (s, 1H), 6.77- |
| | | | | | | 6.72 (m, 2H), 5.60 (s, 1H), |
| | | | | | | 4.68-4.58 (m, 4H), 3.19 (s, |
| | | | | | | 4H), 3.07 (m, 4H). |
| 26 | ++++ | 8-11, 3-3 | D | (R)-{2-[4-(1,3-oxazol-2- | 518.0 | (DMSO-d6 400 MHz) δ |
| (1^{st} | | | | yl)benzenesulfonyl}- | | (ppm): 8.32 (d, J = 4.4 Hz, |
| eluting | | | | 1H,2H,3H-pyrrolo[3,4- | | 2H), 8.16 (d, J = 8.4 Hz, |
| isomer) | | | | c]pyridin-6-yl}[3-(piperazin-1- | | 2H), 8.00 (d, J = 8.4 Hz, |
| 27 | ++++ | | | yl)phenyl]methanol; | | 2H), 7.47 (s, 2H), 7.05-7.01 |
| (2^{nd} | | | | (S)-{2-[4-(1,3-oxazol-2- | | (m, 1H), 6.95 (s, 1H), 6.71- |
| eluting | | | | yl)benzenesulfonyl}- | | 6.66 (m, 2H), 5.97 (d, J = 3.2 |
| isomer) | | | | 1H,2H,3H-pyrrolo[3,4- | | Hz, 1H), 5.59 (s. 1H), 4.69- |
| | | | | c]pyridin-6-yl}[3-(piperazin-1- | | 4.58 (m, 4H), 2.98-2.96 (m, |
| | | | | yl)phenyl]methanol | | 4H), 2.85-2.78 (m, 4H). |
| 28 | ++++ | 8-11, 4-13 | J | [3-(4-methylpiperazin-1- | 532.0 | (DMSO-d6, 400 MHz) δ |
| | | | | yl)phenyl]({2-[4-(1,3-oxazol- | | (ppm): 8.32 (d, J = 4.4 Hz, |
| | | | | 2-yl)benzenesulfonyl}- | | 2H), 8.16 (d, J = 8.0 Hz, |
| | | | | 1H,2H,3H-pyrrolo[3,4- | | 2H), 8.01 (d, J = 8.4 Hz, |
| | | | | c]pyridin-6-yl})methanol | | 2H), 7.47 (s, 2H), 7.06-7.02 |
| | | | | | | (m, 1H), 6.96 (s, 1H), |
| | | | | | | 6.73-6.66 (m, 2H), 5.97 (d, J = |
| | | | | | | 4.0 Hz, 1H), 5.59 (d, J = |
| | | | | | | 4.0 Hz, 1H), 4.65-4.59 (m, |
| | | | | | | 4H), 3.07-3.05 (m, 4H), 2.47- |
| | | | | | | 2.33 (m, 4H), 2.20 (s, 3H). |
| 29 | ++++ | 4-13, 8-11 | J | (R)-[3-(4-methylpiperazin-1- | 532.0 | (DMSO-d6, 400 MHz) δ |
| (1^{st} | | | | yl)phenyl]({2-[4-(1,3-oxazol- | | (ppm): 8.32 (d, J = 4.4 Hz, |
| eluting | | | | 2-yl)benzenesulfonyl}- | | 2H), 8.16 (d, J = 8.0 Hz, |
| isomer) | | | | 1H,2H,3H-pyrrolo[3,4- | | 2H), 8.01 (d, J = 8.4 Hz, |
| 30 | ++++ | | | c]pyridin-6-yl})methanol; | | 2H), 7.47 (s, 2H), 7.06-7.02 |
| (2^{nd} | | | | (S)[3-(4-methylpiperazin-1- | | (m, 1H), 6.96 (s, 1H), 6.73- |
| eluting | | | | yl)phenyl]({2-[4-(1,3-oxazol- | | 6.66 (m, 2H), 5.97 (d, J = 4.0 |
| isomer) | | | | 2-yl)benzenesulfonyl}- | | Hz, 1H), 5.59 (d, J = 4.0 Hz, |
| | | | | 1H,2H,3H-pyrrolo[3,4- | | 1H), 4.65-4.59 (m, 4H), |
| | | | | c]pyridin-6-yl})methanol | | 3.07-3.05 (m, 4H), 2.47-2.33 |
| | | | | | | (m, 4H), 2.20 (s, 3H). |
| 31 | ++++ | 2-1, 8- | | {2-[3-fluoro-4-(1,3-oxazol-2- | 536.0 | (DMSO-d6, 400 MHz) δ |
| | | 14, 4-13 | | yl)benzenesulfonyl]- | | (ppm) 8.35 (d, J = 16.8 Hz, |
| | | | | 1H,2H,3H-pyrrolo[3,4- | | 2H), 8.24-8.22 (m, 1H), |
| | | | | c]pyridin-6-yl}[3-(piperazin-1 - | | 7.90-7.83 (m, 2H), 7.52 (s, |
| | | | | yl)phenyl]methanol | | 1H), 7.47 (s, 1H), 7.06-7.02 |
| | | | | | | (m, 1H), 6.95 (s, 1H), 6.76- |
| | | | | | | 6.67 (m, 2H), 5.98 (d, J = 4.0 |
| | | | | | | Hz, 1H), 5.60 (d, J = 4.0 Hz, |
| | | | | | | 1H), 4.73-4.62 (m, 4H), |
| | | | | | | 2.98-2.81 (m, 4H), 2.80-2.70 |
| | | | | | | (m, 4H), 2.57-2.54 (m, 1H). |
| 32 | −− | 8-8, 4-2 | H | (2-{[1-(1,3-oxazol-2- | 441.0 | (Methanol-d4, 400 MHz) δ |
| | | | | yl)piperidin-4-yl]sulfonyl}- | | (ppm): 8.43 (s, 1H), 7.63 (s, |
| | | | | 1H,2H,3H-pyrrolo[3,4- | | 1H), 7.43-7.41 (m, 3H), |
| | | | | c]pyridin-6- | | 7.33-7.30 (m, 2H), 7.26-7.23 |
| | | | | yl)(phenyl)methanol | | (m, 1H), 6.84 (s, 1H), 5.85 |
| | | | | | | (s, 1H), 4.92-4.83 (s, 4H), |
| | | | | | | 4.15-4.11 (m, 2H), 3.59-3.52 |
| | | | | | | (m, 1H), 3.08-3.01 (m, 2H), |
| | | | | | | 2.16-2.12 (m, 2H), 1.91-1.85 |
| | | | | | | (m, 2H). |
| 33 | ++++ | 18-1, 2-2 | F | {7-methyl-2-[4-(1,3-oxazol-2- | 448.0 | (DMSO-d6, 400 MHz,) δ |
| | | | | yl)benzenesulfonyl]- | | (ppm): 8.33-8.29 (m, 2H), |
| | | | | 1H,2H,3H-pyrrolo[3,4- | | 8.17 (d, J = 8.4 Hz, 2H), |
| | | | | c]pyridin-6- | | 8.04-8.02 (m, 2H), 7.48 (s, |
| | | | | yl}(phenyl)methanol | | 1H), 7.22-7.17 (m, 5H), |
| | | | | | | 5.90-5.86 (m, 2H), 4.69-4.63 |
| | | | | | | m, 4H), 2.06 (s, 3H). |
| 34 | +++ | 19-1, 2-2 | F | {6-[4-(1,3-oxazol-2- | 435.0 | (400 MHz, DMSO-d6) δ |
| | | | | yl)benzenesulfonyl]- | | (ppm): 8.32 (s, 1H), 8.14 (d, |
| | | | | 5H,6H,7H-pyrrolo[3,4- | | J = 8.6 Hz, 2H), 8.03 (d, J = |
| | | | | c]pyridazin-3- | | 8.3 Hz, 2H), 7.69 (s, 1H), |
| | | | | yl}(phenyl)methanol | | 7.47 (s, 1H), 7.34 (d, J = 7.3 |
| | | | | | | Hz, 2H), 7.26 (t, J = 7.6 Hz, |
| | | | | | | 2H), 7.20 (d, J = 6.6 Hz, |
| | | | | | | 1H), 6.39 (d, J = 4.2 Hz, |
| | | | | | | 1H), 5.96 (d, J = 4.0 Hz, |
| | | | | | | 1H), 4.85-4.69 (m, 4H). |
| 35 | ++++ | 21-1, 2-2, | A | 2,3-dihydro-1H-isoindol-4- | 475.0 | (DMSO-d6, 400 MHz) δ |
| | | 8-15 | | yl({2-[4-(1,3-oxazol-2- | | (ppm): 8.32 (d, J = 3.2 Hz, |
| | | | | yl)benzenesulfonyl]- | | 2H), 8.21-8.15 (m, 2H), |
| | | | | 1H,2H,3H-pyrrolo[3,4- | | 8.04-7.97 (m, 2H), 7.48 (d, |
| | | | | c]pyridin-6-yl})methanol | | J = 7.6 Hz, 2H), 7.26-7.03 (m, |
| | | | | | | 3H), 6.14-6.02 (m, 1H), |
| | | | | | | 5.68-5.65 (m, 1H), 4.71-4.56 |
| | | | | | | (m, 5H), 4.15-3.95 (m, 3H). |
| 36 | ++++ | 2-1, 20-1 | F | {6-[3-fluoro-4-(1,3-oxazol-2- | 453.0 | (DMSO-d6, 400 MHz) δ |
| | | | | yl)benzenesulfonyl] - | | (ppm): 8.65 (s, 1H), 8.39 (s, |
| | | | | 5H,6H,7H-pyrrolo[3,4- | | 1H), 8.21 (t, J = 8.0 Hz, 1H), |
| | | | | d]pyrimidin-2- | | 7.96-7.93 (m, 1H), 7.88-7.85 |
| | | | | yl}(phenyl)methanol | | (s, 1H), 7.53 (s, 1H), 7.44- |
| | | | | | | 7.36 (m, 2H), 7.26-7.22 (m, |
| | | | | | | 2H), 7.20-7.15 (m, 1H), |
| | | | | | | 5.98-5.95 (m, 1H), 5.74-5.72 |
| | | | | | | (m, 1H), 4.71-4.66 (m, 4H). |
| 37 | ++++ | | | 1-[3-(1-{2-[3-fluoro-4-(1,3- | 534.0 | (DMSO-d6, 400 MHz) δ |
| | | | | oxazol-2-yl)benzenesulfonyl]- | | (ppm): 8.39 (s, 2H), 8.23- |
| | | | | 1H,2H,3H-pyrrolo[3,4- | | 8.19 (m, 1H), 7.90-7.87 (m, |
| | | | | c]pyridin-6- | | 1H), 7.84-7.81 (m, 1H), 7.53 |
| | | | | yl}ethyl)phenyl]piperazine | | (s, 1H), 7.17 (s, 1H), 7.05- |
| | | | | | | 7.01 (m, 1H), 6.84 (s, 1H), |
| | | | | | | 6.69-6.66 (m, 1H), 6.62-6.59 |
| | | | | | | (m, 1H), 4.65-4.63 (m, 4H), |
| | | | | | | 4.15-4.14 (m, 1H), 2.97 (s, |
| | | | | | | 4H), 2.79 (s, 4H), 1.51 (d, |
| | | | | | | J = 7.2 Hz, 3H). |
| 38 | ++++ | 20-2, 2-1 | F | (S)-{6-[3-fluoro-4-(1,3-oxazol- | 483.0 | (CDCl₃, 400 MHz) δ (ppm): |
| (1^{st} | | | | 2-yl)benzenesulfonyl]- | | 8.63 (br s, 1H), 8.30-8.21 |
| eluting | | | | 5H,6H,7H-pyrrolo[3,4- | | (m, 1H), 7.86 (s, 1H), 7.79- |
| isomer) | | | | d]pyrimidin-2-yl}(3- | | 7.75 (m, 2H), 7.39 (s, 1H), |
| 39 | ++++ | | | methoxyphenyl)methanol; | | 7.29-7.22 (m, 1H), 7.04-7.00 |
| (2^{nd} | | | | (R)-{6-[3-fluoro-4-(1,3-oxazol- | | (m, 2H), 6.81-6.80 (m, 1H), |
| eluting | | | | 2-yl)benzenesulfonyl]- | | 5.96-5.86 (m, 1H), 4.83-4.67 |
| isomer) | | | | 5H,6H,7H-pyrrolo[3,4- | | (m, 4H), 3.79 (s, 3H). |
| | | | | d]pyrimidin-2-yl}(3- | | |
| | | | | methoxyphenyl)methanol | | |
| 40 | ++++ | 2-1, 4- | A | (S)-{6-[3-fluoro-4-(1,3-oxazol- | 537.0 | (DMSO-d6, 400 MHz) δ |
| (1^{st} | | 6, 8-19, | | 2-yl)benzenesulfonyl]- | | (ppm): 8.65 (s, 1H), 8.38 (s, |
| eluting | | 30-1 | | 5H,6H,7H-pyrrolo[3,4- | | 1H), 8.24-8.20 (m, 1H), |
| isomer) | | | | d]pyrimidin-2-yl}[3- | | 7.96-7.93 (m, 1H), 7.88-7.86 |
| 41 | ++++ | | | (piperazin-1- | | (m, 1H), 7.53 (s, 1H), 7.10- |
| (2^{nd} | | | | yl)phenyl]methanol | | 7.00 (m, 2H), 6.78-6.69 (m, |
| eluting | | | | (R)-{6-[3-fluoro-4-(1,3-oxazol- | | 2H), 5.87 (s, 1H), 5.66 (s, |
| isomer) | | | | 2-yl)benzenesulfonyl]- | | 1H), 4.69-4.66 (m, 4H), |
| | | | | 5H,6H,7H-pyrrolo[3,4- | | 3.03-3.01 (m, 4H), 2.87-2.85 |
| | | | | d]pyrimidin-2-yl}[3- | | (m, 4H). |
| | | | | (piperazin-1- | | |
| | | | | yl)phenyl]methanol | | |
| 42 | ++++ | 3-2, 4- | C | (S)-{6-[3-fluoro-4-(1,3-oxazol- | 551.0 | (CDCl₃, 400 MHz) δ (ppm): |
| (1^{st} | | 3, 8-16 | | 2-yl)benzenesulfonyl]- | | 8.60 (s, 1H), 8.31-8.27 (m, |
| eluting | | | | 5H,6H,7H-pyrrolo[3,4- | | 1H), 7.85 (s, 1H), 7.79-7.75 |
| isomer) | | | | d]pyrimidin-2-yl}[3-(4- | | (m, 2H), 7.38 (s, 1H), 7.22- |
| 43 | ++++ | | | methylpiperazin-1- | | 7.18 (m, 1H), 7.06-7.02 (m, |
| (2^{nd} | | | | yl)phenyl]methanol; | | 1H), 6.93 (d, J = 7.6 Hz, |
| eluting | | | | (R)-{6-[3-fluoro-4-(1,3-oxazol- | | 1H), 6.83-6.80 (m, 1H), 5.82 |
| isomer) | | | | 2-yl)benzenesulfonyl]- | | (s, 1H), 4.79-4.63 (m, 5H), |
| | | | | 5H,6H,7H-pyrrolo[3,4- | | 3.25-3.22 (m, 4H), 2.62 (br |
| | | | | d]pyrimidin-2-yl}[3-(4- | | s, 4H), 2.39 (s, 3H). |
| | | | | methylpiperazin-1- | | |
| | | | | yl)phenyl]methanol; | | |
| 44 | ++ | 4-9, 31-2 | F | cyclopentyl({6-[4-(1,3-oxazol- | 427.0 | (DMSO-d6, 400 MHz) δ |
| | | | | 2-yl)benzenesulfonyl]- | | (ppm): 8.63 (s, 1H), 8.32 (s, |
| | | | | 5H,6H,7H-pyrrolo[3,4- | | 1H), 8.18-8.11 (m, 2H), |
| | | | | d]pyrimidin-2-yl})methanol | | 8.08-8.01 (m, 2H), 7.47 (s, |
| | | | | | | 1H), 5.13 (d, J = 8.0 Hz, |
| | | | | | | 1H), 4.72 (s, 2H), 4.64 (s, |
| | | | | | | 2H), 4.32-4.29 (m, 1H), |
| | | | | | | 2.30-3.23 (m, 1H), 1.59-1.32 |
| | | | | | | (m, 6H), 1.21-1.12 (m, 1H), |
| | | | | | | 1.09-1.01 (m, 1H). |
| 45 | ++ | 4-7, 31-1 | F | cyclohexyl({6-[4-(1,3-oxazol- | 441.0 | (DMSO-d6, 400 MHz) δ |
| | | | | 2-yl)benzenesulfonyl]- | | (ppm): 8.64 (s, 1H), 8.32 (s, |
| | | | | 5H,6H,7H-pyrrolo[3,4- | | 1H), 8.19-8.12 (m, 2H), |
| | | | | d]pyrimidin-2-yl})methanol | | 8.08-8.02 (m, 2H), 7.47 (s, |
| | | | | | | 1H), 4.99 (d, J = 8.0 Hz, |
| | | | | | | 1H), 4.72 (s, 2H), 4.64-4.63 |
| | | | | | | (m, 2H), 4.26 (t, J = 8.0 Hz, |
| | | | | | | 1H), 175-7.68 (m, 1H), 1.62- |
| | | | | | | 1.60 (m, 2H), 1.53-1.49 (m, |
| | | | | | | 2H), 1.15-1.07 (m, 2H), |
| | | | | | | 1.07-0.99 (m, 3H), 0.96-0.86 |
| | | | | | | (m, 1H). |
| 46 | ++ | 24-4, | K | (S)-{2-[4-(4,5-dihydro-1,3- | 436.0 | (DMSO-d6, 400 MHz) δ |
| (1^{st} | | 32-1 | | oxazol-2-yl)benzenesulfonyl]- | | (ppm): 8.32 (s, 1H), 8.03 (d, |
| eluting | | | | 1H,2H,3H-pyrrolo[3,4- | | J = 8.0 Hz, 2H), 7.95 (d, J = |
| isomer) | | | | c]pyridin-6- | | 8.0 Hz, 2H), 7.49 (s, 1H), |
| 47 | ++ | | | yl}(phenyl)methanol; | | 7.31 (d, J = 8.0 Hz, 2H), 7.25 |
| (2^{nd} | | | | (R)-{2-[4-(4,5-dihydro-1,3- | | (t, J = 8.0 Hz, 2H), 7.19-7.16 |
| eluting | | | | oxazol-2-yl)benzenesulfonyl]- | | (m, 1H), 6.08 (d, J = 4.0 Hz, |
| isomer) | | | | 1H,2H,3H-pyrrolo[3,4- | | 1H), 5.67 (d, J = 4.0 Hz, |
| | | | | c]pyridin-6- | | 1H), 4.71-4.54 (m, 4H), 4.43 |
| | | | | yl}(phenyl)methanol | | (t, J = 8.0 Hz, 2H), 3.98 (t, |
| | | | | | | J = 8.0 Hz, 2H). |
| 48 | ++++ | 24-3, 8-18 | C | (S)-[4-methyl-3-(4- | 546.0 | (DMSO-d6, 400 MHz) δ |
| (1^{st} | | | | methylpiperazin-1- | | (ppm): 8.32 (s, 2H), 8.16 (d, |
| eluting | | | | yl)phenyl]({2-[4-(1,3-oxazol- | | J = 8.4 Hz, 2H), 8.00 (d, J = |
| isomer) | | | | 2-yl)benzenesulfonyl]- | | 8.4 Hz, 2H),7.46 (d, J = 8.4 |
| 49 | ++++ | | | 1H,2H,3H-pyrrolo[3,4- | | Hz, 2H), 7.05 (s, 1H), 6.96 |
| (2^{nd} | | | | c]pyridin-6-yl})methanol; | | (d, J = 7.6 Hz, 1H), 6.79 (d, |
| eluting | | | | (R)-[4-methyl-3-(4- | | J = 7.6 Hz, 1H), 5.99 (d, J = |
| isomer) | | | | methylpiperazin-1- | | 4.0 Hz, 1H), 5.60 (d, J = 4.0 |
| | | | | yl)phenyl]({2-[4-(1,3-oxazol- | | Hz, 1H), 4.68-4.59 (m, 4H), |
| | | | | 2-yl)benzenesulfonyl]- | | 2.76-2.74 (m, 4H), 2.54-2.50 |
| | | | | 1H,2H,3H-pyrrolo[3,4- | | (m, 4H), 2.22 (s, 3H), 2.13 |
| | | | | c]pyridin-6-yl})methanol | | (s, 3H). |
| 50 | ++++ | 27-1, | F | (S)-{3-[(3S)-3,4- | 547.0 | 1^{st}: (CDCl₃, 400 MHz) δ |
| (1^{st} | | 25-3, 3- | | dimethylpiperazin-1- | | (ppm): 8.57 (s, 1H), 8.28- |
| eluting | | 5,4-11, | | yl]phenyl]}({6-[4-(1,3-oxazol- | | 8.23 (m, 2H), 8.00 (d, J = 8.0 |
| isomer) | | 33-1 | | 2-yl)benzenesulfonyl]- | | Hz, 2H), 7.80 (s, 1H), 7.32 |
| 51 | ++++ | | | 5H,6H,7H-pyrrolo[3,4- | | (s, 1H), 7.29-7.18 (m, 1H), |
| (2^{nd} | | | | d]pyrimidin-2-yl})methanol; | | 7.02 (s, 1H), 6.93 (s, 1H), |
| eluting | | | | (R)-{3-[(3S)-3,4- | | 6.80 (d, J = 8.4 Hz, 1H), 5.81 |
| isomer) | | | | dimethylpiperazin-1- | | (s, 1H), 4.77-4.62 (m, 4H), |
| | | | | yl]phenyl}({6-[4-(1,3-oxazol- | | 4.53 (s, 1H), 3.56-3.47 (m, |
| | | | | 2-yl)benzenesulfonyl]- | | 2H), 3.02 (s, 2H), 2.42 (s, |
| | | | | 5H,6H,7H-pyrrolo[3,4- | | 6H), 1.21 (s, 3H). |
| | | | | d]pyrimidin-2-yl})methanol; | | 2^{nd}: (CDCl₃, 400 MHz) δ |
| | | | | (S)-{3-[(3R)-3,4- | | (ppm): 8.58 (s, 1H), 8.25- |
| | | | | dimethylpiperazin-1- | | 8.23 (m, 2H), 8.00 (d, J = 8.0 |
| | | | | yl]phenyl}({6-[4-(1,3-oxazol- | | Hz, 2H), 7.80 (s, 1H), 7.32 |
| | | | | 2-yl)benzenesulfonyl]- | | (s, 1H), 7.28-7.19 (m, 1H), |
| | | | | 5H,6H,7H-pyrrolo[3,4- | | 7.04 (s, 1H), 6.96 (s, 1H), |
| | | | | d]pyrimidin-2-yl})methanol; | | 6.80 (d, J = 8.4 Hz, 1H), 5.82 |
| | | | | (R)-{3-[(3R)-3,4- | | (s, 1H), 4.73-4.62 (m, 4H), |
| | | | | dimethylpiperazin-1- | | 4.55 (s, 1H), 3.55-3.52 (m, |
| | | | | yl]phenyl}({6-[4-(1,3-oxazol- | | 2H), 3.02 (s, 2H), 2.42 (s, |
| | | | | 2-yl)benzenesulfonyl]- | | 6H), 1.21 (s, 3H). |
| | | | | 5H,6H,7H-pyrrolo[3,4- | | |
| | | | | d]pyrimidin-2-yl})methanol | | |
| 52 | ++++ | 27-1, 3- | C | (S)-{3-[(8aS)- | 559.0 | 1^{st}: (CDCl₃, 400 MHz) δ |
| (1^{st} | | 6, 4-12, | | octahydropyrrolo[1,2- | | (ppm): 8.58 (s, 1H), 8.24 (d, |
| eluting | | 8-20, | | a]piperazin-2-yl]phenyl}({6- | | J = 8.4 Hz, 2H), 8.00 (d, J = |
| isomer) | | 33-1 | | [4-(1,3-oxazol-2- | | 8.4 Hz, 2H), 7.80 (s, 1H), |
| 53 | ++++ | | | yl)benzenesulfonyl]- | | 7.32 (s, 1H), 7.22-7.18 (m, |
| (2^{nd} | | | | 5H,6H,7H-pyrrolo[3,4- | | 1H), 7.07 (s, 1H), 6.93 (s, |
| eluting | | | | d]pyrimidin-2-yl})methanol; | | 1H), 6.83-6.81 (m, 1H), 5.81 |
| isomer) | | | | (R)-{3-[(8aS)- | | (s, 1H), 4.77-4.71 (m, 2H), |
| | | | | octahydropyrrolo[1,2- | | 4.67-4.62 (m, 2H), 4.51 (s, |
| | | | | a]piperazin-2-yl]phenyl}({6- | | 1H), 3.75 (s, 1H), 3.60 (d, |
| | | | | [4-(1,3-oxazol-2- | | J = 12.4 Hz, 1H), 3.17 (s, 2H), |
| | | | | yl)benzenesulfonyl]- | | 3.10-2.85 (m, 1H), 1.96-1.92 |
| | | | | 5H,6H,7H-pyrrolo[3,4- | | (m, 3H), 1.59 (s, 4H), 1.218 |
| | | | | d]pyrimidin-2-yl})methanol; | | (s, 1H). |
| | | | | (S)-{3-[(8aR)- | | 2^{nd}: (CDCl₃, 400 MHz) δ |
| | | | | octahydropyrrolo[1,2- | | (ppm): 8.57 (s, 1H), 8.24 (d, |
| | | | | a]piperazin-2-yl]phenyl}({6- | | J = 8.4 Hz, 2H), 8.00 (d, J = |
| | | | | [4-(1,3-oxazol-2- | | 8.4 Hz, 2H), 7.80 (s, 1H), |
| | | | | yl)benzenesulfonyl]- | | 7.32 (s, 1H), 7.22-7.18 (m, |
| | | | | 5H,6H,7H-pyrrolo[3,4- | | 1H), 7.07 (s, 1H), 6.92 (d, |
| | | | | d]pyrimidin-2-yl})methanol; | | J = 7.2 Hz, 1H), 6.83-6.81 (m, |
| | | | | (R)-{3-[(8aR)- | | 1H), 5.81 (s, 1H), 4.77-4.73 |
| | | | | octahydropyrrolo[1,2- | | (m, 2H), 4.67-4.62 (m, 2H), |
| | | | | a]piperazin-2-yl]phenyl}({6- | | 4.51 (s, 1H), 3.76-3.71 (m, |
| | | | | [4-(1,3-oxazol-2- | | 1H), 3.63 (d, J = 12.0 Hz, |
| | | | | yl)benzenesulfonyl]- | | 1H), 3.18 (s, 2H), 3.03-2.90 |
| | | | | 5H,6H,7H-pyrrolo[3,4- | | (m, 1H), 2.76-2.25 (m, 2H), |
| | | | | d]pyrimidin-2-yl})methanol | | 1.96-1.88 (m, 3H), 1.84-1.63 |
| | | | | | | (m, 3H). |
| 54 | ++++ | | | 2-[3-(4-methylpiperazin-1- | 541.0 | (DMSO-d6, 400 MHz) δ |
| | | | | yl)phenyl]-2-{2-[4-(1,3- | | (ppm): 8.47 (s, 1H), 8.32 (s, |
| | | | | oxazol-2-yl)benzenesulfonyl]- | | 1H), 8.15 (d, J = 8.4 Hz, |
| | | | | 1H,2H,3H-pyrrolo[3,4- | | 2H), 8.00 (d, J = 8.4 Hz, |
| | | | | c]pyridin-6-yl}acetonitrile | | 2H), 7.47 (s, 1H), 7.35 (s, |
| | | | | | | 1H), 7.17-7.15 (m, 1H), 6.97 |
| | | | | | | (s, 1H), 6.85-6.83 (m, 1H), |
| | | | | | | 6.72-6.70 (m, 1H), 5.75 (s, |
| | | | | | | 1H), 4.66 (s, 4H), 3.10-3.08 |
| | | | | | | (m, 4H), 2.43-2.40 (m, 4H), |
| | | | | | | 2.21 (s, 3H). |
| 55 | +++ | | | (2S)-2-(3-methoxyphenyl)-N- | 506.0 | (DMSO-d6, 400 MHz) δ |
| (1^{st} | | | | methyl-2-{6-[4-(1,3-oxazol-2- | | (ppm): 8.62 (s, J = 0.8 Hz, |
| eluting | | | | yl)benzenesulfonyl]- | | 1H), 8.33 (s, J = 1.2 Hz, 1H), |
| isomer) | | | | 5H,6H,7H-pyrrolo[3,4- | | 8.17-8.15 (m, 2H), 8.06-8.04 |
| 56 | +++ | | | d]pyrimidin-2-yl}acetamide; | | (m, 2H), 7.94-7.92 (m, 1H), |
| (2^{nd} | | | | (2R)-2-(3-methoxyphenyl)-N- | | 7.47 (s, J = 0.8 Hz, 1H), |
| eluting | | | | methyl-2-{6-[4-(1,3-oxazol-2- | | 7.19-7.16 (m, 1H), 6.93-6.89 |
| isomer) | | | | yl)benzenesulfonyl]- | | (m, 1H), 6.87-6.81 (m, 1H), |
| | | | | 5H,6H,7H-pyrrolo[3,4- | | 6.80-6.79 (m, 1H), 5.10 (s, |
| | | | | d]pyrimidin-2-yl}acetamide | | J = 0.8 Hz, 1H), 4.67-4.61 (m, |
| | | | | | | 4H), 3.71-3.68 (m, 3H), 2.52 |
| | | | | | | (s, J = 1.6 Hz, 3H). |
| 57 | +++ | 2-1, 27- | D | (S)-{6-[3-fluoro-4-(1,3-oxazol- | 547.0 | (CDCl₃, 400 MHz) δ (ppm): |
| (1^{st} | | 1, 8-21, | | 2-yl)benzenesulfonyl]- | | 8.68 (s, 1H), 8.26-8.23 (m, |
| eluting | | 34-1, | | 5H,6H,7H-pyrrolo[3,4- | | 1H), 8.15 (d, J = 8.8 Hz, |
| isomer) | | 35-1 | | d]pyrimidin-2-yl}({2- | | 1H), 7.84-7.72 (m, 5H), |
| 58 | ++++ | | | [(methylamino)methyl]quinolin- | | 7.57-7.53 (m, 1H), 7.37-7.32 |
| (2^{nd} | | | | 8-yl})methanol; | | (m, 3H), 6.57 (s, 1H), 4.71 |
| eluting | | | | (R)-{6-[3-fluoro-4-(1,3-oxazol- | | (s, 2H), 4.64 (s, 2H), 4.23- |
| isomer) | | | | 2-yl)benzenesulfonyl]- | | 4.11 (m, 2H), 2.69 (s, 2H). |
| | | | | 5H,6H,7H-pyrrolo[3,4- | | |
| | | | | d]pyrimidin-2-yl}({2- | | |
| | | | | [(methylamino)methyll]quinolin- | | |
| | | | | 8-yl})methanol | | |
| 59 | ++++ | 2-1, 27- | A | (S)-{6-[3-fluoro-4-(1,3-oxazol- | 524.0 | (DMSO-d6, 400 MHz) δ |
| (1^{st} | | 1, 33-2, | | 2-yl)benzenesulfonyl]- | | (ppm): 8.63 (s, 1H), 8.39 (s, |
| eluting | | 32-2 | | 5H,6H,7H-pyrrolo[3,4- | | 1H), 8.24-8.21 (m, 1H), |
| isomer) | | | | d]pyrimidin-2-yl}(2,3,4,5- | | 7.98-7.95 (m, 1H), 7.89-7.87 |
| 60 | +++ | | | tetrahydro-1,4-benzoxazepin-9- | | (m, 1H), 7.53 (s, 1H), 7.39- |
| (2^{nd} | | | | yl)methanol; | | 7.36 (m, 1H), 7.06-7.04 (m, |
| eluting | | | | (R)-{6-[3-fluoro-4-(1,3-oxazol- | | 1H), 6.96-6.92 (m, 1H), 5.98 |
| isomer) | | | | 2-yl)benzenesulfonyl]- | | (d, J = 5.6 Hz, 1H), 5.85 (d, |
| | | | | 5H,6H,7H-pyrrolo[3,4- | | J = 6.0 Hz, 1H), 4.72-4.63 (m, |
| | | | | d]pyrimidin-2-yl}(2,3,4,5- | | 4H), 3.71 (s, 2H), 3.52-3.45 |
| | | | | tetrahydro-1,4-benzoxazepin-9- | | (m, 1H), 3.34-3.25 (m, 2H), |
| | | | | yl)methanol | | 2.90-2.81 (m, 2H). |
| 61 | ++++ | 2-1, 22- | F | (S)-[(3S)-2-[3-fluoro-4-(1,3- | 564.0 | 1^{st}: (DMSO-d6, 400 MHz) δ |
| (1^{st} | | 2, 3-7, | | oxazol-2-yl)benzenesulfonyl]- | | (ppm): 8.36 (s, 1H), 8.33 (s, |
| eluting | | 36-1 | | 3-methyl-1H,2H,3H- | | 1H), 8.18 (t, J = 8.0 Hz, 1H), |
| isomer) | | | | pyrrolo[3,4-c]pyridin-6-yl][3- | | 7.90-7.87 (m, 1H), 7.84-7.82 |
| | | | | (4-methylpiperazin-1- | | (m, 1H), 7.51 (s, 1H), 7.44 |
| | | | | yl)phenyl]methanol; | | (s, 1H), 7.03-6.93 (m, 2H), |
| | | | | (R)-[(3R)-2-[3-fluoro-4-(1,3- | | 6.71-6.68 (m, 1H), 6.65-6.62 |
| | | | | oxazol-2-yl)benzenesulfonyl]- | | (m, 1H), 6.00 (d, J = 4.0 Hz, |
| | | | | 3-methyl-1H,2H,3H- | | 1H), 5.59 (d, J = 4.0 Hz, |
| | | | | pyrrolo[3,4-c]pyridin-6-yl][3- | | 1H), 5.07-5.03 (m, 1H), |
| | | | | (4-methylpiperazin-1- | | 4.77-4.74 (m, 1H), 4.68-4.64 |
| | | | | yl)phenyl]methanol; | | (m, 1H), 3.07 (br s, 4H), |
| | | | | (R)-[(3S)-2-[3-fluoro-4-(1,3- | | 2.50-2.48 (m, 4H), 2.26 (s, |
| | | | | oxazol-2-yl)benzenesulfonyl]- | | 3H), 1.58 (d, J = 6.4 Hz, |
| | | | | 3-methy1-1H,2H,3H- | | 3H). |
| 62 | ++++ | | | pyrrolo[3,4-c]pyridin-6-yl][3- | | 2^{nd}: (DMSO-d6, 400 MHz) δ |
| (2^{nd} | | | | (4-methylpiperazin-1- | | (ppm): 8.36 (s, 1H), 8.32 (s, |
| eluting | | | | yl)phenyl]methanol; | | 1H), 8.18 (t, J = 8.0 Hz, 1H), |
| isomer) | | | | (S)-[(3R)-2-[3-fluoro-4-(1,3- | | 7.90-7.87 (m, 1H), 7.84-7.82 |
| | | | | oxazol-2-yl)benzenesulfonyl]- | | (m, 1H), 7.51 (s, 1H), 7.46 |
| | | | | 3-methyl-1H,2H,3H- | | (s, 1H), 7.03-6.93 (m, 2H), |
| | | | | pyrrolo[3,4-c]pyridin-6-yl][3- | | 6.71-6.68 (m, 1H), 6.65-6.62 |
| | | | | (4-methylpiperazin-1- | | (m, 1H), 6.02 (d, J = 4.0 Hz, |
| | | | | yl)phenyl]methanol | | 1H), 5.59 (d, J = 4.0 Hz, |
| | | | | | | 1H), 5.07-5.03 (m, 1H), |
| | | | | | | 4.77-4.74 (m, 1H), 4.68-4.64 |
| | | | | | | (m, 1H), 3.06-3.04 (m, 4H), |
| | | | | | | 2.43 (br s, 4H), 2.22 (s, 3H), |
| | | | | | | 1.57 (d, J = 6.4 Hz, 3H). |
| 63 | ++++ | | | | | 3^{rd}: (DMSO-d6, 400 MHz) δ |
| (3^{rd} | | | | | | (ppm): 8.37 (s, 1H), 8.32 (s, |
| eluting | | | | | | 1H), 8.18 (t, J = 8.0 Hz, 1H), |
| isomer) | | | | | | 7.90-7.87 (m, 1H), 7.84-7.82 |
| | | | | | | (m, 1H), 7.51 (s, 1H), 7.46 |
| | | | | | | (s, 1H), 7.03-6.93 (m, 2H), |
| | | | | | | 6.71-6.68 (m, 1H), 6.65-6.62 |
| | | | | | | (m, 1H), 6.02 (d, J = 4.0 Hz, |
| | | | | | | 1H), 5.59 (d, J = 4.0 Hz, |
| | | | | | | 1H), 5.07-5.03 (m, 1H), |
| | | | | | | 4.79-4.68 (m, 2H), 3.15 (br |
| | | | | | | s, 4H), 2.72 (br s, 4H), 2.42 |
| | | | | | | (s, 3H), 1.57 (d, J = 6.4 Hz, |
| | | | | | | 3H). |
| 64 | ++++ | | | | | 4^{th}: (DMSO-d6, 400 MHz) δ |
| (4^{th} | | | | | | (ppm): 8.36 (s, 1H), 8.33 (s, |
| eluting | | | | | | 1H), 8.18 (t, J = 8.0 Hz, 1H), |
| isomer) | | | | | | 7.90-7.87 (m, 1H), 7.84-7.82 |
| | | | | | | (m, 1H), 7.51 (s, 1H), 7.44 |
| | | | | | | (s, 1H), 7.03-6.93 (m, 2H), |
| | | | | | | 6.71-6.68 (m, 1H), 6.65-6.62 |
| | | | | | | (m, 1H), 6.00 (d, J = 4.0 Hz, |
| | | | | | | 1H), 5.59 (d, J = 4.0 Hz, |
| | | | | | | 1H), 5.07-5.03 (m, 1H), |
| | | | | | | 4.77-4.74 (m, 1H), 4.68-4.64 |
| | | | | | | (m, 1H), 3.07 (br s, 4H), |
| | | | | | | 2.50-2.48 (m, 4H), 2.26 (s, |
| | | | | | | 3H), 1.58 (d, J = 6.4 Hz, |
| | | | | | | 3H). |
| 65 | ++++ | 2-1, 22- | F | (S)-[(1R)-2-[3-fluoro-4-(1,3- | 564.0 | 1^{st}: (DMSO-d6, 400 MHz) δ |
| (1^{st} | | 2, 3-7, | | oxazol-2-yl)benzenesulfonyl]- | | (ppm): 8.36 (s, 1H), 8.33 (s, |
| eluting | | 36-1 | | 1-methyl-1H,2H,3H- | | 1H), 8.18 (t, J = 8.0 Hz, 1H), |
| isomer) | | | | pyrrolo[3,4-c]pyridin-6-yl][3- | | 7.90-7.87 (m, 1H), 7.84-7.82 |
| | | | | (4-methylpiperazin-1- | | (m, 1H), 7.51 (s, 1H), 7.44 |
| | | | | yl)phenyl]methanol; | | (s, 1H), 7.10-7.05 (m, 1H), |
| | | | | (R)-[(1S)-2-[3-fluoro-4-(1,3- | | 6.99 (s, 1H), 6.76-6.72 (m, |
| | | | | oxazol-2-yl)benzenesulfonyl]- | | 1H), 6.65-6.62 (m, 1H), 6.00 |
| | | | | 1-methyl-1H,2H,3H- | | (d, J = 4.0 Hz, 1H), 5.59 (d, |
| | | | | pyrrolo[3,4-c]pyridin-6-yl][3- | | J = 4.0 Hz, 1H), 5.07-5.03 (m, |
| | | | | (4-methylpiperazin-1- | | 1H), 4.77-4.74 (m, 1H), |
| | | | | yl)phenyl]methanol; | | 4.68-4.64 (m, 1H), 3.07 (br |
| | | | | (S)-[(1R)-2-[3-fluoro-4-(1,3- | | s, 4H), 2.43-2.41 (m, 4H), |
| | | | | oxazol-2-yl)benzenesulfonyl]- | | 2.21 (s, 3H), 1.58 (d, J = 6.4 |
| | | | | 1-methyl-1H,2H,3H- | | Hz, 3H). |
| 66 | ++++ | | | pyrrolo[3,4-c]pyridin-6-yl][3- | | 2^{nd}: (DMSO-d6, 400 MHz) δ |
| (2^{nd} | | | | (4-methylpiperazin-1- | | (ppm): 8.36 (s, 1H), 8.33 (s, |
| eluting | | | | yl)phenyl]methanol; | | 1H), 8.18 (t, J = 8.0 Hz, 1H), |
| isomer) | | | | (S)-[(3R)-2-[3-fluoro-4-(1,3- | | 7.90-7.87 (m, 1H), 7.84-7.82 |
| | | | | oxazol-2-yl)benzenesulfonyl]- | | (m, 1H), 7.52 (s, 1H), 7.47 |
| | | | | 3-methyl-1H,2H,3H- | | (s, 1H), 7.10-7.05 (m, 1H), |
| | | | | pyrrolo[3,4-c]pyridin-6-yl][3- | | 6.99 (s, 1H), 6.76-6.72 (m, |
| | | | | (4-methylpiperazin-1- | | 1H), 6.65-6.62 (m, 1H), 6.00 |
| | | | | yl)phenyl]methanol | | (d, J = 4.0 Hz, 1H), 5.59 (d, |
| | | | | | | J = 4.0 Hz, 1H), 5.07-5.03 (m, |
| | | | | | | 1H), 4.77-4.74 (m, 1H), |
| | | | | | | 4.68-4.64 (m, 1H), 3.07-3.05 |
| | | | | | | (m, 4H), 2.43-2.41 (m, 4H), |
| | | | | | | 2.21 (s, 3H), 1.58 (d, J = 6.4 |
| | | | | | | Hz, 3H). |
| 67 | ++++ | | | | | 3^{rd}: (DMSO-d6, 400 MHz) δ |
| (3^{rd} | | | | | | (ppm): 8.36 (s, 1H), 8.33 (s, |
| eluting | | | | | | 1H), 8.18 (t, J = 8.0 Hz, 1H), |
| isomer) | | | | | | 7.90-7.87 (m, 1H), 7.84-7.82 |
| | | | | | | (m, 1H), 7.52 (s, 1H), 7.47 |
| | | | | | | (s, 1H), 7.10-7.05 (m, 1H), |
| | | | | | | 6.99 (s, 1H), 6.76-6.72 (m, |
| | | | | | | 1H), 6.65-6.62 (m, 1H), 6.00 |
| | | | | | | (d, J = 4.0 Hz, 1H), 5.59 (d, |
| | | | | | | J = 4.0 Hz, 1H), 5.07-5.03 (m, |
| | | | | | | 1H), 4.77-4.74 (m, 1H), |
| | | | | | | 4.68-4.64 (m, 1H), 3.08 (br |
| | | | | | | s, 4H), 2.50-2.48 (m, 4H), |
| | | | | | | 2.24 (s, 3H), 1.54 (d, J = 6.4 |
| | | | | | | Hz, 3H). |
| 68 | ++++ | | | | | 4^{th}: (DMSO-d6, 400 MHz) δ |
| (4^{th} | | | | | | (ppm): 8.36 (s, 1H), 8.33 (s, |
| eluting | | | | | | 1H), 8.18 (t, J = 8.0 Hz, 1H), |
| isomer) | | | | | | 7.90-7.87 (m, 1H), 7.84-7.82 |
| | | | | | | (m, 1H), 7.52 (s, 1H), 7.44 |
| | | | | | | (s, 1H), 7.10-7.05 (m, 1H), |
| | | | | | | 6.99 (s, 1H), 6.76-6.72 (m, |
| | | | | | | 1H), 6.65-6.62 (m, 1H), 6.00 |
| | | | | | | (d, J = 4.0 Hz, 1H), 5.59 (d, |
| | | | | | | J = 4.0 Hz, 1H), 5.07-5.03 (m, |
| | | | | | | 1H), 4.77-4.74 (m, 1H), |
| | | | | | | 4.68-4.64 (m, 1H), 3.08 (br |
| | | | | | | s, 4H), 2.44-2.41 (m, 4H), |
| | | | | | | 2.21 (s, 3H), 1.59 (d, J = 6.4 |
| | | | | | | Hz, 3H). |
| 69 | ++++ | 2-1, 12- | F | (S)-{3-[(3aR,6aS)-5-methyl- | 576.0 | 1^{st}: (CD₃OD, 400 MHz) δ |
| (1^{st} | | 1, 3-8, | | octahydropyrrolo[3,4-c]pyrrol- | | (ppm): 8.35-8.33 (m, 1H), |
| eluting | | 4-14 | | 2-yl]phenyl}({2-[3-fluoro-4- | | 8.26-8.22 (m, 1H), 8.14-8.12 |
| isomer) | | | | (1,3-oxazol-2- | | (m, 1H), 7.87-7.84 (m, 2H), |
| | | | | yl)benzenesulfonyl]- | | 7.52 (s, 1H), 7.42 (s, 1H), |
| | | | | 1H,2H,3H-pyrrolo[3,4- | | 7.10-7.06 (m, 1H), 6.78-6.76 |
| | | | | c]pyridin-6-yl})methanol; | | (m, 1H), 6.69-6.67 (m, 1H), |
| | | | | (R)-{3-[(3aR,6aS)-5-methyl- | | 6.58-6.56 (m, 1H), 5.71 (s, |
| | | | | octahydropyrrolo[3,4-c]pyrrol- | | 1H), 4.74-4.72(m, 4H), 3.24- |
| | | | | 2-yl]phenyl}({2-[3-fluoro-4- | | 3.14 (m, 4H), 2.95-2.93 (m, |
| | | | | (1,3-oxazol-2- | | 4H), 2.40-2.29 (m, 5H). |
| 70 | ++++ | | | yl)benzenesulfonyl]- | | 2^{nd} (CD₃OD, 400 MHz) δ |
| (2^{nd} | | | | 1H,2H,3H-pyrrolo[3,4- | | (ppm): 8.35-8.33 (m, 1H), |
| eluting | | | | c]pyridin-6-yl})methanol | | 8.26-8.22 (m, 1H), 8.14-8.12 |
| isomer) | | | | | | (m, 1H), 7.87-7.84 (m, 2H), |
| | | | | | | 7.52 (s, 1H), 7.42 (s, 1H), |
| | | | | | | 7.10-7.06 (m, 1H), 6.77-6.76 |
| | | | | | | (m, 1H), 6.70-6.67 (m, 1H), |
| | | | | | | 6.58-6.56 (m, 1H), 5.71 (s, |
| | | | | | | 1H), 4.74-4.68(m, 4H), 3.25- |
| | | | | | | 3.17 (m, 4H), 2.96-2.94 (m, |
| | | | | | | 4H), 2.41-2.31 (m, 5H). |
| 71 | ++++ | 2-1, 4- | L | (S)-{2-[3-fluoro-4-(1,3-oxazol- | 537.0 | (DMSO-d6, 400 MHz) δ |
| (1^{st} | | 15, 37- | | 2-yl)benzenesulfonyl]- | | (ppm): 8.38 (s, 1H), 8.34 (s, |
| eluting | | 1, 8-22 | | 1H,2H,3H-pyrrolo[3,4- | | 1H), 8.24-8.20 (m, 1H), |
| isomer) | | | | c]pyridin-6-yl}({3-[(1- | | 7.90-7.83 (m, 2H), 7.53 (s, |
| 72 | ++++ | | | methylazetidin-3- | | 1H), 7.47 (s, 1H), 7.14-7.10 |
| (2^{nd} | | | | yl)oxy]phenyl})methanol; | | (m, 1H), 6.88 (d, J = 7.6 Hz, |
| eluting | | | | (R)-{2-[3-fluoro-4-(1,3-oxazol- | | 1H), 6.81 (s, 1H), 6.60-6.57 |
| isomer) | | | | 2-yl)benzenesulfonyl]- | | (m, 1H), 6.10 (d, J = 4.0 Hz, |
| | | | | 1H,2H,3H-pyrrolo[3,4- | | 1H), 5.63 (d, J = 4.4 Hz, 1H), |
| | | | | c]pyridin-6-yl}({3-[(1- | | 4.70-4.63 (m, 5H), 3.71-3.68 |
| | | | | methylazetidin-3- | | (m, 2H), 2.92-2.89 (m, 2H), |
| | | | | yl)oxy]phenyl})methanol | | 2.27 (s, 3H). |
| 73 | ++++ | 38-1, 8- | L | (S)-(3-{5-methyl- | 556.0 | (DMSO-d6, 400 MHz) δ |
| (1^{st} | | 11,3-9, | | 1H,2H,3H,4H,5H,6H- | | (ppm): 8.31 (d, J = 4.0 Hz, |
| eluting | | 4-16 | | pyrrolo[3,4-c]pyrrol-2- | | 2H), 8.15 (d, J = 8.4 Hz, |
| isomer) | | | | yl}phenyl)({2-[4-(1,3-oxazol- | | 2H), 8.00 (d, J = 8.4 Hz, |
| 74 | ++++ | | | 2-yl)benzenesulfonyl]- | | 2H), 7.45 (s, 2H), 7.00 (t, J = |
| (2^{nd} | | | | 1H,2H,3H-pyrrolo[3,4- | | 8.0 Hz, 1H), 6.54-6.49 (m, |
| eluting | | | | c]pyridin-6-yl})methanol; | | 2H), 6.29 (d, J = 7.2 Hz, |
| isomer) | | | | (R)-(3-{5-methyl- | | 1H), 5.95 (s, 1H), 5.56 (s, |
| | | | | 1H,2H,3H,4H,5H,6H- | | 1H), 4.64-4.62 (m, 4H), 3.92 |
| | | | | pyrrolo[3,4-c]pyrrol-2- | | (s, 4H), 3.46 (s, 4H), 2.46 (s, |
| | | | | yl}phenyl)({2-[4-(1,3-oxazol- | | 3H). |
| | | | | 2-yl)benzenesulfonyl]- | | |
| | | | | 1H,2H,3H-pyrrolo[3,4- | | |
| | | | | c]pyridin-6-yl})methanol | | |
| 75 | ++++ | 8-11 | J | (S)-{3-[3- | 532.0 | (DMSO-d6, 400 MHz) δ |
| (2^{nd} | | | | (dimethylamino)azetidin-1- | | (ppm): 8.32 (s, 2H), 8.16 (d, |
| eluting | | | | yl]phenyl}({2-[4-(1,3-oxazol- | | J = 8.4 Hz, 2H), 8.01 (d, J = |
| isomer) | | | | 2-yl)benzenesulfonyl]- | | 8.4 Hz, 2H), 7.47-7.44 (m, |
| | | | | 1H,2H,3H-pyrrolo[3,4- | | 2H), 6.99 (t, J = 8.0 Hz, 1H), |
| | | | | c]pyridin-6-yl})methanol; or | | 6.57 (d, J = 7.6 Hz, 1H), 6.46 |
| | | | | (R)-{3-[3- | | (s, 1H), 6.263-6.20 (m, 1H), |
| | | | | (dimethylamino)azetidin-1- | | 5.97 (d, J = 4.0 Hz, 1H), 5.56 |
| | | | | yl]phenyl}({2-[4-(1,3-oxazol- | | (d, J = 4.0 Hz, 1H), 4.65- |
| | | | | 2-yl)benzenesulfonyl]- | | 4.58 (m, 4H), 3.85-3.82 (m, |
| | | | | 1H,2H,3H-pyrrolo[3,4- | | 2H), 3.48-3.40 (m, 2H), |
| | | | | c]pyridin-6-yl})methanol | | 3.11-3.09 (m, 1H), 2.08 (s, |
| | | | | | | 6H). |
| 76 | ++++ | 22-1, | J | 1-{3-[(R)-methoxy({2-[4-(1,3- | 546.0 | (DMSO-d6, 400 MHz) δ |
| (1^{st} | | 23-1, 8-17 | | oxazol-2-yl)benzenesulfonyl]- | | (ppm): 8.33 (d, J = 8.8 Hz, |
| eluting | | | | 1H,2H,3H-pyrrolo[3,4- | | 2H), 8.15 (d, J = 8.4 Hz, |
| isomer) | | | | c]pyridin-6- | | 2H), 8.00 (d, J = 8.4 Hz, |
| 77 | ++++ | | | yl})methyl]phenyl}-N,N- | | 2H), 7.47 (d, J = 0.8 Hz, |
| (2^{nd} | | | | dimethylazetidin-3-amine; | | 1H), 7.41 (s, 1H), 7.02 (t, J = |
| eluting | | | | 1-{3-[(S)-methoxy({2-[4-(1,3- | | 8.0 Hz, 1H), 6.54 (d, J = 7.6 |
| isomer) | | | | oxazol-2-yl)benzenesulfonyl]- | | Hz, 1H), 6.40 (s, 1H), 6.26- |
| | | | | 1H,2H,3H-pyrrolo[3,4- | | 6.23 (m, 1H), 5.20 (s, 1H), |
| | | | | c]pyridin-6- | | 4.70-4.58 (m, 4H), 3.86-3.81 |
| | | | | yl})methyl]phenyl}-N,N- | | (m, 2H), 3.48-3.43 (m, 2H), |
| | | | | dimethylazetidin-3-amine | | 3.22 (s, 3H), 3.14-3.10 (m, |
| | | | | | | 1H), 2.08 (s, 6H). |
| 78 | ++++ | 24-2, | C | (S)-{3-[(1-methylpiperidin-4- | 547.0 | (DMSO-d6, 400 MHz) δ |
| (1^{st} | | 26-1 | | yl)oxy]phenyl}({2-[4-(1,3- | | (ppm): 8.35-8.31 (m, 2H), |
| eluting | | | | oxazol-2-yl)benzenesulfonyl]- | | 8.19-8.13 (m, 2H), 8.04-7.98 |
| isomer) | | | | 1H,2H,3H-pyrrolo[3,4- | | (m, 2H), 7.50-7.45 (m, 2H), |
| 79 | ++++ | | | c]pyridin-6-yl})methanol; | | 7.12-7.09 (m, 1H), 6.90-6.89 |
| (2^{nd} | | | | (R)-{3-[(1-methylpiperidin-4- | | (m, 1H), 6.83-6.81 (m, 1H), |
| eluting | | | | yl)oxy]phenyl}({2-[4-(1,3- | | 6.75-6.72 (m, 1H), 6.07-6.06 |
| isomer) | | | | oxazol-2-yl)benzenesulfonyl]- | | (m, 1H), 5.62-5.61 (m, 1H), |
| | | | | 1H,2H,3H-pyrrolo[3,4- | | 4.68-4.56 (m, 4H), 4.29-4.25 |
| | | | | c]pyridin-6-yl})methanol | | (m, 1H), 2.60-2.53 (m, 2H), |
| | | | | | | 2.16-2.09 (m, 5H), 1.88-1.84 |
| | | | | | | (m, 2H), 1.62-1.51 (m, 2H). |
| 80 | ++ | | | 6-[(S)- | 450.0 | (DMSO-d6, 400 MHz) δ |
| (1^{st} | | | | hydroxy(phenyl)methyl]-2-[4- | | (ppm): 8.36 (s, 1H), 8.18- |
| eluting | | | | (1,3-oxazol-2- | | 8.13 (m, 3H), 8.02-7.99 (m, |
| isomer) | | | | yl)benzenesulfonyl]- | | 2H), 7.63 (s, 1H), 7.49 (s, |
| 81 | ++ | | | 1H,2H,3H-pyrrolo[3,4- | | 1H), 7.28-7.16 (m, 5H), 6.97 |
| (2^{nd} | | | | c]pyridin-5-ium-5-olate; | | (s, 1H), 4.69-4.56 (m, 4H). |
| eluting | | | | 6-[(R)- | | |
| isomer) | | | | hydroxy(phenyl)methyl]-2-[4- | | |
| | | | | (1,3-oxazol-2- | | |
| | | | | yl)benzenesulfonyl]- | | |
| | | | | 1H,2H,3H-pyrrolo[3,4- | | |
| | | | | c]pyridin-5-ium-5-olate | | |
| 82 | ++++ | 28-1 | F | (S)-{6-[3-fluoro-4-(1,3-oxazol- | 565.0 | (DMSO-d6, 400 MHz) δ |
| (1^{st} | | | | 2-yl)benzenesulfonyl]-4- | | (ppm): 8.38 (s, 1H), 8.24- |
| eluting | | | | methyl-5H,6H,7H-pyrrolo[3,4- | | 8.20 (m, 1H), 7.97-7.94 (m, |
| isomer) | | | | d]pyrimidin-2-yl}[3-(4- | | 1H), 7.89-7.87 (m, 1H), 7.53 |
| 83 | +++ | | | methylpiperazin-1- | | (s, 1H), 7.06-7.02 (m, 2H), |
| (2^{nd} | | | | yl)phenyl]methanol; | | 6.74-6.72 (m, 2H), 5.79-5.78 |
| eluting | | | | (R)-{6-[3-fluoro-4-(1,3-oxazol- | | (m, 1H), 5.61-5.60 (m, 1H), |
| isomer) | | | | 2-yl)benzenesulfonyl]-4- | | 4.73-4.70 (m, 2H), 4.69-4.65 |
| | | | | methyl-5H,6H,7H-pyrrolo[3,4- | | (m, 2H), 3.10-3.07 (m, 4H), |
| | | | | d]pyrimidin-2-yl}[3-(4- | | 2.50-2.45 (m, 4H), 2.37-2.33 |
| | | | | methylpiperazin-1- | | (m, 3H), 2.25-2.23 (m, 3H). |
| | | | | yl)phenyl]methanol | | |
| 84 | ++++ | 8-11 | J | (S)-(3-{6-methy1-2,6- | 544.0 | (DMSO-d6, 400 MHz) δ |
| (1^{st} | | | | diazaspiro[3.3]heptan-2- | | (ppm): 8.32 (s, 2H), 8.16 (d, |
| eluting | | | | yl}phenyl)({2-[4-(1,3-oxazol- | | J = 8.8 Hz, 2H), 8.01 (d, J = |
| isomer) | | | | 2-yl)benzenesulfonyl]- | | 8.4 Hz, 2H), 7.47-7.44 (m, |
| 85 | ++++ | | | 1H,2H,3H-pyrrolo[3,4- | | 2H), 7.00-6.96 (m, 1H), 6.58 |
| (2^{nd} | | | | c]pyridin-6-yl})methanol; | | (d, J = 8.0 Hz, 1H), 6.44 (s, |
| eluting | | | | (R)-(3-{6-methyl-2,6- | | 1H), 6.20 (d, J = 8.4 Hz, |
| isomer) | | | | diazaspiro[3.3]heptan-2- | | 1H), 5.96 (d, J = 4.0 Hz, |
| | | | | yl}phenyl)({2-[4-(1,3-oxazol- | | 1H), 5.56 (d, J = 3.6 Hz, |
| | | | | 2-yl)benzenesulfonyl]- | | 1H), 4.64-4.58 (m, 4H), 3.78 |
| | | | | 1H,2H,3H-pyrrolo[3,4- | | (s, 4H), 3.33-3.27 (m, 4H), |
| | | | | c]pyridin-6-yl})methanol | | 2.20 (s, 3H). |
| 86 | ++++ | 8-11 | B | (S)-{3-[(4aS,7aS)- | 560.0 | 1^{st}: (DMSO-d6, 400 MHz) δ |
| (1^{st} | | | | octahydropyrrolo[3,4- | | (ppm): 8.32-8.30 (m, 2H), |
| eluting | | | | b]morpholin-6-yl]phenyl}({2- | | 8.21-8.15 (m, 2H), 8.02-7.98 |
| isomer) | | | | [4-(1,3-oxazol-2- | | (m, 2H), 7.48-7.44 (m, 2H), |
| | | | | yl)benzenesulfonyl]- | | 7.02-6.98 (m, 1H), 6.57-6.51 |
| | | | | 1H,2H,3H-pyrrolo[3,4- | | (m, 2H), 6.33 (t, J = 8.4 Hz, |
| | | | | c]pyridin-6-yl})methanol; | | 1H), 5.96-5.92 (m, 1H), |
| | | | | (R)-{3-[(4aS,7aS)- | | 5.59-5.55 (m, 1H), 4.63-4.58 |
| | | | | octahydropyrrolo[3,4- | | (m, 4H), 4.10-3.82 (m, 2H), |
| | | | | b]morpholin-6-yl]phenyl}({2- | | 3.70-3.45 (m, 3H), 3.20-2.99 |
| | | | | [4-(1,3-oxazol-2- | | (m, 4H), 2.35-2.30 (m, 1H), |
| | | | | yl)benzenesulfonyl]- | | 2.17-2.12 (m, 1H). |
| 87 | ++++ | | | 1H,2H,3H-pyrrolo[3,4- | | 2^{nd}: (DMSO-d6, 400 MHz) δ |
| (2^{nd} | | | | c]pyridin-6-yl})methanol; | | (ppm): 8.32 (s, 2H), 8.16 (d, |
| eluting | | | | (S)-{3-[(4aR,7aR)- | | J = 8.8 Hz, 2H), 8.01 (d, J = |
| isomer) | | | | octahydropyrrolo[3,4- | | 8.4 Hz, 2H), 7.46 (d, J = 4.0 |
| | | | | b]morpholin-6-yl]phenyl}({2- | | Hz, 2H), 7.00 (t, J = 8.0 Hz, |
| | | | | [4-(1,3-oxazol-2- | | 1H), 6.55-6.51(m, 2H), 6.30 |
| | | | | yl)benzenesulfonyl]- | | (d, J = 8.0 Hz, 1H), 5.95 (d, |
| | | | | 1H,2H,3H-pyrrolo[3,4- | | J = 4.0 Hz, 1H), 5.58 (d, J = |
| | | | | c]pyridin-6-yl})methanol; | | 4.0 Hz, 1H), 4.68-4.61 (m, |
| | | | | (R)-{3-[(4aR,7aR)- | | 4H), 3.95-3.91 (m, 1H), |
| | | | | octahydropyrrolo[3,4- | | 3.70-3.61 (m, 2H), 3.48-3.42 |
| | | | | b]morpholin-6-yl]phenyl}({2- | | (m, 2H), 3.05-2.91 (m, 5H). |
| 88 | ++++ | | | [4-(1,3-oxazol-2- | | 3^{rd}: (DMSO-d6, 400 MHz) δ |
| (3^{rd} | | | | yl)benzenesulfonyl]- | | (ppm): 8.32-8.31 (m, 2H), |
| eluting | | | | 1H,2H,3H-pyrrolo[3,4- | | 8.16 (d, J = 8.4 Hz, 2H), |
| isomer) | | | | c]pyridin-6-yl})methanol | | 8.04-7.96 (m, 2H), 7.49-7.44 |
| | | | | | | (m, 2H), 6.99 (t, J = 8.0 Hz, |
| | | | | | | 1H), 6.57-6.50 (m, 2H), |
| | | | | | | 6.32-6.28 (m, 1H), 5.95 (d, J = |
| | | | | | | 4.4 Hz, 1H), 5.58 (d, J = |
| | | | | | | 4.4 Hz, 1H), 4.62-4.57 (m, |
| | | | | | | 4H), 3.96-3.93 (m, 1H), |
| | | | | | | 3.65-3.53 (m, 3H), 3.46-3.40 |
| | | | | | | (m, 1H), 3.08-3.02 (m, 3H), |
| | | | | | | 2.93-2.85 (m, 2H), 2.35-2.30 |
| | | | | | | (m, 1H), 2.15-2.10 (m, 1H). |
| 89 | ++++ | | | | | 4^{th}: (DMSO-d6, 400 MHz) δ |
| (4^{th} | | | | | | (ppm): 8.32 (d, J = 6.4 Hz, |
| eluting | | | | | | 2H), 8.17-8.15 (m, 2H), |
| isomer) | | | | | | 8.02-7.98 (m, 2H), 7.46 (d, |
| | | | | | | J = 6.0 Hz, 2H), 7.01-6.98 (m, |
| | | | | | | 1H), 6.59-6.48 (m, 2H), |
| | | | | | | 6.32-6.30 (m, 1H), 5.97-5.94 |
| | | | | | | (m, 1H), 5.58-5.56 (m, 1H), |
| | | | | | | 4.64-4.62 (m, 4H), 4.04-3.95 |
| | | | | | | (m, 1H), 3.85-3.44 (m, 4H), |
| | | | | | | 3.30-3.28 (m, 1H), 3.08-3.03 |
| | | | | | | (m, 4H). |
| 90 | +++ | | | (S)-[4-methyl-3-(4- | 552.0 | (Methanol-d4, 400 MHz) δ |
| (1^{st} | | | | methylpiperazin-1- | | (ppm): 8.38 (s, 1H), 8.01- |
| eluting | | | | yl)phenyl](2-{[5-(1,3-oxazol- | | 8.00 (m, 1H), 7.73-7.72 (m, |
| somer) | | | | 2-yl)thiophen-2-yl]sulfonyl}- | | 2H), 7.56 (s, 1H), 7.32-7.30 |
| 91 | ++++ | | | 1H,2H,3H-pyrrolo[3,4- | | (m, 1H), 7.14-7.12 (m, 1H), |
| (2^{nd} | | | | c]pyridin-6-yl)methanol; | | 7.09-7.08 (m, 1H), 6.95-6.92 |
| eluting | | | | (R)-[4-methyl-3-(4- | | (m, 1H), 5.74 (s, 1H), 4.76 |
| isomer) | | | | methylpiperazin-1- | | (s, 4H), 3.00 (s, 8H), 2.63 (s, |
| | | | | yl)phenyl](2-{[5-(1,3-oxazol- | | 3H), 2.15 (s, 3H). |
| | | | | 2-yl)thiophen-2-yl]sulfonyl}- | | |
| | | | | 1H,2H,3H-pyrrolo[3,4- | | |
| | | | | c]pyridin-6-yl)methanol | | |
| 92 | ++++ | 8-11 | B | (S)-{3-[(3aR,6aR)- | 544.0 | 1^{st}: (DMSO-d6, 400 MHz) δ |
| (1^{st} | | | | octahydropyrrolo[3,4-b]pyrrol- | | (ppm): 8.32 (s, 2H), 8.16 (d, |
| eluting | | | | 5-yl]phenyl}({2-[4-(1,3- | | J = 8.4 Hz, 2H), 8.02 (d, J = |
| isomer) | | | | oxazol-2-yl)benzenesulfonyl]- | | 8.4 Hz, 2H), 7.46 (d, J = 6.8 |
| | | | | 1H,2H,3H-pyrrolo[3,4- | | Hz, 2H), 7.03-6.99 (m, 1H), |
| | | | | c]pyridin-6-yl})methanol; | | 6.62-6.55 (m, 2H), 6.43-6.41 |
| | | | | (R)-{3-[(3aR,6aR)- | | (m, 1H), 5.98-5.97 (m, 1H), |
| | | | | octahydropyrrolo[3,4-b]pyrrol- | | 5.58-5.57 (m, 1H), 4.69-4.62 |
| | | | | 5-yl]phenyl}({2-[4-(1,3- | | (m, 4H), 4.00-3.95 (m, 1H), |
| | | | | oxazol-2-yl)benzenesulfonyl]- | | 3.32-3.29 (m, 4H), 3.10-3.07 |
| | | | | 1H,2H,3H-pyrrolo[3,4- | | (m, 1H), 3.00-2.97 (m, |
| | | | | c]pyridin-6-yl})methanol; | | 3H),1.98-1.94 (m, 1H), 1.70- |
| | | | | (S)-{3-[(3aS,6aS)- | | 1.68 (m, 1H). |
| 93 | ++++ | | | octahydropyrrolo[3,4-b]pyrrol- | | 2^{nd}: (DMSO-d6, 400 MHz) δ |
| (2nd | | | | 5-yl]phenyl}({2-[4-(1,3- | | (ppm): 8.32 (s, 2H), 8.16 (d, |
| eluting | | | | oxazol-2-yl)benzenesulfonyl]- | | J = 8.0 Hz, 2H), 8.01 (d, J = |
| isomer) | | | | 1H,2H,3H-pyrrolo[3,4- | | 8.4 Hz, 2H), 7.46 (d, J = 4.8 |
| | | | | c]pyridin-6-yl})methanol; | | Hz, 2H), 7.03-6.98 (m, 1H), |
| | | | | (R)-{3-[(3aS,6aS)- | | 6.64-6.62 (m, 1H), 6.58-6.55 |
| | | | | octahydropyrrolo[3,4-b]pyrrol- | | (m, 1H), 6.43-6.40 (m, 1H), |
| | | | | 5-yl]phenyl}({2-[4-(1,3- | | 5.99-5.97 (m, 1H), 5.58-5.56 |
| | | | | oxazol-2-yl)benzenesulfonyl]- | | (m, 1H), 4.69-4.63 (m, 4H), |
| | | | | 1H,2H,3H-pyrrolo[3,4- | | 3.99-3.94 (m, 1H), 3.31-3.28 |
| | | | | c]pyridin-6-yl})methanol; | | (m, 4H), 3.09-3.06 (m, 1H), |
| | | | | | | 3.00-2.95 (m, 3H), 2.00-1.95 |
| | | | | | | (m, 1H), 1.69-1.67 (m, 1H). |
| 94 | | | | | | 3^{rd}: (DMSO-d6, 400 MHz) δ |
| (3^{rd} | | | | | | (ppm): 8.32 (s, 2H), 8.16 (d, |
| eluting | | | | | | J = 8.4 Hz, 2H), 8.00 (d, J = |
| isomer) | | | | | | 8.4 Hz, 2H), 7.46 (d, J = 7.2 |
| | | | | | | Hz, 2H), 7.00-6.98 (m, 1H), |
| | | | | | | 6.63-6.61 (m, 1H), 6.58-6.56 |
| | | | | | | (m, 1H), 6.40-6.37 (m, 1H), |
| | | | | | | 5.94 (d, J = 4.0 Hz, 1H), 5.56 |
| | | | | | | (d, J = 4.0 Hz, 1H), 4.68- |
| | | | | | | 4.63 (m, 4H), 3.85-3.81 (m, |
| | | | | | | 1H), 3.31-3.26 (m, 3H), |
| | | | | | | 3.04-3.02 (m, 1H), 2.98-2.95 |
| | | | | | | (m, 1H), 2.81-2.74 (m, 3H), |
| | | | | | | 2.00-1.95 (m, 1H), 1.69-1.67 |
| | | | | | | (m, 1H). |
| 95 | ++++ | | | | | 4^{th}: (DMSO-d6, 400 MHz) δ |
| (4^{th} | | | | | | (ppm): 8.32 (s, 2H), 8.16 (d, |
| eluting) | | | | | | J = 8.0 Hz, 2H), 8.00 (d, J = |
| isomer | | | | | | 8.0 Hz, 2H), 7.46 (d, J = 5.2 |
| | | | | | | Hz, 2H), 7.01-6.96 (m, 1H), |
| | | | | | | 6.63-6.61 (m, 1H), 6.58-6.56 |
| | | | | | | (m, 1H), 6.40-6.37 (m, 1H), |
| | | | | | | 5.95 (d, J = 3.6 Hz, 1H), 5.56 |
| | | | | | | (d, J = 3.6 Hz, 1H), 4.69- |
| | | | | | | 4.63 (m, 4H), 4.00-3.95 (m, |
| | | | | | | 1H), 3.30-3.26 (m, 3H), |
| | | | | | | 3.05-3.03 (m, 1H), 2.97-2.94 |
| | | | | | | (m, 1H), 2.80-2.73 (m, 3H), |
| | | | | | | 2.00-1.95 (m, 1H), 1.69-1.67 |
| | | | | | | (m, 1H). |
| 96 | ++++ | 4-13, 8- | B | (S)-(3-{3,6- | 530.0 | (CD₃OD, 400 MHz) δ |
| (1^{st} | | 11 | | diazabicyclo[3.1.1]heptan-3- | | (ppm): 8.31 (s, 1H), 8.21 (d, |
| eluting | | | | yl}phenyl)({2-[4-(1,3-oxazol- | | J = 8.8 Hz, 2H), 8.07-8.03 |
| isomer) | | | | 2-yl)benzenesulfonyl]- | | (m, 3H), 7.53 (s, 1H), 7.38 |
| 97 | ++++ | | | 1H,2H,3H-pyrrolo[3,4- | | (s, 1H), 7.16-7.12 (m, 1H), |
| (2^{nd} | | | | c]pyridin-6-yl})methanol; | | 6.84 (s, 1H), 6.70-6.63 (m, |
| eluting | | | | (R)-(3-{3,6- | | 2H), 5.74 (s, 1H), 4.72 (s, |
| isomer) | | | | diazabicyclo[3.1.1]heptan-3- | | 4H), 4.02-3.99 (m, 2H), |
| | | | | yl}phenyl)({2-[4-(1,3-oxazol- | | 3.65-3.55 (m, 4H), 2.82-2.80 |
| | | | | 2-yl)benzenesulfonyl]- | | (m, 1H), 1.75-1.73 (m, 1H). |
| | | | | 1H,2H,3H-pyrrolo[3,4- | | |
| | | | | c]pyridin-6-yl})methanol | | |
| 98 | ++++ | 4-5, 25- | F | (S)-[3-(4-methylpiperazin-1- | 533.0 | (DMSO-d6, 400 MHz) δ |
| (1^{st} | | 2, 27-1, | | yl)phenyl]({6-[4-(1,3-oxazol- | | (ppm): 8.64 (s, 1H), 8.33 (s, |
| eluting | | 3-2, 33-1 | | 2-yl)benzenesulfonyl]- | | 1H), 8.15 (d, J = 8.4 Hz, |
| isomer) | | | | 5H,6H,7H-pyrrolo[3,4- | | 2H), 8.04 (d, J = 8.4 Hz, |
| 99 | ++++ | | | d]pyrimidin-2-yl})methanol; | | 2H), 7.47 (s, 1H), 7.06-7.02 |
| (2^{nd} | | | | (R)-[3-(4-methylpiperazin-1- | | (m, 2H), 6.74-6.71 (m, 2H), |
| eluting | | | | yl)phenyl]({6-[4-(1,3-oxazol- | | 5.87 (d, J = 6.0 Hz, 1H), 5.64 |
| isomer) | | | | 2-yl)benzenesulfonyl]- | | (d, J = 5.6 Hz, 1H), 4.67- |
| | | | | 5H,6H,7H-pyrrolo[3,4- | | 4.63 (m, 4H), 3.07-3.05 (m, |
| | | | | d]pyrimidin-2-yl})methanol | | 4H), 2.42-2.40 (m, 4H), 2.21 |
| | | | | | | (s, 3H). |
| 100 | ++++ | 4-13, 8-11 | J | (S)-(3-{5-methyl- | 558.0 | (DMSO-d6, 400 MHz) δ |
| (1^{st} | | | | octahydropyrrolo[3,4-c]pyrrol- | | (ppm): 8.32 (s, 2H), 8.16 (d, |
| eluting | | | | 2-yl}phenyl)({2-[4-(1,3- | | J = 8.4 Hz, 2H), 8.01 (d, J = |
| isomer) | | | | oxazol-2-yl)benzenesulfonyl]- | | 8.8 Hz, 2H), 7.47 (d, J = 5.6 |
| 101 | ++++ | | | 1H,2H,3H-pyrrolo[3,4- | | Hz, 2H), 6.99-6.97 (m, 1H), |
| (2^{nd} | | | | c]pyridin-6-yl})methanol; | | 6.65-6.63 (m, 1H), 6.57-6.55 |
| eluting) | | | | (R)-(3-{5-methyl- | | (m, 1H), 6.43-6.42 (m, 1H), |
| isomer) | | | | octahydropyrrolo[3,4-c]pyrrol- | | 5.95 (d, J = 4.0 Hz, 1H), 5.57 |
| | | | | 2-yl}phenyl)({2-[4-(1,3- | | (d, J = 4.0 Hz, 1H), 4.65- |
| | | | | oxazol-2-yl)benzenesulfonyl]- | | 4.62 (m, 4H), 3.29-3.26 (m, |
| | | | | 1H,2H,3H-pyrrolo[3,4- | | 2H), 3.01-2.99 (m, 2H), 2.84 |
| | | | | c]pyridin-6-yl})methanol | | (s, 2H), 2.55-2.52 (m, 2H), |
| | | | | | | 2.39-2.37 (m, 2H), 2.21 (s, |
| | | | | | | 3H). |
| 102 | ++++ | 24-1, 3- | F | (S)-[2-(4-methylpiperazin-1- | 532.0 | (DMSO-d6, 400 MHz) δ |
| (1^{st} | | 4, 4-4, | | yl)phenyl]({2-[4-(1,3-oxazol- | | (ppm): 8.33 (s, 1H), 8.29 (s, |
| eluting | | 25-1 | | 2-yl)benzenesulfonyl]- | | 1H), 8.16 (d, J = 8.4 Hz, |
| isomer) | | | | 1H,2H,3H-pyrrolo[3,4- | | 2H), 8.02 (d, J = 8.4 Hz, |
| 103 | ++++ | | | c]pyridin-6-yl})methanol; | | 2H), 7.52-7.46 (m, 2H), |
| 2^{nd} | | | | (R)-[2-(4-methylpiperazin-1- | | 7.18-7.08 (m, 3H), 6.97-6.93 |
| eluting | | | | yl)phenyl]({2-[4-(1,3-oxazol-2- | | (m, 1H), 6.12 (d, J = 4.8 Hz, |
| isomer) | | | | 2-yl)benzenesulfonyl]- | | 1H), 5.90 (d, J = 5.2 Hz, |
| | | | | 1H,2H,3H-pyrrolo[3,4- | | 1H), 4.72-4.61 (m, 4H), |
| | | | | c]pyridin-6-yl})methanol | | 2.87-2.85 (m, 4H), 2.37 (s, |
| | | | | | | 4H), 2.18 (s, 3H). |
| 104 | −− | | | N-[4-({6-[(S)-hydroxy[4- | 562.0 | CD3OD-d4, 400 MHz) δ |
| (1^{st} | | | | methyl-3-(4-methylpiperazin- | | (ppm): 8.31 (s, 1H), 7.83 (d, |
| eluting | | | | 1-yl)phenyl]methyl]- | | J = 8.8 Hz, 1H), 7.64 (d, J = |
| isomer) | | | | 1H,2H,3H-pyrrolo[3,4- | | 2.1 Hz, 1H), 7.49 (d, J = 8.8 |
| 105 | +++ | | | c]pyridin-2- | | Hz, 1H), 7.45 (s, 1H), 7.17 |
| (2^{nd} | | | | yl}sulfonyl)phenyl]cyclopro- | | (d, J = 8.1 Hz, 2H), 7.07 (d, |
| eluting | | | | panecarboxamide; | | J = 7.9 Hz, 2H), 5.99 (d, J = |
| isomer) | | | | N-[4-({6-[(R)-hydroxy[4- | | 4.2 Hz, 1H), 5.63 (d, J = 4.0 |
| | | | | methyl-3-(4-methylpiperazin- | | Hz, 1H), 4.80 (d, J = 15.5 |
| | | | | 1-yl)phenyl]methyl]- | | Hz, 1H), 4.77-4.66 (m, |
| | | | | 1H,2H,3H-pyrrolo[3,4- | | 2H), 4.62 (d, J = 14.3 Hz, |
| | | | | c]pyridin-2- | | 1H), 2.78 (s, 4H), 2.75- |
| | | | | yl}sulfonyl)phenyl]cyclopro- | | 2.69 (m, 3H), 2.26 (d, J = |
| | | | | panecarboxamide | | 14.3 Hz, 6H), 1.83-1.72 |
| | | | | | | (m, 1H), 1.24 (s, 1H), 0.88- |
| | | | | | | 0.79 (m, 4H). |
| 106 | ++++ | 29-1 | F | (S)-{4-methoxy-6-[4-(1,3- | 563.0 | (DMSO-d6, 400 MHz) δ |
| (1^{st} | | | | oxazol-2-yl)benzenesulfonyl]- | | (ppm): 8.32 (s, 1H), 8.16 (d, |
| eluting | | | | 5H,6H,7H-pyrrolo[3,4- | | J = 8.4 Hz, 2H), 8.05 (d, J = |
| isomer) | | | | d]pyrimidin-2-yl}[3-(4- | | 8.4 Hz, 2H), 7.47 (s, 1H), |
| 107 | ++++ | | | methylpiperazin-1- | | 7.07-7.04 (m, 2H), 6.80-6.78 |
| (2^{nd} | | | | yl)phenyl]methanol; | | (m, 2H), 5.77 (d, J = 6.0 Hz, |
| eluting | | | | (R)-{4-methoxy-6-[4-(1,3- | | 1H), 5.53 (d, J = 6.0 Hz, |
| isomer) | | | | oxazol-2-yl)benzenesulfonyl]- | | 1H), 4.62-4.52 (m, 4H), 3.95 |
| | | | | 5H,6H,7H-pyrrolo[3,4- | | (s, 3H), 3.08-3.06 (m, 4H), |
| | | | | d]pyrimidin-2-yl}[3-(4- | | (s, 4H), 2.21 (s, 3H). |
| | | | | methylpiperazin-1- | | |
| | | | | yl)phenyl]methanol | | |
| 108 | ++++ | | | (S)-(3-(6-methyl-2,6- | 527.2 | (DMSO-d6, 400 MHz) δ |
| (1^{st} | | | | diazaspiro[3.3]heptan-2- | | (ppm): 8.55 (s, 1H), 8.30 (s, |
| eluting | | | | yl)phenyl)(2-(naphthalen-2- | | 1H), 8.20 (d, 1H), 8.11 (d, |
| isomer) | | | | ylsulfonyl)-2,3-dihydro-1H- | | 1H), 8.03 (dd, 1H), 7.87 (dd, |
| | | | | pyrrolo[3,4-c]pyridin-6- | | 1H), 7.73-7.65 (m, 2H), 7.42 |
| | | | | yl)methanol | | (s, 1H), 6.98 (t, 1H), 6.58- |
| | | | | | | 6.56 (m, 1H), 6.42 (s, 1H), |
| | | | | | | 6.22-6.19 (m, 1H), 5.92 (d, |
| | | | | | | 1H), 5.54 (d, 1H), 4.68-4.64 |
| | | | | | | (m, 4H), 3.80-3.76 (m, 4H), |
| | | | | | | 3.31 (s, 4H), 2.25 (s, 3H). |
| 109 | ++++ | | | (R)-(3-(6-methyl-2,6- | 527.2 | (DMSO-d6, 400 MHz) δ |
| (2^{nd} | | | | diazaspiro[3.3]heptan-2- | | (ppm): 8.55 (s, 1H), 8.30 (s, |
| eluting | | | | yl)phenyl)(2-(naphthalen-2- | | 1H), 8.20 (d, 1H), 8.11 (d, |
| isomer) | | | | ylsulfonyl)-2,3-dihydro-1H- | | 1H), 8.03 (dd, 1H), 7.87 (dd, |
| | | | | pyrrolo[3,4-c]pyridin-6- | | 1H), 7.73-7.66 (m, 2H), 7.42 |
| | | | | yl)methanol | | (s, 1H), 6.99 (t, 1H), 6.58- |
| | | | | | | 6.56 (m, 1H), 6.42 (s, 1H), |
| | | | | | | 6.22-6.19 (m, 1H), 5.92 (d, |
| | | | | | | 1H), 5.54 (d, 1H), 4.68-4.64 |
| | | | | | | (m, 4H), 3.80-3.76 (m, 4H), |
| | | | | | | 3.32 (s, 4H), 2.25 (s, 3H). |
| 110 | +++ | | | (S)-(2-((6-methoxynaphthalen- | 557.3 | (DMSO-d6, 400 MHz) δ |
| | | | | 2-yl)sulfonyl)-2,3-dihydro-1H- | | (ppm): 8.45 (s, 1H), 8.30 (s, |
| | | | | pyrrolo[3,4-c]pyridin-6-yl)(3- | | 1H), 8.10 (d, 1H), 7.97 (d, |
| | | | | (6-methyl-2,6- | | 1H), 7.83-7.80 (m, 1H), |
| | | | | diazaspiro[3.3]heptan-2- | | 7.44-7.42 (m, 1H), 7.32-7.29 |
| | | | | yl)phenyl)methanol | | (m, 2H), 6.99 (t, 1H), 6.58- |
| | | | | | | 6.56 (m, 1H), 6.42 (s, 1H), |
| | | | | | | 6.22-6.19 (m, 1H), 5.92 (d, |
| | | | | | | 1H), 5.53 (d, 1H), 4.65-4.58 |
| | | | | | | (m, 4H), 3.90 (s, 3H), 3.80- |
| | | | | | | 3.76 (m, 4H), 3.32 (s, 4H), |
| | | | | | | 2.23 (s, 3H). |

TABLE 17
Biological Data of Additional Compounds

| Cmpd. No. | Structure | IC₅₀ (μM) |
|---|---|---|
| A-1 | 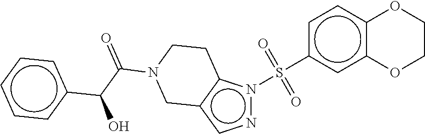 | — |
| A-2 | 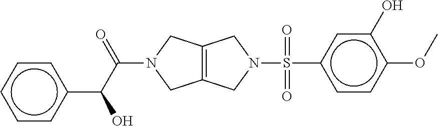 | — |
| A-3 | 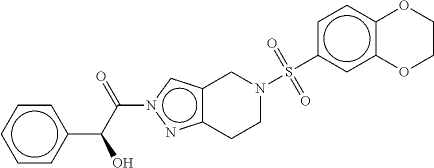 | — |
| A-4 | 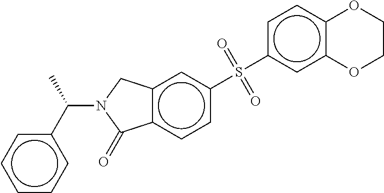 | — |
| A-5 | 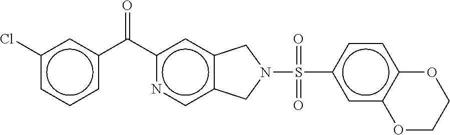 | — |
| A-6 | 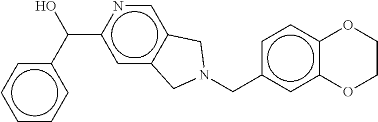 | — |
| A-7 | 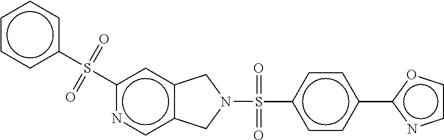 | — |

### EMBODIMENTS

Embodiment 1. A compound of Formula I:

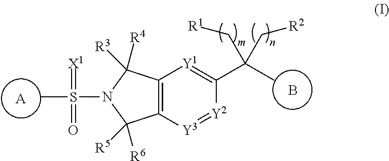

or a pharmaceutically acceptable salt thereof, wherein:

X¹ is NR or O;

Y¹ is CR⁷ or N;

Y² is CR⁸ or N;

Y³ is CR⁹ or N;

wherein the heteroaryl formed when at least one of Y¹, Y², or Y³ is N may comprise an N-oxide;

Ring A is a monocyclic or bicyclic 3- to 12-membered ring,

wherein the ring is saturated, fully or partially unsaturated, or aromatic, and

wherein the ring contains 0-4 heteroatoms independently selected from the group consisting of N, O, and S, and

wherein Ring A is optionally substituted with one or more R^{a};

each R^{a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,

wherein an optionally substituted R^{a} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;

Ring B is a monocyclic or bicyclic 3- to 12-membered ring,

wherein the ring is saturated, fully or partially unsaturated, or aromatic, and

wherein the ring contains 0-4 heteroatoms independently selected from the group consisting of N, O, and S, and

wherein Ring B is optionally substituted with one or more R^{b};

each R^{b} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,

wherein an optionally substituted R^{b} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;

R¹ and R² are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,

or R¹ and R² combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,

wherein an optionally substituted R¹ and R² group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;

R³, R⁴, R⁵, and R⁶ are each independently selected from the group consisting of —H, optionally substituted C₁-C₆aliphatic, optionally substituted C₃-C₈cycloalkyl, and optionally substituted 3- to 8-membered heterocyclyl containing 1-4 heteroatoms independently selected from the group consisting of N, O, and S

or R³ and R⁴, or R⁵ and R⁶, or a combination thereof, combine with the carbon to which they are attached to form an optionally substituted C₃-C₅cycloalkyl or an optionally substituted 3- to 8-membered heterocyclyl containing 1-4 heteroatoms independently selected from the group consisting of N, O, and S,

wherein an optionally substituted R³, R⁴, R⁵, and R⁶ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆ aliphatic;

R⁷, R⁸, and R⁹ are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, and optionally substituted C₁-C₆aliphatic,

wherein an optionally substituted R⁷, R⁸, and R⁹ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;

each R is independently selected from the group consisting of —H, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,

wherein an optionally substituted R group may be optionally substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;

each R′ is independently selected from the group consisting of optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,

wherein an optionally substituted R′ group may be substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;

m is 0, 1, or 2; and

n is 0, 1, or 2.

Embodiment 2. The compound of embodiment 1, wherein:

Ring A is:

(i) a monocyclic ring selected from C₃-C₈carbocyclyl, 3- to 8-membered heterocyclyl, phenyl, or 5- to 8-membered heteroaryl,

wherein the monocyclic ring contains 0-4 heteroatoms independently selected from the group consisting of N, O, and S, and

wherein the monocyclic ring is optionally substituted with one or more R^{a}; or

(ii) a bicyclic 6- to 12-membered ring comprising a C₃-C₁₀carbocyclyl, 3- to 10-membered heterocyclyl, phenyl, or 5- to 8-membered heteroaryl ring,

wherein the C₃-C₁₀carbocyclyl, 3- to 10-membered heterocyclyl, phenyl, or 5- to 8-membered heteroaryl ring is fused to an aromatic, saturated, or partially unsaturated 3- to 8-membered carbocyclic or heterocyclic ring,

wherein the bicyclic ring contains 0-4 heteroatoms independently selected from the group consisting of N, O, and S, and

wherein the bicyclic ring is optionally substituted with one or more R^{a}; and

Ring B is:

(i) a monocyclic ring selected from C₃-C₈carbocyclyl, 3- to 8-membered heterocyclyl, phenyl, or 5- to 8-membered heteroaryl,

wherein the monocyclic ring contains 0-4 heteroatoms independently selected from the group consisting of N, O, and S, and

wherein the monocyclic ring is optionally substituted with one or more R^{b}; or

(ii) a bicyclic 6- to 12-membered ring comprising a C₃-C₁₀carbocyclyl, 3- to 10-membered heterocyclyl, phenyl, or 5- to 8-membered heteroaryl ring,

wherein the C₃-C₁₀carbocyclyl, 3- to 10-membered heterocyclyl, phenyl, or 5- to 8-membered heteroaryl ring is fused to an aromatic, saturated, or partially unsaturated 3- to 8-membered carbocyclic or heterocyclic ring,

wherein the bicyclic ring contains 0-4 heteroatoms independently selected from the group consisting of N, O, and S, and

wherein the bicyclic ring is optionally substituted with one or more R^{b}.

Embodiment 3. The compound of any one of the preceding claims, wherein:
X¹ is O;
Y¹ is CR⁷ or N;
Y² is CR⁸ or N;
Y³ is CR⁹ or N;

wherein the heteroaryl formed when at least one of Y¹, Y², or Y³ is N may comprise an N-oxide;

Ring A is:

(i) a monocyclic ring selected from C₃-C₈carbocyclyl, phenyl, or 5- to 8-membered heteroaryl,

wherein the monocyclic ring contains 0-4 heteroatoms independently selected from the group consisting of O, N, and S, and

wherein the monocyclic ring is optionally substituted with one or more R^{a}; or

(ii) a bicyclic 9- to 12-membered ring, comprising a phenyl ring,

wherein the phenyl ring is fused to an aromatic or partially unsaturated 3- to 8-membered carbocyclic or heterocyclic ring,

wherein the bicyclic ring contains 0-4 heteroatoms independently selected from the group consisting of O, N, and S, and

wherein the bicyclic ring is optionally substituted with one or more R^{a};

each R^{a} is independently selected from the group consisting of halogen, —OR, —NRC(O)R′, optionally substituted 3- to 10-membered heterocyclyl containing 1-4 heteroatoms independently selected from N, O, and S, and optionally substituted 5- to 10-membered heteroaryl containing 1-4 heteroatoms independently selected from N, O, and S,

wherein an optionally substituted R^{a} group may be substituted with one or more halogen;

Ring B is:

(i) a monocyclic ring selected from C₃-C₈carbocyclyl or phenyl ring,

wherein the monocyclic ring is optionally substituted with one or more R^{b}; or

(ii) a bicyclic 9- to 12-membered ring, comprising a phenyl ring,

wherein the phenyl ring is fused to an aromatic or partially unsaturated 3- to 8-membered carbocyclic or heterocyclic ring,

wherein the bicyclic ring contains 0-4 heteroatoms independently selected from the group consisting of O, N, and S, and

wherein the bicyclic ring is optionally substituted with one or more R^{b};

each R^{b} is independently selected from the group consisting of halogen, —OR, optionally substituted C₁-C₆ aliphatic, and optionally substituted 3- to 10-membered heterocyclyl containing 1-4 heteroatoms independently selected from N, O, and S,

wherein an optionally substituted R^{b} group may be substituted with one or more substituents independently selected from the group consisting of —NR₂ and C₁-C₆ aliphatic;

R¹ and R² are each independently selected from the group consisting of —H, —OR, —NR₂, —CN, —C(O)NR₂, and C₁-C₆aliphatic;

R³, R⁴, R⁵, and R⁶ are each independently selected from the group consisting of —H and C₁-C₆aliphatic;

R⁷, R⁸, and R⁹ are each independently selected from the group consisting of —H, —OR, and C₁-C₆aliphatic;

each R is independently selected from the group consisting of —H, optionally substituted C₁-C₆aliphatic, and optionally substituted 3- to 10-membered heterocyclyl containing 1-4 heteroatoms independently selected from N, O, and S,

wherein an optionally substituted R group may be optionally substituted with one or more C₁-C₆aliphatic;

each R′ is independently C₃-C₁₀cycloalkyl;

m is 0, 1, or 2; and

n is 0.

Embodiment 4. The compound of any one of the preceding embodiments, wherein X¹ is O.

Embodiment 5. The compound of any one of the preceding embodiments, wherein the compound is of Formula II:

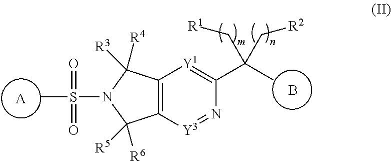

or a pharmaceutically acceptable salt thereof.

Embodiment 6. The compound of any one of the preceding embodiments, wherein the compound is of Formula III:

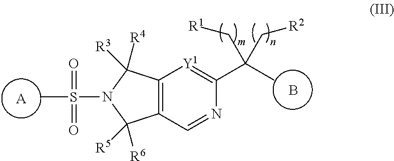

or a pharmaceutically acceptable salt thereof.

Embodiment 7. The compound of any one of the preceding embodiments, wherein R¹ and R² are each independently selected from the group consisting of —H, —OR, —NR₂, —CN, —C(O)NR₂, and C₁-C₆aliphatic.

Embodiment 8. The compound of any one of the preceding embodiments, wherein R¹ and R² are each independently selected from the group consisting of —H and —OR.

Embodiment 9. The compound of any one of the preceding embodiments, wherein R² is —H.

Embodiment 10. The compound of any one of the preceding embodiments, wherein R¹ is selected from the group consisting of —OR, —NR₂, —CN, —C(O)NR₂, and C₁-C₆aliphatic.

Embodiment 11. The compound of any one of the preceding embodiments, wherein R¹ is —OH.

Embodiment 12. The compound of any one of the preceding embodiments, wherein m is 0 or 1.

Embodiment 13. The compound of any one of the preceding embodiments, wherein m is 0.

Embodiment 14. The compound of any one of the preceding embodiments, wherein n is 0 or 1.

Embodiment 15. The compound of any one of the preceding embodiments, wherein n is 0.

Embodiment 16. The compound of any one of the preceding embodiments, wherein R³, R⁴, R⁵, and R⁶ are each independently selected from the group consisting of —H and C₁-C₆aliphatic.

Embodiment 17. The compound of any one of the preceding embodiments, wherein R³, R⁴, R⁵, and R⁶ are each independently selected from the group consisting of —H and methyl.

Embodiment 18. The compound of any one of the preceding embodiments, wherein R³ is methyl, and R⁴, R⁵, and R⁶ are each —H.

Embodiment 19. The compound of any one of the preceding embodiments, wherein R⁵ is methyl, and R³, R⁴, and R⁶ are each —H.

Embodiment 20. The compound of any one of the preceding embodiments, wherein R³, R⁴, R⁵, and R⁶ are each —H.

Embodiment 21. The compound of any one of the preceding embodiments, wherein the compound is of Formula IV:

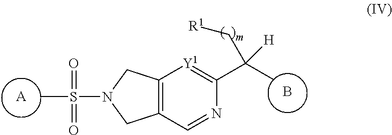

or a pharmaceutically acceptable salt thereof.

Embodiment 22. The compound of any one of the preceding embodiments, wherein the compound is of Formula IV-a:

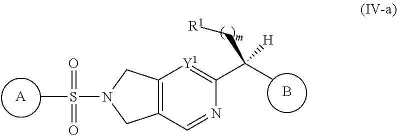

or a pharmaceutically acceptable salt thereof.

Embodiment 23. The compound of any one of the preceding embodiments, wherein the compound is of Formula IV-b:

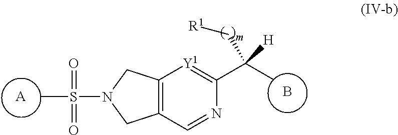

or a pharmaceutically acceptable salt thereof.

Embodiment 24. The compound of any one of the preceding embodiments, wherein the compound is of Formula V:

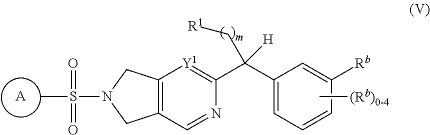

or a pharmaceutically acceptable salt thereof.

Embodiment 25. The compound of any one of the preceding embodiments, wherein the compound is of Formula VI:

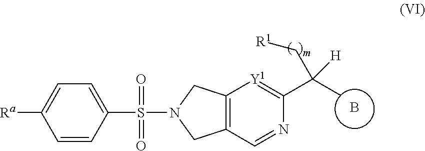

or a pharmaceutically acceptable salt thereof.

Embodiment 26. The compound of any one of the preceding embodiments, wherein Ring A is:

(i) a monocyclic ring selected from C₃-C₈carbocyclyl, phenyl, or 5- to 8-membered heteroaryl,

wherein the monocyclic ring contains 0-4 heteroatoms independently selected from the group consisting of O, N, and S, and

wherein the monocyclic ring is optionally substituted with one or more R^{a}; or

(ii) a bicyclic 9- to 12-membered ring, comprising a phenyl ring,

wherein the phenyl ring is fused to an aromatic or partially unsaturated 3- to 8-membered carbocyclic or heterocyclic ring,

wherein the bicyclic ring contains 0-4 heteroatoms independently selected from the group consisting of O, N, and S, and

wherein the bicyclic ring is optionally substituted with one or more R^{a}.

Embodiment 27. The compound of any one of the preceding embodiments, wherein Ring A is:

(i) a phenyl ring,

(ii) 5- to 6-membered monocyclic heteroaryl ring containing 1-4 heteroatoms independently selected from the group consisting of N, O, and S, or

(iii) a 9- to 10-membered bicyclic ring comprising a phenyl ring, wherein the phenyl ring is fused to an aromatic or partially unsaturated 5- to 6-membered carbocyclic ring or 5- to 6-membered heterocyclic ring containing 1-4 heteroatoms independently selected from the group consisting of N, O, and S,

wherein Ring A is optionally substituted with one or more R^{a}.

Embodiment 28. The compound of any one of the preceding embodiments, wherein Ring A is phenyl optionally substituted with one or more R^{a}.

Embodiment 29. The compound of any one of the preceding embodiments, wherein Ring A is:

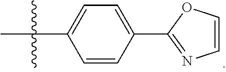 [SMILES: *c1ccc(-c2ncco2)cc1]

Embodiment 30. The compound of any one of the preceding embodiments, wherein Ring B is:

(i) a monocyclic ring selected from C₃-C₈carbocyclyl or phenyl ring,

wherein the monocyclic ring is optionally substituted with one or more R^{b}; or

(ii) a bicyclic 9- to 12-membered ring, comprising a phenyl ring,

wherein the phenyl ring is fused to an aromatic or partially unsaturated 3- to 8-membered carbocyclic or heterocyclic ring,

wherein the bicyclic ring contains 0-4 heteroatoms independently selected from the group consisting of O, N, and S, and

wherein the bicyclic ring is optionally substituted with one or more R^{b}.

Embodiment 31. The compound of any one of the preceding embodiments, wherein Ring B is:

(i) phenyl ring, or

(ii) a 9- to 10-membered bicyclic ring comprising a phenyl ring, wherein the phenyl ring is fused to an aromatic or partially unsaturated 5- to 7-membered carbocyclic ring or 5- to 7-membered heterocyclic ring containing 1-4 heteroatoms independently selected from the group consisting of N, O, and S,

wherein Ring B is optionally substituted with one or more R^{b}.

Embodiment 32. The compound of any one of the preceding embodiments, wherein Ring B is a phenyl ring optionally substituted with one or more R^{b}.

Embodiment 33. The compound of any one of the preceding embodiments, wherein the compound is of Formula VII:

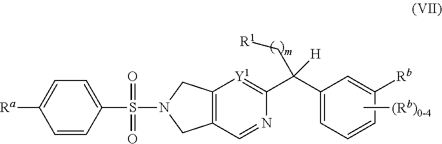

or a pharmaceutically acceptable salt thereof.

Embodiment 34. The compound of any one of the preceding embodiments, wherein R¹ is selected from the group consisting of —OR, —NR₂, —CN, —C(O)NR₂, and C₁-C₆aliphatic.

Embodiment 35. The compound of any one of the preceding embodiments, wherein R¹ is —OH.

Embodiment 36. The compound of any one of the preceding embodiments, wherein m is 0 or 1.

Embodiment 37. The compound of any one of the preceding embodiments, wherein m is 0.

Embodiment 38. The compound of any one of the preceding embodiments, wherein the compound is of Formula VIII:

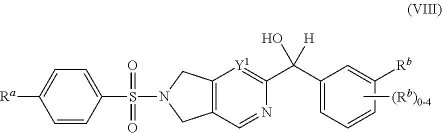

or a pharmaceutically acceptable salt thereof.

Embodiment 39. The compound of any one of the preceding embodiments, wherein the compound is of Formula VIII-a:

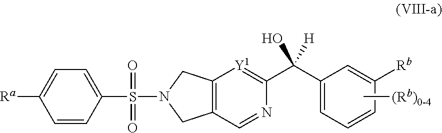

or a pharmaceutically acceptable salt thereof.

Embodiment 40. The compound of any one of the preceding embodiments, wherein the compound is of Formula VIII-b:

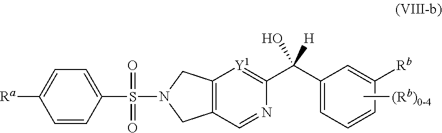

or a pharmaceutically acceptable salt thereof.

Embodiment 41. The compound of any one of the preceding embodiments, wherein R⁷, R⁸, and R⁹, if present, are each —H, halogen, —OR, —NR₂, —CN, and C₁-C₆aliphatic optionally substituted with halogen.

Embodiment 42. The compound of any one of the preceding embodiments, wherein R⁷, R⁸, and R⁹, if present, are each independently selected from the group consisting of —H, —OR, and C₁-C₆aliphatic.

Embodiment 43. The compound of any one of the preceding embodiments, wherein R⁷, R⁸, and R⁹, if present, are each —H.

Embodiment 44. The compound of any one of the preceding embodiments, wherein Y¹, Y², and Y³ are each CH or N.

Embodiment 45. The compound of any one of the preceding embodiments, wherein Y¹ is CH or N.

Embodiment 46. The compound of any one of the preceding embodiments, wherein Y¹ is CH.

Embodiment 47. The compound of any one of the preceding embodiments, wherein Y¹ is N.

Embodiment 48. The compound of any one of the preceding embodiments, wherein each R^{a} is independently halogen, —OR, —NRC(O)R′, optionally substituted 3- to 10-membered heterocyclyl containing 1-4 heteroatoms independently selected from N, O, and S, or optionally substituted 5- to 10-membered heteroaryl containing 1-4 heteroatoms independently selected from N, O, and S, wherein an optionally substituted R^{a} group may be substituted with one or more halogen.

Embodiment 49. The compound of any one of the preceding embodiments, wherein each R^{a} is independently halogen or optionally substituted 5-membered heteroaryl containing 1-4 heteroatoms independently selected from N, O, and S, wherein an optionally substituted R^{a} group may be substituted with one or more halogen.

Embodiment 50. The compound of any one of the preceding embodiments, wherein R^{a} is

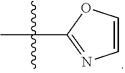 [SMILES: *c1ncco1]

Embodiment 51. The compound of any one of the preceding embodiments, wherein each R^{b} is independently selected from the group consisting of —OR, optionally substituted C₁-C₆ aliphatic, and optionally substituted 3- to 10-membered heterocyclyl containing 1-4 heteroatoms independently selected from N, O, and S, wherein an optionally substituted R^{b} group may be substituted with one or more substituents independently selected from the group consisting of —NR₂ and C₁-C₆ aliphatic.

Embodiment 52. The compound of any one of the preceding embodiments, wherein each R^{b} is independently selected from the group consisting of methyl,

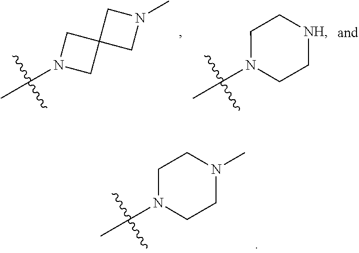

Embodiment 53. The compound of any one of the preceding embodiments, wherein each R is independently selected from the group consisting of —H, optionally substituted C₁-C₆aliphatic, and optionally substituted 3- to 10-membered heterocyclyl containing 1-4 heteroatoms independently selected from N, O, and S, wherein an optionally substituted R group may be optionally substituted with one or more C₁-C₆aliphatic.

Embodiment 54. The compound of any one of the preceding embodiments, wherein each R is —H.

Embodiment 55. The compound of any one of the preceding embodiments, wherein each R′ is independently C₁-C₆aliphatic or C₃-C₁₀cycloalkyl.

Embodiment 56. A compound selected from Table 1.

Embodiment 57. A compound of any one of the preceding embodiments, wherein the compound is a USP9X Inhibitor having an IC₅₀ value ≤0.1 μM in the assay of Example 3.

Embodiment 58. A compound of any one of the preceding embodiments, wherein the compound is a USP9X Inhibitor having an IC₅₀ value ≤1 μM in the assay of Example 3.

Embodiment 59. A pharmaceutical composition, comprising a compound of any one of the preceding embodiments, or a pharmaceutically acceptable salt thereof, and a pharmaceutically acceptable excipient.

Embodiment 60. A compound of the formula

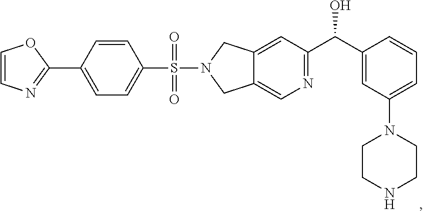 [SMILES: O=S(=O)(c1ccc(-c2ncco2)cc1)N1Cc2cnc([C@H](O)c3cccc(N4CCNCC4)c3)cc2C1]

or a pharmaceutically-acceptable salt thereof.

Embodiment 61. A compound of the formula

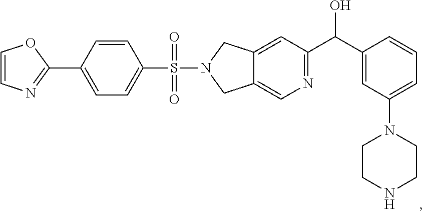 [SMILES: O=S(=O)(c1ccc(-c2ncco2)cc1)N1Cc2cnc(C(O)c3cccc(N4CCNCC4)c3)cc2C1]

or a pharmaceutically acceptable salt thereof, wherein the compound has the absolute stereochemistry of the first eluting isomer when a racemic mixture of enantiomers is separated by preparative supercritical fluid chromatography as described in Method D-2.

Embodiment 62. A compound of the formula

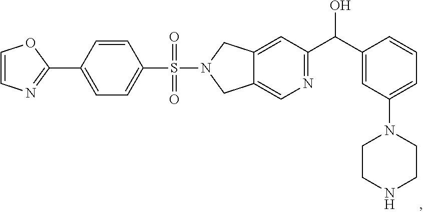 [SMILES: O=S(=O)(c1ccc(-c2ncco2)cc1)N1Cc2cnc(C(O)c3cccc(N4CCNCC4)c3)cc2C1]

or a pharmaceutically acceptable salt thereof, wherein the compound has the absolute stereochemistry of the second eluting isomer when a racemic mixture of enantiomers is separated by preparative supercritical fluid chromatography as described in Method D-2.

Embodiment 63. A compound of the formula

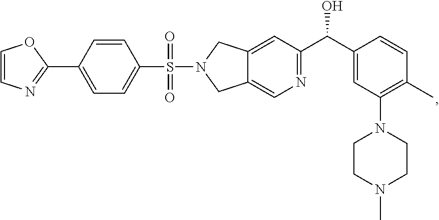 [SMILES: Cc1ccc([C@@H](O)c2cc3c(cn2)CN(S(=O)(=O)c2ccc(-c4ncco4)cc2)C3)cc1N1CCN(C)CC1]

or a pharmaceutically-acceptable salt thereof.

Embodiment 64. A compound of the formula

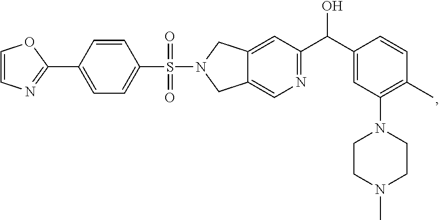 [SMILES: Cc1ccc(C(O)c2cc3c(cn2)CN(S(=O)(=O)c2ccc(-c4ncco4)cc2)C3)cc1N1CCN(C)CC1]

or a pharmaceutically acceptable salt thereof, wherein the compound has the absolute stereochemistry of the first eluting isomer when a racemic mixture of enantiomers is separated by preparative supercritical fluid chromatography as described in Method M.

Embodiment 65. A compound of the formula

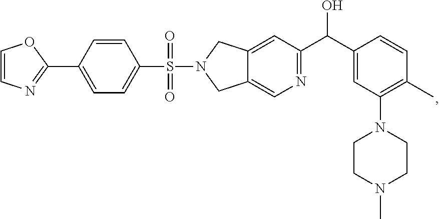 [SMILES: Cc1ccc(C(O)c2cc3c(cn2)CN(S(=O)(=O)c2ccc(-c4ncco4)cc2)C3)cc1N1CCN(C)CC1]

or a pharmaceutically acceptable salt thereof, wherein the compound has the absolute stereochemistry of the second eluting isomer when a racemic mixture of enantiomers is separated by preparative supercritical fluid chromatography as described in Method M

Embodiment 66. A compound of the formula

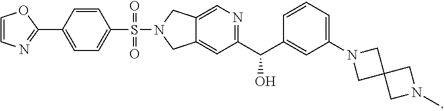 [SMILES: CN1CC2(C1)CN(c1cccc([C@H](O)c3cc4c(cn3)CN(S(=O)(=O)c3ccc(-c5ncco5)cc3)C4)c1)C2]

or a pharmaceutically-acceptable salt thereof.

Embodiment 67. A compound of the formula

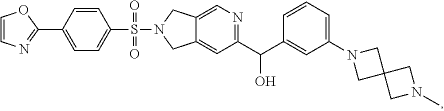 [SMILES: CN1CC2(C1)CN(c1cccc(C(O)c3cc4c(cn3)CN(S(=O)(=O)c3ccc(-c5ncco5)cc3)C4)c1)C2]

or a pharmaceutically acceptable salt thereof, wherein the compound has the absolute stereochemistry of the first eluting isomer when a racemic mixture of enantiomers is separated by preparative supercritical fluid chromatography as described in Method N.

Embodiment 68. A compound of the formula

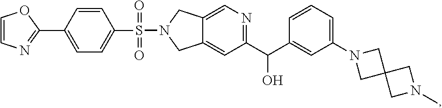 [SMILES: CN1CC2(C1)CN(c1cccc(C(O)c3cc4c(cn3)CN(S(=O)(=O)c3ccc(-c5ncco5)cc3)C4)c1)C2]

or a pharmaceutically acceptable salt thereof, wherein the compound has the absolute stereochemistry of the second eluting isomer when a racemic mixture of enantiomers is separated by preparative supercritical fluid chromatography as described in Method N.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A compound of Formula I: 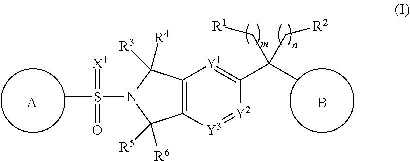
or a pharmaceutically acceptable salt thereof, wherein:
X¹ is NR or O;
Y¹ is CR⁷ or N;
Y² is CR⁸ or N;
Y³ is CR⁹ or N;
wherein the heteroaryl formed when at least one of Y¹, Y², or Y³ is N may comprise an N-oxide;
Ring A is a monocyclic or bicyclic 3- to 12-membered ring,
wherein the ring is saturated, fully or partially unsaturated, or aromatic, and
wherein the ring contains 0-4 heteroatoms independently selected from the group consisting of N, O, and S, and
wherein Ring A is optionally substituted with one or more R^{a};
each R^{a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
wherein an optionally substituted R^{a} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
Ring B is a monocyclic or bicyclic 3- to 12-membered ring,
wherein the ring is saturated, fully or partially unsaturated, or aromatic, and
wherein the ring contains 0-4 heteroatoms independently selected from the group consisting of N, O, and S, and
wherein Ring B is optionally substituted with one or more R^{b};
each R^{b} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
wherein an optionally substituted R^{b} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
R¹ and R² are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
or R¹ and R² combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
wherein an optionally substituted R¹ and R² group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
R³, R⁴, R⁵, and R⁶ are each independently selected from the group consisting of —H, optionally substituted C₁-C₆aliphatic, optionally substituted C₃-C₈cycloalkyl, and optionally substituted 3- to 8-membered heterocyclyl containing 1-4 heteroatoms independently selected from the group consisting of N, O, and S
or R³ and R⁴, or R⁵ and R⁶, or a combination thereof, combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
wherein an optionally substituted R³, R⁴, R⁵, and R⁶ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆ aliphatic;
R⁷, R⁸, and R⁹ are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, and optionally substituted C₁-C₆aliphatic,
wherein an optionally substituted R⁷, R⁸, and R⁹ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
each R is independently selected from the group consisting of —H, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
wherein an optionally substituted R group may be optionally substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;
each R′ is independently selected from the group consisting of optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
wherein an optionally substituted R′ group may be substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;
m is 0, 1, or 2; and
n is 0, 1, or 2.

2. The compound of claim 1, wherein:
Ring A is:
(i) a monocyclic ring selected from C₃-C₈carbocyclyl, 3- to 8-membered heterocyclyl, phenyl, or 5- to 8-membered heteroaryl,
wherein the monocyclic ring contains 0-4 heteroatoms independently selected from the group consisting of N, O, and S, and
wherein the monocyclic ring is optionally substituted with one or more R^{a}; or
(ii) a bicyclic 6- to 12-membered ring comprising a C₃-C₁₀carbocyclyl, 3- to 10-membered heterocyclyl, phenyl, or 5- to 8-membered heteroaryl ring,
wherein the C₃-C₁₀carbocyclyl, 3- to 10-membered heterocyclyl, phenyl, or 5- to 8-membered heteroaryl ring is fused to an aromatic, saturated, or partially unsaturated 3- to 8-membered carbocyclic or heterocyclic ring,
wherein the bicyclic ring contains 0-4 heteroatoms independently selected from the group consisting of N, O, and S, and
wherein the bicyclic ring is optionally substituted with one or more R^{a}; and
Ring B is:
(i) a monocyclic ring selected from C₃-C₈carbocyclyl, 3- to 8-membered heterocyclyl, phenyl, or 5- to 8-membered heteroaryl,
wherein the monocyclic ring contains 0-4 heteroatoms independently selected from the group consisting of N, O, and S, and
wherein the monocyclic ring is optionally substituted with one or more R^{b}; or
(ii) a bicyclic 6- to 12-membered ring comprising a C₃-C₁₀carbocyclyl, 3- to 10-membered heterocyclyl, phenyl, or 5- to 8-membered heteroaryl ring,
wherein the C₃-C₁₀carbocyclyl, 3- to 10-membered heterocyclyl, phenyl, or 5- to 8-membered heteroaryl ring is fused to an aromatic, saturated, or partially unsaturated 3- to 8-membered carbocyclic or heterocyclic ring,
wherein the bicyclic ring contains 0-4 heteroatoms independently selected from the group consisting of N, O, and S, and
wherein the bicyclic ring is optionally substituted with one or more R^{b}.

3. The compound of any one of the preceding claims, wherein:
X¹ is O;
Y¹ is CR⁷ or N;
Y² is CR⁸ or N;
Y³ is CR⁹ or N;
wherein the heteroaryl formed when at least one of Y¹, Y², or Y³ is N may comprise an N-oxide;
Ring A is:
(i) a monocyclic ring selected from C₃-C₈carbocyclyl, phenyl, or 5- to 8-membered heteroaryl,
wherein the monocyclic ring contains 0-4 heteroatoms independently selected from the group consisting of O, N, and S, and
wherein the monocyclic ring is optionally substituted with one or more R^{a}; or
(ii) a bicyclic 9- to 12-membered ring, comprising a phenyl ring,
wherein the phenyl ring is fused to an aromatic or partially unsaturated 3- to 8-membered carbocyclic or heterocyclic ring,
wherein the bicyclic ring contains 0-4 heteroatoms independently selected from the group consisting of O, N, and S, and
wherein the bicyclic ring is optionally substituted with one or more R^{a};
each R^{a} is independently selected from the group consisting of halogen, —OR, —NRC(O)R′, optionally substituted 3- to 10-membered heterocyclyl containing 1-4 heteroatoms independently selected from N, O, and S, and optionally substituted 5- to 10-membered heteroaryl containing 1-4 heteroatoms independently selected from N, O, and S,
wherein an optionally substituted R^{a} group may be substituted with one or more halogen;
Ring B is:
(i) a monocyclic ring selected from C₃-C₈carbocyclyl or phenyl ring,
wherein the monocyclic ring is optionally substituted with one or more R^{b}; or
(ii) a bicyclic 9- to 12-membered ring, comprising a phenyl ring,
wherein the phenyl ring is fused to an aromatic or partially unsaturated 3- to 8-membered carbocyclic or heterocyclic ring,
wherein the bicyclic ring contains 0-4 heteroatoms independently selected from the group consisting of O, N, and S, and
wherein the bicyclic ring is optionally substituted with one or more R^{b};
each R^{b} is independently selected from the group consisting of halogen, —OR, optionally substituted C₁-C₆ aliphatic, and optionally substituted 3- to 10-membered heterocyclyl containing 1-4 heteroatoms independently selected from N, O, and S,
wherein an optionally substituted R^{b} group may be substituted with one or more substituents independently selected from the group consisting of —NR₂ and C₁-C₆ aliphatic;
R¹ and R² are each independently selected from the group consisting of —H, —OR, —NR₂, —CN, —C(O)NR₂, and C₁-C₆aliphatic;
R³, R⁴, R⁵, and R⁶ are each independently selected from the group consisting of —H and C₁-C₆aliphatic;
R⁷, R⁸, and R⁹ are each independently selected from the group consisting of —H, —OR, and C₁-C₆aliphatic;
each R is independently selected from the group consisting of —H, optionally substituted C₁-C₆aliphatic, and optionally substituted 3- to 10-membered heterocyclyl containing 1-4 heteroatoms independently selected from N, O, and S,
wherein an optionally substituted R group may be optionally substituted with one or more C₁-C₆aliphatic;
each R′ is independently C₃-C₁₀cycloalkyl;
m is 0, 1, or 2; and
n is 0.

4. The compound of any one of the preceding claims, wherein the compound is of Formula IV: 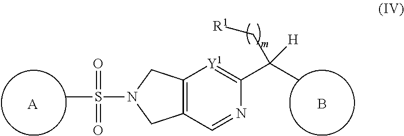
or a pharmaceutically acceptable salt thereof.

5. The compound of any one of the preceding claims, wherein the compound is of Formula V: 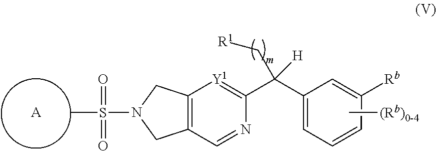
or a pharmaceutically acceptable salt thereof.

6. The compound of any one of the preceding claims, wherein the compound is of Formula VI: 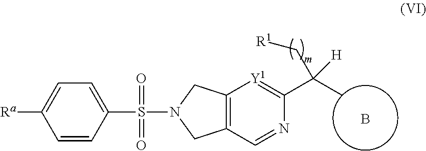
or a pharmaceutically acceptable salt thereof.

7. The compound of any one of the preceding claims, wherein Ring A is:
(i) a monocyclic ring selected from C₃-C₈carbocyclyl, phenyl, or 5- to 8-membered heteroaryl,
wherein the monocyclic ring contains 0-4 heteroatoms independently selected from the group consisting of O, N, and S, and
wherein the monocyclic ring is optionally substituted with one or more R^{a}; or
(ii) a bicyclic 9- to 12-membered ring, comprising a phenyl ring,
wherein the phenyl ring is fused to an aromatic or partially unsaturated 3- to 8-membered carbocyclic or heterocyclic ring,
wherein the bicyclic ring contains 0-4 heteroatoms independently selected from the group consisting of O, N, and S, and
wherein the bicyclic ring is optionally substituted with one or more R^{a}.

8. The compound of any one of the preceding claims, wherein Ring B is:
(i) a monocyclic ring selected from C₃-C₈carbocyclyl or phenyl ring,
wherein the monocyclic ring is optionally substituted with one or more R^{b}; or
(ii) a bicyclic 9- to 12-membered ring, comprising a phenyl ring,
wherein the phenyl ring is fused to an aromatic or partially unsaturated 3- to 8-membered carbocyclic or heterocyclic ring,
wherein the bicyclic ring contains 0-4 heteroatoms independently selected from the group consisting of O, N, and S, and
wherein the bicyclic ring is optionally substituted with one or more R^{b}.

9. The compound of any one of the preceding claims, wherein R^{a} is

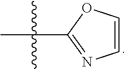 [SMILES: *c1ncco1]

10. The compound of any one of the preceding claims, wherein each R^{b} is independently selected from the group consisting of methyl,

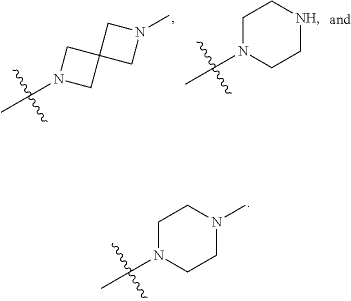

11. The compound of any one of the preceding claims, wherein the compound is: 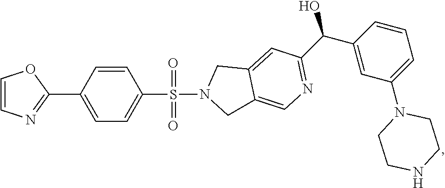 [SMILES: O=S(=O)(c1ccc(-c2ncco2)cc1)N1Cc2cnc([C@@H](O)c3cccc(N4CCNCC4)c3)cc2C1]
or a pharmaceutically acceptable salt thereof.

12. The compound of claim 11, wherein the pharmaceutically acceptable salt thereof is a hydrochloride salt.

13. The compound of any one of claims 1-10, wherein the compound is: 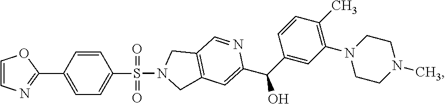 [SMILES: Cc1ccc([C@@H](O)c2cc3c(cn2)CN(S(=O)(=O)c2ccc(-c4ncco4)cc2)C3)cc1N1CCN(C)CC1]
or a pharmaceutically acceptable salt thereof.

14. The compound of any one of claims 1-10, wherein the compound is: 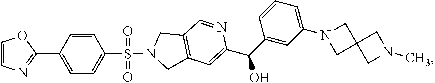 [SMILES: CN1CC2(C1)CN(c1cccc([C@@H](O)c3cc4c(cn3)CN(S(=O)(=O)c3ccc(-c5ncco5)cc3)C4)c1)C2]
or a pharmaceutically acceptable salt thereof.

15. A pharmaceutical composition, comprising a compound of any one of the preceding claims, or a pharmaceutically acceptable salt thereof, and a pharmaceutically acceptable excipient.
